(19) United States
(12) Patent Application Publication
(10) Pub. No.: US 2025/0129419 A1
(43) Pub. Date: Apr. 24, 2025

(54) REAGENTS FOR LABELING BIOMOLECULES AND USES THEREOF

(71) Applicant: Ultima Genomics, Inc., Fremont, CA (US)

(72) Inventors: Charles FRANCAVILLA, Fremont, CA (US); Linda G. LEE, Palo Alto, CA (US); Abhisek RAY, Santa Clara, CA (US); Steven MENCHEN, Fremont, CA (US); Stephanie WANG, Los Altos, CA (US); Florian OBERSTRASS, Menlo Park, CA (US); Dancan NJERI, Newark, CA (US); Maodie WANG, Fremont, CA (US); Theo NIKIFOROV, Carlsbad, CA (US)

(21) Appl. No.: 18/803,016

(22) Filed: Aug. 13, 2024

Related U.S. Application Data
(63) Continuation of application No. PCT/US2023/013634, filed on Feb. 22, 2023.
(60) Provisional application No. 63/414,398, filed on Oct. 7, 2022.
(60) Provisional application No. 63/313,191, filed on Feb. 23, 2022.

(51) Int. Cl. C12Q 1/6876; G01N 33/533

(52) U.S. Cl. CPC C12Q 1/6876; G01N 33/533

(57) ABSTRACT

The present disclosure provides labeling reagents for labeling substrates such as nucleotides, proteins, antibodies, lipids, and cells. The labeling reagents provided herein may comprise fluorescent labels and semi-rigid linkers. Methods for nucleic acid sequencing using materials comprising such labeling reagents are also provided herein.

### CROSS-REFERENCE

This application is a continuation of International Patent Application No. PCT/US2023/013634, which claims the benefit of U.S. Provisional Patent App. No. 63/313,191, filed Feb. 23, 2022 and U.S. Provisional Patent App. No. 63/414,398, filed Oct. 7, 2022, each of which is entirely incorporated herein by reference.

### BACKGROUND

The detection, quantification, and sequencing of cells and biological molecules may be important for molecular biology and medical applications, such as diagnostics. Genetic testing may be useful for a number of diagnostic methods. For example, disorders that are caused by rare genetic alterations (e.g., sequence variants) or changes in epigenetic markers, such as cancer and partial or complete aneuploidy, may be detected or more accurately characterized with deoxyribonucleic acid (DNA) or ribonucleic acid (RNA) sequence information.

Nucleic acid sequencing is a process that can be used to provide sequence information for a nucleic acid sample. Such sequence information may be helpful in diagnosing and/or treating a subject with a condition. For example, the nucleic acid sequence of a subject may be used to identify, diagnose, and potentially develop treatments for genetic diseases. As another example, research into pathogens may lead to treatment of contagious diseases.

Nucleic acid sequencing may comprise the use of fluorescently labeled moieties. Such moieties may be labeled with organic fluorescent dyes. The sensitivity of a detection scheme can be improved by using dyes with both a high extinction coefficient and quantum yield, where the product of these characteristics may be termed the dye's “brightness.” Dye brightness may be attenuated by quenching phenomena, including quenching by biological materials, quenching by proximity to other dyes, and quenching by solvent. Other routes to brightness loss include photobleaching, reactivity to molecular oxygen, and chemical decomposition.

### SUMMARY

The present disclosure provides improved optical (e.g., fluorescent) labeling reagents and methods of nucleic acid processing comprising the use of optically (e.g., fluorescently) labeled moieties. The materials and methods provided herein may comprise the use of organic fluorescent dyes. The materials provided herein may allow for optimized molecular quenching to facilitate efficient nucleic acid processing and detection. Molecular quenching mechanisms can include photoinduced electron transfer, photoinduced hole transfer, Forster energy transfer, Dexter quenching, and the like. A general solution to many types of quenching requires physical separation of the dye from the quencher moiety, but existing solutions all have advantages and disadvantages in terms of ease of use, cost, solvent-dependence and polydispersity. Accordingly, the present disclosure recognizes the need for materials and methods that address these limitations and provides materials comprising improved linker moieties. Provided herein are detectable reagents.

In an aspect, provided herein is a labeled substrate comprising: a substrate; a linker; and a plurality of dye moieties attached to the substrate via the linker, wherein the linker comprises a cleavable portion and a poly-hydroxyproline portion, wherein the poly-hydroxyproline portion comprises a first hydroxyproline portion comprising a first one or more hydroxyproline residues, a set of hydroxyproline or amino-proline residues that attach to the plurality of dye moieties, and one or more second hydroxyproline portions each comprising a second one or more hydroxyproline residues, each of the one or more second hydroxyproline portions disposed between different hydroxyproline or amino-proline residues of the set of hydroxyproline or amino-proline residues.

In some embodiments, the substrate comprises a nucleotide base. In some embodiments, the substrate comprises a protein. In some embodiments, a second hydroxyproline portion of the one or more second hydroxyproline portions comprises at least two hydroxyproline residues. In some embodiments, a second hydroxyproline portion of the one or more second hydroxyproline portions comprises at least ten hydroxyproline residues. In some embodiments, each second hydroxyproline portion of the one or more second hydroxyproline portions comprises a number of hydroxyproline residue that is not 3 or an integer multiple of 3.

In an aspect, provided herein is a labeled substrate comprising: labeling reagent, comprising: a linker; and a plurality of dye moieties attached to linker, wherein the linker comprises a cleavable portion and a poly-hydroxyproline portion, wherein the poly-hydroxyproline portion comprises a first hydroxyproline portion comprising a first one or more hydroxyproline residues, a set of hydroxyproline or amino-proline residues that attach to the plurality of dye moieties, and one or more second hydroxyproline portions each comprising a second one or more hydroxyproline residues, each of the one or more second hydroxyproline portions disposed between different hydroxyproline or amino-proline residues of the set of hydroxyproline or amino-proline residues.

In some embodiments, a second hydroxyproline portion of the one or more second hydroxyproline portions comprises at least two hydroxyproline residues. In some embodiments, wherein a second hydroxyproline portion of the one or more second hydroxyproline portions comprises at least ten hydroxyproline residues. In some embodiments, each second hydroxyproline portion of the one or more second hydroxyproline portions comprises a number of hydroxyproline residue that is not 3 or an integer multiple of 3.

In an aspect, provided herein is a method, comprising: (a) providing a primer-hybridized template nucleic acid molecule; and (b) contacting the primer-hybridized template nucleic acid molecule with a mixture of nucleotides, wherein the mixture of nucleotides comprises a first type of labeled nucleotide and a second type of labeled nucleotide, wherein the first type of labeled nucleotide comprises a first linker of a first length or which provides a first distance between a substrate and a label of the first type of labeled nucleotide and the second type of labeled nucleotide comprises a second linker of a second length or which provides a second distance between a substrate and a label of the second type of labeled nucleotide, and wherein the first length is different from the second length and the first distance is different from the second distance.

In some embodiments, the method further comprises (c) detecting one or more signals from the primer-hybridized template nucleic acid molecule. 308. In some embodiments, wherein the first type of labeled nucleotide and the second type of labeled nucleotide are of a same canonical base type.

In an aspect, provided herein is a method, comprising: (a) providing a primer-hybridized template nucleic acid molecule; and (b) contacting the primer-hybridized template nucleic acid molecule with a mixture of terminated nucleotides, wherein the mixture of terminated nucleotides comprises a first type of labeled nucleotide comprising a first number of dyes and a second type of labeled nucleotide comprising a second number of dyes, wherein the first number is different than the second number, wherein the first type of labeled nucleotide is a first canonical base and the second type of labeled nucleotide is a second canonical base different from the first canonical base, and wherein the first type of labeled nucleotide and the second type of labeled nucleotide are detectable at a same or substantially same frequency.

In some embodiments, the first type of labeled nucleotide comprises the labeled substrate described herein, wherein the substrate is a terminated nucleotide. In some embodiments, the method further comprises (c) detecting one or more signals from the primer-hybridized template nucleic acid molecule. In some embodiments, the mixture of terminated nucleotides further comprises a third type of labeled nucleotide of a third canonical base comprising a third number of dyes, wherein the third number is different from the first number and the second number, and wherein the third canonical base is different from the first canonical base and the second canonical base. In some embodiments, the mixture of terminated nucleotides further comprises a fourth type of labeled nucleotide of a fourth canonical base comprising a fourth number of dyes, wherein the fourth number is different from the first number, the second number, and the third number, and wherein the fourth canonical base is different from the first canonical base, the second canonical base, and the third canonical base. In some embodiments, the mixture of terminated nucleotides further comprises a fourth type of unlabeled nucleotide of a fourth canonical base type, wherein the fourth canonical base is different from the first canonical base, the second canonical base, and the third canonical base.

In an aspect, provided herein is a method, comprising: (a) providing a primer-hybridized template nucleic acid molecule; and (b) contacting the primer-hybridized template nucleic acid molecule with a mixture of terminated nucleotides, wherein the mixture of terminated nucleotides comprises a first type of labeled nucleotide and a second type of labeled nucleotide, wherein the first type of labeled nucleotide is a first canonical base and the second type of labeled nucleotide is a second canonical base different from the first canonical base, wherein the first type of labeled nucleotide and the second type of labeled nucleotide are detectable at different signal intensities, and wherein the first type of labeled nucleotide and the second type of labeled nucleotide are detectable at a same or substantially same frequency.

In some embodiments, the first type of labeled nucleotide comprises the labeled substrate of embodiment 296, wherein the substrate is a terminated nucleotide. In some embodiments, the method further comprises (c) detecting one or more signals form the primer-hybridized template nucleic acid molecule.

In an aspect, provided herein is a labeling reagent comprising: (a) a detectable moiety; and (b) a linker that is coupled to the detectable moiety, wherein the linker comprises at least one cleavable group and at least one non-proteinogenic amino acid, wherein the at least one non-proteinogenic amino acid does not comprise hydroxyproline, and wherein when the at least one non-proteinogenic amino acid comprises 6-aminohexanoic acid, the detectable moiety is not a Cyanide 5 (Cy5) or an ATTO 647 NHS Ester (ATTO 647N).

In an aspect, provided herein is a labeling reagent comprising a compound of Formula I:

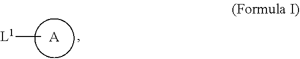

wherein: A is a detectable moiety; and L¹ is a linker comprising at least one cleavable group and at least one non-proteinogenic amino acid, wherein the non-proteinogenic amino acid does not comprise hydroxyproline, and wherein when the at least one non-proteinogenic amino acid comprises 6-aminohexanoic acid, the detectable moiety is not a Cyanide 5 (Cy5) or an ATTO 647 NHS Ester (ATTO 647N).

In an aspect, provided herein is a labeling reagent comprising: (a) a detectable moiety; and (b) a linker that is coupled to the detectable moiety, wherein the linker comprises at least one cleavable group and at least one non-proteinogenic amino acid, wherein the at least one non-proteinogenic amino acid does not comprise hydroxyproline, and wherein when the at least one non-proteinogenic amino acid comprises 6-aminohexanoic acid, the linker is not coupled to

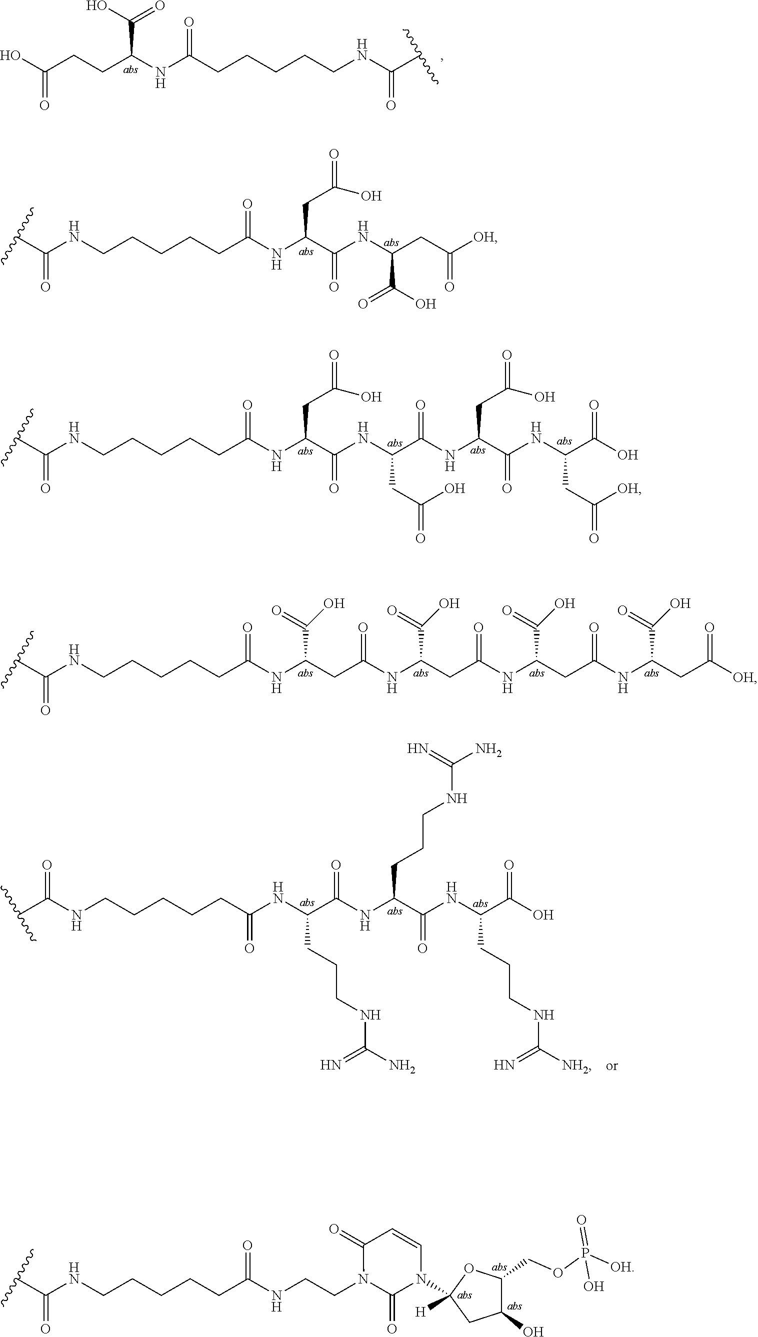 [SMILES: *C(=O)NCCCCCC(=O)N[C@@H](CCC(=O)O)C(=O)O ; *C(=O)NCCCCCC(=O)N[C@@H](CC(=O)O)C(=O)N[C@@H](CC(=O)O)C(=O)O ; *C(=O)NCCCCCC(=O)N[C@@H](CC(=O)O)C(=O)N[C@@H](CC(=O)O)C(=O)N[C@@H](CC(=O)O)C(=O)N[C@@H](CC(=O)O)C(=O)O ; *C(=O)NCCCCCC(=O)N[C@@H](CC(=O)N[C@@H](CC(=O)N[C@@H](CC(=O)N[C@@H](CC(=O)O)C(=O)O)C(=O)O)C(=O)O)C(=O)O ; *C(=O)NCCCCCC(=O)N[C@@H](CCCNC(=N)N)C(=O)N[C@@H](CCCNC(=N)N)C(=O)N[C@@H](CCCNC(=N)N)C(=O)O ; *C(=O)NCCCCCC(=O)NCCn1c(=O)ccn([C@H]2C[C@H](O)[C@@H](COP(=O)(O)O)O2)c1=O]

In an aspect, provided herein is a labeling reagent comprising a compound of Formula I:

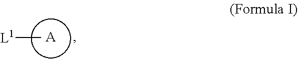

wherein: A is a detectable moiety; and L¹ is a linker comprising at least one cleavable group and at least one non-proteinogenic amino acid, wherein the at least one non-proteinogenic amino acid does not comprise hydroxyproline, and wherein when the at least one non-proteinogenic amino acid comprises 6-aminohexanoic acid, the linker is not coupled to

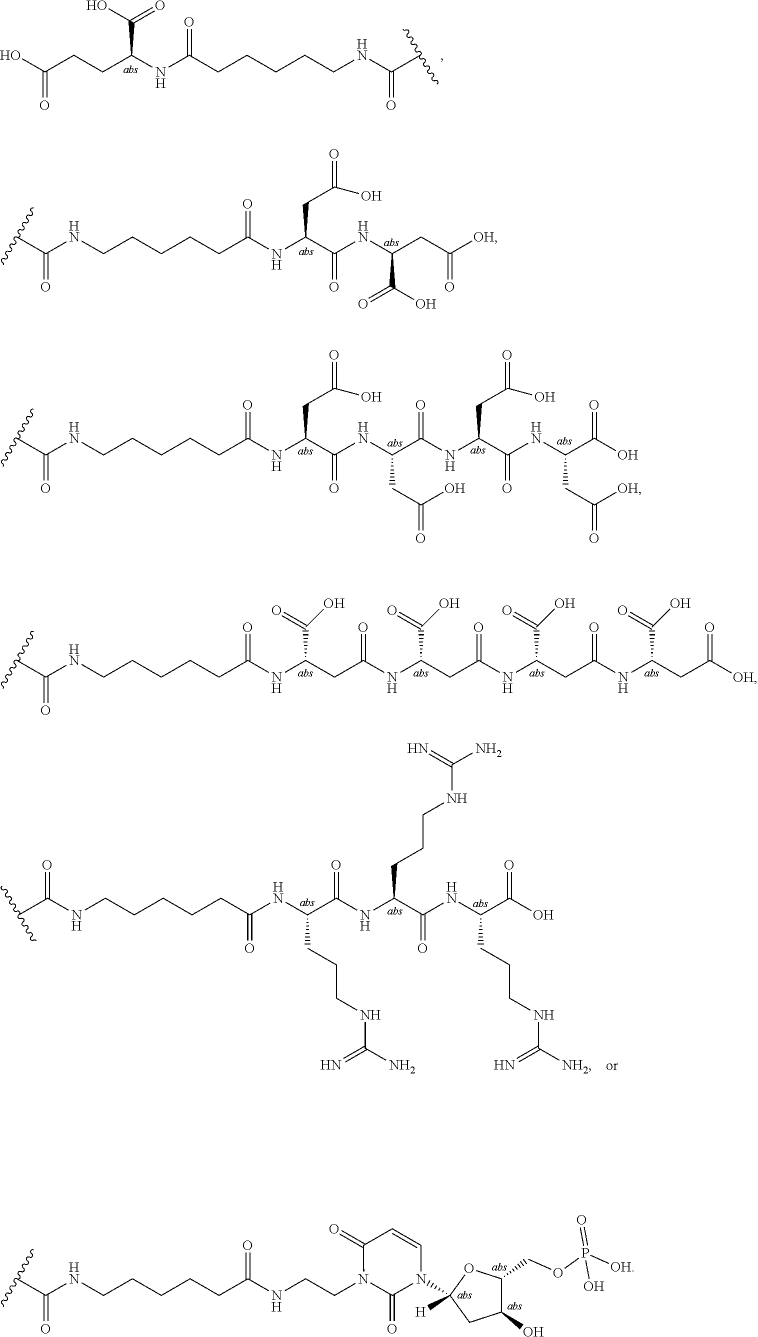 [SMILES: *C(=O)NCCCCCC(=O)N[C@@H](CCC(=O)O)C(=O)O ; *C(=O)NCCCCCC(=O)N[C@@H](CC(=O)O)C(=O)N[C@@H](CC(=O)O)C(=O)O ; *C(=O)NCCCCCC(=O)N[C@@H](CC(=O)O)C(=O)N[C@@H](CC(=O)O)C(=O)N[C@@H](CC(=O)O)C(=O)N[C@@H](CC(=O)O)C(=O)O ; *C(=O)NCCCCCC(=O)N[C@@H](CC(=O)N[C@@H](CC(=O)N[C@@H](CC(=O)N[C@@H](CC(=O)O)C(=O)O)C(=O)O)C(=O)O)C(=O)O ; *C(=O)NCCCCCC(=O)N[C@@H](CCCNC(=N)N)C(=O)N[C@@H](CCCNC(=N)N)C(=O)N[C@@H](CCCNC(=N)N)C(=O)O ; *C(=O)NCCCCCC(=O)NCCn1c(=O)ccn([C@H]2C[C@H](O)[C@@H](COP(=O)(O)O)O2)c1=O]

In an aspect, provided herein is a labeling reagent comprising: (a) a detectable moiety; and (b) a linker that is coupled to the detectable moiety, wherein the linker comprises at least one cleavable group and at least one non-proteinogenic amino acid, wherein the at least one non-proteinogenic amino acid comprises cysteic acid, 5-amino-5-carboxy-N,N,N-trimethylpentan-1-aminium or a salt thereof, or 6-aminohexanoic acid, or a combination thereof, wherein when the at least one non-proteinogenic amino acid comprises the cysteic acid, the linker does not comprise hydroxyproline, and wherein when the at least one non-proteinogenic amino acid comprises the 6-aminohexanoic acid, the detectable moiety is not a Cyanide 5 (Cy5) or an ATTO 647 NHS Ester (ATTO 647N).

In an aspect, provided herein is a labeling reagent comprising a compound of Formula I:

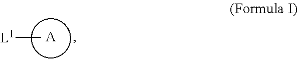

wherein: A is a detectable moiety; and L¹ is a linker comprising at least one cleavable group and at least one non-proteinogenic amino acid, wherein the at least one non-proteinogenic amino acid non-proteinogenic amino acid comprises cysteic acid, 5-amino-5-carboxy-N,N,N-trimethylpentan-1-aminium or a salt thereof, or 6-aminohexanoic acid, or a combination thereof, wherein when the at least one non-proteinogenic amino acid comprises the cysteic acid, L¹ does not comprise hydroxyproline, and wherein when the at least one non-proteinogenic amino acid comprises the 6-aminohexanoic acid, the detectable moiety is not a Cyanide 5 (Cy5) or an ATTO 647 NHS Ester (ATTO 647N).

In an aspect, provided herein is a labeling reagent comprising: (a) a detectable moiety; and (b) a linker that is coupled to the detectable moiety, wherein the linker comprises at least one cleavable group and at least one non-proteinogenic amino acid, wherein the at least one non-proteinogenic amino acid comprises cysteic acid, 5-amino-5-carboxy-N,N,N-trimethylpentan-1-aminium or a salt thereof, or 6-aminohexanoic acid, or a combination thereof, wherein when the at least one non-proteinogenic amino acid comprises the cysteic acid, the linker does not comprise hydroxyproline, and wherein when the at least one non-proteinogenic amino acid comprises the 6-aminohexanoic acid, the linker is not coupled to

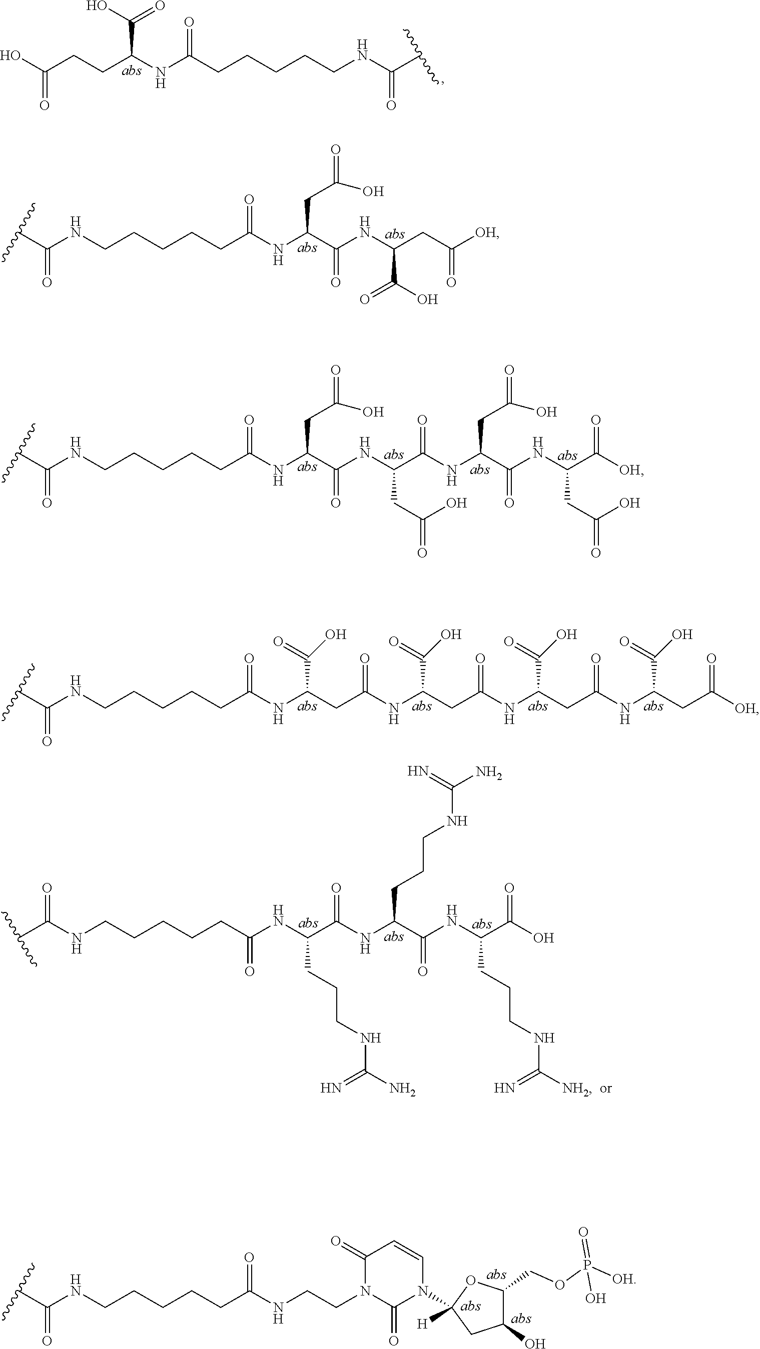

In an aspect, provided herein is a labeling reagent comprising a compound of Formula I:

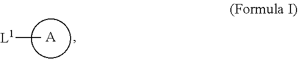

wherein: A is a detectable moiety; and L¹ is a linker comprising at least one cleavable group and at least one non-proteinogenic amino acid, wherein the at least one non-proteinogenic amino acid comprises cysteic acid, 5-amino-5-carboxy-N,N,N-trimethylpentan-1-aminium, or 6-aminohexanoic acid, or a combination thereof, wherein when the at least one non-proteinogenic amino acid comprises the cysteic acid, L¹ does not comprise hydroxyproline, and wherein when the at least one non-proteinogenic acid comprises the 6-aminohexanoic acid, L¹ is not coupled to

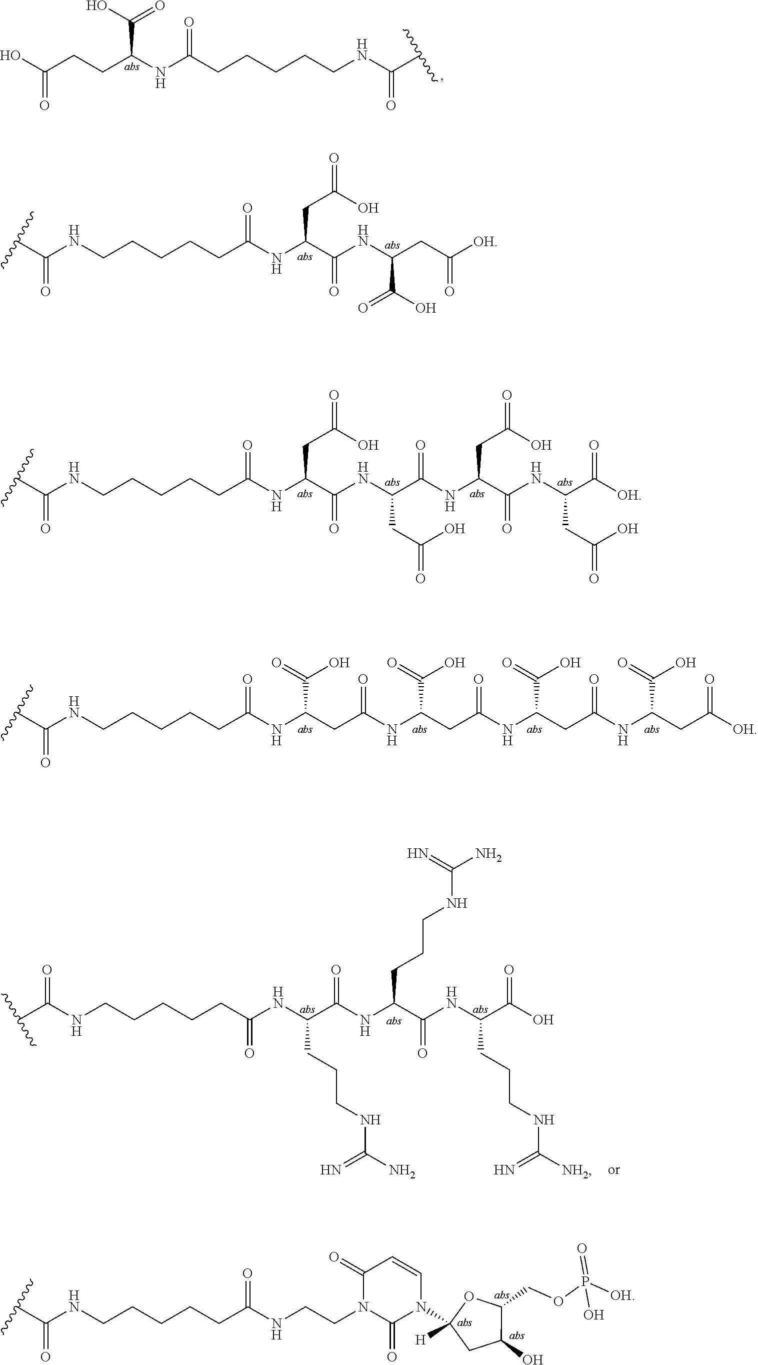 [SMILES: *C(=O)NCCCCCC(=O)N[C@@H](CCC(=O)O)C(=O)O ; *C(=O)NCCCCCC(=O)N[C@@H](CC(=O)O)C(=O)N[C@@H](CC(=O)O)C(=O)O ; *C(=O)NCCCCCC(=O)N[C@@H](CC(=O)O)C(=O)N[C@@H](CC(=O)O)C(=O)N[C@@H](CC(=O)O)C(=O)N[C@@H](CC(=O)O)C(=O)O ; *C(=O)NCCCCCC(=O)N[C@@H](CC(=O)N[C@@H](CC(=O)N[C@@H](CC(=O)N[C@@H](CC(=O)O)C(=O)O)C(=O)O)C(=O)O)C(=O)O ; *C(=O)NCCCCCC(=O)N[C@@H](CCCNC(=N)N)C(=O)N[C@@H](CCCNC(=N)N)C(=O)N[C@@H](CCCNC(=N)N)C(=O)O ; *C(=O)NCCCCCC(=O)NCCn1c(=O)ccn([C@H]2C[C@H](O)[C@@H](COP(=O)(O)O)O2)c1=O]

In some embodiment, the linker is not coupled to a terminator group. In some embodiment, the detectable moiety does not comprise the Cy5 or the ATTO 647N. 328. In some embodiment, the at least one non-proteinogenic amino acid comprises at most about 50 atoms. In some embodiment, the at least one non-proteinogenic amino acid comprises at most about 20 atoms In some embodiment, the at least one non-proteinogenic amino acid comprises about 10-20 atoms. In some embodiment, the at least one non-proteinogenic amino acid comprises cysteic acid. In some embodiment, the at least one non-proteinogenic amino acid comprises 5-amino-5-carboxy-N,N,N-trimethylpentan-1-aminium or a salt thereof. In some embodiment, the at least one non-proteinogenic amino acid comprises 6-aminohexanoic acid. In some embodiment, the at least one non-proteinogenic amino acid comprises a quaternary amine. In some embodiment, the detectable moiety comprises a fluorescent dye In some embodiment, the fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, or Kam. In some embodiment, the fluorescent dye comprises ATTO 633. In some embodiment, the at least one cleavable group is configured to be cleaved to separate a portion of the detectable moiety from the labeling reagent. In some embodiment, the at least one cleavable group is selected from the group consisting of an azidomethyl group, a disulfide bond, a hydrocarbyldithiomethyl group, and a 2-nitrobenzyloxy group. In some embodiment, the at least one cleavable group is the disulfide bond. In some embodiment, the at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof. In some embodiment, the labeling reagent comprises a moiety selected from the group consisting of

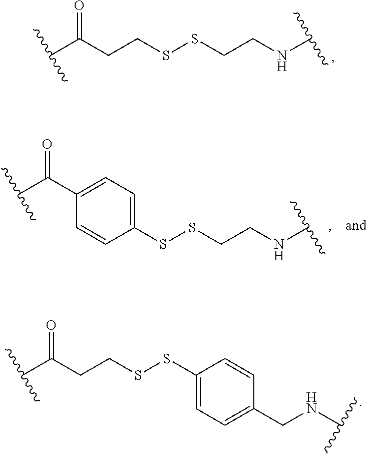 [SMILES: *NCCSSCCC(*)=O ; *NCCSSc1ccc(C(*)=O)cc1 ; *NCc1ccc(SSCCC(*)=O)cc1]

In an aspect, provided herein is a detectably labeled substrate comprising a compound described herein, wherein the compound is a compound of Formula Ia:

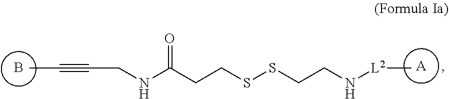

wherein: B is a substrate, A is the detectable moiety, and L² comprises the at least one non-proteinogenic amino acid.

In an aspect, provided herein is a detectably labeled substrate comprising: (a) a detectable moiety; (b) a linker that is coupled to the detectable moiety, wherein the linker comprises at least one non-proteinogenic amino acid, wherein the at least one non-proteinogenic amino acid comprises cysteic acid, 5-amino-5-carboxy-N,N,N-trimethylpentan-1-aminium or a salt thereof, or 6-aminohexanoic acid, or a combination thereof, and wherein when the at least one non-proteinogenic amino acid comprises the 6-aminohexanoic acid, the detectable moiety is not a Cyanide 5 (Cy5) or an ATTO 647 NHS Ester (ATTO 647N); and (c) a substrate comprising a nucleobase, wherein the substrate is coupled to the linker, and wherein the nucleobase does not comprise guanine.

In an aspect, provided herein is a detectably labeled substrate comprising a compound of Formula II:

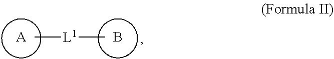

wherein: A comprises a nucleobase, wherein the nucleobase is not guanine; B is a detectable moiety; and L¹ is a linker comprising at least one non-proteinogenic amino acid, wherein the at least one non-proteinogenic amino acid comprises cysteic acid, 5-amino-5-carboxy-N,N,N-trimethylpentan-1-aminium or a salt thereof, or 6-aminohexanoic acid, or a combination thereof, and wherein when the at least one non-proteinogenic amino acid comprises the 6-aminohexanoic acid, the detectable moiety is not a Cyanide 5 (Cy5) or an ATTO 647 NHS Ester (ATTO 647N).

In some embodiments, the nucleobase is adenine, cytosine, thymine, or uracil. In some embodiments, the detectable moiety does not comprise the Cy5 or the ATTO 647N. In some embodiments, the linker comprises at least one cleavable group. In some embodiments, the at least one non-proteinogenic amino acid comprises the cysteic acid. In some embodiments, the at least one non-proteinogenic amino acid comprises the 5-amino-5-carboxy-N,N,N-trimethylpentan-1-aminium. In some embodiments, the at least one non-proteinogenic amino acid comprises the 6-aminohexanoic acid. In some embodiments, the at least one non-proteinogenic amino acid comprises a quaternary amine. In some embodiments, the detectable moiety comprises a fluorescent dye. In some embodiments, the fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, or Kam. In some embodiments, the fluorescent dye comprises ATTO 633. In some embodiments, the at least one cleavable group is configured to be cleaved to separate a portion of the detectable moiety from the detectably labeled substrate. In some embodiments, the at least one cleavable group is selected from the group consisting of an azidomethyl group, a disulfide bond, a hydrocarbyldithiomethyl group, and a 2-nitrobenzyloxy group In some embodiments, the at least one cleavable group is the disulfide bond. In some embodiments, the at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof. In some embodiments, the detectably labeled substrate comprises a moiety selected from the group consisting of

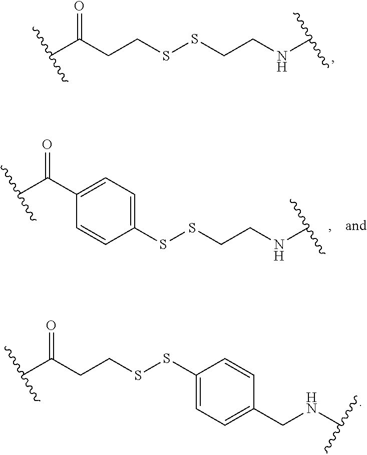 [SMILES: *NCCSSCCC(*)=O ; *NCCSSc1ccc(C(*)=O)cc1 ; *NCc1ccc(SSCCC(*)=O)cc1]

In some embodiments, the detectably labeled substrate comprises a compound of Formula IIa:

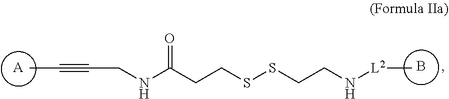

wherein: A is a deoxyribose nucleotide triphosphate; B is a detectable moiety; and L² comprises the at least one non-proteinogenic amino acid. In some embodiments, the detectably labeled substrate is a compound of Formula IIb, Formula IIc, Formula IId, Formula IIe, Formula IIf, or Formula IIg:

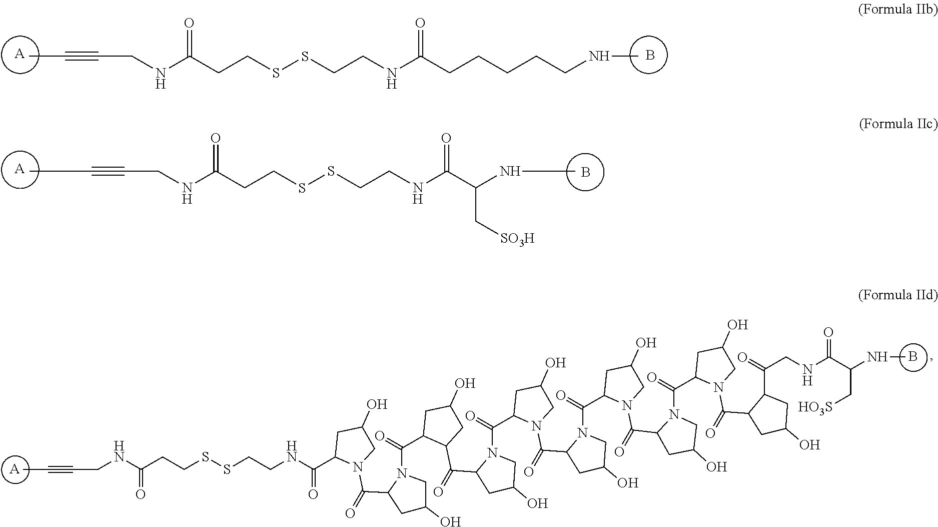

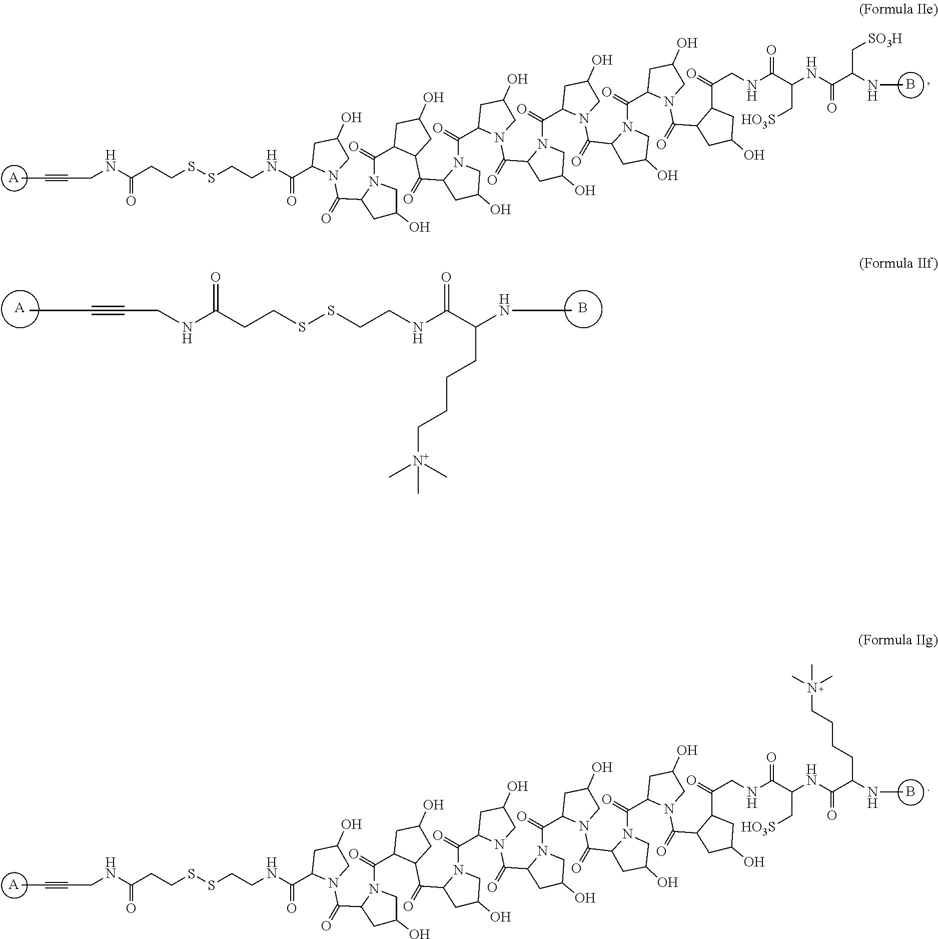

In an aspect, provided herein is a substrate comprising: (a) a nucleobase, wherein the nucleobase is not a guanine; and (b) a linker coupled to the nucleobase, wherein the linker comprises at least a first non-proteinogenic amino acid and at least a second non-proteinogenic amino acid, wherein the first non-proteinogenic amino acid and the second non-proteinogenic amino acid are different.

In an aspect, provided herein is a substrate comprising a compound of Formula III:

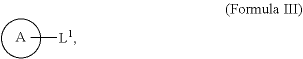

wherein: A comprises a nucleobase; and L¹ is a linker comprising at least a first non-proteinogenic amino acid and at least a second non-proteinogenic amino acid, wherein the nucleobase is not a guanine, and wherein the first non-proteinogenic amino acid and the second non-proteinogenic amino acid are different.

In some embodiments, the first non-proteinogenic amino acid comprises hydroxyproline. In some embodiments, the first non-proteinogenic amino acid comprises at least about 10 hydroxyprolines. In some embodiments, the second non-proteinogenic amino acid comprises cysteic acid, 5-amino-5-carboxy-N,N,N-trimethylpentan-1-aminium or a salt thereof, a quaternary amine, or 6-aminohexanoic acid, or a combination thereof. In some embodiments, the nucleobase comprises adenine, cytosine, thymine, or uracil. In some embodiments, the linker comprises at least one cleavable group. In some embodiments, the detectable moiety comprises at least one fluorescent dye. In some embodiments, the substrate comprises a moiety selected from the group consisting of

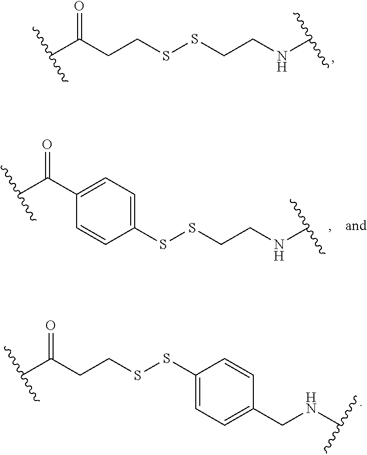 [SMILES: *NCCSSCCC(*)=O ; *NCCSSc1ccc(C(*)=O)cc1 ; *NCc1ccc(SSCCC(*)=O)cc1]

In an aspect, provided herein is a detectably labeled substrate comprising the substrate described herein, wherein the detectably labeled substrate comprises a compound of Formula IIIa:

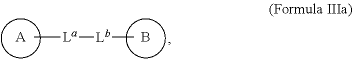

wherein: A comprises the nucleobase; B comprises a detectable moiety; L^{a} is a first linker; and L^{b} is a second linker. In some embodiments, L^{b} comprises the first non-proteinogenic amino acid or the second non-proteinogenic amino acid In some embodiments, L^{b} comprises the first non-proteinogenic amino acid and the second non-proteinogenic amino acid. In some embodiments, L^{a} comprises at least one cleavable group. In some embodiments, the detectably labeled substrate comprises a compound of Formula IIIb or a compound of Formula IIIc:

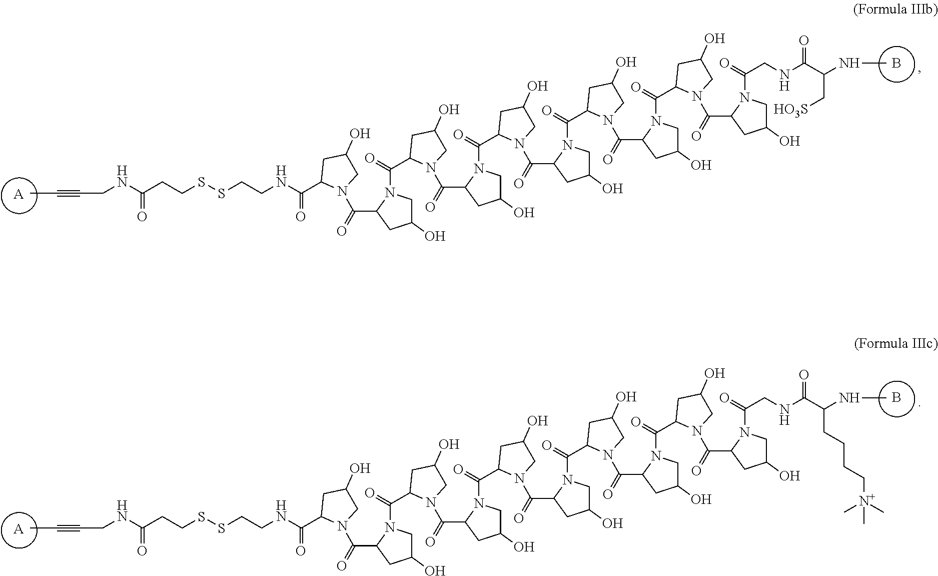

In an aspect, provided herein is a substrate comprising: (a) a nucleobase wherein the nucleobase is not a guanine; and (b) a linker coupled to the nucleobase, wherein the linker comprises at least two non-proteinogenic amino acids, wherein the at least two non-proteinogenic amino acids are a same type.

In an aspect, provided herein is a substrate comprising a compound of Formula IV:

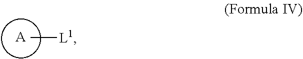

wherein: A comprises a nucleobase, wherein the nucleobase is not a guanine; and L¹ is a linker comprising at least two non-proteinogenic amino acids, wherein the at least two non-proteinogenic amino acids are a same type.

In some embodiments, the at least two non-proteinogenic amino acids are cysteic acids. In some embodiments, the at least two non-proteinogenic amino acids are 5-amino-5-carboxy-N,N,N-trimethylpentan-1-aminiums. In some embodiments, the substrate further comprises a third non-proteinogenic amino acid different from the at least two non-proteinogenic amino acids. In some embodiments, the third non-proteinogenic amino acid comprises hydroxyproline. In some embodiments, the nucleobase comprises adenine, cytosine, thymine, or uracil. In some embodiments, the linker comprises at least one cleavable group. In some embodiments, the detectable moiety comprises a fluorescent dye. In some embodiments, the substrate comprises a moiety selected from the group consisting of

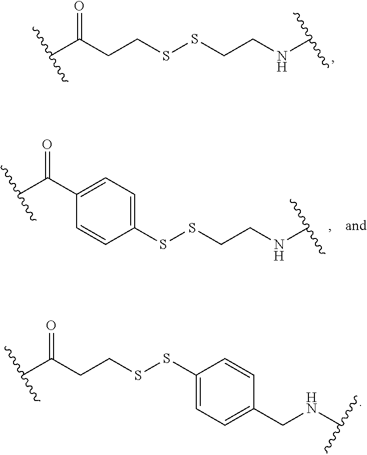 [SMILES: *NCCSSCCC(*)=O ; *NCCSSc1ccc(C(*)=O)cc1 ; *NCc1ccc(SSCCC(*)=O)cc1]

In an aspect, provided herein is a detectably labeled substrate comprising the substrate described herein, wherein the detectably labeled substrate is a compound of Formula IVa:

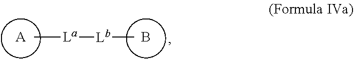

wherein: A comprises the nucleobase, wherein the nucleobase is not a guanine; B comprises a detectable moiety; L^{a} is a first linker; and L^{b} is a second linker. In some embodiments, L^{b} comprises the at least two non-proteinogenic amino acids. In some embodiments, L^{b} comprises a third non-proteinogenic amino acid. In some embodiments, the detectably labeled substrate is a compound of Formula IVc or a compound of Formula IVd

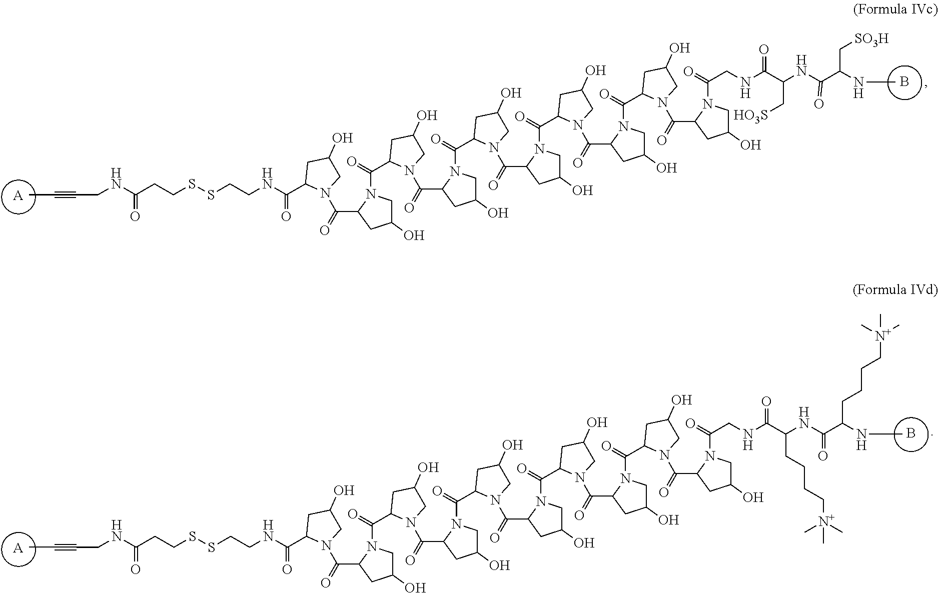

In an aspect, provided herein is a composition comprising a solution comprising a plurality of the labeled substrate, labeling reagent, and/or detectably labeled substrate described herein. In some embodiments, the solution further comprises a plurality of unlabeled substrates, wherein each substrate of the plurality of unlabeled substrates is of a same type as each the labeled substrate, labeling reagent, and/or detectably labeled substrate. In some embodiments, a ratio of the plurality of the labeled substrate, labeling reagent, and/or detectably labeled substrate to the plurality of unlabeled substrates in the solution is at least about 10:1. In some embodiments, the ratio is at least about 5:1. In some embodiments, the ratio is at least about 3:1.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a method for constructing a labeled nucleotide comprising a propargyl-derivatized nucleotide, a linker, and a dye.

FIGS. 2A and 2B show an example method for preparing a labeled nucleotide comprising a dGTP analog.

FIGS. 3A-3C show an example method for preparing a labeled nucleotide comprising a guanine analog.

FIG. 4 shows components that may be used to construct dye-labeled nucleotides.

FIG. 5 shows an example fluorescent labeling reagent.

FIG. 6 shows an example sequencing procedure.

FIG. 7 shows a schematic of a bead-based assay for evaluating labeled nucleotides.

FIG. 8 shows results of a bead-based assay for different labeled dUTPs.

FIG. 9 shows results of a bead-based assay for different labeled dATPs.

FIG. 10 shows results of a bead-based assay for different labeled dGTPs.

FIG. 11 shows tolerances of different labeled nucleotides.

FIG. 12 shows a schematic of an assay for evaluating quenching.

FIG. 13 shows quenching results for red dye linkers.

FIG. 14 shows quenching results for green dye linkers.

FIG. 15 shows example results of a sequencing analysis utilizing populations of nucleotides comprising 100% fluorophore labeled dNTPs.

FIG. 16 shows fluorescence of bovine serum albumin labeled with different fluorescent labeling moieties.

FIG. 17 shows example dye structures for inclusion in optical labeling reagents.

FIGS. 18A-18C show brightness (left panel) and homopolymeric incorporation (right panel) for different labeled uracil-containing nucleotides.

FIGS. 19A-19C show sequencing data for sequencing assays performed with varying labeling fractions.

FIG. 20A shows example results of a sequencing analysis utilizing populations of detectably labeled nucleotides with linkers comprising non-proteinogenic amino acids relative to a control. FIG. 20B shows summaries of the example results of FIG. 20A.

FIG. 21A shows an example result of a sequencing analysis utilizing a population of with linkers comprising non-proteinogenic amino acids relative to the control.

FIG. 21B shows another example result of a sequencing analysis utilizing a population of with linkers comprising non-proteinogenic amino acids relative to the control.

FIG. 22 shows an example synthesis for a fluorophore.

FIGS. 23A-23E show example schematics of attaching multiple dyes to poly-hydroxyprolines at different angles; FIG. 23A shows an example side view of a substrate attached to a linker attached to multiple dyes; FIG. 23B shows an example top view of the linker attached to multiple dyes; FIG. 23C shows an example top view of instances of multiple adjacent substrates each attached to a linker attached to multiple dyes. FIG. 23D shows an example nucleotide attached to a linker attached to multiple ATTO 532 dyes. FIG. 23E shows an example nucleotide attached to a linker attached to multiple ATTO 633 dyes.

FIG. 23F shows an example schematic for nucleotides with variable length linkers.

FIG. 24 shows an example process for synthesizing a fluorescently labeled nucleotide.

FIGS. 25A-25C show an example process for synthesizing a fluorescently labeled dUTP nucleotide with a cysteic acid at the C-terminus end of GlyHyp10.

FIG. 26A shows an example process for synthesizing a fluorescently labeled dGTP nucleotide with a cysteic acid at the N-terminus end of GlyHyp10. FIG. 26B shows an example process for synthesizing a fluorescently labeled dGTP nucleotide with a cysteic acid at both the C- and N-termini ends of GlyHyp10.

FIG. 27A shows an example process for synthesizing a fluorescently labeled dGTP nucleotide with 3 cysteic acids. FIG. 27B shows an example process for synthesizing a fluorescently labeled dGTP nucleotide with 2 cysteic acids at N-termini ends of Gly-Hyp6.

FIG. 28A-28C show an example process for synthesizing a fluorescently labeled dUTP nucleotide with a dimethyl ammonium.

FIGS. 29A and 29B show an example process for synthesizing a fluorescently labeled dUTP nucleotide with a trimethyl ammonium lysine.

FIGS. 30A-30C show an example process for synthesizing a fluorescently labeled dUTP nucleotide with an E-cleavable linker, 2 non-terminal ATTO 633 dye segments and a terminal ATTO 633 dye segment, each separated by a Hyp10.

FIGS. 31A and 31B show an example process for synthesizing a fluorescently labeled dUTP nucleotide with a Y-cleavable linker, a non-terminal ATTO 532 dye segment and a terminal ATTO 532 dye segment, each separated by a Hyp10.

FIGS. 32A-32B show plate-based kinetics assay data for labeled dUTP and labeled dATP linkers that contain quaternary amines.

FIG. 33 shows fluorescence comparison data for different multiply labeled dUTP compounds.

FIG. 34A shows one example structure of an adjustably labeled substrate. FIG. 34B shows an example labeled dUTP, labeled per the structure of FIG. 34A.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “about” and “approximately” shall generally mean an acceptable degree of error or variation for a given value or range of values, such as, for example, a degree of error or variation that is within 20 percent (%), within 15%, within 10%, or within 5% of a given value or range of values.

The term “subject,” as used herein, generally refers to an individual or entity from which a biological sample (e.g., a biological sample that is undergoing or can undergo processing or analysis) may be derived. A subject may be an animal (e.g., mammal or non-mammal) or plant. The subject may be a human, dog, cat, horse, pig, bird, non-human primate, simian, farm animal, companion animal, sport animal, or rodent. A subject may be a patient. The subject may have or be suspected of having a disease or disorder, such as cancer (e.g., breast cancer, colorectal cancer, brain cancer, leukemia, lung cancer, skin cancer, liver cancer, pancreatic cancer, lymphoma, esophageal cancer or cervical cancer) or an infectious disease. Alternatively or additionally, a subject may be known to have previously had a disease or disorder. The subject may have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease. A subject may be undergoing treatment for a disease or disorder. A subject may be symptomatic or asymptomatic of a given disease or disorder. A subject may be healthy (e.g., not suspected of having disease or disorder). A subject may have one or more risk factors for a given disease. A subject may have a given weight, height, body mass index, or other physical characteristics. A subject may have a given ethnic or racial heritage, place of birth or residence, nationality, disease or remission state, family medical history, or other characteristics.

As used herein, the term “biological sample” generally refers to a sample obtained from a subject. The biological sample may be obtained directly or indirectly from the subject. A sample may be obtained from a subject via any suitable method, including, but not limited to, spitting, swabbing, blood draw, biopsy, obtaining excretions (e.g., urine, stool, sputum, vomit, or saliva), excision, scraping, and puncture. A sample may be obtained from a subject by, for example, intravenously or intraarterially accessing the circulatory system, collecting a secreted biological sample (e.g., stool, urine, saliva, sputum, etc.), breathing, or surgically extracting a tissue (e.g., biopsy). The sample may be obtained by non-invasive methods including but not limited to: scraping of the skin or cervix, swabbing of the cheek, or collection of saliva, urine, feces, menses, tears, or semen. Alternatively, the sample may be obtained by an invasive procedure such as biopsy, needle aspiration, or phlebotomy. A sample may comprise a bodily fluid such as, but not limited to, blood (e.g., whole blood, red blood cells, leukocytes or white blood cells, platelets), plasma, serum, sweat, tears, saliva, sputum, urine, semen, mucus, synovial fluid, breast milk, colostrum, amniotic fluid, bile, bone marrow, interstitial or extracellular fluid, or cerebrospinal fluid. For example, a sample may be obtained by a puncture method to obtain a bodily fluid comprising blood and/or plasma. Such a sample may comprise both cells and cell-free nucleic acid material. Alternatively, the sample may be obtained from any other source including but not limited to blood, sweat, hair follicle, buccal tissue, tears, menses, feces, or saliva. The biological sample may be a tissue sample, such as a tumor biopsy. The sample may be obtained from any of the tissues provided herein including, but not limited to, skin, heart, lung, kidney, breast, pancreas, liver, intestine, brain, prostate, esophagus, muscle, smooth muscle, bladder, gall bladder, colon, or thyroid. The methods of obtaining provided herein include methods of biopsy including fine needle aspiration, core needle biopsy, vacuum assisted biopsy, large core biopsy, incisional biopsy, excisional biopsy, punch biopsy, shave biopsy, or skin biopsy. The biological sample may comprise one or more cells. A biological sample may comprise one or more nucleic acid molecules such as one or more deoxyribonucleic acid (DNA) and/or ribonucleic acid (RNA) molecules (e.g., included within cells or not included within cells). Nucleic acid molecules may be included within cells. Alternatively or additionally, nucleic acid molecules may not be included within cells (e.g., cell-free nucleic acid molecules). The biological sample may be a cell-free sample.

The term “cell-free sample,” as used herein, generally refers to a sample that is substantially free of cells (e.g., less than 10% cells on a volume basis). A cell-free sample may be derived from any source (e.g., as described herein). For example, a cell-free sample may be derived from blood, sweat, urine, or saliva. For example, a cell-free sample may be derived from a tissue or bodily fluid. A cell-free sample may be derived from a plurality of tissues or bodily fluids. For example, a sample from a first tissue or fluid may be combined with a sample from a second tissue or fluid (e.g., while the samples are obtained or after the samples are obtained). In an example, a first fluid and a second fluid may be collected from a subject (e.g., at the same or different times) and the first and second fluids may be combined to provide a sample. A cell-free sample may comprise one or more nucleic acid molecules such as one or more DNA or RNA molecules.

A sample that is not a cell-free sample (e.g., a sample comprising one or more cells) may be processed to provide a cell-free sample. For example, a sample that includes one or more cells as well as one or more nucleic acid molecules (e.g., DNA and/or RNA molecules) not included within cells (e.g., cell-free nucleic acid molecules) may be obtained from a subject. The sample may be subjected to processing (e.g., as described herein) to separate cells and other materials from the nucleic acid molecules not included within cells, thereby providing a cell-free sample (e.g., comprising nucleic acid molecules not included within cells). The cell-free sample may then be subjected to further analysis and processing (e.g., as provided herein). Nucleic acid molecules not included within cells (e.g., cell-free nucleic acid molecules) may be derived from cells and tissues. For example, cell-free nucleic acid molecules may derive from a tumor tissue or a degraded cell (e.g., of a tissue of a body). Cell-free nucleic acid molecules may comprise any type of nucleic acid molecules (e.g., as described herein). Cell-free nucleic acid molecules may be double-stranded, single-stranded, or a combination thereof. Cell-free nucleic acid molecules may be released into a bodily fluid through secretion or cell death processes, e.g., cellular necrosis, apoptosis, or the like. Cell-free nucleic acid molecules may be released into bodily fluids from cancer cells (e.g., circulating tumor DNA (ctDNA)). Cell free nucleic acid molecules may also be fetal DNA circulating freely in a maternal blood stream (e.g., cell-free fetal nucleic acid molecules such as cffDNA). Alternatively or additionally, cell-free nucleic acid molecules may be released into bodily fluids from healthy cells.

A biological sample may be obtained directly from a subject and analyzed without any intervening processing, such as, for example, sample purification or extraction. For example, a blood sample may be obtained directly from a subject by accessing the subject's circulatory system, removing the blood from the subject (e.g., via a needle), and transferring the removed blood into a receptacle. The receptacle may comprise reagents (e.g., anti-coagulants) such that the blood sample is useful for further analysis. Such reagents may be used to process the sample or analytes derived from the sample in the receptacle or another receptacle prior to analysis. In another example, a swab may be used to access epithelial cells on an oropharyngeal surface of the subject. Following obtaining the biological sample from the subject, the swab containing the biological sample may be contacted with a fluid (e.g., a buffer) to collect the biological fluid from the swab.

Any suitable biological sample that comprises one or more nucleic acid molecules may be obtained from a subject. A sample (e.g., a biological sample or cell-free biological sample) suitable for use according to the methods provided herein may be any material comprising tissues, cells, degraded cells, nucleic acids, genes, gene fragments, expression products, gene expression products, and/or gene expression product fragments of an individual to be tested. A biological sample may be solid matter (e.g., biological tissue) or may be a fluid (e.g., a biological fluid). In general, a biological fluid may include any fluid associated with living organisms. Non-limiting examples of a biological sample include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues. Methods for determining sample suitability and/or adequacy are provided. A sample may include, but is not limited to, blood, plasma, tissue, cells, degraded cells, cell-free nucleic acid molecules, and/or biological material from cells or derived from cells of an individual such as cell-free nucleic acid molecules. The sample may be a heterogeneous or homogeneous population of cells, tissues, or cell-free biological material. The biological sample may be obtained using any method that can provide a sample suitable for the analytical methods described herein.

A sample (e.g., a biological sample or cell-free biological sample) may undergo one or more processes in preparation for analysis, including, but not limited to, filtration, centrifugation, selective precipitation, permeabilization, isolation, agitation, heating, purification, and/or other processes. For example, a sample may be filtered to remove contaminants or other materials. In an example, a sample comprising cells may be processed to separate the cells from other material in the sample. Such a process may be used to prepare a sample comprising only cell-free nucleic acid molecules. Such a process may consist of a multi-step centrifugation process. Multiple samples, such as multiple samples from the same subject (e.g., obtained in the same or different manners from the same or different bodily locations, and/or obtained at the same or different times (e.g., seconds, minutes, hours, days, weeks, months, or years apart)) or multiple samples from different subjects may be obtained for analysis as described herein. In an example, the first sample is obtained from a subject before the subject undergoes a treatment regimen or procedure and the second sample is obtained from the subject after the subject undergoes the treatment regimen or procedure. Alternatively or additionally, multiple samples may be obtained from the same subject at the same or approximately the same time. Different samples obtained from the same subject may be obtained in the same or different manner. For example, a first sample may be obtained via a biopsy and a second sample may be obtained via a blood draw. Samples obtained in different manners may be obtained by different medical professionals, using different techniques, at different times, and/or at different locations. Different samples obtained from the same subject may be obtained from different areas of a body. For example, a first sample may be obtained from a first area of a body (e.g., a first tissue) and a second sample may be obtained from a second area of the body (e.g., a second tissue).

A biological sample as used herein (e.g., a biological sample comprising one or more nucleic acid molecules) may not be purified when provided in a reaction vessel. Furthermore, for a biological sample comprising one or more nucleic acid molecules, the one or more nucleic acid molecules may not be extracted when the biological sample is provided to a reaction vessel. For example, ribonucleic acid (RNA) and/or deoxyribonucleic acid (DNA) molecules of a biological sample may not be extracted from the biological sample when providing the biological sample to a reaction vessel. Moreover, a target nucleic acid (e.g., a target RNA or target DNA molecules) present in a biological sample may not be concentrated when providing the biological sample to a reaction vessel. Alternatively, a biological sample may be purified and/or nucleic acid molecules may be isolated from other materials in the biological sample.

A biological sample as described herein may contain a target nucleic acid. As used herein, the terms “template nucleic acid,” “target nucleic acid,” “nucleic acid molecule,” “nucleic acid sequence,” “nucleic acid fragment,” “oligonucleotide,” “polynucleotide,” and “nucleic acid” generally refer to polymeric forms of nucleotides of any length, such as deoxyribonucleotides (dNTPs) or ribonucleotides (rNTPs), or analogs thereof, and may be used interchangeably. Nucleic acids may have any three-dimensional structure, and may perform any function, known or unknown. A nucleic acid molecule may have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. An oligonucleotide is typically composed of a specific sequence of four nucleotide bases: adenine (A); cytosine (C); guanine (G); and thymine (T) (uracil (U) for thymine (T) when the polynucleotide is RNA). Oligonucleotides may include one or more nonstandard nucleotide(s), nucleotide analog(s) and/or modified nucleotides. Non-limiting examples of nucleic acids include DNA, RNA, genomic DNA (e.g., gDNA such as sheared gDNA), cell-free DNA (e.g., cfDNA), synthetic DNA/RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, complementary DNA (cDNA), recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or following assembly of the nucleic acid. The sequence of nucleotides of a nucleic acid may be interrupted by non-nucleotide components. A nucleic acid may be further modified following polymerization, such as by conjugation or binding with a reporter agent.

A target nucleic acid or sample nucleic acid as described herein may be amplified to generate an amplified product. A target nucleic acid may be a target RNA or a target DNA. When the target nucleic acid is a target RNA, the target RNA may be any type of RNA, including types of RNA described elsewhere herein. The target RNA may be viral RNA and/or tumor RNA. A viral RNA may be pathogenic to a subject. Non-limiting examples of pathogenic viral RNA include human immunodeficiency virus I (HIV I), human immunodeficiency virus n (HIV 11), orthomyxoviruses, Ebola virus. Dengue virus, influenza viruses (e.g., H1N1, H3N2, H7N9, or H5N1), herpesvirus, hepatitis A virus, hepatitis B virus, hepatitis C (e.g., armored RNA-HCV virus) virus, hepatitis D virus, hepatitis E virus, hepatitis G virus, Epstein-Barr virus, mononucleosis virus, cytomegalovirus, SARS virus, West Nile Fever virus, polio virus, and measles virus.

A biological sample may comprise a plurality of target nucleic acid molecules. For example, a biological sample may comprise a plurality of target nucleic acid molecules from a single subject. In another example, a biological sample may comprise a first target nucleic acid molecule from a first subject and a second target nucleic acid molecule from a second subject.

The term “nucleotide,” as used herein, generally refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a modified or engineered nucleotide).

The term “nucleotide analog,” as used herein, may include, but is not limited to, a nucleotide that may or may not be a naturally occurring nucleotide. For example, a nucleotide analog may be derived from and/or include structural similarities to a canonical nucleotide such as adenine—(A), thymine—(T), cytosine—(C), uracil—(U), or guanine—(G) including nucleotide. A nucleotide analog may comprise one or more differences or modifications relative to a natural nucleotide. Examples of nucleotide analogs include inosine, diaminopurine, 5-fluorouracil, 5-bromouracil, 5-chlorouracil, 5-iodouracil, hypoxanthine, xanthine, deazaxanthine, deazaguanine, isocytosine, isoguanine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxy acetic acid methylester, uracil-5-oxyacetic acid (v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids, and modified versions thereof (e.g., by oxidation, reduction, and/or addition of a substituent such as an alkyl, hydroxyalkyl, hydroxyl, or halogen moiety). Nucleic acid molecules (e.g., polynucleotides, double-stranded nucleic acid molecules, single-stranded nucleic acid molecules, primers, adapters, etc.) may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. In some cases, a nucleotide may include a modification in its phosphate moiety, including a modification to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates), and modifications with selenium moieties (e.g., phosphoroselenoate nucleic acids). A nucleotide or nucleotide analog may comprise a sugar selected from the group consisting of ribose, deoxyribose, and modified versions thereof (e.g., by oxidation, reduction, and/or addition of a substituent such as an alkyl, hydroxyalkyl, hydroxyl, or halogen moiety). A nucleotide analog may also comprise a modified linker moiety (e.g., in lieu of a phosphate moiety). Nucleotide analogs may also contain amine-modified groups, such as aminoallyl-dUTP (aa-dUTP) and aminohexylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure may provide, for example, higher density in bits per cubic mm, higher safety (resistant to accidental or purposeful synthesis of natural toxins), easier discrimination in photo-programmed polymerases, and/or lower secondary structure. Nucleotide analogs may be capable of reacting or bonding with detectable moieties for nucleotide detection.

The term “homopolymer,” as used herein, generally refers to a polymer or a portion of a polymer comprising identical monomer units. A homopolymer may have a homopolymer sequence. A nucleic acid homopolymer may refer to a polynucleotide or an oligonucleotide comprising consecutive repetitions of a same nucleotide or any nucleotide variants thereof. For example, a homopolymer can be poly(dA), poly(dT), poly(dG), poly(dC), poly(rA), poly(U), poly(rG), or poly(rC). A homopolymer can be of any length. For example, the homopolymer can have a length of at least 2, 3, 4, 5, 10, 20, 30, 40, 50, 100, 200, 300, 400, 500, or more nucleic acid bases. The homopolymer can have from 10 to 500, or 15 to 200, or 20 to 150 nucleic acid bases. The homopolymer can have a length of at most 500, 400, 300, 200, 100, 50, 40, 30, 20, 10, 5, 4, 3, or 2 nucleic acid bases. A molecule, such as a nucleic acid molecule, can include one or more homopolymer portions and one or more non-homopolymer portions. The molecule may be entirely formed of a homopolymer, multiple homopolymers, or a combination of homopolymers and non-homopolymers. In nucleic acid sequencing, multiple nucleotides can be incorporated into a homopolymeric region of a nucleic acid strand. Such nucleotides may be non-terminated to permit incorporation of consecutive nucleotides (e.g., during a single nucleotide flow).

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. An amplicon may be a single-stranded or double-stranded nucleic acid molecule that is generated by an amplification procedure from a starting template nucleic acid molecule. Such an amplification procedure may include one or more cycles of an extension or ligation procedure. The amplicon may comprise a nucleic acid strand, of which at least a portion may be substantially identical or substantially complementary to at least a portion of the starting template. Where the starting template is a double-stranded nucleic acid molecule, an amplicon may comprise a nucleic acid strand that is substantially identical to at least a portion of one strand and is substantially complementary to at least a portion of either strand. The amplicon can be single-stranded or double-stranded irrespective of whether the initial template is single-stranded or double-stranded. Amplification of a nucleic acid may linear, exponential, or a combination thereof. Amplification may be emulsion based or may be non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification, and multiple displacement amplification (MDA). Where PCR is used, any form of PCR may be used, with non-limiting examples that include real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR and touchdown PCR. Moreover, amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C.C. PNAS, 1989, 86, 4076-4080 and U.S. Pat. Nos. 5,409,811 and 5,674,716, each of which is herein incorporated by reference in its entirety.

Amplification may be clonal amplification. The term “clonal,” as used herein, generally refers to a population of nucleic acids for which a substantial portion (e.g., greater than about 50%, 60%, 70%, 80%, 90%, 95%, or 99%) of its members have sequences that are at least about 50%, 60%, 70%, 80%, 90%, 95%, or 99% identical to one another. Members of a clonal population of nucleic acid molecules may have sequence homology to one another. Such members may have sequence homology to a template nucleic acid molecule. The members of the clonal population may be double stranded or single stranded. Members of a population may not be 100% identical or complementary, e.g., “errors” may occur during the course of synthesis such that a minority of a given population may not have sequence homology with a majority of the population. For example, at least 50% of the members of a population may be substantially identical to each other or to a reference nucleic acid molecule (i.e., a molecule of defined sequence used as a basis for a sequence comparison). At least 60%, at least 70%, at least 80%, at least 90%, at least 95%, at least 99%, or more of the members of a population may be substantially identical to the reference nucleic acid molecule. Two molecules may be considered substantially identical (or homologous) if the percent identity between the two molecules is at least 60%, 70%, 75%, 80%, 85%, 90%, 95%, 98%, 99%, 99.9% or greater. Two molecules may be considered substantially complementary if the percent complementarity between the two molecules is at least 60%, 70%, 75%, 80%, 85%, 90%, 95%, 98%, 99%, 99.9% or greater. A low or insubstantial level of mixing of non-homologous nucleic acids may occur, and thus a clonal population may contain a minority of diverse nucleic acids (e.g., less than 30%, e.g., less than 10%).

Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). The enhanced signal-to-noise ratio provided by clonal amplification more than outweighs the disadvantages of the cyclic sequencing requirement.

The term “polymerizing enzyme” or “polymerase,” as used herein, generally refers to any enzyme capable of catalyzing a polymerization reaction. A polymerizing enzyme may be used to extend a nucleic acid primer paired with a template strand by incorporation of nucleotides or nucleotide analogs. A polymerizing enzyme may add a new strand of DNA by extending the 3′ end of an existing nucleotide chain, adding new nucleotides matched to the template strand one at a time via the creation of phosphodiester bonds. The polymerase used herein can have strand displacement activity or non-strand displacement activity. Examples of polymerases include, without limitation, a nucleic acid polymerase. An example polymerase is a 029 DNA polymerase or a derivative thereof. A polymerase can be a polymerization enzyme. In some cases, a transcriptase or a ligase is used (i.e., enzymes which catalyze the formation of a bond). Examples of polymerases include a DNA polymerase, an RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, Pwo polymerase, VENT polymerase, DEEPVENT polymerase, EX-Taq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tea polymerase, Tih polymerase, Tfi polymerase, Platinum Taq polymerases, Tbr polymerase, Tfl polymerase, Pfu-turbo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. In some cases, the polymerase is a single subunit polymerase. The polymerase can have high processivity, namely the capability of the polymerase to consecutively incorporate nucleotides into a nucleic acid template without releasing the nucleic acid template. In some cases, a polymerase is a polymerase modified to accept dideoxynucleotide triphosphates, such as for example, Taq polymerase having a 667Y mutation (see e.g., Tabor et al, PNAS, 1995, 92, 6339-6343, which is herein incorporated by reference in its entirety for all purposes). In some cases, a polymerase is a polymerase having a modified nucleotide binding, which may be useful for nucleic acid sequencing, with non-limiting examples that include ThermoSequenas polymerase (GE Life Sciences), AmpliTaq FS (ThermoFisher) polymerase and Sequencing Pol polymerase (Jena Bioscience). In some cases, the polymerase is genetically engineered to have discrimination against dideoxynucleotides, such as for example, Sequenase DNA polymerase (ThermoFisher).

A polymerase may be a Family A polymerase or a Family B DNA polymerase. Family A polymerases include, for example, Taq, Klenow, and Bst polymerases. Family B polymerases include, for example, Vent(exo-) and Therminator polymerases. Family B polymerases are known to accept more varied nucleotide substrates than Family A polymerases. Family A polymerases are used widely in sequencing by synthesis methods, likely due to their high processivity and fidelity.

The term “complementary sequence,” as used herein, generally refers to a sequence that hybridizes to another sequence. Hybridization between two single-stranded nucleic acid molecules may involve the formation of a double-stranded structure that is stable under certain conditions. Two single-stranded polynucleotides may be considered to be hybridized if they are bonded to each other by two or more sequentially adjacent base pairings. A substantial proportion of nucleotides in one strand of a double-stranded structure may undergo Watson-Crick base-pairing with a nucleoside on the other strand. Hybridization may also include the pairing of nucleoside analogs, such as deoxyinosine, nucleosides with 2-aminopurine bases, and the like, that may be employed to reduce the degeneracy of probes, whether or not such pairing involves formation of hydrogen bonds.

The term “denaturation,” as used herein, generally refers to separation of a double-stranded molecule (e.g., DNA) into single-stranded molecules. Denaturation may be complete or partial denaturation. In partial denaturation, a single-stranded region may form in a double-stranded molecule by denaturation of the two deoxyribonucleic acid (DNA) strands flanked by double-stranded regions in DNA.

The term “melting temperature” or “melting point,” as used herein, generally refers to the temperature at which at least a portion of a strand of a nucleic acid molecule in a sample has separated from at least a portion of a complementary strand. The melting temperature may be the temperature at which a double-stranded nucleic acid molecule has partially or completely denatured. The melting temperature may refer to a temperature of a sequence among a plurality of sequences of a given nucleic acid molecule, or a temperature of the plurality of sequences. Different regions of a double-stranded nucleic acid molecule may have different melting temperatures. For example, a double-stranded nucleic acid molecule may include a first region having a first melting point and a second region having a second melting point that is higher than the first melting point. Accordingly, different regions of a double-stranded nucleic acid molecule may melt (e.g., partially denature) at different temperatures. The melting point of a nucleic acid molecule or a region thereof (e.g., a nucleic acid sequence) may be determined experimentally (e.g., via a melt analysis or other procedure) or may be estimated based upon the sequence and length of the nucleic acid molecule. For example, a software program such as MELTING may be used to estimate a melting temperature for a nucleic acid sequence (Dumousseau M, Rodriguez N, Juty N, Le Novere N, MELTING, a flexible platform to predict the melting temperatures of nucleic acids. BMC Bioinformatics. 2012 May 16; 13:101. doi: 10.1186/1471-2105-13-101, which is incorporated by reference by its entirety). Accordingly, a melting point as described herein may be an estimated melting point. A true melting point of a nucleic acid sequence may vary based upon the sequences or lack thereof adjacent to the nucleic acid sequence of interest as well as other factors.

The term “sequencing,” as used herein, generally refers to a process for generating or identifying a sequence of a biological molecule, such as a nucleic acid molecule or a polypeptide. Such sequence may be a nucleic acid sequence, which may include a sequence of nucleic acid bases (e.g., nucleobases). Sequencing may be, for example, single molecule sequencing, sequencing by synthesis, sequencing by hybridization, or sequencing by ligation. Sequencing may be performed using template nucleic acid molecules immobilized on a support, such as a flow cell or one or more beads. A sequencing assay may yield one or more sequencing reads corresponding to one or more template nucleic acid molecules.

The term “read,” as used herein, generally refers to a nucleic acid sequence, such as a sequencing read. A sequencing read may be an inferred sequence of nucleic acid bases (e.g., nucleotides) or base pairs obtained via a nucleic acid sequencing assay. A sequencing read may be generated by a nucleic acid sequencer, such as a massively parallel array sequencer (e.g., Illumina or Pacific Biosciences of California). A sequencing read may correspond to a portion, or in some cases all, of a genome of a subject. A sequencing read may be part of a collection of sequencing reads, which may be combined through, for example, alignment (e.g., to a reference genome), to yield a sequence of a genome of a subject.

The term “detector,” as used herein, generally refers to a device that is capable of detecting or measuring a signal, such as a signal indicative of the presence or absence of an incorporated nucleotide or nucleotide analog. A detector may include optical and/or electronic components that may detect and/or measure signals. Non-limiting examples of detection methods involving a detector include optical detection, spectroscopic detection, electrostatic detection, and electrochemical detection. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products.

The term “support,” as used herein, generally refers to any solid or semi-solid article on which reagents such as nucleic acid molecules may be immobilized. Nucleic acid molecules may be synthesized, attached, ligated, or otherwise immobilized. Nucleic acid molecules may be immobilized on a support by any method including, but not limited to, physical adsorption, by ionic or covalent bond formation, or combinations thereof. A support may be 2-dimensional (e.g., a planar 2D support) or 3-dimensional. In some cases, a support may be a component of a flow cell and/or may be included within or adapted to be received by a sequencing instrument. A support may include a polymer, a glass, or a metallic material. Examples of supports include a membrane, a planar support, a microtiter plate, a bead (e.g., a magnetic bead), a filter, a test strip, a slide, a cover slip, and a test tube. A support may comprise organic polymers such as polystyrene, polyethylene, polypropylene, polyfluoroethylene, polyethyleneoxy, and polyacrylamide (e.g., polyacrylamide gel), as well as co-polymers and grafts thereof. A support may comprise latex or dextran. A support may also be inorganic, such as glass, silica, gold, controlled-pore-glass (CPG), or reverse-phase silica. The configuration of a support may be, for example, in the form of beads, spheres, particles, granules, a gel, a porous matrix, or a support. In some cases, a support may be a single solid or semi-solid article (e.g., a single particle), while in other cases a support may comprise a plurality of solid or semi-solid articles (e.g., a collection of particles). Supports may be planar, substantially planar, or non-planar. Supports may be porous or non-porous. Supports may have swelling or non-swelling characteristics. A support may be shaped to comprise one or more wells, depressions, or other containers, vessels, features, or locations. A plurality of supports may be configured in an array at various locations. A support may be addressable (e.g., for robotic delivery of reagents), or by detection approaches, such as scanning by laser illumination and confocal or deflective light gathering. For example, a support may be in optical and/or physical communication with a detector. Alternatively, a support may be physically separated from a detector by a distance. An amplification support (e.g., a bead) can be placed within or on another support (e.g., within a well of a second support).

The term “coupled to,” as used herein, generally refers to an association between two or more objects that may be temporary or substantially permanent. A first object may be reversibly or irreversibly coupled to a second object. For example, a nucleic acid molecule may be reversibly coupled to a particle. A reversible coupling may comprise, for example, a releasable coupling (e.g., in which a first object may be released from a second object to which it is coupled). A first object releasably coupled to a second object may be separated from the second object, e.g., upon application of a stimulus, which stimulus may comprise a photostimulus (e.g., ultraviolet light), a thermal stimulus, a chemical stimulus (e.g., reducing agent), or any other useful stimulus. Coupling may encompass immobilization to a support (e.g., as described herein). Similarly, coupling may encompass attachment, such as attachment of a first object to a second object. A coupling may comprise any interaction that affects an association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-t interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. For example, a particle may be coupled to a planar support via an electrostatic interaction. In another example, a particle may be coupled to a planar support via a magnetic interaction. In another example, a particle may be coupled to a planar support via a covalent interaction. Similarly, a nucleic acid molecule may be coupled to a particle via a covalent interaction. Alternatively or additionally, a nucleic acid molecule may be coupled to a particle via a non-covalent interaction. A coupling between a first object and a second object may comprise a labile moiety, such as a moiety comprising an ester, vicinal diol, phosphodiester, peptidic, glycosidic, sulfone, Diels-Alder, or similar linkage. The strength of a coupling between a first object and a second object may be indicated by a dissociation constant, Kd, that indicates the inclination of a coupled object comprising a first object and a second object to dissociate into the uncoupled first and second objects and may be expressed as a ratio of dissociated (e.g., uncoupled) objects to coupled objects. A smaller dissociation constant is generally indicative of a stronger coupling between coupled objects.

Coupled objects and their corresponding uncoupled components may exist in dynamic equilibrium with one another. For example, a solution comprising a plurality of coupled objects each comprising a first object and a second object may also include a plurality of first objects and a plurality of second objects. At a given point in time, a given first object and a given second object may be coupled to one another or the objects may be uncoupled; the relative concentrations of coupled and uncoupled components throughout the solution can depend upon the strength of the coupling between the first and second objects (reflected in the dissociation constant). For example, a binding moiety may be coupled to a nucleic acid molecule to provide a binding complex. In a solution comprising a plurality of binding complexes each comprising a binding moiety coupled to a nucleic acid molecule, the plurality of binding complexes may exist in equilibrium with their constituent nucleic acid molecules and binding moieties. The association between a given nucleic acid molecule and a given binding moiety may be such that, at a given point in time, at least 50%, such as at least 60%, 65%, 70%, 75%, 80%, 85%, 90%, 95%, 98%, or more, of the nucleic acid molecules may be components of a binding complex of the plurality of binding complexes.

The term “label,” as used herein, generally refers to a moiety that is capable of coupling with a species, such as, for example a nucleotide analog. A label may include an affinity moiety. In some cases, a label may be a detectable moiety that emits a signal (or reduces an already emitted signal) that can be detected. In some cases, a labeling reagent may comprise a label. In some cases, such a signal may be indicative of incorporation of one or more nucleotides or nucleotide analogs. In some cases, a label may be coupled to a nucleotide or nucleotide analog, which nucleotide or nucleotide analog may be used in a primer extension reaction. In some cases, the label may be coupled to a nucleotide analog after a primer extension reaction. The label, in some cases, may be reactive specifically with a nucleotide or nucleotide analog. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP), tris(hydroxypropyl)phosphine (THP) or enzymatically cleavable (e.g., via an esterase, lipase, peptidase or protease). In some cases, the label may be luminescent; that is, fluorescent or phosphorescent. For example, the label may be or comprise a fluorescent moiety (e.g., a dye). Dyes and labels may be incorporated into nucleic acid sequences. Dyes and labels may also be incorporated into or attached to linkers, such as linkers for linking one or more beads to one another. For example, labels such as fluorescent moieties may be linked to nucleotides or nucleotide analogs via a linker (e.g., as described herein). Non-limiting examples of dyes include SYBR green, SYBR blue, DAPI, propidium iodine, Hoechst, SYBR gold, ethidium bromide, acridine, proflavine, acridine orange, acriflavine, fluorocoumarin, ellipticine, daunomycin, chloroquine, distamycin D, chromomycin, homidium, mithramycin, ruthenium polypyridyls, anthramycin, phenanthridines and acridines, propidium iodide, hexidium iodide, dihydroethidium, ethidium homodimer-1 and -2, ethidium monoazide, ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO labels (e.g., SYTO-40, -41, -42, -43, -44, and -45 (blue); SYTO-13, -16, -24, -21, -23, -12, -11, -20, -22, -15, -14, and -25 (green); SYTO-81, -80, -82, -83, -84, and-85 (orange); and SYTO-64, -17, -59, -61, -62, -60, and -63 (red)), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor labels (e.g., AlexaFluor 350, 405, 430, 488, 532, 546, 555, 568, 594, 610, 633, 635, 647, 660, 680, 700, 750, and 790 dyes), DyLight labels (e.g., DyLight 350, 405, 488, 550, 594, 633, 650, 680, 755, and 800 dyes), Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), ATTO fluorescent quenchers (ATTO-TEC GmbH) (e.g., ATTO 540Q, ATTO 580Q, ATTO 612Q, Atto532 [e.g., ATTO 532 succinimidyl ester], and Atto633), Kam, and other fluorophores and/or quenchers. Additional examples are included in structures provided herein. Dyes included in structures provided herein are contemplated for use in combination with any linker and substrate described herein. A fluorescent dye may be excited by application of energy corresponding to the visible region of the electromagnetic spectrum (e.g., between about 430-770 nanometers (nm)). Excitation may be done using any useful apparatus, such as a laser and/or light emitting diode. Optical elements including, but not limited to, mirrors, waveplates, filters, monochromators, gratings, beam splitters, and lenses may be used to direct light to or from a fluorescent dye. A fluorescent dye may emit light (e.g., fluoresce) in the visible region of the electromagnetic spectrum ((e.g., between about 430-770 nm). A fluorescent dye may be excited over a single wavelength or a range of wavelengths. A fluorescent dye may be excitable by light in the red region of the visible portion of the electromagnetic spectrum (about 625-740 nm) (e.g., have an excitation maximum in the red region of the visible portion of the electromagnetic spectrum). Alternatively or additionally, fluorescent dye may be excitable by light in the green region of the visible portion of the electromagnetic spectrum (about 500-565 nm) (e.g., have an excitation maximum in the green region of the visible portion of the electromagnetic spectrum). A fluorescent dye may emit signal in the red region of the visible portion of the electromagnetic spectrum (about 625-740 nm) (e.g., have an emission maximum in the red region of the visible portion of the electromagnetic spectrum). Alternatively or additionally, fluorescent dye may emit signal in the green region of the visible portion of the electromagnetic spectrum (about 500-565 nm) (e.g., have an emission maximum in the green region of the visible portion of the electromagnetic spectrum).

Labels may be quencher molecules. The term “quencher,” as used herein, generally refers to molecules that may be energy acceptors. A quencher may be a molecule that can reduce an emitted signal. For example, a template nucleic acid molecule may be designed to emit a detectable signal. Incorporation of a nucleotide or nucleotide analog comprising a quencher can reduce or eliminate the signal, which reduction or elimination is then detected. Luminescence from labels (e.g., fluorescent moieties, such as fluorescent moieties linked to nucleotides or nucleotide analogs) may also be quenched (e.g., by incorporation of other nucleotides that may or may not comprise labels). In some cases, as described elsewhere herein, labelling with a quencher can occur after nucleotide or nucleotide analog incorporation (e.g., after incorporation of a nucleotide or nucleotide analog comprising a fluorescent moiety). In some cases, the label may be a type that does not self-quench or exhibit proximity quenching. Non-limiting examples of a label type that does not self-quench or exhibit proximity quenching include Bimane derivatives such as Monobromobimane. The term “proximity quenching,” as used herein, generally refers to a phenomenon where one or more dyes near each other may exhibit lower fluorescence as compared to the fluorescence they exhibit individually. In some cases, the dye may be subject to proximity quenching wherein the donor dye and acceptor dye are within 1 nm to 50 nm of each other. Examples of quenchers include, but are not limited to, Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), and ATTO fluorescent quenchers (ATTO-TEC GmbH) (e.g., ATTO 540Q, ATTO 580Q, and ATTO 612Q). Fluorophore donor molecules may be used in conjunction with a quencher. Examples of fluorophore donor molecules that can be used in conjunction with quenchers include, but are not limited to, fluorophores such as Cy3B, Cy3, or Cy5; Dy-Quenchers (Dyomics) (e.g., DYQ-660 and DYQ-661); and ATTO fluorescent quenchers (ATTO-TEC GmbH) (e.g., ATTO 540Q, 580Q, and 612Q).

The term “labeling fraction,” as used herein, generally refers to the ratio of dye-labeled nucleotide or nucleotide analog to natural/unlabeled nucleotide or nucleotide analog of a single canonical type in a flow solution. The labeling fraction can be expressed as the concentration of the labeled nucleotide or nucleotide analog divided by the sum of the concentrations of labeled and unlabeled nucleotide or nucleotide analog. The labeling fraction may be expressed as a % of labeled nucleotides included in a solution (e.g., a nucleotide flow). The labeling fraction may be at least about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or higher. For example, the labeling fraction may be at least about 20%. The labeling fraction may be about 100%. The labeling fraction may also be expressed as a ratio of labeled nucleotides to unlabeled nucleotides included in a solution. For example, the ratio of labeled nucleotides to unlabeled nucleotides may be at least about 1:10, 1:5, 1:4, 1:3, 1:2, 1:1, 2:1, 3:1, 4:1, 5:1, 10:1, or higher. For example, the ratio of labeled nucleotides to unlabeled nucleotides may be at least about at least about 1:10, 1:5, 1:4, 1:3, 1:2, 1:1, 2:1, 3:1, 4:1, 5:1, or 10:1. For example, the ratio of labeled nucleotides to unlabeled nucleotides may be at least about 1:1. The ratio of labeled nucleotides to unlabeled nucleotides may be at least about 10:1. The ratio of labeled nucleotides to unlabeled nucleotides may be at least about 5:1. The ratio of labeled nucleotides to unlabeled nucleotides may be at least about 3:1.

The term “labeled fraction,” as used herein, generally refers to the actual fraction of labeled nucleic acid (e.g., DNA) resulting after treatment of a primer-template with a mixture of the dye-labeled and natural nucleotide or nucleotide analog. The labeled fraction may be about the same as the labeling fraction. For example, if 20% of nucleotides in a nucleotide flow are labeled, about 20% of nucleotides incorporated into a growing nucleic acid strand (e.g., during nucleic acid sequencing) may be labeled. Alternatively, the labeled fraction may be greater than the labeled fraction. For example, if 20% of nucleotides in a nucleotide flow are labeled, greater than 20% of nucleotides incorporated into a growing nucleic acid strand (e.g., during nucleic acid sequencing) may be labeled. Alternatively, the labeled fraction may be less than the labeled fraction. For example, if 20% of nucleotides in a nucleotide flow are labeled, less than 20% of nucleotides incorporated into a growing nucleic acid strand (e.g., during nucleic acid sequencing) may be labeled.

When a solution including less than 100% labeled nucleotides or nucleotide analogs is used in an incorporation process such as a sequencing process (e.g., as described herein), both labeled (“bright”) and unlabeled (“dark”) nucleotides or nucleotide analogs may be incorporated into a growing nucleic acid strand. The term “tolerance,” as used herein, generally refers to the ratio of the labeled fraction (e.g., “bright” incorporated fraction) to the labeling fraction (e.g., “bright” fraction in solution). For example, if a labeling fraction of 0.2 is used resulting in a labeled fraction of 0.4 the tolerance is 2. Similarly, if an incorporation process such as a sequencing process is performed using 2.5% labeled fraction in solution (b_{f}, bright solution fraction) and 5% is labeled (bᵢ, bright incorporated fraction), the tolerance may be 2 (e.g., tolerance). This model may be linear for low labeling fractions (e.g., 10% or lower labeling fraction). For higher labeling fractions, tolerance may take into account competing dark incorporation. Tolerance may refer to a comparison of the ratio of bright incorporated fraction to dark incorporated fraction (bᵢ/dᵢ) to the ratio of bright solution fraction to dark solution fraction (b_{f}/d_{f}):

$$Tolerance = \frac{{b}_{i} / {d}_{i}}{{b}_{f} / {d}_{f}}$$

where dᵢ=1−bᵢ (e.g., dark incorporated fraction and bright incorporated fraction sum to 1 assuming 100% bright fraction is normalized to 1)

Though dᵢ cannot easily be measured, bᵢ, the bright incorporated fraction, can be measured (e.g., as described herein) and used to determine tolerance by fitting a curve of bright solution fraction (b_{f}) vs. bright incorporated fraction (bᵢ):

$${b}_{i} = \frac{tol ( {b}_{f} / {d}_{f} )}{1 + tol ( {b}_{f} / {d}_{f} )}$$

A “positive” tolerance number (>1) indicates that at 50% labeling fraction, more than 50% is labeled. A “negative” tolerance number (<1) indicates that at 50% labeling fraction, less than 50% is labeled.

The term “context,” as used herein, generally refers to the sequence of the neighboring nucleotides, or context, has been observed to affect the tolerance in an incorporation reaction. The nature of the enzyme, the pH, and other factors may also affect the tolerance. Reducing context effects to a minimum greatly simplifies base determination.

The term “scar,” as used herein, generally refers to a residue left on a previously labeled nucleotide or nucleotide analog after cleavage of an optical (e.g., fluorescent) dye and, optionally, all or a portion of a linker attaching the optical dye to the nucleotide or nucleotide analog. Examples of scars include, but are not limited to, hydroxyl moieties (e.g., resulting from cleavage of an azidomethyl group, hydrocarbyldithiomethyl linkage, or 2-nitrobenzyloxy linkage), thiol moieties (e.g., resulting from cleavage of a disulfide linkage), and benzyl moieties. For example, a scar may comprise an aromatic group such as a phenyl or benzyl group. The size and nature of a scar may affect subsequent incorporations.

The term “misincorporation,” as used herein, generally refers to occurrences when the DNA polymerase incorporates a nucleotide, either labeled or unlabeled, that is not the correct Watson-Crick partner for the template base. Misincorporation can occur more frequently in methods that lack competition of all four bases in an incorporation event, and leads to strand loss, and thus limits the read length of a sequencing method.

The term “mispair extension,” as used herein, generally refers to occurrences when the DNA polymerase incorporates a nucleotide, either labeled or unlabeled, that is not the correct Watson-Crick partner for the template base, then subsequently incorporates the correct Watson-Crick partner for the following base. Mispair extension generally results in lead phasing and limits the read length of a sequencing method.

Regarding quenching, dye-dye quenching between two dye moieties linked to different nucleotides (e.g., adjacent nucleotides in a growing nucleic acid strand, or nucleotides in a nucleic acid strand that are separated by one or more other nucleotides) may be strongly dependent on the distance between the two dye moieties. The distance between two dye moieties may be at least partially dependent on the properties of linkers connecting the two dye moieties to respective nucleotides or nucleotide analogs, including the linker compositions and functional lengths. Features of the linkers, including composition and functional length, may be affected by temperature, solvent, pH, and salt concentration (e.g., within a solution). Quenching may also vary based on the nature of the dyes used. Quenching may also take place between dye moieties and nucleobase moieties (e.g., between a fluorescent dye and a nucleobase of a nucleotide with which it is associated). Controlling quenching phenomena may be a key feature of the methods described herein.

Regarding flows, a nucleotide flow can consist of a mixture of labeled and unlabeled nucleotides or nucleotide analogs (e.g., nucleotides or nucleotide analogs of a single canonical type). For example, a solution comprising a plurality of optically (e.g., fluorescently) labeled nucleotides and a plurality of unlabeled nucleotides may be contacted with, e.g., a sequencing template (as described herein). The plurality of optically labeled nucleotides and a plurality of unlabeled nucleotides may each comprise the same canonical nucleotide or nucleotide analog. A flow may include only labeled nucleotides or nucleotide analogs. Alternatively, a flow may include only unlabeled nucleotides or nucleotide analogs. A flow may include a mixture of nucleotide or nucleotide analogs of different types (e.g., A and G).

A wash flow (e.g., a solution comprising a buffer) may be used to remove any nucleotides that are not incorporated into a nucleic acid complex (e.g., a sequencing template, as described herein). A cleavage flow (e.g., a solution comprising a cleavage reagent) may be used to remove dye moieties (e.g., fluorescent dye moieties) from optically (e.g., fluorescently) labeled nucleotides or nucleotide analogs. In some cases, different dyes (e.g., fluorescent dyes) may be removable using different cleavage reagents. In other cases, different dyes (e.g., fluorescent dyes) may be removable using the same cleavage reagents. Cleavage of dye moieties from optically labeled nucleotides or nucleotide analogs may comprise cleavage of all or a portion of a linker connecting a nucleotide or nucleotide analog to a dye moiety.

The term “cycle,” as used herein, generally refers to a process in which a nucleotide flow, a wash flow, and a cleavage flow corresponding to each canonical nucleotide (e.g., dATP, dCTP, dGTP, and dTTP or dUTP, or modified versions thereof) are used (e.g., provided to a sequencing template, as described herein). Multiple cycles may be used to sequence and/or amplify a nucleic acid molecule. The order of nucleotide flows can be varied.

Phasing can be lead or lag phasing. Lead phasing generally refers to the phenomenon in which a population of strands show incorporation of a nucleotide a flow ahead of the expected cycle (e.g., due to contamination in the system). Lag phasing refers to the phenomenon in which a population of strands shows incorporation of a nucleotide a flow behind the expected cycle (e.g., due to incompletion of extension in an earlier cycle).

Compounds and chemical moieties described herein, including linkers, may contain one or more asymmetric centers and thus give rise to enantiomers, diastereomers, and other stereoisomeric forms that are defined, in terms of absolute stereochemistry, as (R)- or (S)-, and, in terms of relative stereochemistry, as (D)- or (L)-. The D/L system relates molecules to the chiral molecule glyceraldehyde and is commonly used to describe biological molecules including amino acids. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a phenyl ring. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions,” John Wiley and Sons, Inc., 1981, herein incorporated by reference for this disclosure). Stereoisomers may also be obtained by stereoselective synthesis.

Compounds and chemical moieties described herein, including linkers, may exist as tautomers. A “tautomer” refers to a molecule wherein a proton shift from one atom of a molecule to another atom of the same molecule is possible. In circumstances where tautomerization is possible, a chemical equilibrium of the tautomers may exist. Unless otherwise stated, chemical structures depicted herein are intended to include structures which are different tautomers of the structures depicted. For example, the chemical structure depicted with an enol moiety also includes the keto tautomer form of the enol moiety. The exact ratio of the tautomers depends on several factors, including physical state, temperature, solvent, and pH. Some examples of tautomeric equilibrium include:

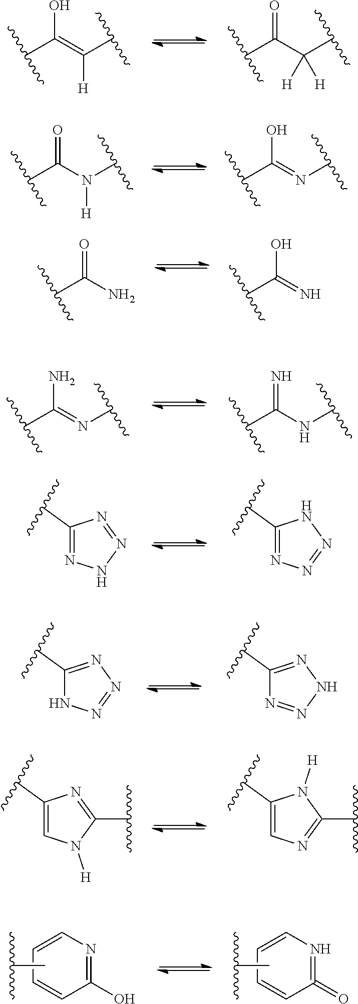

Compounds and chemical moieties described herein, including linkers and dyes, may be provided in different enriched isotopic forms. For example, compounds may be enriched in the content of ²H, ³H, ¹¹C, ¹³C and/or ¹⁴C. For example, a linker, substrate (e.g., nucleotide or nucleotide analog), or dye may be deuterated in at least one position. In some examples, a linker, substrate (e.g., nucleotide or nucleotide analog), or dye may be fully deuterated. Such deuterated forms can be made by the procedure described in U.S. Pat. Nos. 5,846,514 and 6,334,997, each of which are herein incorporated by reference in their entireties. As described in U.S. Pat. Nos. 5,846,514 and 6,334,997, deuteration can improve the metabolic stability and or efficacy, thus increasing the duration of action of drugs.

Unless otherwise stated, structures depicted and described herein are intended to include compounds which differ only in the presence of one or more isotopically enriched atoms. For example, compounds and chemical moieties having the present structures except for the replacement of a hydrogen by a deuterium or tritium, or the replacement of a carbon by ¹³C- or ¹⁴C-enriched carbon are within the scope of the present disclosure.

The compounds and chemical moieties of the present disclosure may contain unnatural proportions of atomic isotopes at one or more atoms that constitute such compounds. For example, a compound or chemical moiety such as a linker, substrate (e.g., nucleotide or nucleotide analog), or dye, or a combination thereof, may be labeled with one or more isotopes, such as deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds and chemical moieties described herein, whether radioactive or not, are encompassed within the scope of the present disclosure.

### Linkers

The present disclosure provides linkers for coupling a labeling reagent and a substrate. The present disclosure also provides an optical (e.g., fluorescent) labeling reagent comprising a dye (e.g., fluorescent dye) and a linker that is connected to the dye and configured to couple to a substrate for optically (e.g., fluorescently) labeling the substrate. A substrate may comprise a detectably labeled substrate. The substrate may comprise a labeling reagent. For example, the substrate may be coupled to the labeling reagent. In some cases, a substrate may be modified. In some cases, the substrate may comprise a linker. In some cases, the substrate may comprise the linker and the labeling reagent. The substrate can be any suitable molecule, analyte, cell, tissue, or surface that is to be optically labeled. Examples include cells, including eukaryotic cells, prokaryotic cells, healthy cells, and diseased cells; cellular receptors; antibodies; proteins; lipids; metabolites; saccharides; polysaccharides; probes; reagents; nucleotides and nucleotide analogs (e.g., as described herein); polynucleotides; and nucleic acid molecules. For example, the substrate may be a nucleotide or nucleotide analog. In another example, the substrate may be a protein such as an antibody, such as a protein (e.g., antibody) that is a component of a cell. An association between a linker and a substrate can be any suitable association including a covalent or non-covalent bond. For example, a linker of an optical labeling reagent may be coupled to a substrate (e.g., nucleotide or nucleotide analog) via a nucleobase of a nucleotide, such as a nucleotide in a nucleic acid molecule, via, e.g., a propargyl or propargylamino moiety. In another example, a linker of an optical labeling reagent may be coupled to a substrate (e.g., protein, such as an antibody) via an amino acid of a polypeptide or protein. In some cases, an association between a linker and a substrate may be a biotin-avidin interaction. In other cases, an association between a linker and a substrate may be via a propargylamino moiety. In some cases, an association between a linker and a substrate may be via an amide bond (e.g., a peptide bond). A labeling reagent may comprise a cleavable moiety configured to be cleaved to separate the labeling reagent or a portion thereof from a substrate to which it is attached. Various linkers, labeling reagents, labels, substrates, and combinations thereof are described in further detail in U.S. Pat. No. 11,377,680B2 and International Patent Pub. No. WO2022/040213A1, each of which is entirely incorporated by reference herein for all purposes.

In an aspect, the present disclosure provides a labeling reagent. The labeling agent (e.g., a fluorescent labeling reagent) may comprise an optically detectable moiety such as a fluorescent dye moiety. A labeling reagent may comprise multiple optically detectable moieties, such as multiple fluorescent dye moieties, that may have the same or different chemical structures and may generate signal (e.g., fluoresce) at the same or different wavelengths. A labeling reagent may also comprise a linker that is coupled to label or detectable moiety. A labeling reagent may also comprise a linker that is coupled to or connected to an optically detectable moiety (e.g., a fluorescent dye moiety). The linker may comprise one or more components, including one or more semi-rigid portions, spacer portions, cleavable portions, etc. The linker may comprise a first linker and/or a second linker. A linker may comprise at least about one non-proteinogenic amino acid. A linker may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-proteinogenic amino acids. A linker may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 non-proteinogenic amino acids.

A non-proteinogenic amino acid may comprise (all-S,all-E)-3-amino-9-methoxy-2,6,8-trimethyl-10-phenyldeca-4,6-dienoic acid (ADDA), 2-aminoisobutyric acid, 4-aminobenzoic acid, 4-hydroxyphenylglycine, 6-aminohexanoic acid, aminolevulinic acid, azetidine-2-carboxylic acid, canaline, canavanine, carboxyglutamic acid, chloroalanine, citrulline, cysteic acid, 5-amino-5-carboxy-N,N,N-trimethylpentan-1-aminium (also known as 2-amino-6-(trimethylammonio)hexanoate), dehydroalanine, diaminopimelic acid, dihydroxyphenylglycine, enduracididine, gamma-aminobutyric acid, hawkinsin, homocysteine, homoserine, hydroxyproline, hypusine, lanthionine, norleucine, norvaline, nv-5138, omithine, penicillamine, plakohypaphorine, pyroglutamic acid, quisqualic acid, s-aminoethyl-l-cysteine, sarcosine, theanine, tranexamic acid, tricholomic acid, β-alanine, or β-leucine.

A non-proteinogenic amino acid may be aliphatic, branched, or cyclic. In some cases, the non-proteinogenic amino acid may be aliphatic. In some cases, the non-proteinogenic amino acid may be branched. In some cases, the non-proteinogenic amino acid may be cyclic. In other cases, the non-proteinogenic amino acid may be non-cyclic. In some cases, the non-proteinogenic amino acid may be positively charged. In some cases, the non-proteinogenic amino acid may carry at least 1, 2, 3, 4, 5, or more positive charges. In some cases, the non-proteinogenic amino acid may be negatively charged. the non-proteinogenic amino acid may carry at least 1, 2, 3, 4, 5, or more negative charges. The non-proteinogenic amino acid may also be neutral or not carry a charge. A non-proteinogenic amino acid may comprise at least one side-chain chemical moiety. A non-proteinogenic amino acid may comprise at least 1, 2, 3, 4, 5, or more side chain chemical moieties. In some cases, the side-chain chemical moiety may be aliphatic, branched, or cyclic. In some cases, the side-chain chemical moiety may be aliphatic. In some cases, the side-chain chemical moiety may be branched. In some cases, the side-chain chemical moiety may be cyclic. In other cases, the side-chain chemical moiety may be non-cyclic. In some cases, the side-chain chemical moiety may be positively charged. In some cases, the side-chain chemical moiety may carry at least 1, 2, 3, 4, 5, or more positive charges. In some cases, the side-chain chemical moiety may be negatively charged. In some cases, the side-chain chemical moiety may carry at least 1, 2, 3, 4, 5, or more negative charges. The side-chain chemical moiety may also be neutral or not carry a charge.

A non-proteinogenic amino acid may comprise cysteic acid. Cysteic acid may have a structure below:

A cysteic acid, when coupled to a substrate (e.g., a nucleotide) may reduce an enzyme affinity to the substrate. For example, cysteic acid may decrease or lower the affinity of a polymerase described herein to a nucleotide. The decreased or lower affinity of the polymerase to the nucleotide may reduce the enzymatic processing of the nucleotide by the polymerase. In some cases, cysteic acid may decrease or lower the affinity of Bst polymerase and a nucleotide coupled to the cysteic acid. In some cases, the lower affinity may comprise at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 95%, 96%, 97%, 98%, 99% of the affinity when compared to the affinity of the polymerase to the natural nucleotide substrate. In some cases, the lower affinity may comprise at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 95%, 96%, 97%, 98%, or 99% of the affinity when compared to the affinity of the polymerase to the natural nucleotide substrate.

A non-proteinogenic amino acid may comprise 5-amino-5-carboxy-N,N,N-trimethylpentan-1-aminium or a salt thereof. 5-Amino-5-carboxy-N,N,N-trimethylpentan-1-aminium may have a structure below:

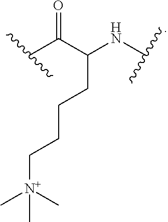 [SMILES: *NC(CCCC[N+](C)(C)C)C(*)=O]

A 5-amino-5-carboxy-N,N,N-trimethylpentan-1-aminium, when coupled to a substrate (e.g., a nucleotide) may increase an enzyme affinity to the substrate. For example, the presence of 5-amino-5-carboxy-N,N,N-trimethylpentan-1-aminium in the substrate may increase the affinity of a polymerase described herein to a nucleotide. The increased or higher affinity of the polymerase to the nucleotide may increase the enzymatic processing of the nucleotide by the polymerase. In some cases, 5-amino-5-carboxy-N,N,N-trimethylpentan-1-aminium may increase the affinity of Bst polymerase to a nucleotide coupled to the 5-amino-5-carboxy-N,N,N-trimethylpentan-1-aminium. In some cases, the higher affinity may comprise at least about 101%, 102%, 103%, 104%, 105%, 106%, 107%, 108%, 109%, 110%, 115%, 120%, 125%, 130%, 135%, 140%, 145%, 150%, 160%, 170%, 180%, 190%, 195%, 196%, 197%, 198%, 199% or higher the affinity when compared to the affinity of the polymerase to the natural nucleotide substrate. In some cases, the higher affinity may comprise at most about 101%, 102%, 103%, 104%, 105%, 106%, 107%, 108%, 109%, 110%, 115%, 120%, 125%, 130%, 135%, 140%, 145%, 150%, 160%, 170%, 180%, 190%, 195%, 196%, 197%, 198%, 199% of the affinity when compared to the affinity of the polymerase to the natural nucleotide substrate.

A linker may have a cleavable moiety. A linker may have a cleavable moiety and at least about one cysteic acid. A linker may have a cleavable moiety and one cysteic acid. A linker may have a structure below:

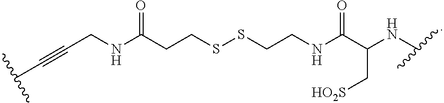

A linker may have a cleavable moiety and at least about one 5-amino-5-carboxy-N,N,N-trimethylpentan-1-aminium or a salt thereof. A linker may have a cleavable moiety and one 5-amino-5-carboxy-N,N,N-trimethylpentan-1-aminium or a salt thereof. A linker may have a structure below:

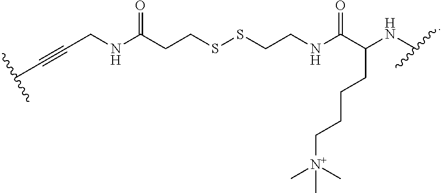 [SMILES: *C#CCNC(=O)CCSSCCNC(=O)C(CCCC[N+](C)(C)C)N*]

A linker may have a cleavable moiety and at least about one 5-amino-5-carboxy-N,N,N-trimethylpentan-1-aminium or a salt thereof and at least one cysteic acid. A linker may have a cleavable moiety, one 5-amino-5-carboxy-N,N,N-trimethylpentan-1-aminium or a salt thereof, and one cysteic acid. A linker may have a structure below:

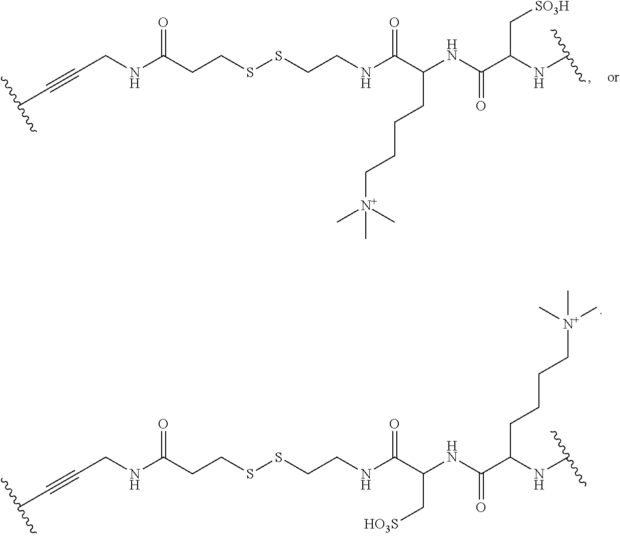 [SMILES: *C#CCNC(=O)CCSSCCNC(=O)C(CCCC[N+](C)(C)C)NC(=O)C(CS(=O)(=O)O)N* ; *C#CCNC(=O)CCSSCCNC(=O)C(CS(=O)(=O)O)NC(=O)C(CCCC[N+](C)(C)C)N*]

A non-proteinogenic amino acid may comprise 6-aminohexanoic acid. 6-aminohexanoic acid may have a structure below:

The linker may have a cleavable moiety and at least about one 6-aminohexanoic acid. The linker may have a cleavable moiety and one 6-aminohexanoic acid. The linker may have a structure below:

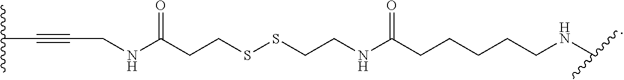 [SMILES: *C#CCNC(=O)CCSSCCNC(=O)CCCCCN*]

A linker may have a chemical formula below:

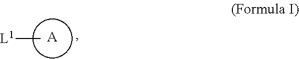

wherein: A is a detectable moiety; and L¹ is a linker comprising at least one cleavable group and at least one non-proteinogenic amino acid.

In some cases, L¹ may comprise a linker described herein. In some cases, the non-proteinogenic amino acid may not comprise hydroxyproline. In some cases, the non-proteinogenic amino acid may comprise cysteic acid, 5-amino-5-carboxy-N,N,N-trimethylpentan-1-aminium or a salt thereof, or 6-aminohexanoic acid. In some cases, the non-proteinogenic amino acid may comprise at least about one cysteic acid. In some cases, the non-proteinogenic amino acid may comprise at least about two cysteic acids. A linker may comprise at least about 1, 2, 3, 4, 5, 6, 7, 8, 9, 10 or more cysteic acids. A linker may comprise at most about 1, 2, 3, 4, 5, 6, 7, 8, 9, or 10 cysteic acids. In some cases, the non-proteinogenic amino acid may comprise at least about one 5-amino-5-carboxy-N,N,N-trimethylpentan-1-aminium or a salt thereof. In some cases, the non-proteinogenic amino acid may comprise at least about two 5-amino-5-carboxy-N,N,N-trimethylpentan-1-aminium or a salt thereof. A linker may comprise at least about 1, 2, 3, 4, 5, 6, 7, 8, 9, 10 or more 5-amino-5-carboxy-N,N,N-trimethylpentan-1-aminium or a salt thereof. A linker may comprise at most about 1, 2, 3, 4, 5, 6, 7, 8, 9, or 10 5-amino-5-carboxy-N,N,N-trimethylpentan-1-aminium or a salt thereof. In some cases, the non-proteinogenic amino acid may comprise at least about one 6-aminohexanoic acid. A linker may comprise at least about 1, 2, 3, 4, 5, 6, 7, 8, 9, 10 or more 6-aminohexanoic acids. A linker may comprise at most about 1, 2, 3, 4, 5, 6, 7, 8, 9, or 10 6-aminohexanoic acids. In some cases, when the non-proteinogenic amino acid comprises 6-aminohexanoic acid, the detectable moiety may not comprise a Cyanide 5 (Cy5) or an ATTO 647 NHS Ester (ATTO 647N). In some cases, when the at least one non-proteinogenic amino acid comprises 6-aminohexanoic acid, the linker may not be coupled to a terminator. Such a terminator may comprise a chemical entity that can block a nucleotide polymerization reaction (e.g., a nucleotide polymerization reaction in a sequencing reaction). In some cases, when the at least one non-proteinogenic amino acid comprises 6-aminohexanoic acid, the linker may not be coupled to the structures below:

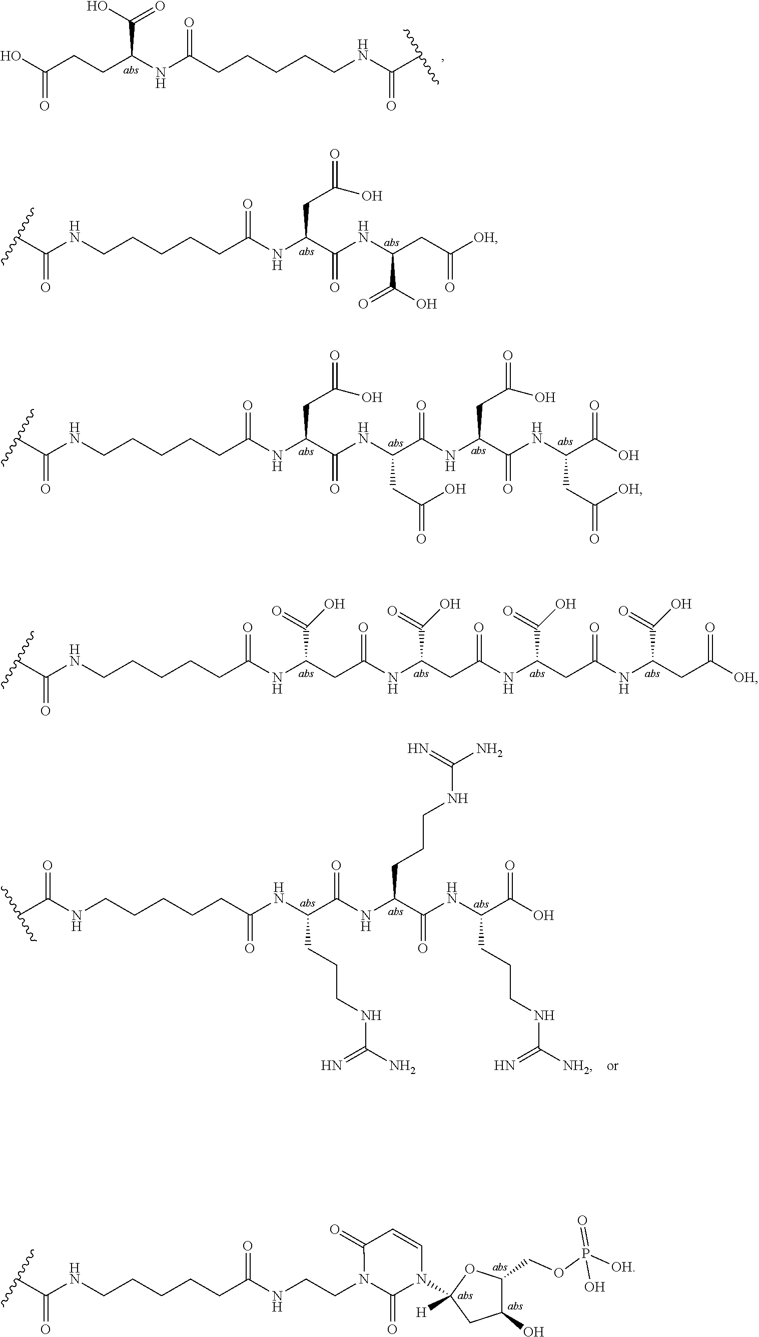 [SMILES: *C(=O)NCCCCCC(=O)N[C@@H](CCC(=O)O)C(=O)O ; *C(=O)NCCCCCC(=O)N[C@@H](CC(=O)O)C(=O)N[C@@H](CC(=O)O)C(=O)O ; *C(=O)NCCCCCC(=O)N[C@@H](CC(=O)O)C(=O)N[C@@H](CC(=O)O)C(=O)N[C@@H](CC(=O)O)C(=O)N[C@@H](CC(=O)O)C(=O)O ; *C(=O)NCCCCCC(=O)N[C@@H](CC(=O)N[C@@H](CC(=O)N[C@@H](CC(=O)N[C@@H](CC(=O)O)C(=O)O)C(=O)O)C(=O)O)C(=O)O ; *C(=O)NCCCCCC(=O)N[C@@H](CCCNC(=N)N)C(=O)N[C@@H](CCCNC(=N)N)C(=O)N[C@@H](CCCNC(=N)N)C(=O)O ; *C(=O)NCCCCCC(=O)NCCn1c(=O)ccn([C@H]2C[C@H](O)[C@@H](COP(=O)(O)O)O2)c1=O]

In some cases, L¹ may comprise a linker described herein. In some case, when the at least one non-proteinogenic amino acid comprises 6-aminohexanoic acid, the linker may not be coupled to a terminator moiety of a sequencing or a nucleic acid polymerization reaction. In some cases, L¹ may comprise a cleavable group or moiety.

In some cases, a detectably labeled substrate may comprise a chemical formula below:

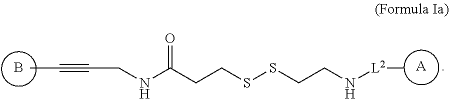

In some cases, a detectably labeled substrate may comprise chemical Formula Ia. In some cases, B may comprise a substrate. In some cases, B may comprise a nucleobase. In some cases, B may comprise a nucleoside. In some cases, B may comprise a nucleotide. In some cases, B may comprise a deoxyribose nucleotide triphosphate. In some cases, B may comprise a ribose nucleotide triphosphate. In some cases, L² is a linker and may comprise a non-proteinogenic amino acid. In some cases, the non-proteinogenic amino acid may comprise any non-proteinogenic amino acids described herein (e.g., cysteic acid, 5-amino-5-carboxy-N,N,N-trimethylpentan-1-aminium or a salt thereof, 6-aminohexanoic acid, and/or hydroxyproline). In some cases, A may be the detectable moiety described herein. As described herein, A may comprise a ring structure. In some cases, A may be a ring structure. In some cases, B may comprise a ring structure. In some cases, B may be a ring structure.

The linker may comprise at least about one non-proteinogenic amino acid. The linker may comprise one non-proteinogenic amino acid. The linker may comprise at least about two non-proteinogenic amino acids. The linker may comprise two non-proteinogenic amino acids. The two non-proteinogenic amino acids may be different. The two non-proteinogenic amino acids may be the same.

A non-proteinogenic amino acid may comprise hydroxyproline. The linker may comprise at least one non-proteinogenic amino acid, such as at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more hydroxyprolines. For example, the linker may comprise at least about 10 non-proteinogenic amino acids, such as at least 10 hydroxyprolines and at least one different non-proteinogenic amino acid. In some cases, the linker may comprise at least about 10 non-proteinogenic amino acids, such as at least about 10 hydroxyprolines and at least about two additional non-proteinogenic amino acids. The two additional non-proteinogenic amino acids may be a same type. The two additional non-proteinogenic amino acids may be the same. The two additional non-proteinogenic amino acids may also be different. The linker may comprise at least about 10 hydroxyprolines and at least about one cysteic acid. The linker may comprise about 10 hydroxyprolines and one cysteic acid. The linker may comprise at least about 10 hydroxyprolines and at least about two cysteic acids. The linker may comprise about 10 hydroxyprolines and about two cysteic acids.

The linker may comprise at least about 10 hydroxyprolines and at least about one 5-amino-5-carboxy-N,N,N-trimethylpentan-1-aminium or a salt thereof. The linker may comprise about 10 hydroxyprolines and one 5-amino-5-carboxy-N,N,N-trimethylpentan-1-aminium or a salt thereof. The linker may comprise at least about 10 hydroxyprolines and at least about two 5-amino-5-carboxy-N,N,N-trimethylpentan-1-aminium or a salt thereof. The linker may comprise about 10 hydroxyprolines and about two 5-amino-5-carboxy-N,N,N-trimethylpentan-1-aminium or a salt thereof.

The linker may comprise at least about 10 hydroxyprolines and at least two additional non-proteinogenic amino acids that may be different. The at least two additional non-proteinogenic amino acids are at least one cysteic acid and at least one 5-amino-5-carboxy-N,N,N-trimethylpentan-1-aminium or a salt thereof. The linker may comprise about 10 hydroxyprolines, one cysteic acid and one 5-amino-5-carboxy-N,N,N-trimethylpentan-1-aminium or a salt thereof. The linker may comprise at least about 10 hydroxyprolines and at least about two cysteic acids and at least one 5-amino-5-carboxy-N,N,N-trimethylpentan-1-aminium or a salt thereof. The linker may comprise at least about 10 hydroxyprolines and at least one cysteic acid and at least two 5-amino-5-carboxy-N,N,N-trimethylpentan-1-aminium or a salt thereof. The linker may comprise about 10 hydroxyprolines, one cysteic acid, and about two 5-amino-5-carboxy-N,N,N-trimethylpentan-1-aminium or a salt thereof. The linker may comprise about 10 hydroxyprolines, about two cysteic acid, and one 5-amino-5-carboxy-N,N,N-trimethylpentan-1-aminium or a salt thereof.

The linker may comprise at least about 10 hydroxyprolines and at least about one 6-aminohexanoic acid. The linker may comprise about 10 hydroxyprolines and about one 6-aminohexanoic acid.

In another example, the linker may comprise at least about 20 non-proteinogenic amino acids and at least one different non-proteinogenic amino acid. The linker may comprise at least about 20 hydroxyprolines and at least about one cysteic acid. The linker may comprise about 20 hydroxyprolines and about one cysteic acid. The linker may comprise at least about 20 hydroxyprolines and at least about two cysteic acids. The linker may comprise about 20 hydroxyprolines and about two cysteic acids.

The linker may comprise at least about 20 hydroxyprolines and at least about one 5-amino-5-carboxy-N,N,N-trimethylpentan-1-aminium or a salt thereof. The linker may comprise about 20 hydroxyprolines and one 5-amino-5-carboxy-N,N,N-trimethylpentan-1-aminium or a salt thereof. The linker may comprise at least about 20 hydroxyprolines and at least about two 5-amino-5-carboxy-N,N,N-trimethylpentan-1-aminium or a salt thereof. The linker may comprise about 20 hydroxyprolines and about two 5-amino-5-carboxy-N,N,N-trimethylpentan-1-aminium or a salt thereof.

The linker may comprise at least about 20 hydroxyprolines and at least about one 6-aminohexanoic acid. The linker may comprise 20 hydroxyprolines and one 6-aminohexanoic acid. Two different non-proteinogenic amino acids may be coupled to each other directly. Two different non-proteinogenic amino acids may be coupled to each other indirectly (e.g., via a chemical moiety). Non-proteinogenic amino acids of a linker may be included in any useful portion of the linker and may be included in sequence or separated by one or more other chemical moieties (e.g., as described herein). The linker may be configured to couple to a substrate for optically (e.g., fluorescently) labeling the substrate. The substrate may be, for example, a nucleotide or nucleotide analog, nucleic acid molecule, polynucleotide, protein, antibody, cell, saccharide, polysaccharide, lipid, or any other substrate described herein. The labeling reagent may comprise a cleavable moiety configured to be cleaved to separate the labeling reagent or a portion thereof from the substrate.

In another aspect, the present disclosure provides a labeling reagent (e.g., a fluorescent labeling reagent) comprising an optically detectable moiety such as a fluorescent dye moiety. A labeling reagent may comprise multiple optically detectable moieties, such as multiple fluorescent dye moieties, that may have the same or different chemical structures and may generate signal (e.g., fluoresce) at the same or different wavelengths. A labeling reagent may also comprise a linker that is connected to an optically detectable moiety (e.g., a fluorescent dye moiety). The linker may comprise one or more components, including one or more semi-rigid portions, spacer portions, cleavable portions, etc. For example, the linker may comprise a semi-rigid portion. The semi-rigid portion of the linker may provide physical separation between a substrate to which the labeling reagent couples and an optically detectable moiety, which physical separation may facilitate, e.g., effective labeling of the substrate with the labeling reagent, effective detection of the labeling reagent coupled to the substrate, effective labeling of the substrate with additional labeling reagents (e.g., in the case of incorporation into homopolymeric regions of a nucleic acid template, as described herein), etc. The semi-rigid portion may provide physical separation of, on average, at least 9 Angstrom (A) between a substrate to which a labeling reagent is coupled and an optically detectable moiety of the labeling reagent. For example, the semi-rigid portion may provide physical separation of, on average, at least 9 Å, 12 Å, 15 Å, 18 Å, 21 Å, 24 Å, 27 Å, 30 Å, 33 Å, 36 Å, 39 Å, 42 Å, 45 Å, 48 Å, 51 Å, 54 Å, 57 Å, 60 Å, 63 Å, 66 Å, 69 Å, 72 Å, 75 Å, 78 Å, 81 Å, 84 Å, 87 Å, 90 Å, or more between a substrate to which a labeling reagent is coupled and an optically detectable moiety of the labeling reagent. This average separation may vary with environmental conditions including, for example, solvents (or lack thereof), temperature, pH, pressure, etc. In an example, a semi-rigid portion of a linker may comprise a secondary structure such as a helical structure that establishes and maintains a degree of physical separation between a substrate and an optically detectable moiety. For example, a semi-rigid portion of a linker may comprise a second structure such as a helical structure comprising 3 or more prolines and/or hydroxyprolines. The linker may comprise at least one non-proteinogenic amino acid, such as at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more non-proteinogenic amino acids. For example, the linker may comprise at least 10 non-proteinogenic amino acids, such as at least 10 hydroxyprolines. In another example, the linker may comprise at least 20 non-proteinogenic amino acids. Non-proteinogenic amino acids of a linker may be included in any useful portion of the linker and may be included in sequence or separated by one or more other chemical moieties (e.g., as described herein). For example, a linker may comprise a first semi-rigid portion and a second semi-rigid portion separated by another moiety, where the first and second semi-rigid portions comprise secondary structures such as helical structures. The linker may be configured to couple to a substrate for optically (e.g., fluorescently) labeling the substrate. The substrate may be, for example, a nucleotide or nucleotide analog, polynucleotide, nucleic acid molecule, protein, antibody, cell, saccharide, polysaccharide, lipid, or any other substrate described herein. The labeling reagent may comprise a cleavable moiety configured to be cleaved to separate the labeling reagent or a portion thereof from the substrate.

In another aspect, the present disclosure provides a labeling reagent (e.g., a fluorescent labeling reagent) comprising an optically detectable moiety such as a fluorescent dye moiety. A labeling reagent may comprise multiple optically detectable moieties, such as multiple fluorescent dye moieties, that may have the same or different chemical structures and may generate signal (e.g., fluoresce) at the same or different wavelengths. A labeling reagent may comprise the general structure: (cleavable linker moiety)-(semi-rigid linker moiety)-(optically detectable moiety). Each component of this general structure may be separated by one or more additional moieties, including one or more spacer moieties. In some cases, a labeling reagent may comprise a scaffold that permits the inclusion of multiple semi-rigid linker moieties and/or optically detectable moieties (e.g., fluorescent dye moieties). For example, a labeling reagent may comprise a branching or dendritic structure. A labeling reagent may also comprise one or more additional features including one or more spacer portions. The labeling reagent may comprise at least one non-proteinogenic amino acid, such as at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more non-proteinogenic amino acids. For example, the linker may comprise at least 10 non-proteinogenic amino acids, such as at least 10 hydroxyprolines. In another example, the linker may comprise at least 20 non-proteinogenic amino acids. Non-proteinogenic amino acids of a linker may be included in any useful portion of the linker and may be included in sequence or separated by one or more other chemical moieties (e.g., as described herein). One or more non-proteinogenic amino acids may be included in a semi-rigid linker portion. For example, a semi-rigid linker portion may comprise a secondary structure such as a helical portion comprising one or more prolines and/or hydroxyprolines. The labeling reagent may be configured to couple to a substrate for optically (e.g., fluorescently) labeling the substrate. The substrate may be, for example, a nucleotide or nucleotide analog, polynucleotide, nucleic acid molecule, protein, antibody, cell, saccharide, polysaccharide, lipid, or any other substrate described herein. The labeling reagent may comprise a cleavable moiety configured to be cleaved to separate the labeling reagent or a portion thereof from the substrate.

A linker may comprise one or more regions having a semi-rigid structure. For example, a linker may comprise at least one region having a semi-rigid structure, such as 1, 2, 3, 4, 5, 6, 7, 8, 9, or more regions having a semi-rigid structure. A region of a linker having a semi-rigid structure may be adjacent to another region of the linker having a semi-rigid structure. Alternatively or in addition, a region of a linker having a semi-rigid structure may be adjacent to another region of the linker that does not have a semi-rigid structure. Similarly, an optical (e.g., fluorescent) labeling reagent may comprise one or more regions having a semi-rigid structure. For example, an optical (e.g., fluorescent) labeling reagent may comprise at least one region having a semi-rigid structure, such as 1, 2, 3, 4, 5, 6, 7, 8, 9, or more regions having a semi-rigid structure. Semi-rigid structures of an optical (e.g., fluorescent) labeling reagent may be included in the same or different linkers. For example, an optical (e.g., fluorescent) labeling reagent may comprise a first linker having a first semi-rigid structure and a second linker having a second semi-rigid structure, where the first and second semi-rigid structures may have the same or different chemical structures. Two or more semi-rigid structures with the same or different chemical structures may be coupled to separate portions of a structure of a labeling reagent. For example, a labeling reagent may comprise a scaffold, such as a scaffold comprising one or more lysine moieties, to which multiple different semi-rigid structures may couple at different locations to provide a branched or dendritic labeling reagent structure. Alternatively or additionally, a given linker of an optical (e.g., fluorescent) labeling reagent may comprise multiple semi-rigid structures (e.g., adjacent to one another or separated by one or more other moieties, such as by one or more amino acids) that do not contribute to a semi-rigid structure. For example, a first semi-rigid structure may be separated from a second semi-rigid structure by at least a glycine moiety.

The semi-rigid nature of a linker or a portion thereof may be attributable, at least in part, to a structure that comprises a series of ring systems (e.g., aliphatic and aromatic rings). As used herein, a ring (e.g., ring structure) is a cyclic moiety comprising any number of atoms connected in a closed, essentially circular fashion, as used in the field of organic chemistry. A ring may be defined by any number of atoms. For example, a ring may include between 3-12 atoms, such as between 3-12 carbon atoms. In certain examples, a ring may be a five-membered ring (i.e., a pentagon) or a six-membered ring (i.e., a hexagon). A ring can be aromatic or non-aromatic. A ring may be aliphatic. A ring may comprise one or more double bonds.

A ring (e.g., ring structure) may be a component of a ring system that may comprise one or more ring structures (e.g., a multi-cycle system). For example, a ring system may comprise a monocycle. In another example, a ring system may be a bicycle or bridged system. A ring structure may be a carbocycle or component thereof formed of carbon atoms. A carbocycle may be a saturated, unsaturated, or aromatic ring in which each atom of the ring is carbon. A carbocycle includes 3- to 10-membered monocyclic rings, 4- to 12-membered bicyclic rings (e.g., 6- to 12-membered bicyclic rings), and 5- to 12-membered bridged rings. Each ring of a bicyclic carbocycle may be selected from saturated, unsaturated, and aromatic rings. For example, a bicyclic carbocycle may include an aromatic ring (e.g., phenyl) fused to a saturated or unsaturated ring (e.g., cyclohexane, cyclopentane, or cyclohexene). A bicyclic carbocycle may include any combination of saturated, unsaturated, and aromatic bicyclic rings, as valence permits. A bicyclic carbocycle may include any combination of ring sizes such as 4-5 fused ring systems, 5-5 fused ring systems, 5-6 fused ring systems, and 6-6 fused ring systems. A carbocycle may be, for example, cyclopropyl, cyclobutyl, cyclopentyl, cyclohexyl, cyclohexenyl, adamantyl, phenyl, indanyl, or naphthyl. A saturated carbocycle includes no multiple bonds (e.g., double or triple bonds). A saturated carbocycle may be, for example, cyclopropane, cyclobutane, cyclopentane, or cyclohexane. An unsaturated carbocycle includes at least one multiple bond (e.g., double or triple bond) but is not an aromatic carbocycle. An unsaturated carbocycle may be, for example, cyclohexadiene, cyclohexene, or cyclopentene. Other examples of carbocycles include, but are not limited to, cyclopropane, cyclobutane, cyclopentane, cyclopentadiene, cyclohexane, cycloheptane, cycloheptene, naphthalene, and adamantine. An aromatic carbocycle (e.g., aryl moiety) may be, for example, phenyl, naphthyl, or dihydronaphthyl.

In some cases, a ring may include one or more heteroatoms, such as one or more oxygen, nitrogen, silicon, phosphorous, boron, or sulfur atoms. A ring may be a heterocycle or component thereof including one or more heteroatoms. A heterocycle may be a saturated, unsaturated, or aromatic ring in which at least one atom is a heteroatom. A heteroatom includes 3- to 10-membered monocyclic rings, 6- to 12-membered bicyclic rings, and 6- to 12-membered bridged rings. A bicyclic heterocycle may include any combination of saturated, unsaturated and aromatic bicyclic rings, as valence permits. For example, a heteroaromatic ring (e.g., pyridyl) may be fused to a saturated or unsaturated ring (e.g., cyclohexane, cyclopentane, morpholine, piperidine or cyclohexene). A bicyclic heterocycle may include any combination of ring sizes such as 4-5 fused ring systems, 5-5 fused ring systems, 5-6 fused ring systems, and 6-6 fused ring systems. An unsaturated heterocycle includes at least one multiple bond (e.g., double or triple bond) but is not an aromatic heterocycle. An unsaturated heterocycle may be, for example, dihydropyrrole, dihydrofuran, oxazoline, pyrazoline, or dihydropyridine. Additional examples of heterocycles include, but are not limited to, indole, benzothiophene, benzothiazole, benzoxazole, benzimidazole, oxazolopyridine, imidazopyridine, thiazolopyridine, furan, oxazole, pyrrole, pyrazole, imidazole, thiophene, thiazole, isothiazole, and isoxazole. A heteroaryl moiety may be an aromatic single ring structure, such as a 5- to 7-membered ring, including at least one heteroatom, such as one to four heteroatoms. Alternatively, a heteroaryl moiety may be a polycyclic ring system having two or more cyclic rings in which two or more atoms are common to two adjoining rings wherein at least one of the rings is heteroaromatic. Heteroaryl groups include, for example, pyrrole, furan, thiophene, imidazole, oxazole, thiazole, pyrazole, pyridine, pyrazine, pyridazine, and pyrimidine, and the like.

A ring can be substituted or un-substituted. A substituent replaces a hydrogen atom on one or more atoms of a ring or a substitutable heteroatom of a ring (e.g., NH or NH₂). Substitution is in accordance with permitted valence of the various components of the ring system and provides a stable compound (e.g., a compound that does not undergo spontaneous transformation by, for example, rearrangement, elimination, or cyclization). A substituent may replace a single hydrogen atom or multiple hydrogen atoms (e.g., on the same ring atom or different ring atoms). A substituent on a ring may be, for example, halogen, hydroxy, oxo, thioxo, thiol, amido, amino, carboxy, nitrilo, cyano, nitro, imino, oximino, hydrazino, alkoxy, alkenyl, alkynyl, aryl, aralkyl, aralkenyl, aralkynyl, cycloalkyl, cycloalkylalkyl, alkylcycloalkyl, heterocycloalkyl, heterocyclyl, alkylheterocyclyl, or any other useful substituent. A substituent may be water-soluble. Examples of water-soluble substituents include, but are not limited to, a pyridinium, an imidazolium, a quaternary ammonium group, a sulfonate, a sulfate, a phosphate, an alcohol, an amine, an imine, a nitrile, an amide, a thiol, a carboxylic acid, a polyether, an aldehyde, a boronic acid, and a boronic ester.

A linker, or a semi-rigid portion thereof, can have any number of rings, including at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more rings. The rings can share an edge in some cases (e.g., be components of a bicyclic ring system). In general, the ring portion of the linker can provide a degree of physical rigidity to the linker and/or can serve to physically separate the dye (e.g., fluorescent dye) on one end of the linker from the substrate to be labeled and/or from a second dye (e.g., fluorescent dye) associated with the substrate and/or associated with the linker. A ring can be a component of an amino acid (e.g., a non-proteinogenic amino acid, as described herein). For example, a linker may comprise a proline moiety. In another example, a linker may comprise a hydroxyproline moiety. For example, a linker, or a semi-rigid portion thereof, may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more proline or hydroxyproline moieties.

In some cases, a linker may comprise a “fully rigid” (e.g., substantially inflexible) portion. For example, a linker may comprise a region including ring systems that may not be separated by any sp² or sp³ carbon atoms. In general, sp² and sp³ carbon atoms (e.g., between ring systems) provide a linker or portion thereof with a degree of physical flexibility. sp³ carbon atoms in particular can confer significant flexibility. Without limitation, flexibility can allow a polymerase to accept a substrate (e.g., a nucleotide or nucleotide analog) modified with the linker and the dye (e.g., fluorescent dye), or otherwise improve the performance of a labeled system. However, in a multiple dye system (e.g., a system comprising multiple fluorescent labeling reagents, such as a polynucleotide including two or more nucleotides coupled to two or more fluorescent labeling reagents), an overly flexible linker may defeat the feature of rigidity and allow two dyes (e.g., fluorescent dyes) to come into close association and be quenched. Accordingly, ring systems of a linker or portion thereof may be connected to each other by a limited number of sp³ bonds, such as by no more than two sp³ bonds (e.g., 0, 1, or 2 sp³ bonds), to, e.g., confer a degree of rigidity to the linker or portion thereof. For example, at least two ring systems of a linker or portion thereof may be connected to each other by no more than two sp³ bonds (e.g., by 0, 1, or 2 sp³ bonds). For example, at least two ring systems of a linker or portion thereof may be connected to each other by a no more than two sp² bonds, such as by no more than 1 sp² bond. Ring systems of a linker or portion thereof may be connected to each other by a limited number of atoms, such as by no more than 2 atoms. For example, at least two ring systems of a linker or portion thereof may be connected to each other by no more than 2 atoms, such as by only 1 atom or by no atoms (e.g., directly connected).

A series of ring systems of a linker or portion thereof may comprise aromatic and/or aliphatic rings. At least two ring systems of a linker or portion thereof may be connected to each other directly without an intervening carbon atom. A linker may comprise at least one amino acid that may comprise a ring system, such as a proline or hydroxyproline moiety. For example, a linker may comprise a hydroxyproline. A linker may comprise at least one non-proteinogenic amino acid (e.g., as described herein), such as a hydroxyproline. A linker may comprise a plurality of amino acids including ring systems in sequence. For example, a linker may comprise at least two amino acids in sequence, where each of the at least two amino acids includes a ring system (e.g., ring systems having the same or different structures). The at least two amino acids may comprise at least two non-proteinogenic amino acids, such as hydroxyprolines. In another example, a linker may comprise at least three amino acids in sequence, where each of the at least three amino acids includes a ring system (e.g., ring systems having the same or different structures). The at least three amino acids may comprise at least three non-proteinogenic amino acids. For example, the linker may comprise at least three hydroxyprolines, such as at least 3, 4, 5, 6, 7, 8, 9, 10, or more hydroxyprolines. Two or more non-proteinogenic amino acids may be included in sequence. For example, two or more non-proteinogenic amino acids may be adjacent to one another without an intervening feature or other chemical structure. For example, 3, 4, 5, 6, 7, 8, 9, 10, 11, 12, 13, 14, 15, 16, 17, 18, 19, 20, 21, 22, 23, 24, 25, 26, 27, 28, 29, 30, or more non-proteinogenic amino acids may be included in sequence. A linker may comprise a first sequence of amino acids including ring systems and a second sequence of amino acids including ring systems, where the first sequence and the second sequence may be separated by one or more moieties that do not include ring systems, such as one or more glycines. For example, a linker may comprise a first sequence of hydroxyprolines and a second sequence of hydroxyprolines, where the first sequence and the second sequence may be separated by at least a glycine. In another example, a linker may comprise a first sequence of amino acids including ring systems, a second sequence of amino acids including ring systems, and a third sequence of amino acids including ring systems, where the first, second, and third sequences may be separated by one or more moieties that do not include ring systems, such as one or more glycines. An optical (e.g., fluorescent) labeling reagent may comprise one or more linkers, such as one or more linkers each comprising two or more amino acids (e.g., non-proteinogenic amino acids). For example, an optical labeling reagent may comprise a first linker comprising a first sequence of amino acids and a second linker comprising a second sequence of amino acids, where the first sequence comprises two or more amino acids (e.g., non-proteinogenic amino acids) comprising ring systems and the second sequence comprises two or more amino acids (e.g., non-proteinogenic amino acids) comprising ring systems. In an example, an optical labeling reagent may comprise a first linker comprising a first sequence of hydroxyprolines and a second linker comprising a second sequence of hydroxyprolines. The first and second linkers may be connected to different portions of a scaffold. The first linker may be coupled, directly or indirectly, to a first optically detectable moiety and the second linker may be coupled, directly or indirectly, to a second optically detectable moiety, where the first and second optically detectable moieties may be of the same or different types.

A linker or portion thereof of a labeling reagent provided herein may comprise a secondary structure, such as a helical structure. For example, a labeling reagent may comprise a polyproline or polyhydroxyproline helix. A helical structure comprising prolines and/or hydroxyprolines may comprise three or more prolines and/or hydroxyprolines in sequence. For example, an optical labeling reagent may comprise a first linker comprising a first secondary structure (e.g., helical structure) comprising a first sequence of hydroxyprolines and a second linker comprising a second secondary structure (e.g., helical structure) comprising a second sequence of hydroxyprolines. The first and second linkers may be connected to different portions of a scaffold. The first linker may be coupled, directly or indirectly, to a first optically detectable moiety and the second linker may be coupled, directly or indirectly, to a second optically detectable moiety, where the first and second optically detectable moieties may be of the same or different types. In a helical structure comprising prolines and/or hydroxyprolines, or derivatives thereof, a given proline, hydroxyproline, or derivative thereof may provide a physical separation of approximately 3 Å between moieties to which it is connected. For example, a helical or semi-helical structure comprising three prolines, hydroxyprolines, or similar structures may provide physical separation of approximately 9 Å between moieties to which they are connected. In some cases, a secondary structure such as a helical structure may provide a physical separation between moieties to which they are connected of at least about 9 Å, such as at least about 9 Å, 12 Å, 15 Å, 18 Å, 21 Å, 24 Å, 27 Å, 30 Å, or more. In some cases, several such secondary structures may be included in a single linker moiety, optionally separated by one or more features such as another chemical moiety. For example, two helical structures comprising prolines, hydroxyprolines, or derivatives thereof may be separated by a glycine. In some cases, multiple secondary structures may be included in an optical labeling reagent but may not necessarily be included in sequence. For example, an optical labeling reagent may comprise a first linker comprising a first helical structure and a second linker comprising a second helical structure. The first linker or the second linker may additionally comprise a third helical structure and, in some cases, a fourth helical structure.

The structural features of a linker, including the number of rings, the rigidity of the linker or a portion thereof, and the like, can combine to establish a functional distance between an optically detectable moiety (e.g., fluorescent dye moiety) and a substrate (e.g., protein, nucleotide or nucleotide analog, cell, etc.) labeled by a labeling reagent. In some cases, the distance corresponds to the length (and/or the functional length) of the linker. A functional length of a labeling reagent or portion thereof may be an average value representing an average over various molecular and solvent motions. In some cases, the functional length varies based on one or more of the temperature, solvent, pH, and/or salt concentration of the solution in which the length is measured or estimated. The functional length can be measured in a solution in which an optical (e.g., fluorescent) signal from the substrate is measured. The functional length may an average or ensemble value of a distribution of functional lengths (e.g., over rotational, vibrational, and translational motions) and may differ based on, e.g., temperature, solvent, pH, and/or salt concentrations. The functional length may be estimated (e.g., based on bond lengths and steric considerations, such as by use of a chemical drawing or modeling program) and/or measured (e.g., using molecular imaging and/or crystallographic techniques). For an optical (e.g., fluorescent) labeling reagent comprising one or more linkers, such as one or more linkers connecting one or more dye moieties to a substrate, one or more different functional distances may be established between dye moieties and a substrate.

A labeling reagent can establish any suitable functional length between an optically detectable moiety (e.g., fluorescent dye) and a substrate (e.g., protein, nucleotide or nucleotide analog, cell, etc.) labeled by the labeling reagent. In some cases, the functional length is at most about 500 nanometers (nm), about 200 nm, about 100 nm, about 75 nm, about 50 nm, about 40 nm, about 30 nm, about 20 nm, about 10 nm, about 5 nm, about 2 nm, about 1.0 nm, about 0.5 nm, about 0.3 nm, about 0.2 nm, or less. In some instances, the functional length is at least about 0.2 nanometers (nm), at least about 0.3 nm, at least about 0.5 nm, at least about 1.0 nm, at least about 2 nm, at least about 5 nm, at least about 10 nm, at least about 20 nm, at least about 30 nm, at least about 40 nm, at least about 50 nm, at least about 75 nm, at least about 100 nm, at least about 200 nm, at least about 500 nm, or more. In some instances, the functional length is between about 0.5 nm and about 50 nm. In some cases, the functional length may be at least about 9 Å, 12 Å, 15 Å, 18 Å, 21 Å, 24 Å, 27 Å, 30 Å, 33 Å, 36 Å, 39 Å, 42 Å, 45 Å, 48 Å, 51 Å, 54 Å, 57 Å, 60 Å, 63 Å, 66 Å, 69 Å, 72 Å, 75 Å, 78 Å, 81 Å, 84 Å, 87 Å, 90 Å, or more.

Many applications of optical (e.g., fluorescent) labeling reagents (e.g., nucleic acid sequencing reactions and protein/cell labeling) can be performed in aqueous solutions. In some cases, a linker that has too high of a proportion of carbon and hydrogen atoms and/or a lack of charged chemical groups can be insufficiently water-soluble to be useful in an aqueous solution. Accordingly, a labeling reagent may comprise one or more water-soluble groups. A water-soluble group may be incorporated into a labeling reagent at any useful position. For example, a linker of a labeling reagent, or a semi-rigid portion thereof, may include one or more water-soluble groups. A labeling reagent may also or alternatively include one or more water-soluble groups at or near a point of attachment to an optically detectable moiety (e.g., a fluorescent dye moiety, as described herein). Alternatively or additionally, a labeling reagent may comprise a water-soluble group at or near a point of attachment to a substrate (e.g., a protein, nucleotide or nucleotide analog, cell, etc.). Alternatively or additionally, a labeling reagent may comprise a water-soluble group between points of attachment to an optically detectable moiety (e.g., fluorescent dye moiety, as described herein) and a substrate (e.g., a protein, nucleotide or nucleotide analog, cell, etc.). One or more rings of a labeling reagent or linker thereof may comprise a water-soluble group incorporated therein or appended thereto. For example, a given ring of a labeling reagent, such as a ring included in a linker portion of a labeling reagent, may comprise one or more water-soluble moieties. For example, a ring of a linker may comprise two water-soluble moieties. A water-soluble group may be a constituent part of the backbone of a ring structure. Alternatively or additionally, a water-soluble group may be appended to a ring structure (e.g., as a substituent). For example, a labeling reagent may comprise at least one hydroxyproline, which hydroxyproline comprises a five-membered ring having a hydroxyl group appended thereto. Water-soluble moieties of a labeling reagent may be of the same or different types. For example, a labeling reagent may comprise at least one water-soluble moiety of a first type and at least one water-soluble moiety of a second type that is different from the first type. In an example, a labeling reagent may comprise multiple water-soluble moieties of a given type, such as multiple hydroxyl moieties. In some cases, a water-soluble group may be positively charged. Examples of suitable water-soluble groups include, but are not limited to, a pyridinium, an imidazolium, a quaternary ammonium group, a sulfonate, a sulfate, a phosphate, an alcohol, an amine, an imine, a nitrile, an amide, a thiol, a carboxylic acid, a polyether, an aldehyde, and a boronic acid or boronic ester.

A water-soluble group can be any functional group that decreases (including making more negative) the Log P of the optical (e.g., fluorescent) labeling reagent. Log P is the partition coefficient for a molecule between water and n-octanol. A greasy molecule is more likely to partition into octanol, giving a positive and large Log P value. A formula for Log P can be represented as Log P_{octanol/water}=log ([solute]_{octanol}/[solute]_{water}), where [solute]_{octanol} is the concentration of the solute (i.e., the labeling reagent) in octanol and [solute]_{water} is the concentration of the solute in water. Therefore, the more a compound partitions into water compared to octanol, the more negative the Log P. Log P can be measured experimentally or predicted using software algorithms. The water-soluble group can have any suitable Log P value. In some cases, the Log P is less than about 2, less than about 1.5, less than about 1, less than about 0.5, less than about 0, less than about −0.5, less than about −1, less than about −1.5, less than about −2, or lower. In some cases, the Log P is between about 2.0 and about −2.0.

A linker may include one or more asymmetric (e.g., chiral) centers (e.g., as described herein). All stereochemical isomers of linkers are contemplated, including racemates and enantiomerically pure linkers.

A labeling reagent or component thereof, and/or a substrate (e.g., protein, nucleotide or nucleotide analog, cell, etc.) to which it may be coupled, may include one or more isotopic (e.g., radio) labels (e.g., as described herein). All isotopic variations of linkers are contemplated.

A labeling reagent may comprise a polymer having a regularly repeating unit. Alternatively, a labeling reagent may comprise a co-polymer without a regularly repeating unit. A repeating unit may comprise a sequence of amino acids (e.g., non-proteinogenic amino acids). For example, a repeating unit may comprise at least 3 prolines, hydroxyprolines, or derivatives thereof, such as at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or more prolines, hydroxyprolines, or derivatives thereof. A repeating unit may comprise two or more different amino acids. For example, a repeating unit may comprise a first amino acid (X) and a second amino acid (Y). One or more of the first or second amino acids may be included. For example, a labeling reagent may comprise a moiety having the formula (XₙYₘ)ᵢ, where n is at least 1, m is at least 1, and i is at least 2 and X and Y are different amino acids. In an example, X may be glycine, n is 1, and Y is hydroxyproline. In such an instance, m may be at least 3 (e.g., 3, 4, 5, 6, 7, 8, 9, 10, 11, 12, 13, 14, 15, 16, 17, 18, 19, 20, or more) and i may be, for example, at least 2 (e.g., 2, 3, 4, 5, 6, 7, 8, or more). An example of such a linker component is shown below:

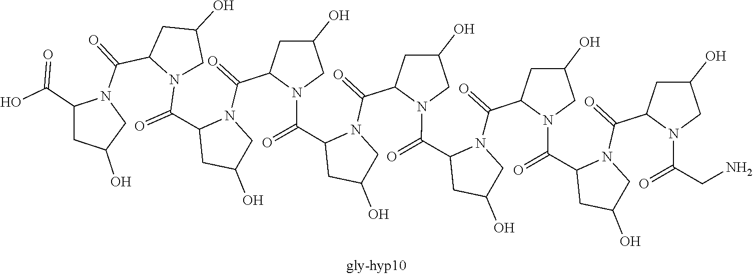 [SMILES: NCC(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)O]

The following labels of: “Hypₙ”, “Hypn”, “hypₙ”, “hypn”, as used herein, which may generally describe a unit of n hydroxyproline moieties, unless explicitly described otherwise (e.g., “gly-”, “Gly-”, “Gly”-, “gly”-, “with glycine”, “without glycine”, as drawn, etc.) may refer to a structure which may or may not have one or more glycine moieties. For example, such labels may describe a structure of n hydroxyproline moieties with a glycine moiety at an end, a structure of n hydroxyproline moieties which may have one or more glycine moieties between hydroxyprolines, or a structure of n hydroxyproline moieties without any glycine moieties. The structure shown above includes 10 hydroxyproline moieties and a glycine moiety and is referred to herein as “H” “gly-hyp10”, GlyHyp10, Gly-Hyp10, glyhyp₁₀, gly-hyp₁₀, hyp10-gly, or similar. One or more such structures may be included in a labeling reagent or linker portion thereof. For example, a gly-hyp10 structure may be a repeating unit in a linker. Two gly-hyp10 structures in sequence may be referred to herein as hyp20 (having two glycines), or gly-hyp10-glyhyp10. Such a structure may include 20 hydroxyproline moieties and, in some cases, one or more (e.g., two) glycines. Similarly, three gly-hyp10 structures in sequence may be referred to herein as gly-hyp30. Such a structure may include 30 hydroxyproline moieties and one or more glycines. For example, a gly-hyp30 sequence may include three sets of ten hydroxyprolines separated by glycines. Alternatively, a hyp30 structure may include thirty hydroxyprolines with no intervening structures. Related structures including different numbers of hydroxyprolines (e.g., hypn or hypₙ) may also be included in a labeling reagent. Additional details of such structures are provided elsewhere herein. As described herein, all stereoisomers of gly-hyp10, gly-hyp20, and hyp30, as well as combinations thereof, are contemplated.

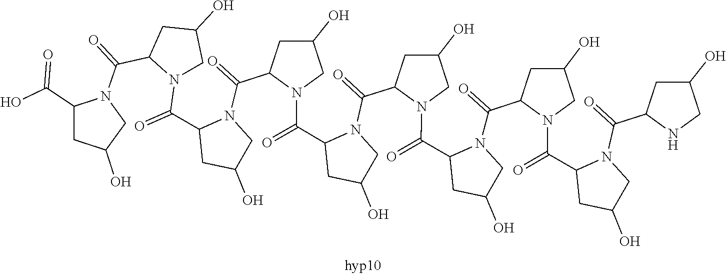 [SMILES: O=C(O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1]

A linker may comprise at least about 10 non-proteinogenic amino acids, such as at least about 10 hydroxyprolines and at least about one different non-proteinogenic amino acid. The linker may comprise at least about 10 hydroxyprolines and at least about one cysteic acid. The linker may comprise about 10 hydroxyprolines and about one cysteic acid. The linker may have a structure below:

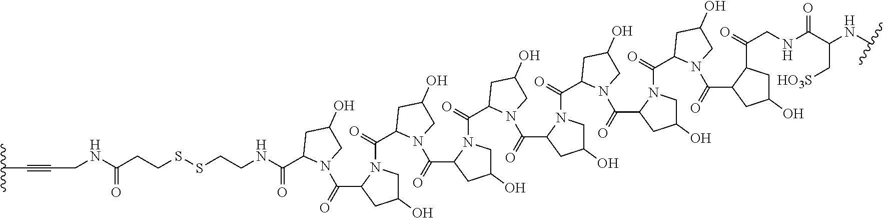 [SMILES: *C#CCNC(=O)CCSSCCNC(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CC1C(=O)CNC(=O)C(CS(=O)(=O)O)N*]

A linker may comprise at least about 20 non-proteinogenic amino acids, such as at least about 20 hydroxyprolines and at least about one different non-proteinogenic amino acid. The linker may comprise at least about 20 hydroxyprolines and at least about one cysteic acid. The linker may comprise about 20 hydroxyprolines and about one cysteic acid. The linker may have a structure below:

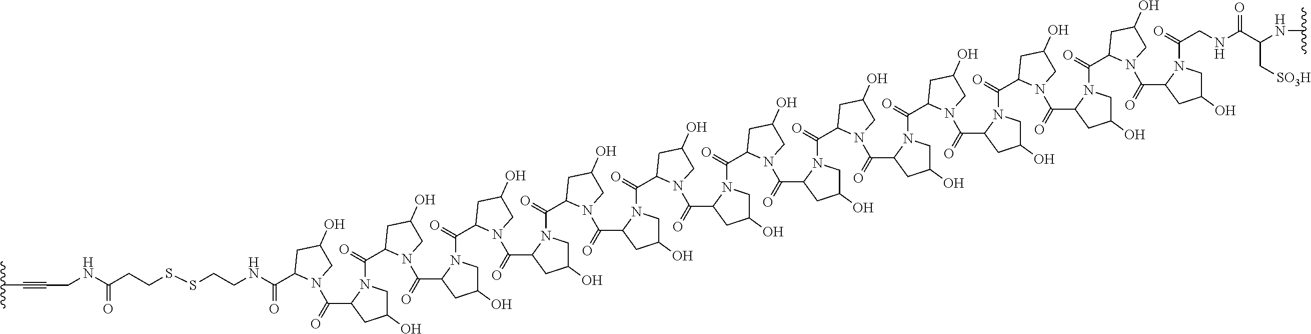 [SMILES: *C#CCNC(=O)CCSSCCNC(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)CNC(=O)C(CS(=O)(=O)O)N*]

A linker may comprise at least about 10 non-proteinogenic amino acids, such as at least about 10 hydroxyprolines and at least about two additional non-proteinogenic amino acids. The two additional non-proteinogenic amino acids may be a same type. The two additional non-proteinogenic amino acids may be the same. The two additional non-proteinogenic amino acids may also be different. The linker may comprise at least about 10 hydroxyprolines and at least about two cysteic acids. The linker may comprise about 10 hydroxyprolines and about two cysteic acids. The linker may have a structure below:

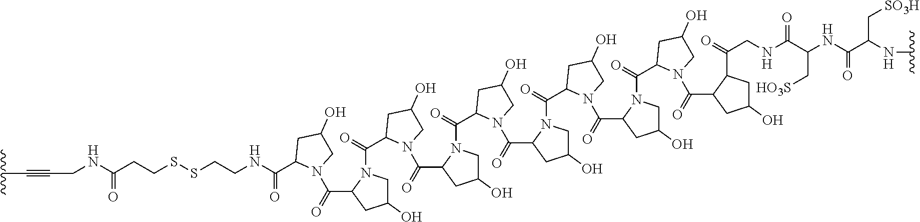 [SMILES: *C#CCNC(=O)CCSSCCNC(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CC1C(=O)CNC(=O)C(CS(=O)(=O)O)NC(=O)C(CS(=O)(=O)O)N*]

A linker may comprise at least about 20 non-proteinogenic amino acids, such as at least about 20 hydroxyprolines and at least about two additional non-proteinogenic amino acids. The linker may comprise at least about 20 hydroxyprolines and at least about two cysteic acids. The linker may comprise about 20 hydroxyprolines and about two cysteic acids. The linker may have a structure below:

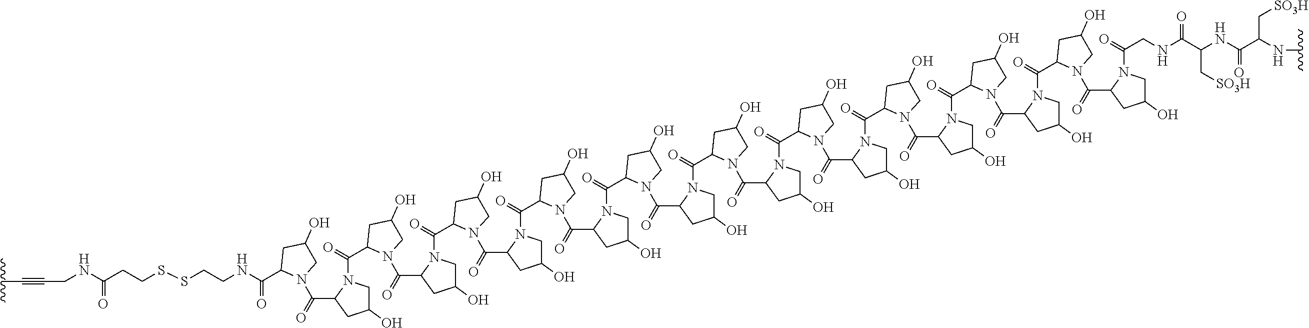 [SMILES: *C#CCNC(=O)CCSSCCNC(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)CNC(=O)C(CS(=O)(=O)O)NC(=O)C(CS(=O)(=O)O)N*]

A linker may comprise at least about 10 non-proteinogenic amino acids, such as at least about 10 hydroxyprolines and at least about one different non-proteinogenic amino acid. The linker may comprise at least about 10 hydroxyprolines and at least about one 5-amino-5-carboxy-N,N,N-trimethylpentan-1-aminium or a salt thereof. The linker may comprise about 10 hydroxyprolines and about one 5-amino-5-carboxy-N,N,N-trimethylpentan-1-aminium or a salt thereof. The linker may have a structure below:

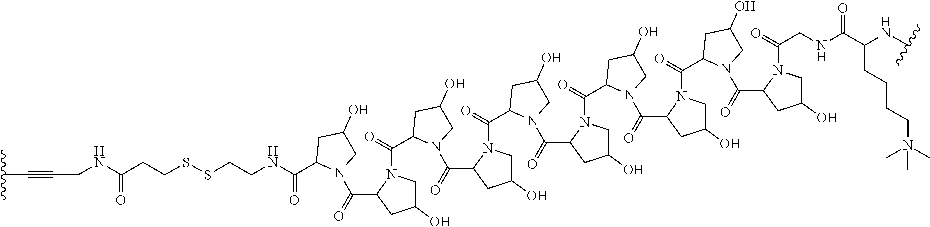 [SMILES: *C#CCNC(=O)CCSSCCNC(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)CNC(=O)C(CCCC[N+](C)(C)C)N*]

A linker may comprise at least about 20 non-proteinogenic amino acids, such as at least about 20 hydroxyprolines and at least about one different non-proteinogenic amino acid. The linker may comprise at least about 20 hydroxyprolines and at least about one 5-amino-5-carboxy-N,N,N-trimethylpentan-1-aminium or a salt thereof. The linker may comprise about 20 hydroxyprolines and about one 5-amino-5-carboxy-N,N,N-trimethylpentan-1-aminium or a salt thereof. The linker may have a structure below:

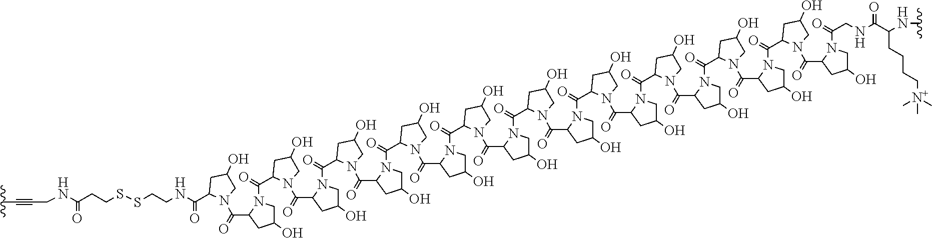 [SMILES: *C#CCNC(=O)CCSSCCNC(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)CNC(=O)C(CCCC[N+](C)(C)C)N*]

A linker may comprise at least about 10 non-proteinogenic amino acids, such as at least about 10 hydroxyprolines and at least about two additional non-proteinogenic amino acids. The two additional non-proteinogenic amino acids may be a same type. The two additional non-proteinogenic amino acids may be the same. The two additional non-proteinogenic amino acids may also be different. The linker may comprise at least about 10 hydroxyprolines and at least about two 5-amino-5-carboxy-N,N,N-trimethylpentan-1-aminium or a salt thereof. The linker may comprise about 10 hydroxyprolines and about two 5-amino-5-carboxy-N,N,N-trimethylpentan-1-aminium or a salt thereof. The linker may have a structure below:

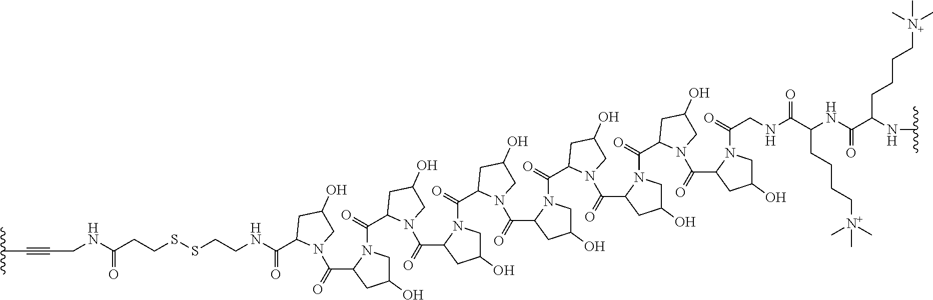 [SMILES: *C#CCNC(=O)CCSSCCNC(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)CNC(=O)C(CCCC[N+](C)(C)C)NC(=O)C(CCCC[N+](C)(C)C)N*]

A linker may comprise at least about 20 non-proteinogenic amino acids, such as at least about 20 hydroxyprolines and at least about two additional non-proteinogenic amino acids. The linker may comprise at least about 20 hydroxyprolines and at least about two 5-amino-5-carboxy-N,N,N-trimethylpentan-1-aminium or a salt thereof. The linker may comprise about 20 hydroxyprolines and about two 5-amino-5-carboxy-N,N,N-trimethylpentan-1-aminium or a salt thereof. The linker may have a structure below:

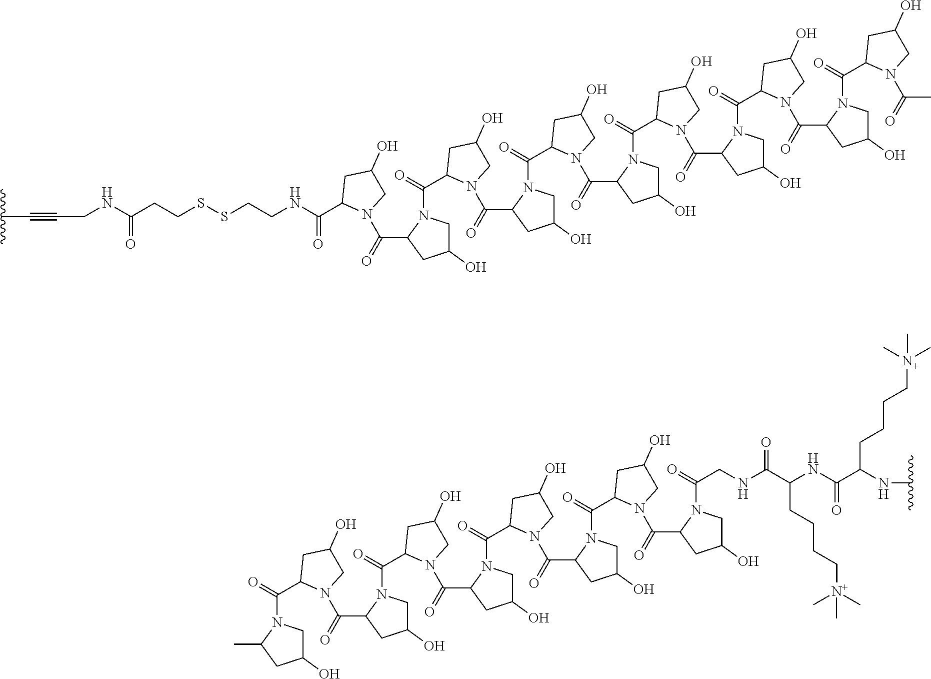

The linker may comprise at least about 10 hydroxyprolines and at least about one 6-aminohexanoic acid. The linker may comprise about 10 hydroxyprolines and about one 6-aminohexanoic acid. The linker may have a structure below:

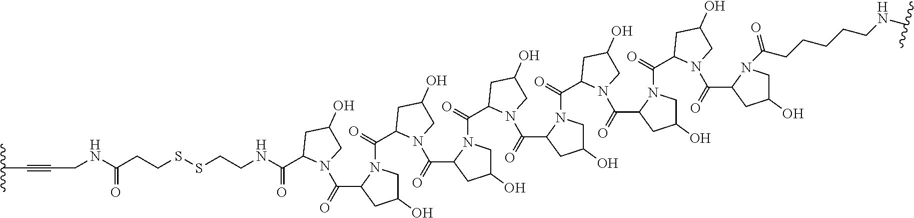 [SMILES: *C#CCNC(=O)CCSSCCNC(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)CCCCCN*]

A linker may comprise at least about 20 hydroxyprolines and at least about one different non-proteinogenic amino acid. The linker may comprise at least about 20 hydroxyprolines and at least about one 6-aminohexanoic acid. The linker may comprise about 20 hydroxyprolines and one 6-aminohexanoic acid. The linker may have a structure below:

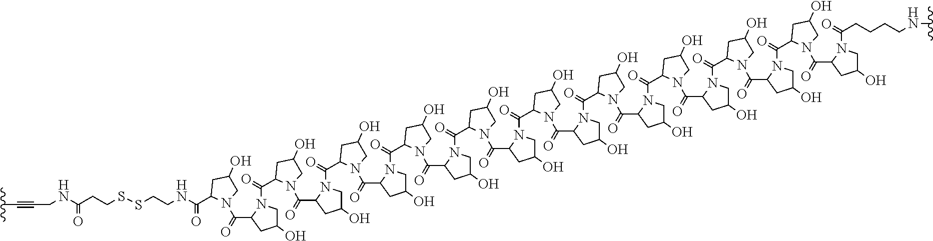 [SMILES: *C#CCNC(=O)CCSSCCNC(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)C1CC(O)CN1C(=O)CCCCN*]

In some cases, a substrate may comprise a chemical formula below:

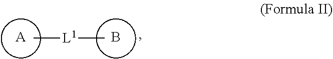

wherein: A comprises a nucleobase; B is a detectable moiety; and L¹ is a linker comprising at least one cleavable group and at least one non-proteinogenic amino acid.

In some cases, the nucleobase comprises adenine, cytosine, thymine, or uracil. In some cases, the nucleobase is adenine. In some cases, the nucleobase is cytosine. In some cases, the nucleobase is thymine. In some cases, the nucleobase is uracil. In some cases, the nucleobase is not guanine. In some cases, the nucleobase is guanine. In some cases, the non-proteinogenic amino acid may not comprise hydroxyproline. In some cases, the non-proteinogenic amino acid may comprise hydroxyproline. In some cases, the non-proteinogenic amino acid may comprise cysteic acid, 5-amino-5-carboxy-N,N,N-trimethylpentan-1-aminium or a salt thereof, or 6-aminohexanoic acid. In some cases, the non-proteinogenic amino acid may comprise cysteic acid. In some cases, the non-proteinogenic amino acid may comprise 5-amino-5-carboxy-N,N,N-trimethylpentan-1-aminium. In some cases, the non-proteinogenic amino acid may comprise 6-aminohexanoic acid. In some cases, L¹ may comprise at least two different non-proteinogenic amino acids. In some cases, L¹ may comprise two different non-proteinogenic amino acids. In some cases, L¹ may comprise at least hydroxyproline and cysteic acid. In some cases, L¹ may comprise at least hydroxyproline and at least two additional non-proteinogenic amino acids. The two additional non-proteinogenic amino acids may be a same type. The two additional non-proteinogenic amino acids may be the same. The two additional non-proteinogenic amino acids may also be different. In some cases, L¹ may comprise at least hydroxyproline and cysteic acid. In some cases, L¹ may comprise at least about 10 hydroxyprolines and at least one cysteic acid. In some cases, L¹ may comprise at least about 10 hydroxyprolines and at least about two cysteic acids. In some cases, L¹ may comprise at least about 20 hydroxyprolines and at least about one cysteic acid. In some cases, L¹ may comprise at least about 20 hydroxyprolines and at least about two cysteic acids. In some cases, L¹ may comprise 10 hydroxyprolines and one cysteic acid. In some cases, L¹ may comprise 20 hydroxyprolines and two cysteic acids. In some cases, L¹ may comprise 10 hydroxyprolines and about two cysteic acids. In some cases, L¹ may comprise 20 hydroxyprolines and two cysteic acids. In some cases, L¹ may comprise at least hydroxyproline and 5-amino-5-carboxy-N,N,N-trimethylpentan-1-aminium or a salt thereof. In some cases, L¹ may comprise at least about 10 hydroxyprolines and at least one 5-amino-5-carboxy-N,N,N-trimethylpentan-1-aminium or a salt thereof. In some cases, L¹ may comprise at least about 10 hydroxyprolines and at least about two 5-amino-5-carboxy-N,N,N-trimethylpentan-1-aminium or a salt thereof. In some cases, L¹ may comprise at least about 20 hydroxyprolines and at least about one 5-amino-5-carboxy-N,N,N-trimethylpentan-1-aminium or a salt thereof. In some cases, L¹ may comprise at least about 20 hydroxyprolines and at least about two 5-amino-5-carboxy-N,N,N-trimethylpentan-1-aminium or a salt thereof. In some cases, L may comprise 10 hydroxyprolines and one 5-amino-5-carboxy-N,N,N-trimethylpentan-1-aminium or a salt thereof. In some cases, L¹ may comprise 20 hydroxyprolines and two 5-amino-5-carboxy-N,N,N-trimethylpentan-1-aminium or a salt thereof. In some cases, L may comprise 10 hydroxyprolines and about two 5-amino-5-carboxy-N,N,N-trimethylpentan-1-aminium or a salt thereof. In some cases, L¹ may comprise 20 hydroxyprolines and two 5-amino-5-carboxy-N,N,N-trimethylpentan-1-aminium or a salt thereof. In some cases, L¹ may comprise at least hydroxyproline and 6-aminohexanoic acid. In some cases, L¹ may comprise at least about 10 hydroxyprolines and at least about one 6-aminohexanoic acid. In some cases, L¹ may comprise at least about 20 hydroxyprolines and at least about one 6-aminohexanoic acid. In some cases, L¹ may comprise 10 hydroxyprolines and one 6-aminohexanoic acid. In some cases, L¹ may comprise 20 hydroxyprolines and one 6-aminohexanoic acid.

In some cases, when the non-proteinogenic amino acid comprises 6-aminohexanoic acid, the detectable moiety may not comprise a Cyanide 5 (Cy5) or an ATTO 647 NHS Ester (ATTO 647N). In some cases, when the at least one non-proteinogenic amino acid comprises 6-aminohexanoic acid, the linker may not be coupled to the below structures:

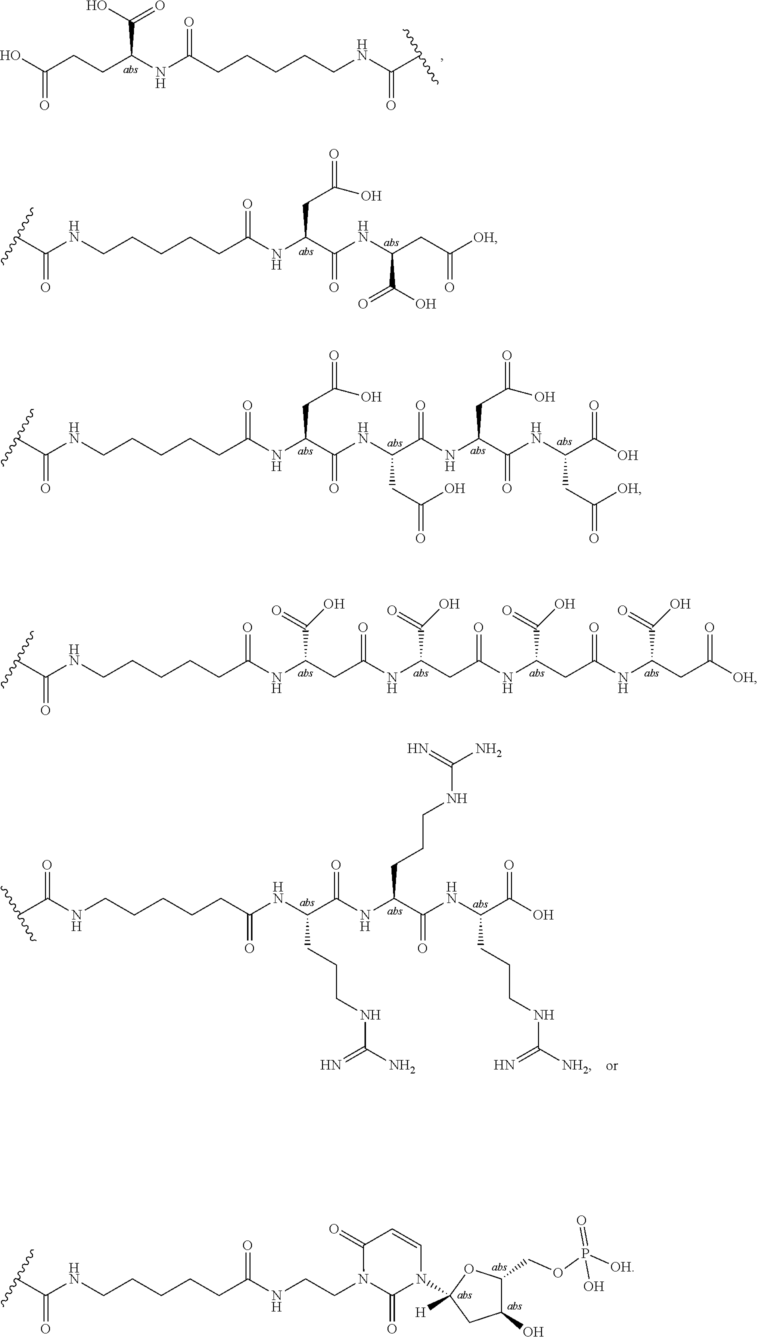 [SMILES: *C(=O)NCCCCCC(=O)N[C@@H](CCC(=O)O)C(=O)O ; *C(=O)NCCCCCC(=O)N[C@@H](CC(=O)O)C(=O)N[C@@H](CC(=O)O)C(=O)O ; *C(=O)NCCCCCC(=O)N[C@@H](CC(=O)O)C(=O)N[C@@H](CC(=O)O)C(=O)N[C@@H](CC(=O)O)C(=O)N[C@@H](CC(=O)O)C(=O)O ; *C(=O)NCCCCCC(=O)N[C@@H](CC(=O)N[C@@H](CC(=O)N[C@@H](CC(=O)N[C@@H](CC(=O)O)C(=O)O)C(=O)O)C(=O)O)C(=O)O ; *C(=O)NCCCCCC(=O)N[C@@H](CCCNC(=N)N)C(=O)N[C@@H](CCCNC(=N)N)C(=O)N[C@@H](CCCNC(=N)N)C(=O)O ; *C(=O)NCCCCCC(=O)NCCn1c(=O)ccn([C@H]2C[C@H](O)[C@@H](COP(=O)(O)O)O2)c1=O]

In some case, when the at least one non-proteinogenic amino acid comprises 6-aminohexanoic acid, the linker may not be coupled to a terminator moiety of a sequencing or a nucleic acid polymerization reaction. In some cases, L may comprise a cleavable group or moiety. In some cases, a substrate may comprise a chemical formula below:

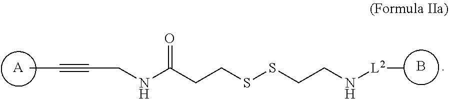

In some cases, B is a detectable moiety. In some cases, A may comprise a substrate. In some cases, A may comprise a nucleobase. In some cases, the nucleobase comprises adenine, cytosine, thymine, or uracil. In some cases, the nucleobase is adenine. In some cases, the nucleobase is cytosine. In some cases, the nucleobase is thymine. In some cases, the nucleobase is uracil. In some cases, the nucleobase is not guanine. In some cases, the nucleobase is guanine. In some cases, A may comprise a nucleoside. In some cases, A may comprise a nucleotide. In some cases, A may comprise a deoxyribose nucleotide triphosphate. In some cases, A may comprise a ribose nucleotide triphosphate. In some cases, L² may comprise a non-proteinogenic amino acid. In some cases, the non-proteinogenic amino acid may comprise any non-proteinogenic amino acids described herein (e.g., cysteic acid, 5-amino-5-carboxy-N,N,N-trimethylpentan-1-aminium or a salt thereof, 6-aminohexanoic acid, and/or hydroxyproline). For example, L² may comprise at least two different non-proteinogenic amino acids. In some cases, L² may comprise two different non-proteinogenic amino acids. In some cases, L² may comprise at least hydroxyproline and cysteic acid. In some cases, L² may comprise at least about 10 hydroxyprolines and at least about one cysteic acid. In some cases, L² may comprise at least about 20 hydroxyprolines and at least about one cysteic acid. In some cases, L² may comprise 10 hydroxyprolines and one cysteic acid. In some cases, L² may comprise 20 hydroxyprolines and one cysteic acid. In some cases, L² may comprise at least about two cysteic acids. In some cases, L² may comprises two cysteic acids. In some cases, L² may comprise at least hydroxyproline and 5-amino-5-carboxy-N,N,N-trimethylpentan-1-aminium or a salt thereof. In some cases, L² may comprise at least about 10 hydroxyprolines and at least about one 5-amino-5-carboxy-N,N,N-trimethylpentan-1-aminium or a salt thereof. In some cases, L² may comprise at least about 20 hydroxyprolines and at least about one 5-amino-5-carboxy-N,N,N-trimethylpentan-1-aminium or a salt thereof. In some cases, L² may comprise 10 hydroxyprolines and one 5-amino-5-carboxy-N,N,N-trimethylpentan-1-aminium or a salt thereof. In some cases, L² may comprise 20 hydroxyprolines and one 5-amino-5-carboxy-N,N,N-trimethylpentan-1-aminium or a salt thereof. In some cases, L² may comprise at least about two 5-amino-5-carboxy-N,N,N-trimethylpentan-1-aminium or a salt thereof. In some cases, L² may comprises two 5-amino-5-carboxy-N,N,N-trimethylpentan-1-aminium or a salt thereof.

In some cases, L² may comprise at least hydroxyproline and 6-aminohexanoic acid. In some cases, L² may comprise at least about 10 hydroxyprolines and at least about one 6-aminohexanoic acid. In some cases, L² may comprise at least about 20 hydroxyprolines and at least about one 6-aminohexanoic acid. In some cases, L² may comprise 10 hydroxyprolines and one 6-aminohexanoic acid. In some cases, L² may comprise 20 hydroxyprolines and one 6-aminohexanoic acid. In some cases, B may be the detectable moiety described herein. In some cases, the substrate may comprise any one of the chemical formulas below:

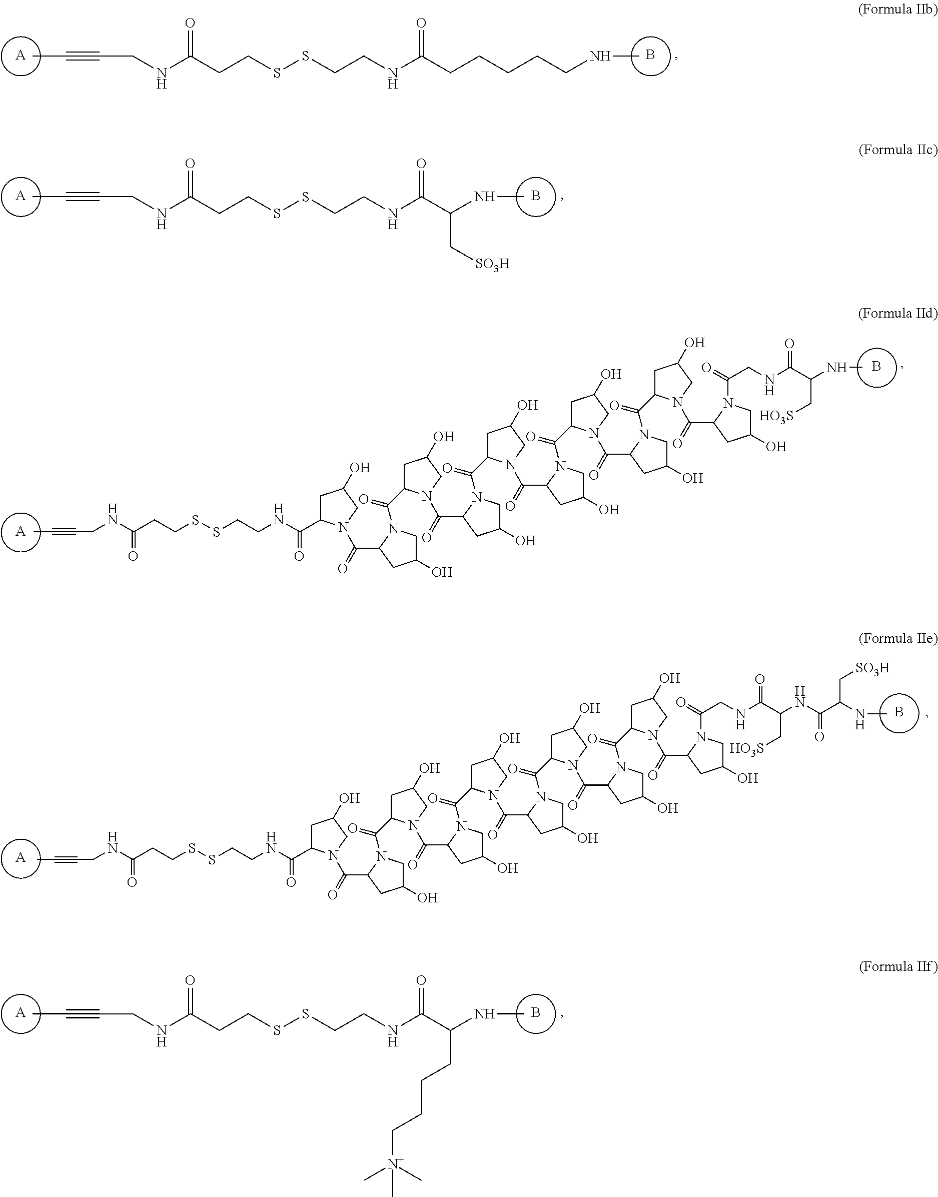

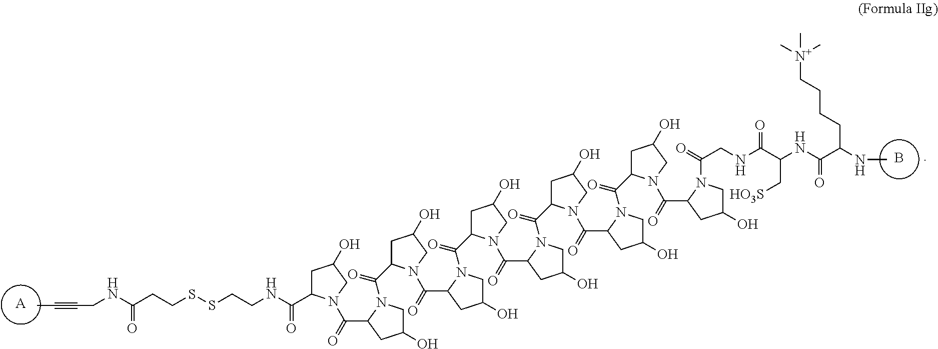

A polymer or co-polymer structure may be included in a linker portion of a labeling reagent. A polymer or co-polymer structure may be prepared according to any useful method and may not be the result of a polymerization process. In general, a polymerization process can generate products having a variety of degrees of polymerization and molecular weights. In contrast, the labeling reagents provided herein may have a defined (i.e., known) molecular weight.

A labeling reagent may comprise a straight and/or contiguous chain. For example, a labeling reagent may have the general structure: (optional cleavable linker portion)-(semi-rigid linker portion)-(optically detectable moiety). Each moiety may be separated by one or more additional features including, e.g., a spacer portion. A labeling reagent may comprise multiple straight and/or contiguous chains linked to a central structure (e.g., scaffold, as described herein). A linker portion of a labeling reagent may comprise a branchpoint that facilitates connection of multiple optically detectable moieties to a given linker portion. Alternatively, a linker portion of a labeling reagent may be configured to connect to a single optically detectable moiety.

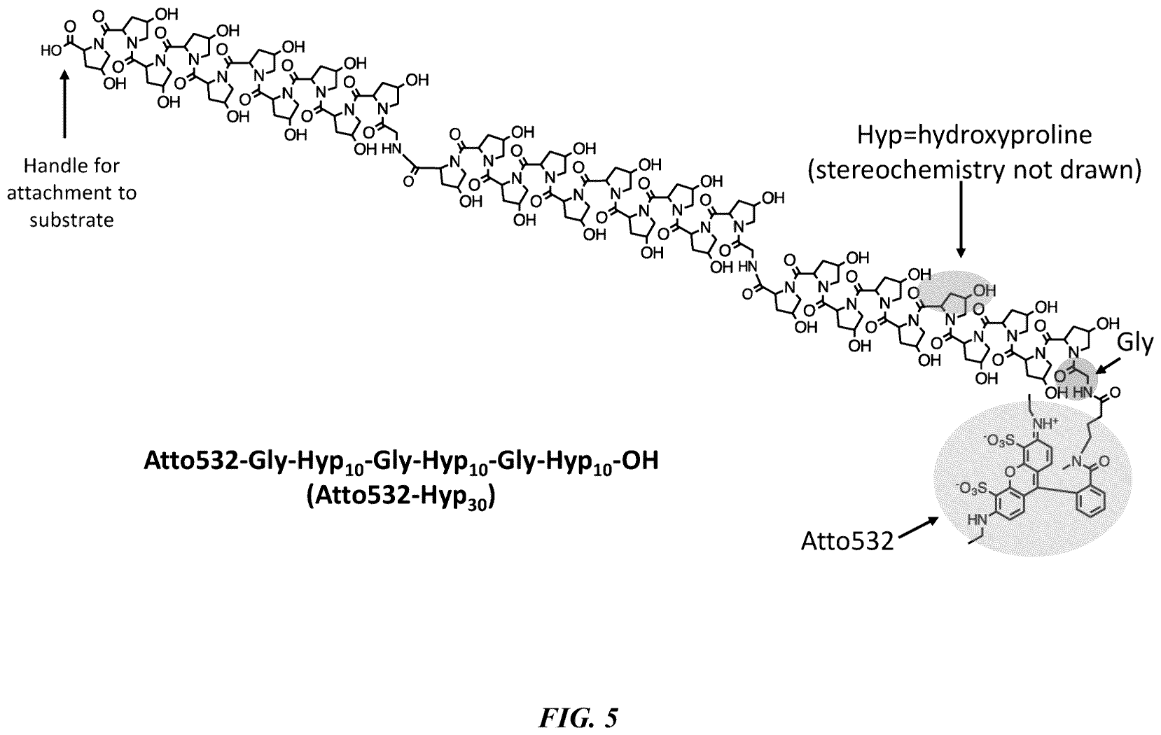

FIG. 5 shows an example structure for inclusion in a labeling reagent. The example structure includes a linker comprising three sequences of ten hydroxyprolines separated by glycines. The ten hydroxyproline portion may be represented herein as Hyp10, hyp10, Hyp₁₀, or hyp₁₀. The linker including the three sequences of ten hydroxyprolines separated by glycines may be represented as, for example, Hyp10-Gly-Hyp10-Gly-Hyp10-Gly or, in the alternative, Gly-Hyp10-Gly-Hyp10-Gly-Hyp10. The linker including the three sequences of ten hydroxyprolines separated by glycines may also be represented as, for example, Hyp30, hyp30, Hyp₃₀, or hyp₃₀. The structure also includes an optical dye moiety coupled to the linker via a glycine. The optical dye moiety, “Atto532”, included in FIG. 5 fluoresces at approximately 532 nanometers (nm). However, any other useful dye moiety may be used (e.g., as described herein). The structure shown in FIG. 5 also includes a handle for attachment to one or more additional moieties, including a cleavable linker moiety and/or spacer moiety via which the structure may be linked to a substrate (e.g., as described herein). In some cases, a linker may not include a cleavable linker moiety and the handle may provide a connection to a substrate. In some cases, the illustrated structure or a similar structure may be connected to a scaffold, optionally with an intervening cleavable moiety, which scaffold may facilitate the inclusion of multiple optically detectable moieties in a single labeling reagent.

### Amino Acids

A labeling reagent may include a plurality of amino acids in one or more portions of the labeling reagent. For example, an amino acid or plurality of amino acids, such as one or more lysines, may serve as a scaffold to which one or more linkers may attach (e.g., as described herein). Alternatively, or additionally, a linker of a labeling reagent may include one or more amino acids (e.g., as described herein). A labeling reagent may include any useful number of amino acids, such as at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more amino acids. At least a subset of the amino acids of a labeling reagent may be included in sequence (e.g., adjacent to one another). A labeling reagent may comprise multiple different subsets of amino acids, such as multiple different sequences of amino acids. As described herein, amino acids may be arranged in a secondary structure such as a helical structure. For example, a labeling reagent (e.g., a linker of a labeling reagent) may comprise a portion comprising a secondary structure such as a helical structure, such as a helical structure comprising a plurality of prolines, hydroxyprolines, or derivatives thereof. A labeling reagent comprising multiple linkers may comprise multiple sets of amino acids, and each linker of a labeling reagent may comprise a shared or different chemical structure (e.g., an identical sequence of amino acids).

An amino acid may be a natural amino acid or a non-natural amino acid. An amino acid may be a proteinogenic amino acid or a non-proteinogenic amino acid. A “proteinogenic amino acid,” as used herein, generally refers to a genetically encoded amino acid that may be incorporated into a protein during translation. Proteinogenic amino acids include arginine, histidine, lysine, aspartic acid, glutamic acid, serine, threonine, asparagine, glutamine, cysteine, selenocysteine, glycine, proline, alanine, isoleucine, leucine, methionine, phenylalanine, tryptophan, tyrosine, valine, selenocysteine, and pyrrolysine. A “non-proteinogenic amino acid,” as used herein, is an amino acid that is not a proteinogenic amino acid. A non-proteinogenic amino acid may be a naturally occurring amino acid or a non-naturally occurring amino acid. Non-proteinogenic amino acids include amino acids that are not found in proteins and/or are not naturally encoded or found in the genetic code of an organism. Examples of non-proteinogenic amino acids include, but are not limited to, hydroxyproline, selenomethionine, hypusine, 2-aminoisobutyric acid, αγ-aminobutyric acid, ornithine, citrulline, β-alanine (3-aminopropanoic acid), δ-aminolevulinic acid, 4-aminobenzoic acid, dehydroalanine, carboxyglutamic acid, pyroglutamic acid, norvaline, norleucine, alloisoleucine, t-leucine, pipecolic acid, allothreonine, homocysteine, homoserine, α-amino-n-heptanoic acid, α,β-diaminopropionic acid, α,γ-diaminobutyric acid, β-amino-n-butyric acid, β-aminoisobutyric acid, isovaline, sarcosine, N-ethyl glycine, N-propyl glycine, N-isopropyl glycine, N-methyl alanine, N-ethyl alanine, N-methyl β-alanine, N-ethyl β-alanine, isoserine, and α-hydroxy-γ-aminobutyric acid. Additional examples of non-proteinogenic amino acids include the non-natural amino acids described herein. A non-proteinogenic amino acid may comprise a ring structure. For example, a non-proteinogenic amino acid may be trans-4-aminomethylcyclohexane carboxylic acid or 4-hydrazinobenzoic acid. Such compounds may be FMOC-protected with FMOC (fluorenylmethoxycarbonyl chloride) and utilized in solid-phase peptide synthesis. The structures of these compounds are shown below:

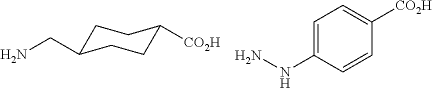 [SMILES: NCC1CCC(C(=O)O)CC1 ; NNc1ccc(C(=O)O)cc1]

Where a labeling reagent or a linker thereof comprises multiple amino acids, such as multiple non-proteinogenic amino acids, an amine moiety adjacent to a ring moiety (e.g., the amine moiety in the hydrazine moiety) can function as a water-solubilizing group. To synthesize a water-soluble peptide, a hybrid linker can be made that comprises alternating non-water-soluble amino acids and water-soluble amino acids (e.g., hydroxyproline). Other moieties can be used to increase water-solubility. For example, linking amino acids with oxamate moieties can provide water-solubility through the additional hydrogen bonding without adding any sp³ linkages. The structure of the oxamate precursor 2-amino-2-oxoacetic acid is shown below:

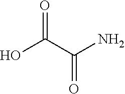

In some cases, a component (e.g., a monomer unit) of a linker may have an amino group, a carboxy group, and a water-solubilizing moiety. In some cases, a monomer may be deconstructed as two “half-monomers.” That is, by using two different units, one that contains two amino groups and another that contains two carboxy groups, an amino acid moiety can be constructed, which amino acid moiety may be a unit (e.g., a repeated unit) of a linker. One or both units may include one or more water solubilizing moieties. For example, at least one unit may include a water-soluble group (e.g., as described herein). For example, 2,5-diaminohydroquinone can be one half-monomer (A), and 2,5-dihydroxyterephthalic acid may be the other half-monomer (B). Such a scheme is shown below:

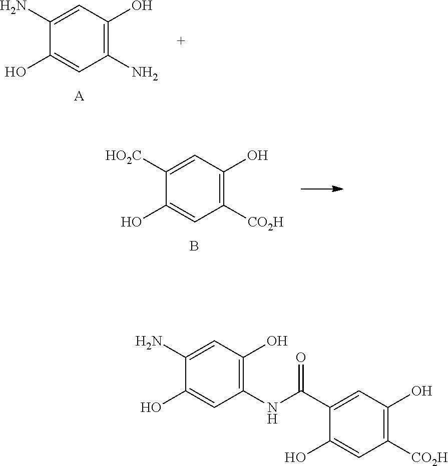

As shown above, A is a diamine and B is a diacid. Accordingly, non-proteinogenic (e.g., non-natural) amino acids may be constructed from diamines and diacids. An additional example of such a construction is shown below:

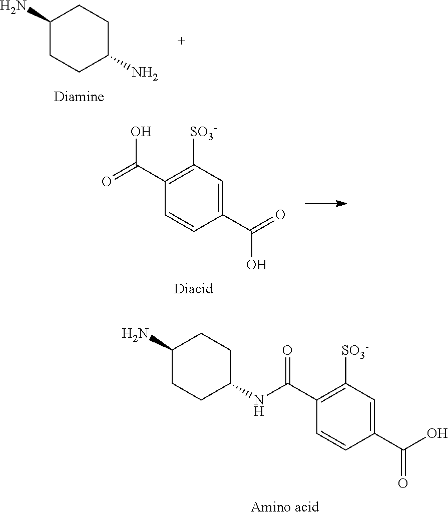

A polymer based on two half-monomers (e.g., as shown above) can be constructed via solid phase synthesis. Because the half-monomers can be homobifunctional in the linking moiety, in some cases no FMOC protection is required. For example, the dicarboxylic acid can be appended to the solid support, then an excess of the diamine added with appropriate coupling reagent (HBTU/HOBT/collidine). After washing away excess reagent, an excess of the dicarboxylic acid can be added with the coupling reagent. Side-products consisting of one molecule of the fluid phase reagent reacting with two solid-phase attached reagent can result in truncation of the synthesis. These side products can be separated from a product after cleavage from the support and purification by HPLC.

An advantage of the half-monomers approach can be increased flexibility in creating polymers. The diamine (A) can be replaced in a subsequent step by a different diamine (A′) to change the properties of the polymer, in a repeating or non-repeating manner. Such a scheme may facilitate construction of a polymer such as ABA′BABA′B.

Additional examples of half-monomers for use according to the schemes described above include 2,5-diaminopyridine and 2,5-dicarboxypyridine, both of which are shown below, as well as the other moieties shown below:

### Diamines:

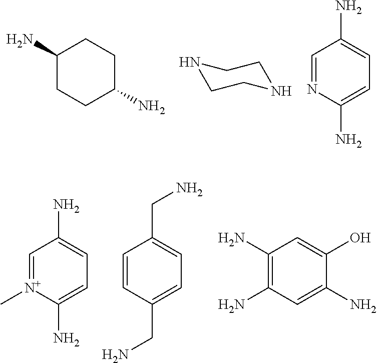

### Dicarboxylic Acids:

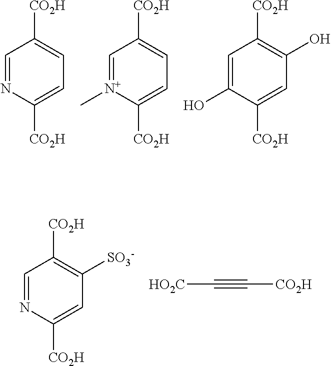

As described above, an amino acid (e.g., a non-proteinogenic amino acid that may be a non-natural amino acid) may be constructed from a diamine and a dicarboxylic acid. An amino acid (e.g., a non-proteinogenic amino acid that may be a non-natural amino acid) may also be constructed from an amino thiol and a thiol carboxylic acid. Examples of amino thiols and thiol carboxylic acids are shown below:

### Amino Thiols:

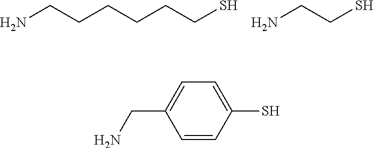

### Thiol Carboxylic Acids:

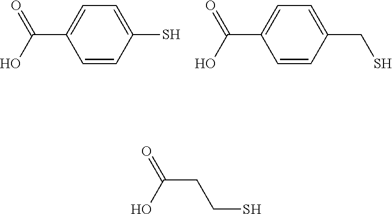 [SMILES: O=C(O)c1ccc(S)cc1 ; O=C(O)c1ccc(CS)cc1 ; O=C(O)CCS]

Examples of amino acids (e.g., non-natural amino acids) constructed from an amino thiol and a thiol carboxylic acid are shown below:

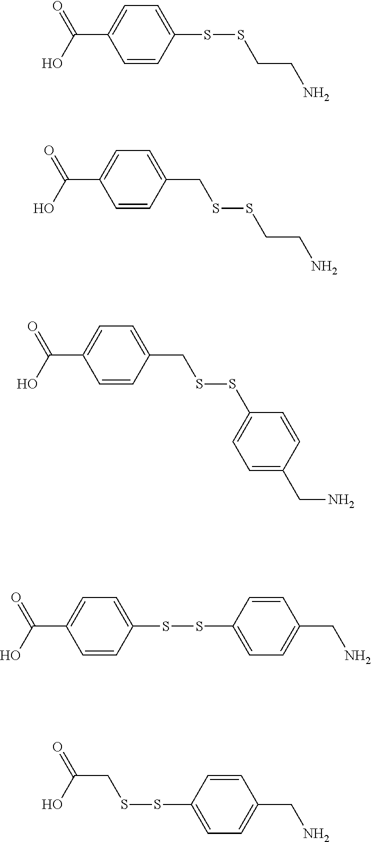 [SMILES: NCCSSc1ccc(C(=O)O)cc1 ; NCCSSCc1ccc(C(=O)O)cc1 ; NCc1ccc(SSCc2ccc(C(=O)O)cc2)cc1 ; NCc1ccc(SSc2ccc(C(=O)O)cc2)cc1 ; NCc1ccc(SSCC(=O)O)cc1]

As shown above, amino acids constructed using an amino thiol and a thiol carboxylic acid may include a disulfide bond. As described elsewhere herein, a disulfide bond may be cleavable using a cleavage reagent (e.g., as described herein). Accordingly, an amino acid constructed from an amino thiol and a thiol carboxylic acid may serve as a cleavable portion of a linker. An amino acid constructed from an amino thiol and a carboxylic acid may be a component of a linker (e.g., as described herein) that may couple labeling moiety (e.g., a fluorescent dye) to a substrate (e.g., a nucleotide or nucleotide analog). The various structures allow different hydrophobicities for incorporation and may provide different “scar” moieties subsequent to interaction with a cleavage reagent (e.g., as described herein). Two or more amino acids, such as two or more amino acids constructed from an amino thiol and a thiol carboxylic acid, may be included in a linker. For example, two or more amino acids may be included in a linker and separated by no more than 2 sp³ carbon atoms, such as by no more than 2 sp² carbon atoms or by no more than 2 atoms. Where two or more amino acids formed of amino thiols and thiol carboxylic acids are connected to one another within a linker, cleavage may be more rapid as there may be multiple possible sites for cleavage. An example of a portion of a linker including such a component is shown below:

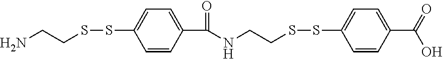 [SMILES: NCCSSc1ccc(C(=O)NCCSSc2ccc(C(=O)O)cc2)cc1]

As described above, two half-monomers may combine to provide an amino acid (e.g., a non-proteinogenic amino acid, such as a non-natural amino acid). Accordingly, a non-natural amino acid may include any known non-natural amino acid, as well as any non-natural amino acid that may be constructed as described herein.

Half-monomers such as those described herein can be constructed into polypeptide polymers. An example of a nucleotide constructed with two repeating units of an amino acid is shown below:

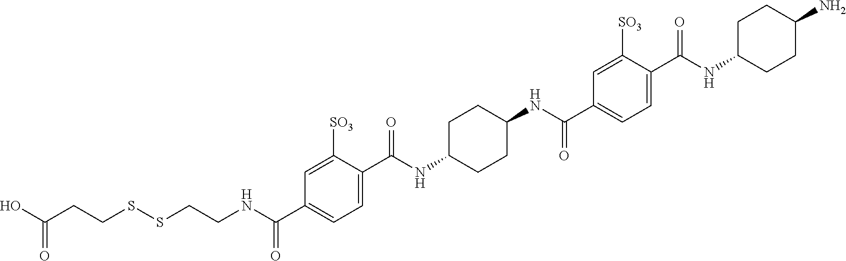

In some cases, before or after peptide coupling, the nitrogen in a nitrogen-containing ring can be quaternized to provide pyridinium moieties, thereby improving water-solubility of the final product. An example linker sequence generated in this manner is shown below:

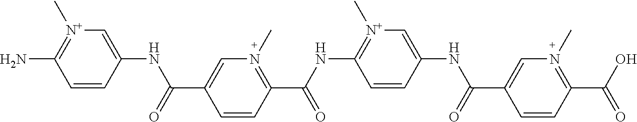

Water-solubilizing linkages that can work with the half-monomer method include, for example, those that have symmetrical functional groups, such as secondary amides, bishydrazides, and ureas. Examples of such moieties are shown below:

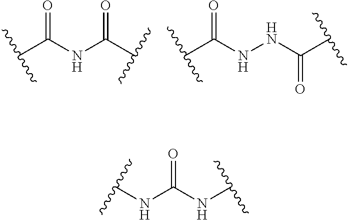 [SMILES: *C(=O)NC(*)=O ; *C(=O)NNC(*)=O ; *NC(=O)N*]

Amino acid linker subunits may be assembled into polymers by peptide synthesis methods. For example, a solid support method known as SPPS (Solid Phase Peptide Synthesis) or by liquid-phase synthesis may be used to assemble amino acids into a linker. SPPS methods can use a solid phase bead where the initial step is attachment of the C-terminal amino acid via its carboxylic acid moiety, leaving its free amine ready for coupling. Peptide synthesis can be initiated by flowing FMOC amine-protected monomers with peptide coupling reagents such as HBTU and an organic base. Excess reagent can be washed away and the next monomer is introduced. After one or more amino acids have been appended the final peptide can be cleaved from the beads and purified by HPLC. Liquid phase synthesis can use the same reagents (except the beads) but purification occurs after each step. The advantage of either stepwise polymerization process is that the resultant linkers can have a defined molecular weight that may be confirmed by mass spectrometry.

A labeling reagent may include any useful combination of amino acids, including any combination of natural and non-natural amino acids and/or proteinogenic and non-proteinogenic amino acids. As described herein, a labeling reagent may comprise a sequence of hydroxyprolines such as a hyp10, hyp20, hyp30, or similar moiety (e.g., hypn).

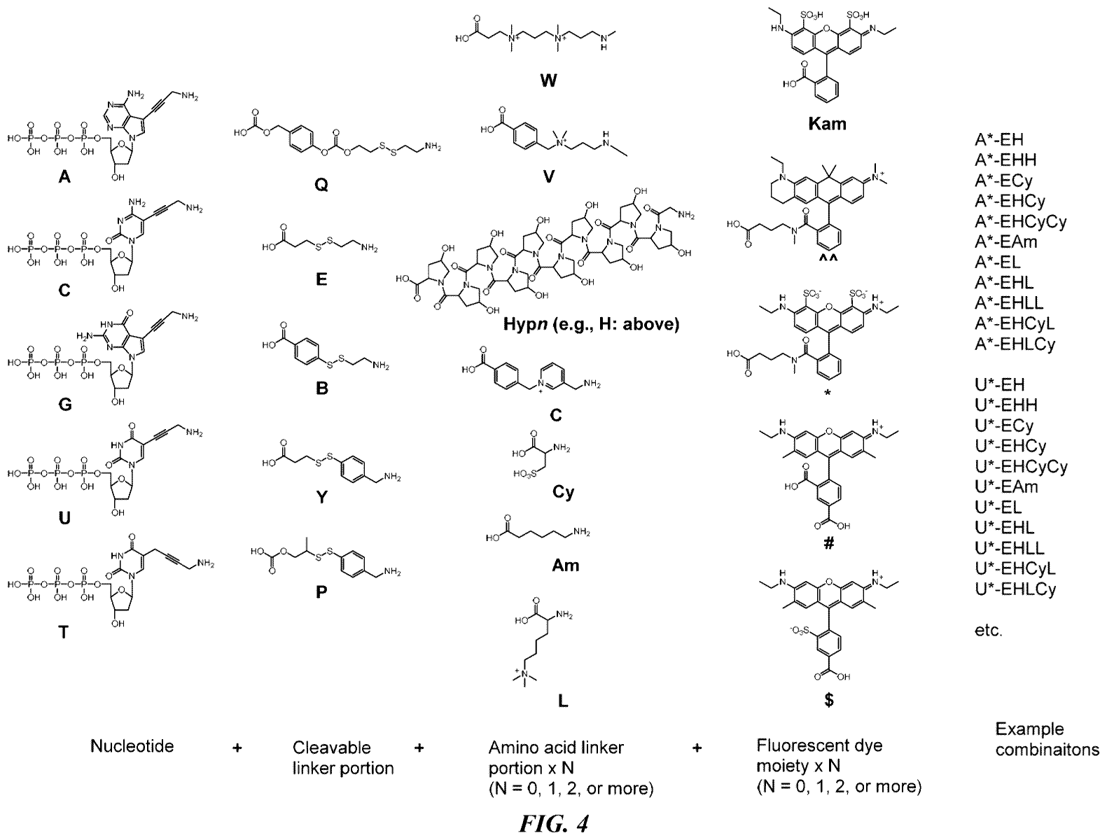

FIG. 4 also illustrates different examples of amino acids that can be a part of a linker, labelled “H”, “C”, “Cy”, “Am”, “V”, “W”, and “L”. A linker may comprise any of, multiples thereof, and/or any combination thereof of these amino acid linker portion examples.

### Quaternary Amines and Cationic Linkers

A labeling reagent may comprise a cationic linker. As an example, a linker may comprise a quaternary amine. Example quaternary amine subunit structures are provided as components “V” and “W” in FIG. 4, or as shown below:

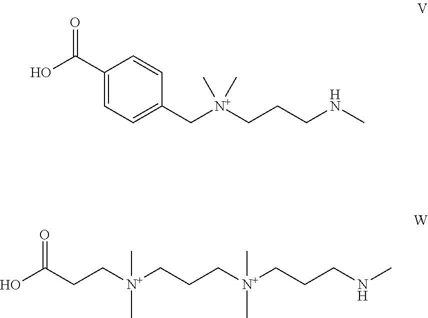 [SMILES: CNCCC[N+](C)(C)Cc1ccc(C(=O)O)cc1 ; CNCCC[N+](C)(C)CCC[N+](C)(C)CCC(=O)O]

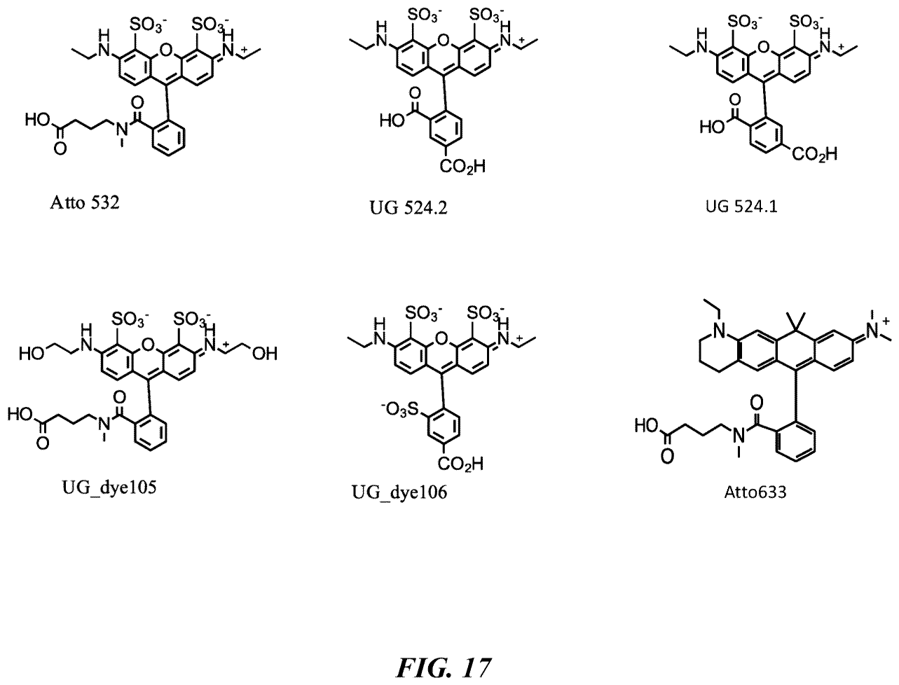

A linker may comprise any number of quaternary amine subunits. For example, a linker may comprise at least 0, 1, 2, 3, 4, 5, 6, 7, 8, 9, 10 or more quaternary amine subunits. Alternatively or in addition, a linker may comprise at most 0, 1, 2, 3, 4, 5, 6, 7, 8, 9, 10 quaternary amine subunits. A linker may comprise one type of quaternary amine subunit or multiple types of quaternary amine subunits. A linker may comprise a quaternary amine at any location of the linker, for example at a location more proximal or more distal to a substrate relative to a different amino acid linker portion. Where multiple quaternary amine subunits are present, in some cases, they may be linked consecutively, or one or more quaternary amine subunits may be separated by other linker subunits (e.g., amino acid subunits, e.g., Hypn). An example structure of a labeling reagent comprising a quaternary amine subunit is outlined below:
[substrate]-[clv]-[quat₁]ₓ-[amino]_{z}-[quat₂]_{y}-[label]; or
   where the [substrate] can be any substrate described herein (e.g., nucleotide bases, proteins, etc.), the [clv] can be any cleavable linker portion described herein (e.g., see “cleavable linker portion” in FIG. 4), the [quat₁] and [quat₂] can be any quaternary amine subunit described herein, the [amino] can be any amino acid linker portion described herein (e.g., see “amino acid linker portion” in FIG. 4), and the [label] can be any label described herein (e.g., dyes, see “fluorescent dye moiety” in FIG. 4 and FIG. 17). x and y can be any non-negative integer such as {0, 1, 2, 3, 4, 5, 6, 7, 8, 9, 10, etc.}. x and y can be the same or different integers. [quat₁] and [quat₂] may be the same or different quaternary amine subunits. z can be any non-negative integer such as {0, 1, 2, 3, 4, 5, 6, 7, 8, 9, 10, etc.}. Where z is 2 or greater, each [amino] of the [amino]_{z} may be the same or different amino acid linker portions. In some examples, [amino]_{z} is a Hypn, such as a Hyp20.

Below are example components of the above structure:
[substrate]=dUTP, dATP, dCTP, dGTP, or dTTP
[clv]=Y cleavable linker (see FIG. 4)
[label]=Kam (see FIG. 4)
[amino]_{z}=Hyp20
[quat₁]=[quat₂]=V (see FIG. 4)
{x, y}={1,0}; {0,1}; {3,0}; or {0,3}
(see below for V₃ structure, where {R1, R2}={[clv], [amino]_{z}} or {[amino]_{z}, [label]}):

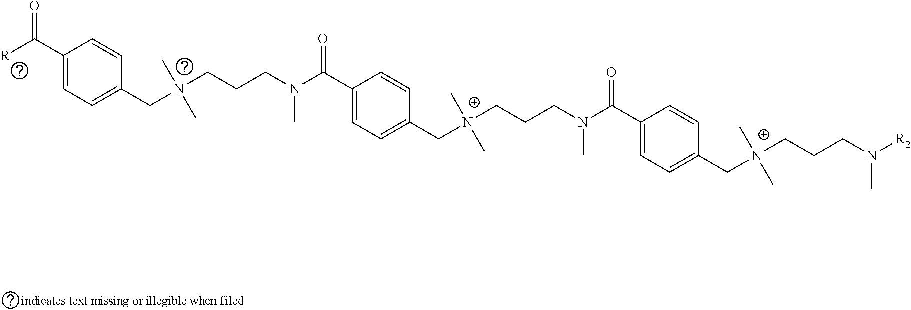

### Cleavable Moieties

A labeling reagent may include one or more cleavable moieties (e.g., as described herein). A cleavable moiety may comprise a cleavable group such as a disulfide moiety. A cleavable moiety may comprise a chemical handle for attachment to a substrate (e.g., as described herein). Accordingly, a cleavable moiety may be included in a labeling reagent at a position adjacent to a substrate to which the labeling reagent is attached. A cleavable moiety may be coupled to a linker component of a labeling reagent via, for example, reaction between a free carboxyl moiety of the linker component and an amino moiety of a cleavable moiety (e.g., cleavable linker portion).

Examples of cleavable linker portions include, but are not limited to, the structures E, B, and Y shown below:

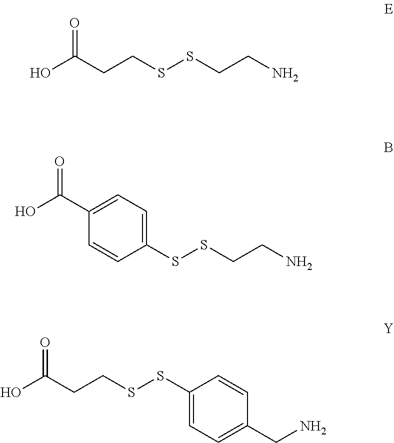 [SMILES: NCCSSCCC(=O)O ; NCCSSc1ccc(C(=O)O)cc1 ; NCc1ccc(SSCCC(=O)O)cc1]

In the structures shown above, the disulfide moieties may be cleaved (e.g., as described herein) to provide thiol scars. Variations of the structures shown above are also contemplated. For example, one or more substituents such as one or more alkyl, hydroxyl, alkoxy, or halo moieties may be attached to a ring structure or an available carbon atom in any of the above structures. Similarly, though para-attachment of carboxyl and disulfide moieties is illustrated, meta- and ortho-attachments may also be used. Moreover, an optionally substituted alkyl group may be incorporated between a ring structure and a disulfide moiety. A cleavable linker portion may be attached to a substrate upon reaction between a carboxyl moiety of the cleavable linker moiety and an amine moiety attached to a substrate (e.g., protein, nucleotide or nucleotide analog, cell, etc., as described herein) to provide the substrate attached to the cleavable linker portion via an amide moiety. For example, the substrate may be a nucleotide or nucleotide analog including a propargylamino moiety, and a fluorescent labeling reagent comprising a dye and a linker described herein may be configured to associate with the substrate via the propargylamino moiety. Examples of such substrates are shown below:

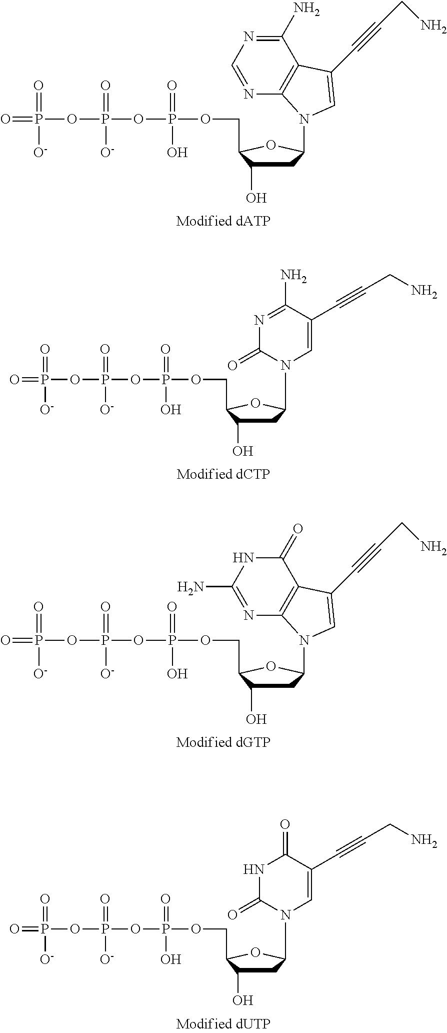

FIG. 4 also illustrates different examples of cleavable groups that can be a part of a linker, labelled “Q,” “E,” “B,” “Y,” and “P”. A linker may comprise any of these cleavable group examples.

### Optically Detectable Moieties

As described herein, a labeling reagent may comprise one or more optically detectable moieties. Multiple optically detectable moieties (e.g., fluorescent dye moieties) included in a given labeling reagent may have the same or different chemical structures. Similarly, multiple optically detectable moieties (e.g., fluorescent dye moieties) included in a given labeling reagent may fluoresce at or near the same wavelengths or may fluoresce at or near different wavelengths. A given linker component (e.g., semi-rigid linker component) may be configured to couple to a single optically detectable moiety. Alternatively, a given linker component (e.g., semi-rigid linker component) may be configured to couple to two or more optically detectable moieties that may have the same or different chemical structures. A labeling reagent may include multiple linkers coupled to multiple optically detectable moieties via, e.g., a scaffold such as a lysine or polylysine scaffold (e.g., as described herein). Optically detectable moieties coupled to a labeling reagent may facilitate optical (e.g., fluorescent) labeling of a substrate to which the labeling reagent may attach. For example, the labeling reagent may be used to optically label a protein, nucleotide, nucleotide analog, polynucleotide, antibody, cell, saccharide, polysaccharide, lipid, cell surface marker, or any other useful substrate (e.g., as described herein) with one or more optically detectable moieties. When coupled to a substrate, a labeling reagent comprising multiple optically detectable moieties configured to provide a similar optical signal (e.g., configured to fluoresce at or near the same wavelengths) may provide an enhanced signal relative to a labeling reagent comprising a single optically detectable moiety.

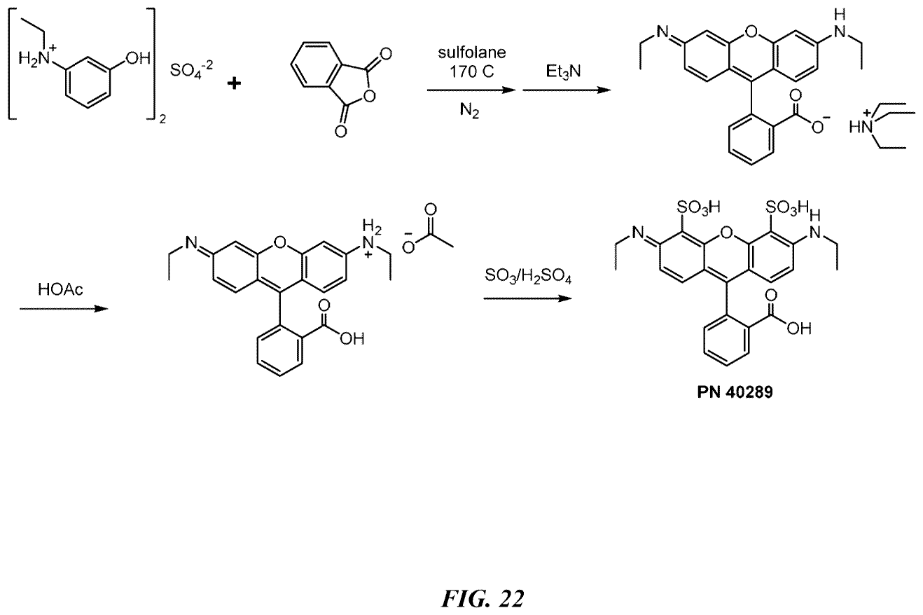

An optically detectable moiety may comprise a dye (e.g., a fluorescent dye). Non-limiting examples of dyes (e.g., fluorescent dyes) include SYBR green, SYBR blue, DAPI, propidium iodine, Hoechst, SYBR gold, ethidium bromide, acridine, proflavine, acridine orange, acriflavine, fluorocoumarin, ellipticine, daunomycin, chloroquine, distamycin D, chromomycin, homidium, mithramycin, ruthenium polypyridyls, anthramycin, phenanthridines and acridines, propidium iodide, hexidium iodide, dihydroethidium, ethidium homodimer-1 and -2, ethidium monoazide, ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO dyes (e.g., SYTO-40, -41, -42, -43, -44, and -45 (blue); SYTO-13, -16, -24, -21, -23, -12, -11, -20, -22, -15, -14, and -25 (green); SYTO-81, -80, -82, -83, -84, and -85 (orange); SYTO-64, -17, -59, -61, -62, -60, and -63 (red)), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosine, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor dyes (e.g., AlexaFluor 350, 405, 430, 488, 532, 546, 555, 568, 594, 610, 633, 635, 647, 660, 680, 700, 750, and 790 dyes), DyLight dyes (e.g., DyLight 350, 405, 488, 550, 594, 633, 650, 680, 755, and 800 dyes), Black Hole Quencher Dyes (Biosearch Technologies) (e.g., BH1-0, BHQ-1, BHQ-3, and BHQ-10), QSY Dye fluorescent quenchers (from Molecular Probes/Invitrogen)(e.g., QSY7, QSY9, QSY21, and QSY35), Dabcyl, Dabsyl. Cy5Q, Cy7Q, Dark Cyanine dyes (GE Healthcare), Dy-Quenchers (Dyomics) (e.g., DYQ-660 and DYQ-661), ATTO fluorescent quenchers (ATTO-TEC GmbH) (e.g., ATTO 540Q, 580Q, 612Q, 532, and 633), Kam, and other fluorophores and quenchers (e.g., as described herein). Additional examples of dyes are shown in FIGS. 17 and 22.

A fluorescent dye may be excited over a single wavelength or a range of wavelengths. In some cases, an optical labeling reagent may comprise an optically detectable moiety configured to fluoresce in the red region of the electromagnetic spectrum (e.g., (about 625-740 nm). For example, a labeling reagent may include a fluorescent dye that may emit signal in the red region of the visible portion of the electromagnetic spectrum (about 625-740 nm) (e.g., have an emission maximum in the red region of the visible portion of the electromagnetic spectrum). Alternatively or additionally, an optical labeling reagent may comprise an optically detectable moiety configured to fluoresce in the green region of the electromagnetic spectrum (e.g., about 500-565 nm). For example, a labeling reagent may include a fluorescent dye that may emit signal in the green region of the visible portion of the electromagnetic spectrum (about 500-565 nm) (e.g., have an emission maximum in the green region of the visible portion of the electromagnetic spectrum). Similarly, a fluorescent dye may be excitable by light in the red region of the visible portion of the electromagnetic spectrum (about 625-740 nm) (e.g., have an excitation maximum in the red region of the visible portion of the electromagnetic spectrum). Alternatively or additionally, fluorescent dye may be excitable by light in the green region of the visible portion of the electromagnetic spectrum (about 500-565 nm) (e.g., have an excitation maximum in the green region of the visible portion of the electromagnetic spectrum). In an example, an optical labeling reagent may include a plurality of optically detectable moieties configured to fluoresce in the red region of the visible portion of the electromagnetic spectrum, which plurality of optically detectable moieties may have the same or different structures. In another example, an optical labeling reagent may include a plurality of optically detectable moieties configured to fluoresce in the green region of the visible portion of the electromagnetic spectrum, which plurality of optically detectable moieties may have the same or different structures.

In some cases, the label may be a type that does not self-quench or exhibit proximity quenching. Non-limiting examples of a label type that does not self-quench or exhibit proximity quenching include Bimane derivatives such as Monobromobimane. Additional dyes included in structures provided herein may also be utilized in combination with any of the linkers provided herein, and with any substrate described herein, regardless of the context of their disclosure. In some cases, an optically detectable moiety may comprise a dye pair (e.g., two or more dye structures). A labeling reagent including any useful optically detectable moiety, or any combination of optically detectable moieties, may be useful in, for example, labeling a nucleotide or nucleotide analog for use in a sequencing assay. For example, a sequencing assay performed with a nucleotide labeled with a red-fluorescing dye and a sequencing assay performed with a nucleotide labeled a green-fluorescing dye may have sequencing quality and signal-to-noise ratios, as well as other performance metrics.

FIG. 4 illustrates different examples of fluorescent dye moieties that can be attached to a linker, labelled “Kam” (PN 40289), “\hat{ }\hat{ }”, “*”, “#”, and “$”. FIG. 17 provides additional examples of dyes. A linker may be attached to any of, multiples thereof, and/or any combination thereof of these fluorescent dye moieties.

### Labeled Substrates

A substrate may be coupled to a labeling reagent. The substrate coupled to the labeling reagent may be a detectably labeled substrate. The labeling reagent may comprise a detectable moiety. The substrate may comprise a nucleobase. In some cases, the nucleobase comprises adenine, cytosine, thymine, or uracil. In some cases, the nucleobase is adenine. In some cases, the nucleobase is cytosine. In some cases, the nucleobase is thymine. In some cases, the nucleobase is uracil. In some cases, the nucleobase is not guanine. The substrate may comprise a nucleoside. The substrate may comprise a nucleotide. The substrate may comprise a deoxyribose nucleotide triphosphate. In some cases, the substrate may comprise a ribose nucleotide triphosphate.

The substrate may comprise a linker. The linker may comprise at least a first non-proteinogenic amino acid and a second non-proteinogenic amino acid. In some cases, the linker may comprise a first non-proteinogenic amino acid and a second non-proteinogenic amino acid. In some cases, the first non-proteinogenic amino acid and the second non-proteinogenic amino acid may be different. In some cases, the first non-proteinogenic amino acid and the second non-proteinogenic amino acid may be the same. In some cases, the first non-proteinogenic amino acid and the second non-proteinogenic amino acid may be a same type. The first non-proteinogenic amino acid may comprise a hydroxyproline. The first non-proteinogenic amino acid may comprise at least about 10 hydroxyprolines. The first non-proteinogenic amino acid may comprise 10 hydroxyprolines. The first non-proteinogenic amino acid may comprise at least about 20 hydroxyprolines. The first non-proteinogenic amino acid may comprise 20 hydroxyprolines. The first non-proteinogenic amino acid may comprise more than 20 hydroxyprolines. The second non-proteinogenic amino acid may comprise cysteic acid. The second non-proteinogenic amino acid may comprise 5-amino-5-carboxy-N,N,N-trimethylpentan-1-aminium or a salt thereof. The second non-proteinogenic amino acid may comprise 6-aminohexanoic acid. The first non-proteinogenic amino acid may comprise hydroxyproline, and the second non-proteinogenic amino acid may comprise cysteic acid. The first non-proteinogenic amino acid may comprise hydroxyproline, and the second non-proteinogenic amino acid may comprise 5-amino-5-carboxy-N,N,N-trimethylpentan-1-aminium or a salt thereof. The first non-proteinogenic amino acid may comprise at least about 10 or 20 hydroxyprolines, and the second non-proteinogenic amino acid may comprise one, two or more 5-amino-5-carboxy-N,N,N-trimethylpentan-1-aminium or a salt thereof. The first non-proteinogenic amino acid may comprise at least about 10 hydroxyprolines, and the second non-proteinogenic amino acid may comprise cysteic acid. The first non-proteinogenic amino acid may comprise 10 hydroxyprolines, and the second non-proteinogenic amino acid may comprise cysteic acid. The first non-proteinogenic amino acid may comprise at least about 20 hydroxyprolines, and the second non-proteinogenic amino acid may comprise cysteic acid. The first non-proteinogenic amino acid may comprise 20 hydroxyprolines, and the second non-proteinogenic amino acid may comprise cysteic acid. The first non-proteinogenic amino acid may comprise more than 20 hydroxyprolines, and the second non-proteinogenic amino acid may comprise cysteic acid. The first non-proteinogenic amino acid may comprise hydroxyproline, and the second non-proteinogenic amino acid may comprise 6-aminohexanoic acid. The first non-proteinogenic amino acid may comprise at least 10 hydroxyprolines, and the second non-proteinogenic amino acid may comprise 6-aminohexanoic acid. The first non-proteinogenic amino acid may comprise 10 hydroxyprolines, and the second non-proteinogenic amino acid may comprise 6-aminohexanoic acid. The first non-proteinogenic amino acid may comprise at least 20 hydroxyprolines, and the second non-proteinogenic amino acid may comprise 6-aminohexanoic acid. The first non-proteinogenic amino acid may comprise 20 hydroxyprolines, and the second non-proteinogenic amino acid may comprise 6-aminohexanoic acid. The first non-proteinogenic amino acid may comprise more than 20 hydroxyprolines, and the second non-proteinogenic amino acid may comprise 6-aminohexanoic acid. The first non-proteinogenic amino acid may comprise any non-proteinogenic amino acids described herein. The second non-proteinogenic amino acid may comprise any non-proteinogenic amino acids described herein.

In some instances, the substrate may comprise at least two non-proteinogenic amino acids. In some cases, the two non-proteinogenic amino acids may be the same. In some cases, the two non-proteinogenic amino acids may be a same type. In some cases, the two non-proteinogenic amino acids may be cysteic acids. In some cases, the two non-proteinogenic amino acids may be 5-amino-5-carboxy-N,N,N-trimethylpentan-1-aminium or a salt thereof.

The substrate may comprise a nucleotide coupled to the first non-proteinogenic amino acid. The substrate may comprise a nucleotide coupled to the second non-proteinogenic amino acid. The substrate may comprise a nucleotide coupled to the first non-proteinogenic amino acid and the first non-proteinogenic amino acid coupled to second non-proteinogenic acid. The substrate may comprise a cleavable group. The substrate may comprise a nucleotide coupled to a cleavable group, the cleavable coupled to the first non-proteinogenic amino acid, and the first non-proteinogenic amino acid coupled to second non-proteinogenic acid. The substrate may comprise a nucleotide coupled to a cleavable group and the cleavable coupled to a non-proteinogenic amino acid. The substrate may comprise a nucleotide coupled to a cleavable group and the cleavable coupled to the first non-proteinogenic amino acid. The substrate may comprise a nucleotide coupled to a cleavable group and the cleavable coupled to the second non-proteinogenic amino acid. The substrate may further be coupled to a detectable moiety. The detectable moiety may be coupled to the non-proteinogenic acid, the first non-proteinogenic amino acid, the second non-proteinogenic amino acid, the cleavable group, the nucleobase, the nucleotide, the nucleoside, or a combination thereof. In some cases, the substrate may comprise a compound of the formula below:

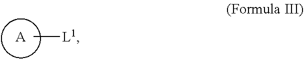

wherein: A comprises a nucleobase; and L¹ is a linker comprising at least a first non-proteinogenic amino acid and a second non-proteinogenic amino acid.

In some cases, A may comprise a nucleobase. In some cases, the nucleobase comprises adenine, cytosine, thymine, or uracil. In some cases, the nucleobase is adenine. In some cases, the nucleobase is cytosine. In some cases, the nucleobase is thymine. In some cases, the nucleobase is uracil. In some cases, the nucleobase is not guanine. A may comprise a nucleoside. A may comprise a nucleotide. A may comprise a deoxyribose nucleotide triphosphate. In some cases, A may comprise a ribose nucleotide triphosphate.

L¹ may comprise a linker. The linker may comprise at least a first non-proteinogenic amino acid and a second non-proteinogenic amino acid. In some cases, L¹ may comprise a first non-proteinogenic amino acid and a second non-proteinogenic amino acid. The first non-proteinogenic amino acid may comprise a hydroxyproline. The first non-proteinogenic amino acid may comprise at least about 10 hydroxyprolines. The first non-proteinogenic amino acid may comprise 10 hydroxyprolines. The first non-proteinogenic amino acid may comprise at least about 20 hydroxyprolines. The first non-proteinogenic amino acid may comprise 20 hydroxyprolines. The first non-proteinogenic amino acid may comprise more than 20 hydroxyprolines. The second non-proteinogenic amino acid may comprise cysteic acid. The second non-proteinogenic amino acid may comprise 5-amino-5-carboxy-N,N,N-trimethylpentan-1-aminium or a salt thereof. The second non-proteinogenic amino acid may comprise 6-aminohexanoic acid. The first non-proteinogenic amino acid may comprise hydroxyproline, and the second non-proteinogenic amino acid may comprise cysteic acid. The first non-proteinogenic amino acid may comprise at least about 10 hydroxyprolines, and the second non-proteinogenic amino acid may comprise cysteic acid. The first non-proteinogenic amino acid may comprise 10 hydroxyprolines, and the second non-proteinogenic amino acid may comprise cysteic acid. The first non-proteinogenic amino acid may comprise at least about 20 hydroxyprolines, and the second non-proteinogenic amino acid may comprise cysteic acid. The first non-proteinogenic amino acid may comprise 20 hydroxyprolines, and the second non-proteinogenic amino acid may comprise cysteic acid. The first non-proteinogenic amino acid may comprise more than 20 hydroxyprolines, and the second non-proteinogenic amino acid may comprise cysteic acid. The first non-proteinogenic amino acid may comprise hydroxyproline, and the second non-proteinogenic amino acid may comprise 5-amino-5-carboxy-N,N,N-trimethylpentan-1-aminium or a salt thereof. The first non-proteinogenic amino acid may comprise at least about 10 hydroxyprolines, and the second non-proteinogenic amino acid may comprise 5-amino-5-carboxy-N,N,N-trimethylpentan-1-aminium or a salt thereof. The first non-proteinogenic amino acid may comprise 10 hydroxyprolines, and the second non-proteinogenic amino acid may comprise 5-amino-5-carboxy-N,N,N-trimethylpentan-1-aminium or a salt thereof. The first non-proteinogenic amino acid may comprise at least about 20 hydroxyprolines, and the second non-proteinogenic amino acid may comprise 5-amino-5-carboxy-N,N,N-trimethylpentan-1-aminium or a salt thereof. The first non-proteinogenic amino acid may comprise 20 hydroxyprolines, and the second non-proteinogenic amino acid may comprise 5-amino-5-carboxy-N,N,N-trimethylpentan-1-aminium or a salt thereof. The first non-proteinogenic amino acid may comprise more than 20 hydroxyprolines, and the second non-proteinogenic amino acid may comprise 5-amino-5-carboxy-N,N,N-trimethylpentan-1-aminium or a salt thereof. The first non-proteinogenic amino acid may comprise hydroxyproline, and the second non-proteinogenic amino acid may comprise 6-aminohexanoic acid. The first non-proteinogenic amino acid may comprise at least about 10 hydroxyprolines, and the second non-proteinogenic amino acid may comprise 6-aminohexanoic acid. The first non-proteinogenic amino acid may comprise 10 hydroxyprolines, and the second non-proteinogenic amino acid may comprise 6-aminohexanoic acid. The first non-proteinogenic amino acid may comprise at least about 20 hydroxyprolines, and the second non-proteinogenic amino acid may comprise 6-aminohexanoic acid. The first non-proteinogenic amino acid may comprise 20 hydroxyprolines, and the second non-proteinogenic amino acid may comprise 6-aminohexanoic acid. The first non-proteinogenic amino acid may comprise more than 20 hydroxyprolines, and the second non-proteinogenic amino acid may comprise 6-aminohexanoic acid. The first non-proteinogenic amino acid may comprise any non-proteinogenic amino acids described herein. The second non-proteinogenic amino acid may comprise any non-proteinogenic amino acids described herein.

In some instances, L¹ may comprise at least two non-proteinogenic amino acids. In some cases, the two non-proteinogenic amino acids may be the same. In some cases, the two non-proteinogenic amino acids may be a same type. In some cases, the two non-proteinogenic amino acids may be cysteic acids. In some cases, the two non-proteinogenic amino acids may be 5-amino-5-carboxy-N,N,N-trimethylpentan-1-aminium or a salt thereof.

In some cases, a detectably labeled substrate may comprise a compound of Formula IIIa below:

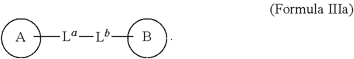

In some cases, A may comprise a nucleobase. In some cases, B may comprise the detectable moiety. In some cases, L^{b} is a linker and may comprise the first non-proteinogenic amino acid or the second non-proteinogenic amino acid. In some cases, L^{b} may comprise the first non-proteinogenic amino acid and the second non-proteinogenic amino acid. In some cases, L² may comprise the first non-proteinogenic amino acid. In some cases, L^{b} may comprise the second non-proteinogenic amino acid. In some cases, L^{b} may comprise a non-proteinogenic amino acid. In some cases, linker L^{a} may comprise the cleavable group. In some instances, the detectably labeled substrate may comprise a compound of formula IIIb or formula IIIc below:

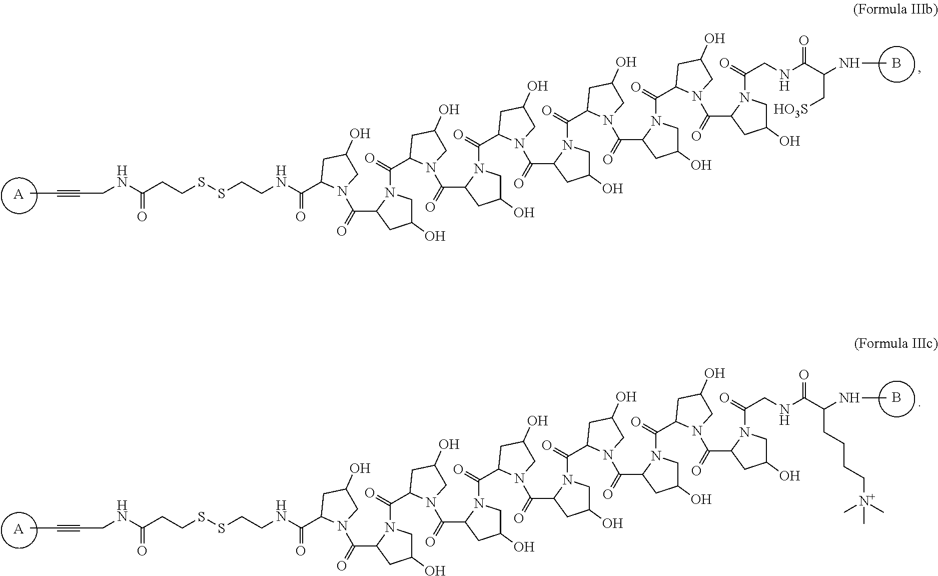

In some cases, L² may comprise at least two non-proteinogenic amino acids. In some cases, L² may comprise two non-proteinogenic amino acids. In some cases, L² may comprise at least two cysteic acids. In some cases, L² may comprise two cysteic acids. In some cases, L² may comprise at least two 5-amino-5-carboxy-N,N,N-trimethylpentan-1-aminium or a salt thereof. In some cases, L² may comprise two 5-amino-5-carboxy-N,N,N-trimethylpentan-1-aminium or a salt thereof. In some cases, L² may comprise a third non-proteinogenic amino acid. In some cases, the third non-proteinogenic amino acid may be different from the at least two non-proteinogenic amino acids. In some cases, the third non-proteinogenic amino acid may comprise hydroxyproline. In some cases, L² may comprise at least about 10 hydroxyprolines. L² may also at least about 10 to about 20 hydroxyprolines. In some instances, the detectably labeled substrate may comprise a compound of Formula Ivc or a compound of Formula Ivd below:

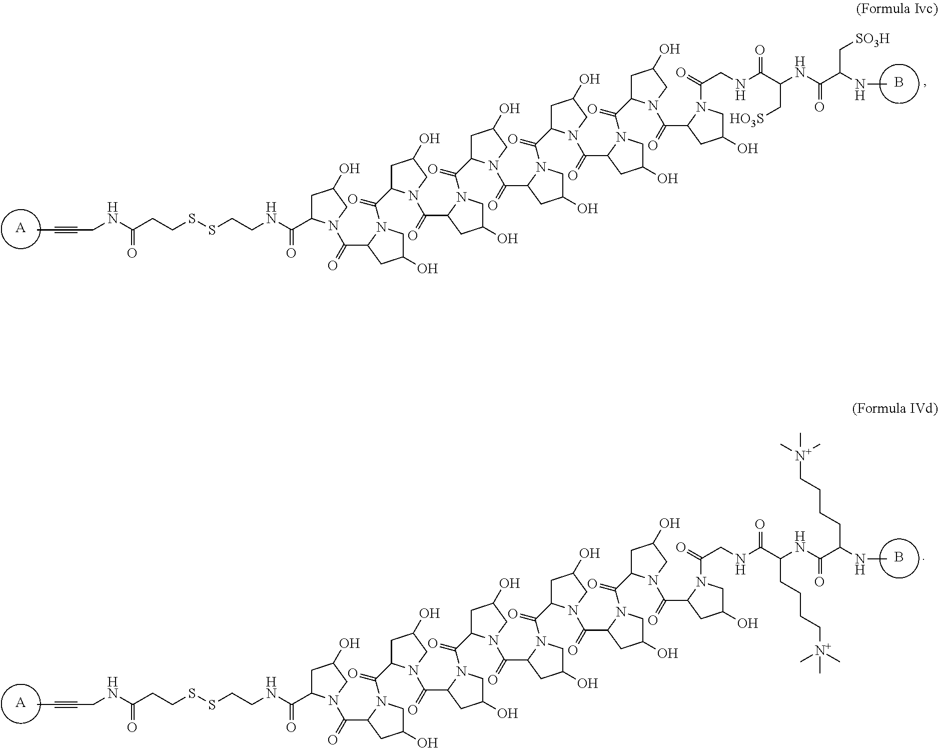

An optical (e.g., fluorescent) labeling reagent may be configured to associate with a substrate such as a nucleotide or nucleotide analog (e.g., as described herein). Alternatively or additionally, an optical (e.g., fluorescent) labeling reagent may be configured to associate with a substrate such as a protein, cell, lipid, or antibody. For example, the optical labeling reagent may be configured to associate with a protein. A protein substrate may be any protein, and may include any useful modification, mutation, or label, including any isotopic label. For example, a protein may be an antibody such as a monoclonal antibody. A protein associated with one or more optical (e.g., fluorescent) labeling reagents (e.g., as described herein) may be, for example, an antibody (e.g., a monoclonal antibody) useful for labeling a cell, which labeled cell may be analyzed and sorted using flow cytometry.

An optical (e.g., fluorescent) labeling reagent (e.g., as described herein) can decrease quenching (e.g., between dyes coupled to nucleotides or nucleotide analogs incorporated into a growing nucleic acid strand, such as during nucleic acid sequencing). For example, an optical (e.g., fluorescent) signal emitted by a substrate (e.g., a nucleotide or nucleotide analog that may be incorporated into a growing nucleic acid strand) can be proportional to the number of optical (e.g., fluorescent) labels associated with the substrate (e.g., to the number of optical labels incorporated adjacent or in proximity to the substrate). For example, multiple optical labeling reagents including substrates of the same or different types (e.g., nucleotides or nucleotide analogs of a same or different type) may be incorporated in proximity to one another in a growing nucleic acid strand (e.g., during nucleic acid sequencing). In such a system, signal emitted by the collective substrates may be approximately proportional (e.g., linearly proportional) to the number of dye-labeled substrates incorporated. In other words, quenching may not significantly impact the signal emitted. This may be observable in a system in which 100% labeling fractions are used. Where less than 100% of substrates are labeled (e.g., less than 100% of nucleotides in a nucleotide flow are labeled), an optical (e.g., fluorescent) signal emitted by substrates (e.g., nucleotides or nucleotide analogs) incorporated into a plurality of growing nucleic acid strands (e.g., a plurality of growing nucleic acid strands coupled to sequencing templates coupled to a support, as described herein) may be proportional to the length of a homopolymer region of the growing nucleic acid strands. Similarly, where less than 100% of substrates are labeled (e.g., less than 100% of nucleotides in each of successive nucleotide flows are labeled), an optical (e.g., fluorescent) signal emitted by substrates (e.g., nucleotides or nucleotide analogs) incorporated into a plurality of growing nucleic acid strands (e.g., a plurality of growing nucleic acid strands coupled to sequencing templates coupled to a support, as described herein) may be proportional to the length of a heteropolymeric and/or homopolymer region of the growing nucleic acid strands. In some such cases, the intensity of a measured optical (e.g., fluorescent) signal may be linearly proportional to the length of a heteropolymeric and/or homopolymeric region into which substrates have incorporated. For example, a measured optical (e.g., fluorescent) signal may be linearly proportional with a slope of approximately 1.0 when optical (e.g., fluorescent) signal is plotted against the length in substrates of a heteropolymeric and/or homopolymeric region into which substrates have incorporated.

An optical (e.g., fluorescent) labeling reagent (e.g., as described herein) can decrease quenching in a protein system. When labeling proteins, quenching may start to happen at a fluorophore to protein ratio (F/P) of around 3. Using optical labeling reagents provided herein, higher F/P ratios, and thus brighter reagents, may be obtained. This may be useful for analyzing proteins (e.g., using imaging) and/or for analyzing cells labeled with proteins (e.g., antibodies) associated with one or more optical (e.g., fluorescent) labeling reagents.

Examples of labeling reagents provided herein, or components thereof, are included in various figures of the present disclosure. Additional examples are included elsewhere herein, including in the Examples below. Any useful labeling reagent may be used to label any substrate of interest.

In an aspect, the present disclosure provides a labeled substrate comprising a substrate (e.g., as described herein) and an optical labeling reagent (e.g., as described herein), or a derivative thereof, where the optical labeling reagent is coupled to the substrate. The substrate may be, for example, a nucleotide, polynucleotide, protein, lipid, cell, saccharide, polysaccharide, or antibody. For example, the substrate may be a protein. Alternatively or additionally, the substrate may be a component of a cell. In another example, the substrate may be a nucleotide or nucleotide analog and the optical labeling reagent may be coupled to the nucleotide via the nucleobase of the nucleotide. The substrate may be a fluorescence quencher, a fluorescence donor, or a fluorescence acceptor. The labeled substrate may reduce quenching relative to another labeled substrate comprising the substrate and another fluorescent labeling reagent that comprises one or more optically detectable moieties but does not include a linker provided herein. Similarly, the labeled substrate may provide a higher signal level upon excitation and optical detection relative to another labeled substrate comprising the substrate and another fluorescent labeling reagent that comprises one or more optically detectable moieties but does not include a linker provided herein.

The substrate may comprise an additional optical labeling reagent (e.g., fluorescent labeling reagent) coupled thereto. The additional optical labeling reagent may comprise an optically detectable moiety (e.g., fluorescent dye moiety) and a linker connected to the optically detectable moiety. The linker and optically detectable moiety of the additional optical labeling reagent may be coupled to the substrate via a cleavable linker portion (e.g., as described herein). The additional optical labeling reagent may include a scaffold to which multiple linkers and optically detectable moieties may be coupled (e.g., as described herein). An optically detectable moiety of a first optical labeling reagent coupled to a substrate and an optically detectable moiety of a second optical labeling reagent coupled to the same substrate may have identical chemical structures. Alternatively or additionally, an optically detectable moiety of a first optical labeling reagent coupled to a substrate and an optically detectable moiety of a second optical labeling reagent coupled to the same substrate may have different chemical structures.

In an aspect, the present disclosure provides an oligonucleotide molecule comprising a fluorescent labeling reagent or derivative thereof (e.g., as described herein). The oligonucleotide molecule may comprise one or more additional fluorescent labeling reagents of a same type (e.g., comprising linkers having the same chemical structure, dyes comprising the same chemical structure, and/or associated with substrates (e.g., nucleotides) of a same type). The fluorescent labeling reagent and one or more additional fluorescent labeling reagents of the oligonucleotide molecule may be associated with nucleotides. For example, the fluorescent labeling reagents may be connected to nucleobases of nucleotides of the oligonucleotide molecule. A fluorescent labeling reagent and one or more additional fluorescent labeling reagent may be connected to adjacent nucleotides of the oligonucleotide molecule. Alternatively or additionally, the fluorescent labeling reagent and the one or more additional fluorescent labeling reagents may be connected to nucleotides of the oligonucleotide molecule that are separated by one or more nucleotides that are not connected to fluorescent labeling reagents. The oligonucleotide molecule may be a single-stranded molecule. Alternatively, the oligonucleotide molecule may be a double-stranded or partially double-stranded molecule. A double-stranded or partially double-stranded molecule may comprise fluorescent labeling reagents associated with a single strand or both strands. The oligonucleotide molecule may be a deoxyribonucleic acid molecule. The oligonucleotide molecule may a ribonucleic acid molecule. The oligonucleotide molecule may be generated and/or modified via a nucleic acid sequencing process (e.g., as described herein).

The fluorescent labeling reagent may comprise a cleavable group (e.g., as described herein) that is configured to be cleaved to separate the fluorescent dye of the fluorescent labeling reagent from a substrate (e.g., nucleotide) with which it is associated. For example, the labeling reagent may comprise a cleavable group comprising an azidomethyl group, a disulfide bond, a hydrocarbyldithiomethyl group, or a 2-nitrobenzyloxy group. The cleavable group may be configured to be cleaved by application of one or more members of the group consisting of tris(2-carboxyethyl)phosphine (TCEP), dithiothreitol (DTT), tetrahydropyranyl (THP), ultraviolet (UV) light, and a combination thereof. The oligonucleotide molecule comprising a fluorescent labeling reagent may be configured to emit a fluorescent signal (e.g., upon excitation at an appropriate range of energy, as described herein).

In another aspect, the present disclosure provides a kit comprising a plurality of linkers (e.g., as described herein). A linker may be a component of an optical labeling reagent provided herein. A linker may be linked to a scaffold such as a lysine or polylysine scaffold. A linker may comprise a cleavable group (e.g., as described herein) configured to be cleaved to separate a linker from a substrate to which it may be attached. A linker may comprise one or more amino acids, such as one or more non-proteinogenic amino acids. For example, a linker may comprise at least one hydroxyproline. A linker may comprise a hyp10, hyp20, hyp30, or other hypn moieties. Alternatively or additionally, a linker may comprise a non-natural amino acid (e.g., as described herein). A linker may be configured to provide a functional separation between an optically detectable moiety and a substrate of at least, e.g., about 9 Å, such as at least 12 Å, 15 Å, 20 Å, 25 Å, 30 Å, 36 Å, or more (e.g., as described herein). A linker may be connected to an optically detectable moiety (e.g., fluorescent dye; as described herein) and/or associated with a substrate (e.g., as described herein). For example, the linker may be connected to a fluorescent dye and coupled to a substrate selected from a nucleotide, a protein, a lipid, a cell, and an antibody. For example, the linker may be connected to an optically detectable moiety (e.g., fluorescent dye) and a substrate such as a nucleotide.

A linker may comprise a plurality of amino acids, such as a plurality of non-proteinogenic (e.g., non-natural) amino acids. For example, the linker may comprise a plurality of hydroxyprolines (e.g., a hyp10 moiety or other hypn moieties). A linker may comprise a cleavable group that is configured to be cleaved to separate a first portion of the linker from a second portion of the linker. The cleavable group may be selected from the group consisting of an azidomethyl group, a disulfide bond, a hydrocarbyldithiomethyl group, and a 2-nitrobenzyloxy group. The cleavable group may be cleavable by application of one or more members of the group consisting of tris(2-carboxyethyl)phosphine (TCEP), dithiothreitol (DTT), tetrahydropyranyl (THP), ultraviolet (UV) light, and a combination thereof. The linker may comprise a cleavable linker portion comprising a moiety selected from the group consisting of

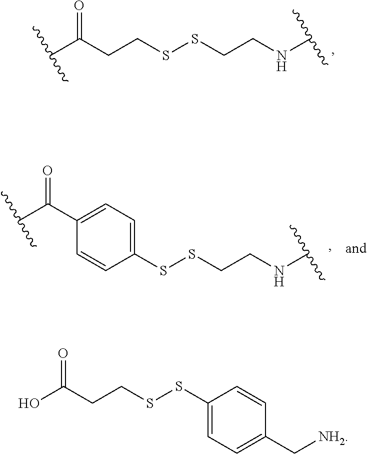 [SMILES: *NCCSSCCC(*)=O ; *NCCSSc1ccc(C(*)=O)cc1 ; NCc1ccc(SSCCC(=O)O)cc1]

The plurality of linkers of the kit may comprise a first linker associated with a first substrate (e.g., a first nucleotide) and a second linker associated with a second substrate (e.g., a second nucleotide). The first substrate and the second substrate may be of different types (e.g., different canonical nucleotides). The first substrate and the second substrate may be nucleotides comprising nucleobases of different types (e.g., A, C, G, U, and T). The first linker and the second linker may comprise the same chemical structure. Similarly, the first linker may be connected to a first fluorescent dye and the second linker may be connected to a second fluorescent dye. The first fluorescent dye and the second fluorescent dye may be of different types. For example, the first and second fluorescent dyes may fluoresce at different wavelengths and/or have different maximum excitation wavelengths. The first and second fluorescent dyes may fluoresce at similar wavelengths and/or have similar maximum excitation wavelengths regardless of whether they share the same chemical structure.

The plurality of linkers of the kit may further comprise a third linker associated with a third substrate and a fourth linker associated with a fourth substrate. The first substrate, the second substrate, the third substrate, and the fourth substrate may be of different types. For example, the first substrate, the second substrate, the third substrate, and the fourth substrate may be nucleotides comprising nucleobases of different types (e.g., A, C, G, and U/T). The first linker and the third linker may comprise different chemical structures. The first and third linker may comprise a same chemical group, such as a same cleavable group (e.g., as described herein). For example, the first linker and the third linker may each comprise a moiety comprising a disulfide bond. Similarly, the first linker and the fourth linker may comprise different chemical structures. The first and fourth linker may comprise a same chemical group, such as a same cleavable group (e.g., as described herein). For example, the first linker and the fourth linker may each comprise a moiety comprising a disulfide bond.

In an example, the first linker comprises a hyp10 moiety and a first cleavable moiety, the second linker comprises a hyp10 moiety and a second cleavable moiety, the third linker comprises a third cleavable moiety and does not comprise a hyp10 moiety, and the fourth linker comprises a fourth cleavable moiety and does not comprise a hyp10 moiety. The second cleavable moiety may have a chemical structure that is different from the first cleavable moiety. Alternatively, the second cleavable moiety and the first cleavable moiety may have the same chemical structures. The third cleavable moiety and the fourth cleavable moiety may have the same chemical structure. Alternatively, the third cleavable moiety and the fourth cleavable moiety may have different chemical structures. In an example, the first linker and the second linker each have a first chemical structure and the third linker and the fourth linker each have a second chemical structure, which second structure is different from the first chemical structure. In another example, the first linker, the second linker, the third linker, and the fourth linker all have the same chemical structure. In another example, the first linker, the second linker, the third linker, and the fourth linker all have different chemical structures.

One or more linkers in a kit may be components of a labeling reagent. Accordingly, in an aspect, the present disclosure provides a kit comprising a plurality of labeling reagents (e.g., as described herein). The plurality of labeling reagents may have identical chemical structures. Alternatively, the plurality of labeling reagents may comprise at least a first plurality of labeling reagents having a first chemical structure and a second plurality of labeling reagents having a second chemical structure different from the first chemical structure. A labeling reagent of a kit may have any useful features, as described herein. For example, a labeling reagent of a kit may comprise a cleavable portion configured to be cleaved to separate a substrate from a portion of the labeling reagent (e.g., as described herein); a semi-rigid linker portion comprising, for example, one or more sequences of hydroxyprolines (e.g., a hyp10, hyp20, or hyp30 moiety, as described herein); an optically detectable moiety (e.g., a fluorescent dye moiety, as described herein); and a scaffold to which a linker may be coupled (e.g., a lysine, dilysine, or other polylysine structure, as described herein).

### Multi-Dye Labeling Using Hydroxyprolines

Polyprolines and poly-hydroxyprolines form helical structures. See Patrick Wilhelm et al., A Crystal Structure of an Oligoproline PPII-Helix, at Last, J. Am. Chem. Soc. 2014 Jul. 14, doi: 10.1021/ja507405j, for a discussion of polyproline II (PPII) helices, which is entirely incorporated herein by reference for all purposes. The helical structure may comprise repeating turns, each turn having proline or hydroxyproline residue(s) (Wilhelm et al. finds distances of 8.98 Å±0.14 Å between every third residue in an oligoproline crystal, i.e., approximately 3 residues per turn, each residue contributing about 3.0 Å). Thus, the relative orientations of dye moieties attached to a polyproline or poly-hydroxyproline linker may be coordinated or otherwise engineered by selecting the respective residues in the linker that the dye moieties are attached to (or selecting a number of residues that are spaced between the dye moieties). In an example, a poly-hydroxyproline linker has 35 hydroxyproline (or combination of hydroxyproline and amino-proline) residues, the helical structure of the linker having approximately 3 residues per turn, a first dye moiety is attached to the twenty first residue, a second dye moiety is attached to the twenty eighth residue, and a third dye moiety is attached to the thirty fifth residue. In this example configuration, from a top view of a plane substantially normal to the helical axis (also referred to herein as screw axis or twist axis), the helical axis being lengthwise of the linker, the first dye moiety is oriented at approximately 120° angular distance from the second dye moiety, and oriented at approximately 240° angular distance from a second dye moiety. As such, dye(s) can be attached to different attachment points of a polyproline or poly-hydroxyproline structure. For example, where the attachment point is on a hydroxyproline residue, the dye can be attached via an ester bond. For example, where the attachment point is on an amino-proline residue, the dye can be attached via an amide bond.

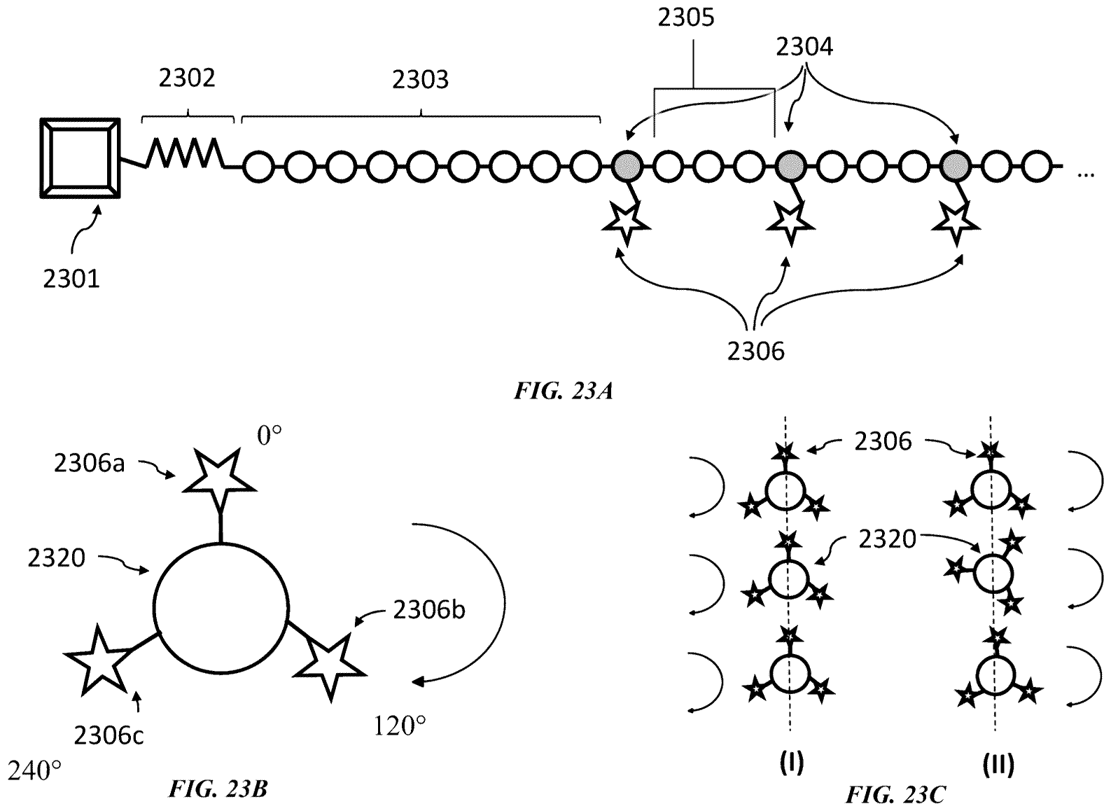

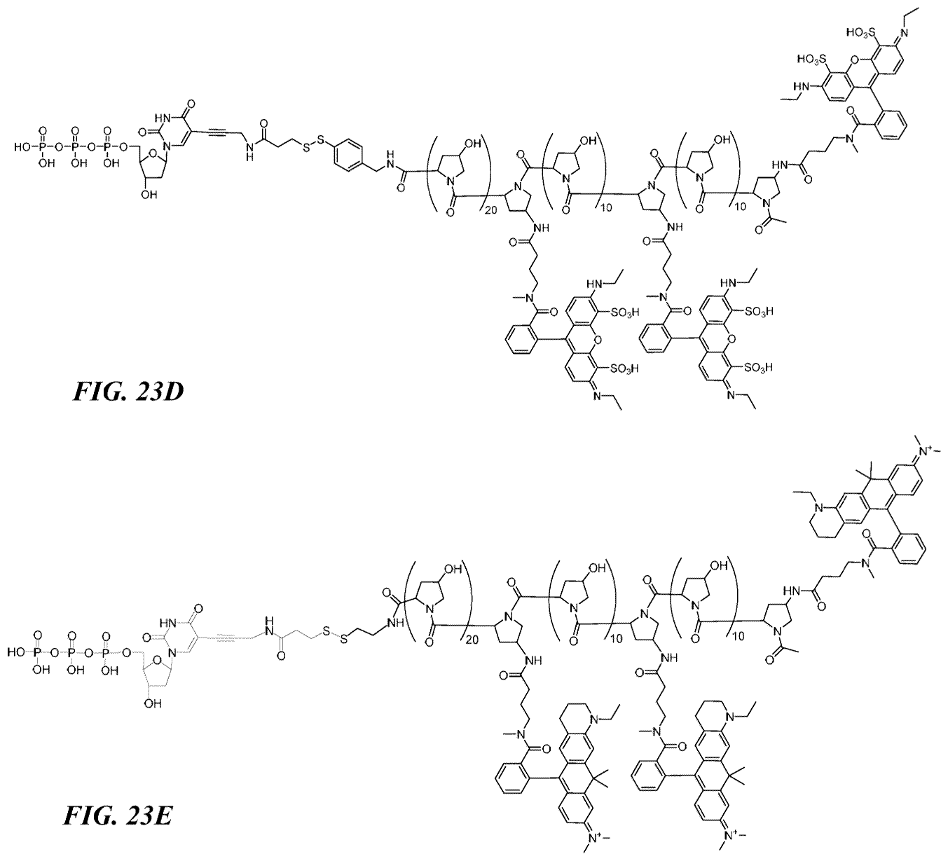

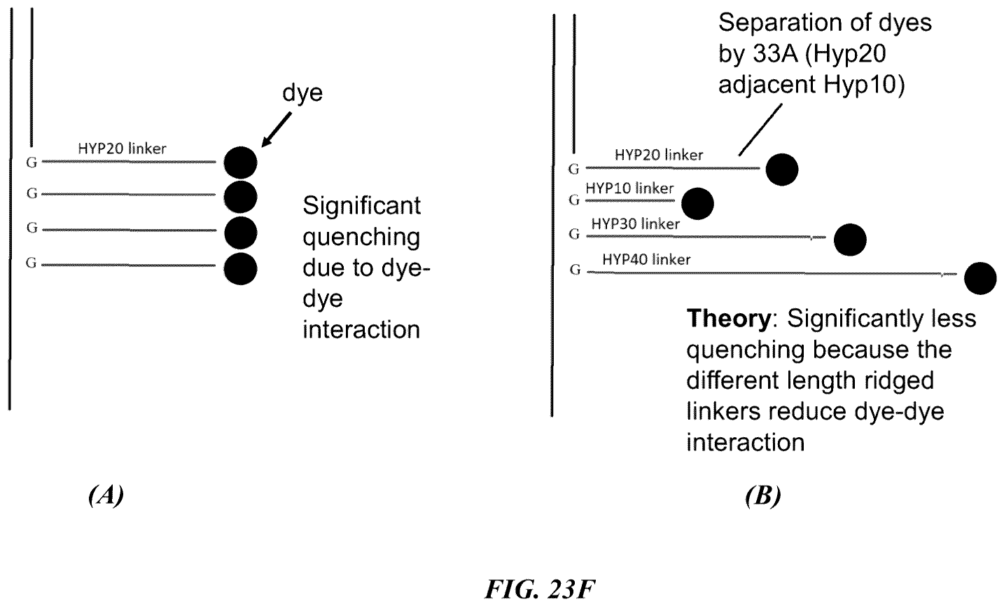

FIGS. 23A-C show example schematics of attaching multiple dyes to poly-hydroxyprolines at different angles; FIG. 23A shows an example side view of a substrate attached to a linker attached to multiple dyes (a multiply labeled substrate); FIG. 23B shows an example top view of the linker attached to multiple dyes; FIG. 23C shows an example top view of instances of multiple adjacent substrates each attached to a linker attached to multiple dyes.

In FIG. 23A, a substrate 2301 (e.g., a nucleobase or any other substrate described herein) may be attached to multiple dyes 2306 (e.g., any of the dyes described herein) via a linker which comprises a cleavable portion 2302 (e.g., comprising disulfide group or any other cleavable groups described herein) and a poly-hydroxyproline portion. The poly-hydroxyproline portion may comprise a first hydroxyproline portion 2303 (e.g., Hyp6, Hyp10, Hyp20), amino-proline or hydroxyproline attachment point residues 2304 which are attachment points of the dyes 2306, and second hydroxyproline portion(s) 2305 (e.g., Hyp6, Hyp10, or Hyp20) which are between the different amino-proline or hydroxyproline attachment point residues 2304. The cleavable portion 2302 may be proximal to the substrate 2301 than the poly-hydroxyproline portion. The first hydroxyproline portion 2303 may be disposed proximal to the substrate 2301 before the location of the first attachment point residue (e.g., 2304) attaching the first dye (e.g., 2306). The first hydroxyproline portion may comprise any number of hydroxyproline residues. The second hydroxyproline portion(s) 2305 may be disposed between attachment point residues 2304 and may comprise the same or different lengths. A second hydroxyproline portion may comprise any number of hydroxyproline residues. In some cases, a second hydroxyproline portion (e.g., 2305) has a number of residues that is different from 3× of hydroxyprolines, where x is an integer. In FIG. 23B, the proline α-helix 2320 tends to have a clockwise orientation (amino to carboxylic acid direction) if the number of residues is around (3n) and a counterclockwise orientation if the number of residues is around (3n+1), if n=0, 1, 2, 3, 4, 5 or greater integer. The poly-hydroxyproline portion may comprise a third hydroxyproline portion which is distal to the substrate 2301 and after the last attachment point residue attaching the last (most distally located) dye (this portion is not labeled in FIG. 23A). The third hydroxyproline portion may comprise any number of hydroxyproline residues. The length of the first hydroxyproline portion may be selected to provide a rigidity that sufficiently prevents the dyes 2306 from folding over and quenching with the substrate (e.g., dNTP base). The attachment point residues 2304 may maintain an α-helix structure, permit direct attachment of a dye of the dyes 2306, and allow an attachment point for later hydroxyproline residues (later helices). It will be appreciated that while FIG. 23A illustrates three dyes, this labeled substrate configuration may be applied to any number of dyes, such as 1, 2, 3, 4, 5, 6, 7, 8, 9, 10, 11, 12, 13, 14, 15, 16, 17, 18, 19, 20 or more dyes. The dyes attached to a same linker may be the same or different type of dyes. It will be appreciated that while FIG. 23A has been described with respect to a labeled substrate configuration comprising a poly-hydroxyproline portion comprising hydroxyproline residues, this configuration may comprise a poly-proline portion comprising poly-proline residues. Beneficially, with this configuration, only a fraction of the dyes can theoretically quench with an adjacent labeled substrate. In cases where the labeled substrates are used for sequencing-by-synthesis, the length of the α-helix may provide substantially separation between the dyes and the DNA template. Further, the multiple dyes may provide significantly stronger signals (e.g., fluorescent signals) per each substrate.

FIG. 23B shows an example top view of a linker having a configuration of FIG. 23A attached to multiple dyes 2306a, 2306b, 2306c. The top view looks down at the proline α-helix 2320 (represented by circle in this schematic) and is at a plane substantially normal to the helical axis, the helical axis being lengthwise of the linker. The linker of FIG. 23B comprises a Hyp20 as the first hydroxyproline portion, a first dye 2306a attached at residue #21, a second dye 2306b attached at residue #28 (around seven residues from residue #21), and a third dye 2306c attached at residue #35 (around seven residues from residue #28). In some cases, the number of hydroxyproline residues separating two adjacent dye molecules is not 3 or an integer multiple of 3. Noting the orientation of the first dye 2306a as 0°, the second dye 2306b which is attached seven residues from the first dye attachment point (or 2.33 turns at 3 residues/turn) is oriented at approximately 120°, and the third dye 2306c which is attached seven residues from the second dye attachment point (or 2.33 turns at 3 residues/turn) is oriented at approximately 240°. It will be appreciated that the first hydroxyproline portion of Hyp20 may be a Hyp10 or other length poly-hydroxyproline that has a sufficient number of prolines to obtain an α-helix structure to prevent bending.

FIG. 23C shows an example top view of instances of multiple adjacent substrates each attached to a linker attached to multiple dyes. Multiple labeled substrates, each with the configuration described in FIG. 23A, comprising a proline α-helix 2320 attached to dyes 2306, may be disposed adjacent to each other. For example, where the substrate is a nucleobase of a single type (e.g., A), during primer extension of a template comprising a homopolymer stretch (poly-T), multiple labeled substrates may be incorporated into the extending primer such that they are aligned adjacent to each other. In each of the left and right instances, the dotted line represents the lengthwise axis of the multiple substrates (e.g., homopolymer), and each helix is rotating clockwise in the N to C terminus direction. In the left instance, (I), each of the multiple dyes on each substrate is sufficiently separated and there is no quenching. The right instance, (II), represents a relatively rare instance in which every third dye is stacked with a neighboring dye of an adjacent substrate. As shown in FIG. 23C, (II), the bottom right dye on the top labeled substrate may quench with the top right dye on the middle labeled substrate, and the bottom dye on the middle labeled substrate may quench with the top dye on the bottom labeled substrate.

Provided herein is a labeled substrate comprising a (i) a substrate, (ii) a linker, and (iii) a plurality of dye moieties attached to the substrate via the linker, wherein the linker comprises a cleavable portion and a poly-hydroxyproline portion, wherein the poly-hydroxyproline portion comprises a first hydroxyproline portion comprising a first one or more hydroxyproline residues, a set of amino-proline (or hydroxyproline dye attachment point) residues that attach to the plurality of dye moieties, and one or more second hydroxyproline portions each comprising a second one or more hydroxyproline residues, each of the one or more second hydroxyproline portions disposed between different amino-proline (or hydroxyproline dye attachment point) residues of the set of amino-proline residues. Provided herein is labelling reagent comprising (i) a linker, and (ii) a plurality of dye moieties attached to the linker, wherein the linker comprises a cleavable portion and a poly-hydroxyproline portion, wherein the poly-hydroxyproline portion comprises a first hydroxyproline portion comprising a first one or more hydroxyproline residues, a set of amino-proline (or hydroxyproline dye attachment point) residues that attach to the plurality of dye moieties, and one or more second hydroxyproline portions each comprising a second one or more hydroxyproline residues, each of the one or more second hydroxyproline portions disposed between different amino-proline (or hydroxyproline dye attachment point) residues of the set of amino-proline residues.

### Methods for Using the Optical Labeling Reagents

There are several different types of quenching that can be reduced and different types of applications that can be performed using the optical (e.g., fluorescent) labeling reagents described herein.

The methods described herein can be used to reduce quenching, including G-quenching. Attachment of dyes (e.g., fluorescent dyes) to nucleotides (e.g., via a linker provided herein) can result in dye-quenching for many dyes, particularly when the dye is attached to a guanosine nucleotide. Dye quenching may take place between a dye and a nucleotide with which it is associated, as well as between dye moieties, such as between dye moieties coupled to different nucleotides (e.g., adjacent nucleotides or nucleotides separated by one or more other nucleotides). Use of the linkers provided herein can alleviate the quenching allowing more sensitive detection of sequences containing G. In addition, a dye-labeled nucleotide in proximity to a G-homopolymer region may show reduced fluorescence. Any nucleic acid sequencing method that requires attachment of a dye to dGTP may benefit from these linkers, including single molecule detection, sequencing using non-terminated nucleotides, sequencing by synthesis, sequencing using 3′-blocked nucleotides, and sequencing by hybridization.

The methods described herein can be used to reduce dye-dye quenching on adjacent or neighboring nucleotides (e.g., nucleotides separated by one, two, or more other nucleotides) on the same DNA strand. Methods that require dyes on adjacent or neighboring nucleotides can result in proximity quenching; that is, two dyes next to each other are less bright than twice the brightness of one dye, or often, less bright than even a single dye. Use of the linkers provided herein may alleviate the quenching, allowing quantitative detection of multiple dyes. For example, in sequencing methods such as mostly natural nucleotide flow sequencing, the fraction of labeled dye is typically less than 5%, since homopolymers are not linear in signal to homopolymer length at higher fractions due to the quenching problem. The reagents described herein can allow more (e.g., more than 5%, in some cases up to 100%) of the nucleotides to be labeled while facilitating sensitive and accurate detection of incorporated nucleotides.

The use of a labeled nucleotide (e.g., dye-linker-nucleotide) provided herein may result in more efficient incorporation into a growing nucleic acid strand (e.g., increased tolerance) by a polymerase (e.g., as described herein), compared to a dye-nucleotide lacking the linker (e.g., during nucleic acid sequencing). The result may be that a lower amount of the dye-labeled nucleotide is used to achieve the same signal.

The use of a labeled nucleotide (e.g., dye-linker-nucleotide) provided herein may result in less misincorporation by a polymerase (e.g., as described herein) (e.g., during nucleic acid sequencing). The result may be less loss of template strands, and thus longer sequencing reads.

The use of a labeled nucleotide (e.g., dye-linker-nucleotide) provided herein may result in less mispair extension (e.g., during nucleic acid sequencing), and thus reduced lead phasing.

The methods described herein can be used to reduce dye-dye quenching in multi-dye applications. Hybridization assays can also benefit from linkers that prevent quenching. Quenching effects may result in non-linearity of target to signal.

The methods described herein can be used in combination with oligomers and dendrimers for signal amplification. Non-quenching linkers may allow the synthesis of very bright polymers for antibody labeling. These bright antibodies may be used for cell-surface labeling in flow cytometry or for antigen detection methods such as lateral flow tests and fluorescent immunoassays.

The optical (e.g., fluorescent) labeling reagent of the present disclosure may be used as a molecular ruler. The substrate can be a fluorescence quencher, a fluorescence donor, or a fluorescence acceptor. In some cases, the substrate is a nucleotide. The linker can be attached to the nucleotide on the nucleobase as shown below, where the dye is ATTO 633:

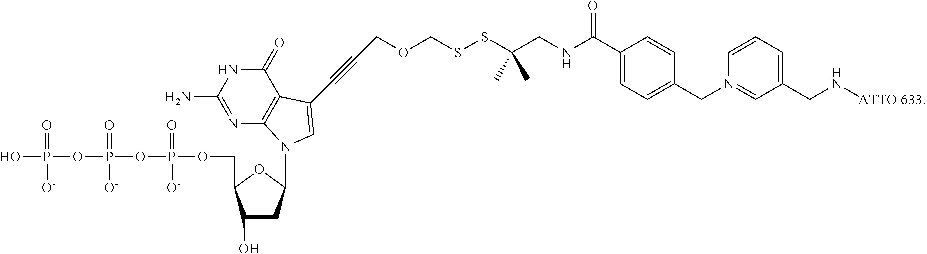

The structure shown above is an optical (e.g., fluorescent) labeling reagent comprising a cleavable (via the disulfide bond) moiety and a fluorescent dye attached via a pyridinium linker to a dGTP analog (dGTP-SS-py-ATTO 633). Additional examples of optical labeling reagents are provided elsewhere herein.

The labeled nucleotides (e.g., dye-linker-nucleotides) described herein can be used in a sequencing by synthesis method using a mixture of dye-labeled and natural nucleotides in a flow-based scheme. Such methods often use a low percentage of labeled nucleotides compared to natural nucleotides. However, using a low percentage of labeled nucleotides compared to natural nucleotides in flow mixtures (e.g., less than 20%) can have multiple drawbacks: (a) since a small fraction of the template provides sequence information, the method requires a high template copy number; (b) variability in DNA polymerase extension rates between labeled and unlabeled nucleotides can result in context-dependent labeling fractions, thus increasing the difficulty of distinguishing a single base incorporation from multiple base incorporations; and (c) the low fraction of labeling moieties can result in high binomial noise in the populations of labeled product. Methods for flow-based sequencing using mostly natural nucleotides are further described in U.S. Pat. No. 8,772,473, which is incorporated herein by reference in its entirety for all purposes.

In general, the use of labeling reagents including multiple optically detectable moieties, and/or high labeling fractions of dye-labeled nucleotides, may improve signal contrast. For example, signal-to-noise effects may decrease significantly as labeling fraction increases. The labeling reagents comprising semi-rigid linkers provided herein may allow a labeled fraction of dye-labeled nucleotide to natural nucleotide in each flow to be sufficiently high (e.g., 20-100% labeling) to avoid or reduce the effect of the aforementioned disadvantages of, e.g., various sequencing schemes. This higher percentage labeling can result in greater optical (e.g., fluorescent) signal and thus a lower template requirement. If 100% labeling is used, the binomial noise and context variation may be essentially eliminated. The key technical barrier overcome by the solution described herein is that the dye-labeled nucleotides on adjacent or nearby nucleotides must show minimal quenching. The overall result of the combined advantages may be more accurate DNA sequencing. The use of high labeling fractions (e.g., 20-100% labeling) may be facilitated by the use of non- or minimally quenching labeled nucleotides (e.g., as described herein). Quenching between dye molecules may be reduced using labeled nucleotides labeled with labeling reagents provided herein.

The present disclosure provides a method for sequencing a nucleic acid molecule. The method can comprise contacting the nucleic acid molecule with a primer under conditions sufficient to hybridize the primer to the nucleic acid molecule, thereby generating a sequencing template. The sequencing template may then be contacted with a polymerase (e.g., as described herein) and a solution (e.g., a nucleotide flow) comprising a plurality of detectably labeled substrates (e.g., as described herein). The detectably labeled substrate may comprise an optically (e.g., fluorescently) labeled nucleotide. Each optically (e.g., fluorescently) labeled nucleotide of the plurality of optically (e.g., fluorescently) labeled nucleotides may comprise the same chemical structure (e.g., each labeled nucleotide may comprise a dye of a same type, a linker of a same type, and a nucleotide or nucleotide analog of a same type). Alternatively, optically labeled nucleotides of the plurality of optically labeled nucleotides may comprise different chemical structures. An optically labeled nucleotide of the plurality of optically labeled nucleotides may be complementary to the nucleic acid molecule at a plurality of positions adjacent to the primer hybridized to the nucleic acid molecule. Accordingly, one or more optically labeled nucleotides of the plurality of optically labeled nucleotides may be incorporated into the sequencing template.

Incorporation of a labeled nucleotide may general a signal detectable by the detector described herein. In some cases, the level of the detected signal (e.g., fluorescent intensity) may be higher than a level (e.g., a threshold level). A detected signal with a higher intensity may signal the incorporation of a detectably labeled substrate (i.e., a labeled nucleotide) into the sequencing template. In some cases, the detector may detect a signal even without an incorporation of the detectably labeled substrate (i.e., a labeled nucleotide) into the sequencing template. Such a signal may have an intensity level that is lower the threshold level. In some cases, a level of a signal detected when there is no incorporation of the detectably labeled substrate into the sequencing template may be referred to as a “floor signal.” In some cases, using a detectably labeled substrate (i.e., a labeled nucleotide) with a linker described herein may generate a floor signal at a lower level than a detectably labeled substrate without such a linker. For example, a labeled nucleotide with a linker comprising a non-proteinogenic amino acid described herein may generate a floor signal at a lower level than a detectably labeled substrate with a linker without such a non-proteinogenic amino acid. In some cases, a labeled nucleotide with a linker comprising cysteic acid or 5-amino-5-carboxy-N,N,N-trimethylpentan-1-aminium or a salt thereof may generate a floor signal at a lower level than a detectably labeled substrate without the cysteic acid or 5-amino-5-carboxy-N,N,N-trimethylpentan-1-aminium or a salt thereof. In some cases, the lower level may comprise at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 95%, 96%, 97%, 98%, 99% or higher. In some cases, the lower level may comprise at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 95%, 96%, 97%, 98%, or 99%.

In some cases, using a detectably labeled substrate (i.e., a labeled nucleotide) with a linker described herein, incorporation of the detectably labeled substrate into the sequencing template may generate a detectable signal at a higher level (e.g., a brighter fluorescent intensity) than a detectably labeled substrate without such a linker. For example, when incorporated into the sequencing template, a labeled nucleotide with a linker comprising a non-proteinogenic amino acid described herein may generate a signal at a higher level than a detectably labeled substrate with a linker without such a non-proteinogenic amino acid. In some cases, when incorporated into the sequencing template, a labeled nucleotide with a linker comprising cysteic acid or 5-amino-5-carboxy-N,N,N-trimethylpentan-1-aminium or a salt thereof may generate a higher level than a detectably labeled substrate without the cysteic acid or 5-amino-5-carboxy-N,N,N-trimethylpentan-1-aminium or a salt thereof. In some cases, the higher level may comprise at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 150%, 200%, 500%, 1000%, 10000% or higher. In some cases, the higher level may comprise at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 150%, 200%, 500%, 1000%, or 10000%.

Where the nucleic acid molecule includes a homopolymeric region, multiple nucleotides (e.g., labeled and unlabeled nucleotides) may be incorporated. Incorporation of multiple nucleotides adjacent to one another may be facilitated by the use of non-terminated nucleotides. The solution comprising the plurality of optically labeled nucleotides may then be washed away from the sequencing template (e.g., using a wash flow, as described herein). An optical (e.g., fluorescent) signal from the sequencing template may be measured. Where two or more labeled nucleotides are incorporated into a homopolymeric region, the intensity of the measured optical (e.g., fluorescent) signal may be greater than an optical (e.g., fluorescent) signal that may be measured if a single optically (e.g., fluorescently) labeled nucleotide of the plurality of optically (e.g., fluorescently) labeled nucleotides had been incorporated into the sequencing template. Such a method may be particularly useful for sequencing of homopolymers or portions of nucleic acids that are homopolymeric (i.e., have a plurality of the same base in a row). An optically labeled nucleotide of the plurality of optically labeled nucleotides may comprise a dye (e.g., fluorescent dye) and a linker connected to the dye and a nucleotide (e.g., as described herein). Any of the linkers described herein may be used.

The intensity of the measured optical (e.g., fluorescent) signal may be proportional to the number of optically (e.g., fluorescently) labeled nucleotides incorporated into the sequencing template (e.g., where 100% labeling fraction is used). In other words, quenching may not significantly impact the signal emitted. For example, the intensity may be linearly proportional to the number of optically (e.g., fluorescently) labeled nucleotides incorporated into the sequencing template. The intensity of the measured optical (e.g., fluorescent) signal may be linearly proportional with a slope of approximately 1.0 when plotted against the number of optically (e.g., fluorescently) labeled nucleotides incorporated into the sequencing template. Where less than 100% of substrates are labeled (e.g., less than 100% of nucleotides in a nucleotide flow are labeled), an optical (e.g., fluorescent) signal emitted by substrates (e.g., nucleotides or nucleotide analogs) incorporated into a plurality of growing nucleic acid strands (e.g., a plurality of growing nucleic acid strands coupled to sequencing templates coupled to a support, as described herein) may be proportional to the length of a homopolymer region of the growing nucleic acid strands. Similarly, where less than 100% of substrates are labeled (e.g., less than 100% of nucleotides in each of successive nucleotide flows are labeled), an optical (e.g., fluorescent) signal emitted by substrates (e.g., nucleotides or nucleotide analogs) incorporated into a plurality of growing nucleic acid strands (e.g., a plurality of growing nucleic acid strands coupled to sequencing templates coupled to a support, as described herein) may be proportional to the length of a heteropolymeric and/or homopolymer region of the growing nucleic acid strands. In such cases, the intensity of a measured optical (e.g., fluorescent) signal may be linearly proportional to the length of a heteropolymeric and/or homopolymeric region into which substrates have incorporated. For example, a measured optical (e.g., fluorescent) signal may be linearly proportional with a slope of approximately 1.0 when optical (e.g., fluorescent) signal is plotted against the length in substrates of a heteropolymeric and/or homopolymeric region into which substrates have incorporated

In some cases, two or more optically (e.g., fluorescently) labeled nucleotides of the plurality of optically (e.g., fluorescently) labeled nucleotides are incorporated into the sequencing template (e.g., into a homopolymeric region). In some cases, three or more optically (e.g., fluorescently) labeled nucleotides of the plurality of optically (e.g., fluorescently) labeled nucleotides are incorporated into the sequencing template. The number of optically labeled nucleotides incorporated into the sequencing template during a given nucleotide flow may depend on the homopolymeric nature of the nucleic acid molecule. In some cases, a first optically (e.g., fluorescently) labeled nucleotide of the plurality of optically (e.g., fluorescently) labeled nucleotides is incorporated within four positions of a second optically (e.g., fluorescently) labeled nucleotide of the plurality of optically (e.g., fluorescently) labeled nucleotides.

An optically (e.g., fluorescently) labeled nucleotide may comprise a cleavable group to facilitate cleavage of the optical (e.g., fluorescent) label (e.g., as described herein). In some cases, a method may further comprise, subsequent to incorporation of the one or more optically (e.g., fluorescently) labeled nucleotides and washing away of residual solution, cleaving optical (e.g., fluorescent) labels of the one or more optically (e.g., fluorescently) labeled nucleotides incorporated into the sequencing template (e.g., as described herein). The cleavage flow may be followed by an additional wash flow. A cycle in which each canonical nucleotide (e.g., A, T, G, C, U) is sequentially provided to the sequencing template, signals detected, and optionally labels cleaved, may be repeated one or more times to sequence the nucleic acid molecule.

In some cases, a nucleotide flow and wash flow may be followed by a “chase” flow comprising unlabeled nucleotides and no labeled nucleotides. The chase flow may be used to complete the sequencing reaction for a given nucleotide position or positions of the sequencing template (e.g., across a plurality of such templates immobilized to a support). The chase flow may precede detection of an optical signal from a template. Alternatively, the chase flow may follow detection of an optical signal from a template. The chase flow may precede a cleavage flow. Alternatively, the chase flow may follow a cleavage flow. The chase flow may be followed by a wash flow.

The methods provided herein can also be used to sequence heteropolymers and/or heteropolymeric regions of a nucleic acid molecule (i.e., portions that are not homopolymeric). Accordingly, the methods described herein can be used to sequence a nucleic acid molecule having any degree of heteropolymeric or homopolymeric nature.

Regarding homopolymers, a nucleotide flow at a homopolymer region may incorporate several nucleotides in a row. Contacting a sequencing template comprising a nucleic acid molecule (e.g., a nucleic acid molecule hybridized to an unextended primer) comprising a homopolymer region with a solution comprising a plurality of nucleotides (e.g., labeled and unlabeled nucleotides), where each nucleotide of the plurality of nucleotides is of a same type, may result in multiple nucleotides of the plurality of nucleotides being incorporated into the sequencing template. In some cases, at least 2, at least 3, at least 4, at least 5, at least 6, at least 7, at least 8, at least 9, or at least 10 nucleotides are incorporated (i.e., in a homopolymeric region of a nucleic acid molecule). The plurality of nucleotides incorporated into the sequencing template may comprise a plurality of labeled nucleotides (e.g., optically labeled, such as fluorescently labeled), as described herein. In such an instance, one or more of said nucleotides incorporated into a homopolymer region may be labeled and may either occupy adjacent or non-adjacent positions to other labeled nucleotides incorporated into the homopolymeric region. The intensity of a signal obtained from a nucleic acid molecule may be proportional to the number of incorporated labeled nucleotides (e.g., where a labeling fraction of 100% is used). For example, the intensity of an optical signal (e.g., fluorescent signal) obtained from a nucleic acid molecule containing two labeled nucleotides may be of greater intensity than the optical signal obtained from a nucleic acid molecule containing one labeled nucleotide. Furthermore, the intensity of a signal obtained from a nucleic acid molecule may depend on the relative positioning of labeled nucleotides within a nucleic acid molecule. For example, a nucleic acid molecule containing two labeled nucleotides in non-adjacent positions may provide a different signal intensity than a nucleic acid molecule containing two labeled nucleotides in adjacent positions. Quenching in such systems may be optimized by careful selection of linkers and dyes (e.g., fluorescent dyes). In some cases, a plot of optical signal (e.g., fluorescence) vs. homopolymer length can be linear. For example, measured optical signal for an ensemble of growing nucleic acid strands including homopolymeric regions into which labeled nucleotides are incorporated may be approximately linearly proportional to the nucleotide length of the homopolymeric region.

As such, a method for sequencing a nucleic acid molecule may comprise subjecting a template nucleic acid molecule, hybridized to a sequencing primer, to multiple and/or repeated interrogating flows of labeled nucleotide solutions, to detect incorporation events. The nucleotides in the labeled nucleotide solutions may be terminated. The nucleotides in the labeled nucleotide solutions may be non-terminated. In some cases, the solution containing an optically (e.g., fluorescently) labeled nucleotide also contains unlabeled nucleotides. The unlabeled nucleotides may comprise the same canonical nucleotide as the labeled nucleotides. In some embodiments, about 5%, about 10%, about 20%, about 30%, about 40%, about 50%, about 60%, about 70%, about 80%, about 90%, or about 100% of nucleotides in the solution are fluorescently labeled. In some cases, at least about 5%, at least about 10%, at least about 20%, at least about 30%, at least about 40%, at least about 50%, at least about 60%, at least about 70%, at least about 80%, at least about 90%, or more of nucleotides in the solution are fluorescently labeled. In some cases, at least about 5%, at least about 10%, at least about 20%, at least about 30%, at least about 40%, at least about 50%, at least about 60%, at least about 70%, at least about 80%, at least about 90%, or more of nucleotides in the solution are not fluorescently labeled.

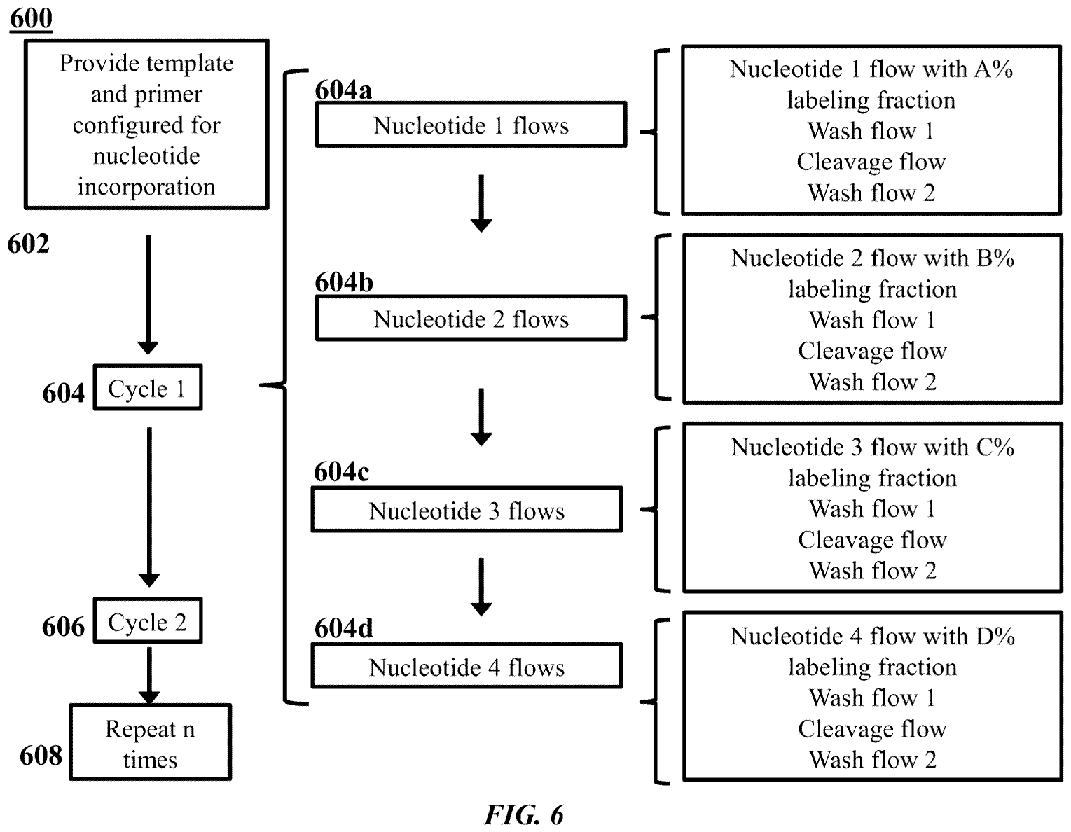

An example sequencing procedure 600 is provided in FIG. 6. In process 602, a template and primer configured for nucleotide incorporation are provided. A first sequencing cycle 604 is subsequently performed. First sequencing cycle 604 includes four nucleotide flow processes 604a, 604b, 604c, and 604d, each of which have multiple flows. Nucleotides 1, 2, 3, and 4 may each include nucleobases of different canonical types (e.g., A, G, C, and U). A given nucleotide flow may include both labeled nucleotides (e.g., nucleotides labeled with an optical labeling reagent provided herein) and unlabeled nucleotides. In some instances, the labeled and unlabeled nucleotides may be of a same canonical base type. In some instances, at least one of the labeled and unlabeled nucleotides may include nucleobases of different canonical types. The labeling fraction of each nucleotide flow may be different. That is, A, B, C, and D in FIG. 6 may be the same or different and may range from 0% to 100% (e.g., as described herein). Labels and linkers used to label nucleotides 1, 2, 3, and 4 may be of the same or different types. For example, nucleotide 1 may have a linker including a cleavable linker and a hyp10 linker and a first green dye, and nucleotide 2 may have a linker including a cleavable linker but not a hyp10 linker and a second green dye. The first green dye may be the same as or different from the first green dye. The cleavable linkers associated with the different nucleotides may be the same or different. Flow process 604a may include a nucleotide flow (e.g., a flow including a plurality of nucleotides of type Nucleotide 1, A % of which may be labeled). During this nucleotide flow, labeled and unlabeled nucleotides may be incorporated into the growing strand (e.g., using a polymerase enzyme). A first wash flow (“wash flow 1”) may be used to remove unincorporated nucleotides and associated reagents. A cleavage flow including a cleavage reagent may be provided to all or portions of the optical labeling reagents attached to incorporated nucleotides. For example, labeled nucleotides may include a cleavable linker portion that may by cleaved upon contact with the cleavage reagent to provide a scarred nucleotide. A second wash flow (“wash flow 2”) may be used to remove the cleavage reagent and cleaved materials. Nucleotide flow process 604a may also include a “chase” process in which a nucleotide flow including only unlabeled nucleotides of type Nucleotide 1 may be flowed. Such a chase process may be followed by a wash flow. The chase process and its accompanying wash flow may take place after the initial nucleotide flow and wash flow 1, or after the cleavage flow and wash flow 2. The next nucleotide flow process 604b may then begin and proceed in similar fashion. Following completion of flow processes 604b, 604c, and 604d, the first sequencing cycle 604 may be complete. A second sequencing cycle 606 may begin. Cycle 706 may include the same flow processes in the same or different order. Additional cycles may be performed until all or a portion of the template has been sequenced. Detection of incorporated nucleotides via emission detection may be performed after nucleotide flows and initial wash flows and before cleavage flows for each nucleotide flow process (e.g., flow process 604a may include a detection process between wash flow 1 and cleavage flow, etc.). A template interrogated by such a sequencing process may be immobilized to a support (e.g., as described herein). A plurality of such templates (e.g., at least about 100, 200, 500, 1000, 10000, 100,000, 500,000, 1,000,000, or more templates) may be interrogated contemporaneously in this fashion (e.g., in clonal fashion). In such a system, incorporation of nucleotides may be detected as an average over the plurality of templates, which may permit the use of labeling fractions of less than 100%.

In some cases, for any of the preceding methods, the nucleotide is guanine (G) and the linker decreases quenching between the nucleotide and the dye (e.g., fluorescent) dye.

In some cases, for any of the preceding methods, an optically (e.g., fluorescently) labeled nucleotide comprising a linker provided herein is more efficiently incorporated into a sequencing template than another optically (e.g., fluorescently) labeled nucleotide that comprises the same nucleotide and optical (e.g., fluorescent) dye but does not include the linker. In some cases, for any of the preceding methods, an optically (e.g., fluorescently) labeled nucleotide comprising a linker provided herein is incorporated into a sequencing template with higher fidelity than another optically (e.g., fluorescently) labeled nucleotide that comprises the same nucleotide and optical (e.g., fluorescent) dye but does not include the linker.

For any of the sequencing methods provided herein, the polymerase used may be a Family A polymerase such as Taq, Klenow, or Bst polymerase. Alternatively, for any of the sequencing methods provided herein, the polymerase may be a Family B polymerase such as Vent(exo-) or Therminator™ polymerase. The polymerase may be, for example, Bst3.0, Pol19, Pol22, Pol47, Pol49, Pol50, or any other useful polymerase.

In an aspect, the present disclosure provides methods for sequencing a nucleic acid molecule using the optically (e.g., fluorescently) labeled nucleotides described herein. A method may comprise providing a plurality of nucleic acid molecules, which plurality of nucleic acid molecules may comprise or be part of a colony or a plurality of colonies. The plurality of nucleic acid molecules may have sequence homology to a template sequence. The method may comprise contacting the plurality of nucleic acid molecules with a solution comprising a plurality of nucleotides (e.g., a solution comprising a plurality of optically labeled nucleotides) under conditions sufficient to incorporate a subset of the plurality of nucleotides into a plurality of growing nucleic acid strands that is complementary to the plurality of nucleic acid molecules. The method may comprise detecting one or more signals or signal changes from the labeled nucleotides incorporated into the plurality of growing nucleic acid strands, wherein the one or more signals or signal changes are indicative of the labeled nucleotides having incorporated into the plurality of growing nucleic acid strands.

The optically (e.g., fluorescently) labeled nucleotides of the plurality of nucleotides may be non-terminated. In such cases, the growing strands may incorporate one or more consecutive nucleotides during (e.g., a complimentary base to the plurality of nucleotides in solution is not present at a plurality of positions adjacent to the primer hybridized to the nucleic acid molecule). The one or more signals or signal changes detected from the optically (e.g., fluorescently) labeled nucleotides may be indicative of consecutive nucleotides having incorporated into the plurality of growing nucleic acid strands. Methods for determining a number of fluorophores from the detected signals or signal changes are described elsewhere herein.

Alternatively, the optically (e.g., fluorescently) labeled nucleotides may be terminated. In such cases, each growing strand may incorporate no more than one nucleotide per flow cycle until synthesis is terminated. The one or more signals or signal changes detected from the optically (e.g., fluorescently) labeled nucleotides may be indicative of nucleotides having incorporated into the plurality of growing nucleic acid strands. Prior to, during, or subsequent to detection, a terminating group of the labeled nucleotides may be cleaved (e.g., to facilitate sequencing of homopolymers, and/or to reduce potential context and/or quenching issues).

Alternatively or additionally, the optically (e.g., fluorescently) labeled nucleotides may include a mixture of terminated and non-terminated nucleotides. In such cases, the growing strands may incorporate one or more consecutive nucleotides generating an extended primer. The solution comprising the plurality of terminated and non-terminated nucleotides may then be washed away from the sequencing template. Unlabeled nucleotides of the plurality of nucleotides may comprise nucleotide moieties of the same type as labeled nucleotides of the plurality of nucleotides (e.g., the same canonical nucleotide).

In an aspect, the present disclosure provides compositions comprising one or more fluorescently labeled nucleotides and methods of using the same. A composition may comprise a solution comprising a fluorescently labeled nucleotide (e.g., as described herein). The fluorescently labeled nucleotide may comprise a fluorescent labeling reagent (e.g., as described herein) comprising a fluorescent dye that is connected to a nucleotide or nucleotide analog (e.g., as described herein) via a linker (e.g., as described herein). The solution (e.g., nucleotide flow) may comprise a plurality of fluorescently labeled nucleotides. The solution may also comprise a plurality of unlabeled nucleotides, in which each nucleotide of the plurality of unlabeled nucleotides is of a same canonical base type as each nucleotide of the plurality of fluorescently labeled nucleotides. The ratio of the plurality of fluorescently labeled nucleotides to the plurality of unlabeled nucleotides in the solution may be any of the ratios described herein. In some cases, the solution may not comprise any unlabeled nucleotides and the labeling fraction may be 100%. The composition may comprise chase flow solutions (e.g., comprising 100% unlabeled nucleotides) configured for use in chase flows.

The solution (e.g., nucleotide flow) may be provided to a template nucleic acid molecule coupled to (e.g., hybridized to) a nucleic acid strand (e.g., sequencing primer, growing strand, etc.). The template nucleic acid molecule may be immobilized to a support (e.g., as described herein). For example, the template nucleic acid molecule may be immobilized to a support via an adapter. For example, the template nucleic acid molecule may be immobilized to a support via a primer to which it is hybridized. The support may be immobilized to a substrate (e.g., a wafer). The composition may comprise a template nucleic acid molecule, nucleic acid strand (e.g., sequencing primer, growing strand, etc.), support, substrates, etc. The composition may comprise a polymerase enzyme (e.g., as described herein). The composition may comprise a wash solution configured for use in wash flows. The composition may comprise any reagent or agent described being used in a method described herein.

Also provided herein are kits that comprise any combination of one or more components of compositions described herein.

In another aspect, the present disclosure provides a method comprising providing a fluorescent labeling reagent (e.g., as described herein). The fluorescent labeling reagent may comprise a fluorescent dye and a linker that is connected to the fluorescent dye. A substrate may be contacted with the fluorescent labeling reagent to generate a fluorescently labeled substrate, in which the linker connected to the fluorescent dye is associated with the substrate. The substrate can be any substrate described herein, such as a nucleotide or nucleotide analog described herein.

The labeled nucleotides of the present disclosure may be used during sequencing operations that involve a high fraction of labeled nucleotides. For example, the present disclosure provides a method comprising contacting a nucleic acid molecule (e.g., a template nucleic acid molecule) with a solution comprising a plurality of nucleotides under conditions sufficient to incorporate a first labeled nucleotide and a second labeled nucleotide of the plurality of nucleotides into a growing strand that is at least partially complementary to the nucleic acid molecule. The first labeled nucleotide and the second labeled nucleotide may be of a same canonical base type. The first nucleotide may comprise a fluorescent dye (e.g., as described herein), which fluorescent dye may be associated with the first nucleotide via a linker (e.g., as described herein). The second nucleotide may comprise the same fluorescent dye (e.g., associated with the second nucleotide via a linker having the same chemical structure of the linker associating the first nucleotide and the fluorescent dye). A fluorescent dye coupled to a nucleotide (e.g., the first and/or second nucleotide) may be cleavable (e.g., upon application of a cleavage reagent). At least about 20% of the plurality of nucleotides may be labeled nucleotides. For example, at least 20% of the plurality of nucleotides may be associated with a fluorescent labeling reagent (e.g., as described herein). For example, at least about 50%, 70%, 80%, 90%, 95%, or 99% of the plurality of nucleotides may be labeled nucleotides. For example, all of the nucleotides of the plurality of nucleotides may be labeled nucleotides (e.g., the labeling fraction may be 100%). One or more signals or signal changes may be detected from the first labeled nucleotide and the second labeled nucleotide (e.g., as described herein). The one or more signals or signal changes may comprise fluorescent signals or signal changes. The one or more signals or signal changes may be indicative of incorporation of the first labeled nucleotide and the second labeled nucleotide. The one or more signals or signal changes may be resolved to determine a sequence of the nucleic acid molecule, or a portion thereof. Resolving the one or more signals or signal changes may comprise determining a number of consecutive nucleotides from the solution that incorporated into the growing strand. The number of consecutive nucleotides may be selected from the group consisting of 2, 3, 4, 5, 6, 7, or 8 nucleotides. Resolving the one or more signals or signal changes may comprise processing a tolerance of the solution. A third nucleotide may also be incorporated into the growing strand (e.g., before or after detection of the one or more signals or signal changes). The third nucleotide may be a nucleotide of the plurality of nucleotides of the solution. Alternatively, the third nucleotide may be provided in a separate solution, such as in a “chase” flow (e.g., as described herein). The third nucleotide may be unlabeled. Alternatively, the third nucleotide may be labeled. The first labeled nucleotide and the third nucleotide may be of a same canonical base type. Alternatively, the first labeled nucleotide and the third nucleotide may be of different canonical base types.

The method may further comprise cleaving the fluorescent dye coupled to the first labeled nucleotide. The fluorescent dye may be cleaved by application of a cleavage reagent configured to cleave a linker associating the first labeled nucleotide and the fluorescent dye. The nucleic acid molecule may be contacted with a second solution comprising a second plurality of nucleotides under conditions sufficient to incorporate a third labeled nucleotide of the second plurality of nucleotides into the growing strand. At least about 20% of the second plurality of nucleotides may be labeled nucleotides (e.g., as described herein). One or more second signals or signal changes may be detected from the third labeled nucleotide (e.g., as described herein). The one or more second signals or signal changes may be resolved to determine a second sequence of the nucleic acid molecule, or a portion thereof. The first labeled nucleotide and the third labeled nucleotide may be different canonical base types (e.g., A, C, U/T, or G). The third labeled nucleotide may comprise the fluorescent dye. The fluorescent dye may be coupled to the third labeled nucleotide via a linker (e.g., as described herein), which linker may have the same chemical structure as the linker connecting the fluorescent dye to the first labeled nucleotide or a different chemical structure.

Alternatively, the method may comprise contacting the nucleic acid molecule with a second solution comprising a second plurality of nucleotides under conditions sufficient to incorporate a third labeled nucleotide of the second plurality of nucleotides into the growing strand. At least about 20% of the second plurality of nucleotides may be labeled nucleotides (e.g., as described herein). One or more second signals or signal changes may be detected from the third labeled nucleotide (e.g., as described herein). The one or more second signals or signal changes may be resolved to determine a second sequence of the nucleic acid molecule, or a portion thereof. The first labeled nucleotide and the third labeled nucleotide may be different canonical base types (e.g., A, C, U/T, or G). The third labeled nucleotide may comprise the fluorescent dye. The fluorescent dye may be coupled to the third labeled nucleotide via a linker (e.g., as described herein), which linker may have the same chemical structure as the linker connecting the fluorescent dye to the first labeled nucleotide or a different chemical structure. Contacting the nucleic acid molecule with the second solution may be performed in absence of cleaving a fluorescent dye from the first labeled nucleotide or the second labeled nucleotide. This process may be repeated one or more times, such as 1, 2, 3, 4, 5, or more times, each with a different solution of nucleotides, in absence of cleaving a fluorescent dye from the first labeled nucleotide or the second labeled nucleotide. One or more of these different solutions of nucleotides may comprise at least 20% labeled nucleotides.

The present disclosure also provides a method comprising contacting a nucleic acid molecule with a solution comprising a plurality of non-terminated nucleotides under conditions sufficient to incorporate a labeled nucleotide and a second nucleotide of the plurality of non-terminated nucleotides into a growing strand that is at least partly complementary to the nucleic acid molecule, or a portion thereof. The labeled nucleotide and the second nucleotide may be of a same canonical base type. Alternatively, the labeled nucleotide and the second nucleotide may be of different canonical base types. The labeled nucleotide may comprise a fluorescent dye (e.g., as described herein), which fluorescent dye may be associated with the labeled nucleotide via a linker (e.g., as described herein). The second nucleotide may be a labeled nucleotide. For example, the second nucleotide may comprise the same fluorescent dye (e.g., associated with the second nucleotide via a linker having the same chemical structure of the linker associating the first nucleotide and the fluorescent dye). Alternatively, the second nucleotide may not be coupled to a fluorescent dye (e.g., the second nucleotide may be unlabeled). A fluorescent dye coupled to a nucleotide (e.g., the first and/or second nucleotide) may be cleavable (e.g., upon application of a cleavage reagent). The plurality of non-terminated nucleotides may comprise nucleotides of a same canonical base type. At least about 20% of said plurality of nucleotides may be labeled nucleotides. For example, at least 20% of the plurality of nucleotides may be associated with a fluorescent labeling reagent (e.g., as described herein). For example, at least about 50%, 70%, 80%, 90%, 95%, or 99% of the plurality of non-terminated nucleotides may be labeled nucleotides. For example, substantially all of the plurality of non-terminated nucleotides may be labeled nucleotides. For example, all of the nucleotides of the plurality of non-terminated nucleotides may be labeled nucleotides (e.g., the labeling fraction may be 100%). One or more signals or signal changes may be detected from the labeled nucleotide (e.g., as described herein). The one or more signals or signal changes may comprise fluorescent signals or signal changes. The one or more signals or signal changes may be indicative of incorporation of the labeled nucleotide. The one or more signals or signal changes may be resolved to determine a sequence of the nucleic acid molecule, or a portion thereof. Resolving the one or more signals or signal changes may comprise determining a number of consecutive nucleotides from the solution that incorporated into the growing strand. The number of consecutive nucleotides may be selected from the group consisting of 2, 3, 4, 5, 6, 7, or 8 nucleotides. Resolving the one or more signals or signal changes may comprise processing a tolerance of the solution. A third nucleotide may also be incorporated into the growing strand (e.g., before or after detection of the one or more signals or signal changes). The third nucleotide may be a nucleotide of the plurality of non-terminated nucleotides of the solution. Alternatively, the third nucleotide may be provided in a separate solution, such as in a “chase” flow (e.g., as described herein). The third nucleotide may be unlabeled. Alternatively, the third nucleotide may be labeled. The labeled nucleotide and the third nucleotide may be of a same canonical base type. Alternatively, the labeled nucleotide and the third nucleotide may be of different canonical base types.

The method may further comprise cleaving the fluorescent dye coupled to the labeled nucleotide. The fluorescent dye may be cleaved by application of a cleavage reagent configured to cleave a linker associating the labeled nucleotide and the fluorescent dye. The nucleic acid molecule may be contacted with a second solution comprising a second plurality of non-terminated nucleotides under conditions sufficient to incorporate a third labeled nucleotide of the second plurality of non-terminated nucleotides into the growing strand. At least about 20% of the second plurality of non-terminated nucleotides may be labeled nucleotides (e.g., as described herein). One or more second signals or signal changes may be detected from the third labeled nucleotide (e.g., as described herein). The one or more second signals or signal changes may be resolved to determine a second sequence of the nucleic acid molecule, or a portion thereof. The first labeled nucleotide and the third labeled nucleotide may be different canonical base types (e.g., A, C, U/T, or G). The third labeled nucleotide may comprise the fluorescent dye. The fluorescent dye may be coupled to the third labeled nucleotide via a linker (e.g., as described herein), which linker may have the same chemical structure as the linker connecting the fluorescent dye to the first labeled nucleotide or a different chemical structure.

Alternatively, the method may comprise contacting the nucleic acid molecule with a second solution comprising a second plurality of non-terminated nucleotides under conditions sufficient to incorporate a third labeled nucleotide of the second plurality of non-terminated nucleotides into the growing strand. At least about 20% of the second plurality of nucleotides may be labeled nucleotides (e.g., as described herein). One or more second signals or signal changes may be detected from the third labeled nucleotide (e.g., as described herein). The one or more second signals or signal changes may be resolved to determine a second sequence of the nucleic acid molecule, or a portion thereof. The first labeled nucleotide and the third labeled nucleotide may be different canonical base types (e.g., A, C, U/T, or G). The third labeled nucleotide may comprise the fluorescent dye. The fluorescent dye may be coupled to the third labeled nucleotide via a linker (e.g., as described herein), which linker may have the same chemical structure as the linker connecting the fluorescent dye to the first labeled nucleotide or a different chemical structure. Contacting the nucleic acid molecule with the second solution may be performed in absence of cleaving a fluorescent dye from the first labeled nucleotide or the second labeled nucleotide. This process may be repeated one or more times, such as 1, 2, 3, 4, 5, or more times, each with a different solution of nucleotides, in absence of cleaving a fluorescent dye from the first labeled nucleotide or the second labeled nucleotide. One or more of these different solutions of nucleotides may comprise at least 20% labeled nucleotides.

Sequencing Methods Using Optical Labeling Reagents with Variable Lengths

Provided herein are systems, methods, kits, and compositions for sequencing using optical labeling reagents with variable lengths (e.g., variable length linkers). A method may comprise (a) providing a primer-hybridized template nucleic acid molecule; and (b) contacting the primer-hybridized template nucleic acid molecule with a mixture of nucleotides, wherein the mixture of nucleotides comprises at least a first type of labeled nucleotide and a second type of labeled nucleotide, wherein the first type of labeled nucleotide comprises a first linker of a first length or which provides a first distance between a substrate and a label of the first type of labeled nucleotide and the second type of labeled nucleotide comprises a second linker of a second length or which provides a second distance between a substrate and a label of the second type of labeled nucleotide, the first length different from the second length and the first distance different from the second distance. The method may further comprise (c) detecting one or more signals from the primer-hybridized template nucleic acid molecule. The mixture of nucleotides may comprise any number of different types of nucleotides with linkers of different lengths or which provide different distances from the respective substrates and respective dyes. For example, the plurality of nucleotides may comprise at least 2, 3, 4, 5, 6, 7, 8, 9, 10, 11, 12, 13, 14, 15, 16, 17, 18, 19, 20, 30, 40, 50 or more types of nucleotides with linkers of different lengths or which provide different distances from the respective substrates and respective dyes. Alternatively or in addition, the plurality of nucleotides may comprise at most 2, 3, 4, 5, 6, 7, 8, 9, 10, 11, 12, 13, 14, 15, 16, 17, 18, 19, 20, 30, 40, or 50 types of nucleotides with linkers of different lengths or which provide different distances from the respective substrates and respective dyes. In some cases, the linkers of the plurality of nucleotides may comprise a Hypn moiety.

Beneficially, labeled nucleotides that are incorporated adjacent to each other by the primer, for example to extend through a homopolymer portion of the template, the variable lengths of the linkers of the incorporated labeled nucleotides may make it less likely for the respective labels (e.g., dyes) of the adjacent labeled nucleotides to quench with each other. This phenomenon is illustrated in the schematic of FIG. 23F. The left panel (A) illustrates 4 labeled G nucleotides that have consecutively incorporated into a primer, each of the labeled G nucleotides having the same length linkers (e.g., HYP20). As the respective linkers position the dyes at approximately the same distance from the primer-template backbone (substrate), after the labeled G nucleotides are incorporated, the dyes are disposed proximally adjacent to each other. A signal detected from such a molecule may thus represent a significantly quenched signal that is difficult to discern or resolve to determine the number of nucleotides incorporated. In contrast, the right panel (B) illustrates 4 labeled G nucleotides that have consecutively incorporated into a primer, each of the labeled G nucleotides having different length linkers (e.g., HYP20, HYP10, HYP30, HYP40). As the linkers position each of the four dyes at a different distance from the primer-template backbone (substrate), after the labeled G nucleotides are incorporated the different dye-dye interactions are reduced and there is significantly less quenching. A signal detected from such a molecule math thus more accurately represent the number of nucleotides that have incorporated.

While the example of FIG. 23F illustrates, in the right panel, a scenario in which each labeled nucleotide that is incorporated has a unique length linker, it will be appreciated that reduced dye-dye interactions can be achieved with a mixture of nucleotides that have as less as only two types of linkers, such as when labeled nucleotides of two types of different length linkers incorporate in an alternating fashion (e.g., in the order of: HYP20, HYP10, HYP20, HYP10). The probability that two directly adjacent labeled, incorporated nucleotides have different length linkers increases with the number of types of labeled nucleotides with different lengths in the mixture.

### Sequencing Methods Using Multi-Labeled Optical Labeling Reagents

Provided herein are systems, methods, kits, and compositions for sequencing using multi-labeled optical labeling reagents.

A method may comprise (a) providing a primer-hybridized template nucleic acid molecule; and (b) contacting the primer-hybridized template nucleic acid molecule with a mixture of terminated nucleotides, wherein the mixture of terminated nucleotides comprises at least a first type of labeled nucleotide comprising a first number of dyes and a second type of labeled nucleotide comprising a second number of dyes, wherein the first number is different than the second number, wherein the first type of labeled nucleotide is a first canonical base and the second type of labeled nucleotide is a second canonical base different from the first canonical base, and wherein the first type of labeled nucleotide and the second type of labeled nucleotide are detectable at a same or substantially same frequency. The method may further comprise (c) detecting one or more signals form the primer-hybridized template nucleic acid molecule. The mixture of nucleotides may comprise three different types of labeled nucleotides with three different canonical base types, each type of canonical base labeled with a different number of dyes. The mixture of nucleotides may comprise four different types of labeled nucleotides with four different canonical base types, each type of canonical base labeled with a different number of dyes. A labeled nucleotide comprising n number of dyes may be any of the labeled nucleotides and any of the multi-labeled nucleotides, such as described with respect to FIGS. 23A-E described herein.

A terminated nucleotide may also be referred to herein as a terminator. The term “terminator” as used herein with respect to a nucleotide may generally refer to a moiety that is capable of terminating primer extension. A terminator may be a reversible terminator. A reversible terminator may comprise a blocking or capping group that is attached to the 3′-oxygen atom of a sugar moiety (e.g., a pentose) of a nucleotide or nucleotide analog. Such moieties are referred to as 3′-O-blocked reversible terminators. Examples of 3′-O-blocked reversible terminators include, for example, 3′-ONH₂ reversible terminators, 3′-O-allyl reversible terminators, and 3′-O-aziomethyl reversible terminators. Alternatively, a reversible terminator may comprise a blocking group in a linker (e.g., a cleavable linker) and/or dye moiety of a nucleotide analog. 3′-unblocked reversible terminators may be attached to both the base of the nucleotide analog as well as a fluorescing group (e.g., label, as described herein). Examples of 3′-unblocked reversible terminators include, for example, the “virtual terminator” developed by Helicos BioSciences Corp. and the “lightning terminator” developed by Michael L. Metzker et al. A terminator may otherwise, such as by steric or structural hindrance, prevent or terminate primer extension. Cleavage of a reversible terminator may be achieved by, for example, irradiating a nucleic acid molecule including the reversible terminator and/or providing a cleavage agent.

Beneficially, labeled nucleotides used in these methods may benefit from (and/or be designed to have) no or reduced quenching between the multiple labels (e.g., dyes) attached to the same linker. Thus, a signal detected from an incorporated, labeled nucleotide (or amplified signals detected from a colony of the template nucleic acid molecule) may be discernible or resolvable for how many dyes there are, and/or discernible or resolvable for which type of labeled nucleotide. As the nucleotides are terminated, at most one nucleotide may be incorporated by the primer, and a signal detected after an incorporation event may be indicative of a single nucleotide that is incorporated. Thus, a signal intensity may be uniquely associated with a type of labeled nucleotide (e.g., canonical base type). Single frequency or greyscale analysis may be sufficient to determine a sequence of the template nucleic acid. In an example, dATPs are labeled with two dyes, dCTPs are labeled with four dyes, dUTPs are labeled with five dyes, and dGTPs are labeled with seven dyes. A mixture of all four labeled nucleotide types is provided to multiple template colonies. A signal that is detected from each template colonies may be matched to dATPs, dCTPs, dUTPs, or dGTPs depending on its intensity. If a scan detects for example a signal intensity of 21 units at location 1, a signal intensity of 21 units at location 2, a signal intensity of 6 units at location 3, a signal intensity of 12 units at location 4, and a signal intensity of 15 units at location 5, one may infer that location 1 incorporated a G base, location 2 incorporated a G base, location 3 incorporated an A base, location 4 incorporated a C base, and location 5 incorporated a U base. In another example, dATPs are labeled with two dyes, dCTPs are labeled with four dyes, dUTPs are labeled with five dyes, and dGTPs are not labeled. A mixture of all four labeled nucleotide types is provided to multiple template colonies. If a scan detects for example a signal intensity of 0 units at location 1, a signal intensity of 0 units at location 2, a signal intensity of 6 units at location 3, a signal intensity of 12 units at location 4, and a signal intensity of 15 units at location 5, one may infer that location 1 incorporated a G base, location 2 incorporated a G base, location 3 incorporated an A base, location 4 incorporated a C base, and location 5 incorporated a U base. While the examples above describe signal intensity units being linearly proportional with the number of dyes on a labeled nucleotide, it will be appreciated that the signal intensity units for a given labeled nucleotide may not be linearly proportional to the number of dyes on the given labeled nucleotide. For example, a labeled nucleotide with 2 dyes may experience a different level of quenching than a labeled nucleotide with 5 dyes. A type of labeled nucleotide may be associated with a unique signal profile or signal intensity.

As such, in some cases, the method may comprise using a mixture of terminated nucleotides, comprising at least a first type of labeled nucleotide comprising a first number of dyes, a second type of labeled nucleotide comprising a second number of dyes, a third type of labeled nucleotide comprising a third number of dyes, and a fourth type of unlabeled nucleotide, wherein the first type of labeled nucleotide, second type of labeled nucleotide, third type of labeled nucleotide and fourth type of labeled nucleotide are four different canonical base types, and wherein the first type of labeled nucleotide, the second type of labeled nucleotide, and the third type of labeled nucleotide are detectable at a same or substantially same frequency.

Alternatively or in addition, the dyes of different labeled nucleotide types may be detectable at different frequencies.

In some cases, different types of labeled nucleotides may be designed to have uniquely associated signal intensities, regardless of number of dyes and/or regardless of length of the linker. Two types of labeled nucleotides that have the same number of dyes may be detected at different signal intensities and/or be associated with different signal profiles. For example, a first type of labeled nucleotide of a first canonical base may have x number of dyes attached to ay length linker and a second type of labeled nucleotide of a second canonical base may have the same x number of dyes attached to a z length linker, where y equals z (same length linkers, and same number of dyes, but between the two types of labeled nucleotides, the x dyes can be attached to different locations with different distance(s) between them in the respective linkers) or y does not equal z (different length linkers, and same number of dyes), and the first type of labeled nucleotide and the second type of labeled nucleotide may be detected at different signal intensities (or be associated with unique signal profiles when detected). Two types of labeled nucleotides that have the same length linker may be detected at different signal intensities and/or be associated with different signal profiles. For example, a first type of labeled nucleotide of a first canonical base may have x number of dyes attached to ay length linker and a second type of labeled nucleotide of a second canonical base may have z number of dyes attached to ay length linker, where x equals z (same length linkers, and same number of dyes, but between the two types of labeled nucleotides, the x=z dyes can be attached to different locations with different distance(s) between them in the respective linkers) or x does not equal z (same length linkers, and different number of dyes), and the first type of labeled nucleotide and the second type of labeled nucleotide may be detected at different signal intensities (or be associated with unique signal profiles when detected).

In some cases, different types of labeled nucleotides may be designed to have uniquely associated signal intensities by adjusting a levels of quenching. A level of quenching for a labeled nucleotide may be adjusted by the presence or absence of one or more quencher moieties, as well as adjusting a distance between the one or more quencher moieties and one or more dye moieties. For example, the distance may be adjusted by using a semi-rigid linker, such as a hydroxyproline linker or any other linker (or linker combination) described elsewhere herein. Generally, quenching increases (and thus signal attenuation increases) as the distance between a dye moiety and a quencher moiety decreases. In some cases, a labeled nucleotide may comprise a dye moiety and a quencher moiety separated by a linker (e.g., hydroxyproline linker) or at least a portion of a linker with a predetermined length (e.g., Hyp20, Hyp10, Hyp6, etc.), with the nucleotide attached to any functional group (e.g., free carboxylate group) suitably located between the dye moiety and the quencher moiety.

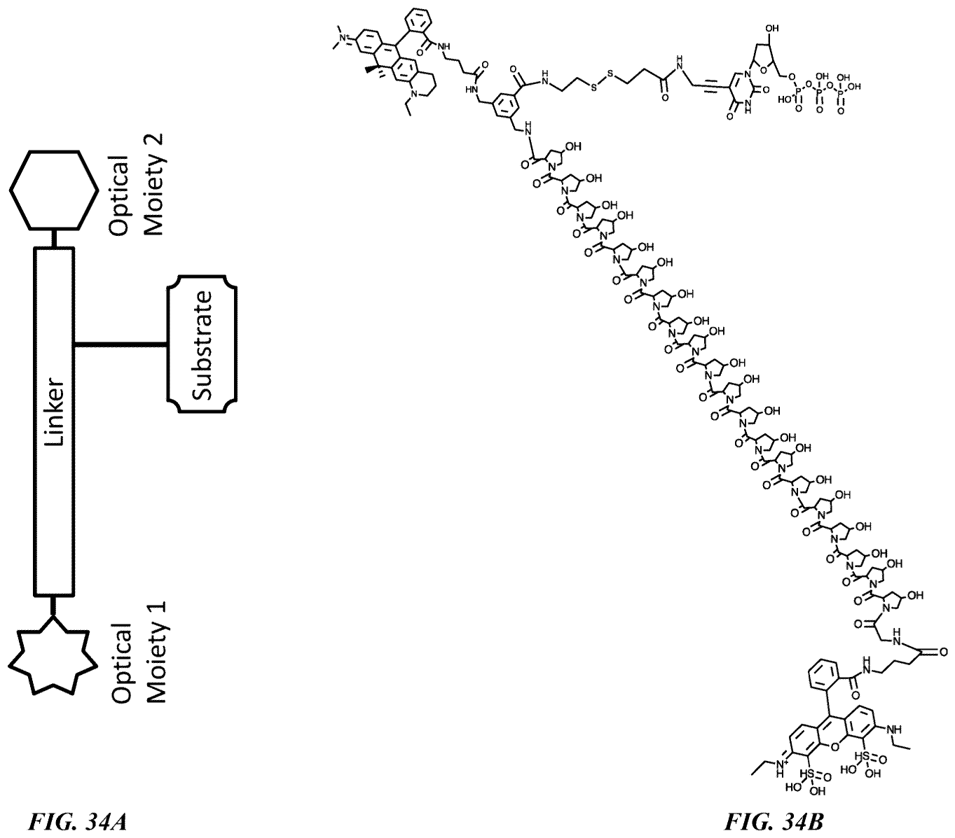

FIG. 34A shows one example structure of an adjustably labeled substrate. Two optical moieties, Optical Moiety 1 and Optical Moiety 2, may be attached to each end of a Linker, and the Substrate may be attached to any suitable location between the two optical moieties. For example, the linker may comprise one or more functional groups (e.g., carboxylate group) that serves as an attachment site for the substrate. Optical Moiety 1 may comprise a dye moiety, which exhibits quenching activity when in proximity with Optical Moiety 2. Optical Moiety 2 may be another dye moiety or any quencher moiety. Alternatively or in addition, Optical Moiety 2 may comprise a dye moiety, which exhibits quenching activity when in proximity with Optical Moiety 1. Optical Moiety 1 may be another dye moiety or any quencher moiety. The length of the Linker may be adjusted to adjust the distance between Optical Moiety 1 and Optical Moiety 2, thereby tuning quenching by Optical Moiety 1 and/or Optical Moiety 2, and thus adjusting the signal intensity or signal profile of the labeled substrate. The substrate may be any substrate described herein such as a nucleotide (e.g., dNTP) or protein. FIG. 34B shows an example of a labeled substrate per the structure of FIG. 34A. The labeled substrate is a dUTP labeled with an Atto532 dye moiety and an Atto633 dye moiety which two dyes are separated by 20 hydroxyproline residues. It will be appreciated that while FIG. 34A and FIG. 34B illustrate two quenching optical moieties disposed at opposing ends of a linker, one or both of the two quenching optical moieties may be disposed in the middle of the linker (e.g., hydroxyproline linker), such as described with respect to FIGS. 23A-E, and the portion of the linker disposed between the two optical moieties may be adjusted to tune the level of quenching. It will be appreciated that any number of quenching moieties and any number of dye moieties may be used, disposed strategically at different locations with respect to the linker, to tune the final level of quenching, and final signal intensity or signal profile associated with a labeled substrate.

Thus, a method may comprise (a) providing a primer-hybridized template nucleic acid molecule; and (b) contacting the primer-hybridized template nucleic acid molecule with a mixture of terminated nucleotides, wherein the mixture of terminated nucleotides comprises at least a first type of labeled nucleotide and a second type of labeled nucleotide, wherein the first type of labeled nucleotide is a first canonical base and the second type of labeled nucleotide is a second canonical base different from the first canonical base, wherein the first type of labeled nucleotide and the second type of labeled nucleotide are detectable at different signal intensities, and wherein the first type of labeled nucleotide and the second type of labeled nucleotide are detectable at a same or substantially same frequency. The method may further comprise (c) detecting one or more signals form the primer-hybridized template nucleic acid molecule. The mixture of nucleotides may comprise three different types of labeled nucleotides with three different canonical base types, each type of canonical base detectable at different signal intensities. The mixture of nucleotides may comprise four different types of labeled nucleotides with four different canonical base types, each type of canonical base detectable at different signal intensities. The mixture of nucleotides may comprise three different types of labeled nucleotides with three different canonical base types and an unlabeled nucleotide of a fourth canonical base type different than the three different canonical base types, each type of labeled nucleotides detectable at different signal intensities.

A labeled nucleotide comprising n number of dyes may be any of the labeled nucleotides and any of the multi-labeled nucleotides, such as described with respect to FIGS. 23A-E and FIGS. 34A-B described herein.

As standard with terminated sequencing protocols, for these methods, after incorporation of the nucleotides, and washing away unincorporated nucleotides from the reaction space, the terminated nucleotides may be unblocked so that the primers may proceed with extension of the next base for sequencing. The labels may be cleaved subsequent to detection. The labels may be cleaved prior to next round of incorporation. The detection may be performed prior to, during, or subsequent to unblocking.

Provided herein are kits and compositions that comprise reagents used for the methods described herein. For example, a kit or composition may comprise a mixture of nucleotides, comprising a first type of labeled nucleotide and a second type of labeled nucleotide, the first type and second type of labeled nucleotides being detectable at different signal intensities or associated with unique signal profiles. The kit or composition may further comprise three types of labeled nucleotides, each type of labeled nucleotide being detectable at different signal intensities or associated with unique signal profiles. The kit or composition may further comprise four types of labeled nucleotides, each type of labeled nucleotide being detectable at different signal intensities or associated with unique signal profiles. The kit or composition may further comprise an unlabeled nucleotide of one or more canonical base types. For example, a kit or composition may comprise a mixture of nucleotides, comprising a first type of nucleotide labeled with a first number of dyes and a second type of nucleotide labeled with a second number of dyes, the first and second numbers being different. The mixture of nucleotides may further comprise a third type of nucleotide labeled with a third number of dyes, the first, second, and third numbers being different. The mixture of nucleotides may further comprise a fourth type of nucleotide labeled with a fourth number of dyes, the first, second, third, and fourth numbers being different. Different types of labeled nucleotides may have different length linkers (e.g., different Hypn) with different number of dyes. Different types of labeled nucleotides may have similar or same length linkers (e.g., different Hypn) but with different number of dyes. Different types of labeled nucleotides may have different length linkers (e.g., different Hypn) with same number of dyes. Different types of nucleotides may have similar or same length linkers (e.g., different Hypn) but with same number of dyes.

### Methods for Synthesis of Optical Labeling Reagents

In some cases, the linkers provided herein may be prepared using peptide synthesis chemistry.

For example, a linker comprising a pyridinium moiety may be prepared using peptide synthesis chemistry. Such a method may use four bifunctional reagents to make the linker, namely: (a) R¹A, (b) BB, (c) AA, and (d) AR². Reagent A reacts with B to form a pyridinium group; R¹ and R² are hetero-bifunctional attachment groups. The synthesis begins with the group R¹A (or R²A). Excess BB is added to R¹A to form R¹A-BB. The product is precipitated and washed in a less polar solvent (such as ethyl acetate or tetrahydrofuran) to remove excess BB. Excess AA is added with heat in N-methylpyrrolidone (NMP) to produce R¹A-BB-AA. The product is precipitated and washed in a less polar solvent. The synthesis proceeds until a linker of a particular length is formed. The group AR² is appended in the final step.
1) R¹A+10BB→R¹A-BB (wash away excess BB)
2) R¹A-BB+10 AA→R¹A-BB-AA (wash away excess AA)
3) R¹A-BB-AA+10 BB→R¹A-BB-AA-BB (wash away excess BB)
4) R¹A-BB-AA-BB+AR²→R¹A-BB-AA-BB-AR² (use terminating reagent)

Synthetic methods for preparing optical labeling reagents (e.g., as described herein) are described elsewhere and in the Examples below.

### Methods for Constructing Labeled Nucleotides

In an aspect, the present disclosure provides methods for constructing labeled nucleotides (e.g., optically labeled nucleotides).

Labeled nucleotides can be constructed using modular chemical building blocks. A nucleotide or nucleotide analog can be derivatized with, e.g., a propargylamino moiety to provide a handle for attachment to a linker or detectable moiety (e.g., dye). One or more detectable moieties, such as one or more dyes, can be attached to a nucleotide or nucleotide analog via a covalent bond. Alternatively or additionally, one or more detectable moieties can be attached to a nucleotide or nucleotide analog via a non-covalent bond. A detectable moiety may be attached to a nucleotide or nucleotide analog via a linker (e.g., as described herein). A linker may include one or more moieties. For example, a linker may include a first moiety including a disulfide bond within it to facilitate cleaving the linker and releasing the detectable moiety (e.g., during a sequencing process). Additional linker moieties can be added using sequential peptide bonds. Linker moieties can have various lengths and charges. A linker moiety may include one or more different components, such as one or more different ring systems, and/or a repeating unit (e.g., as described herein). Examples of linkers include, but are not limited to, aminoethyl-SS-propionic acid (epSS), aminoethyl-SS-benzoic acid, aminohexyl-SS-propionic acid, hyp10, and hyp20.

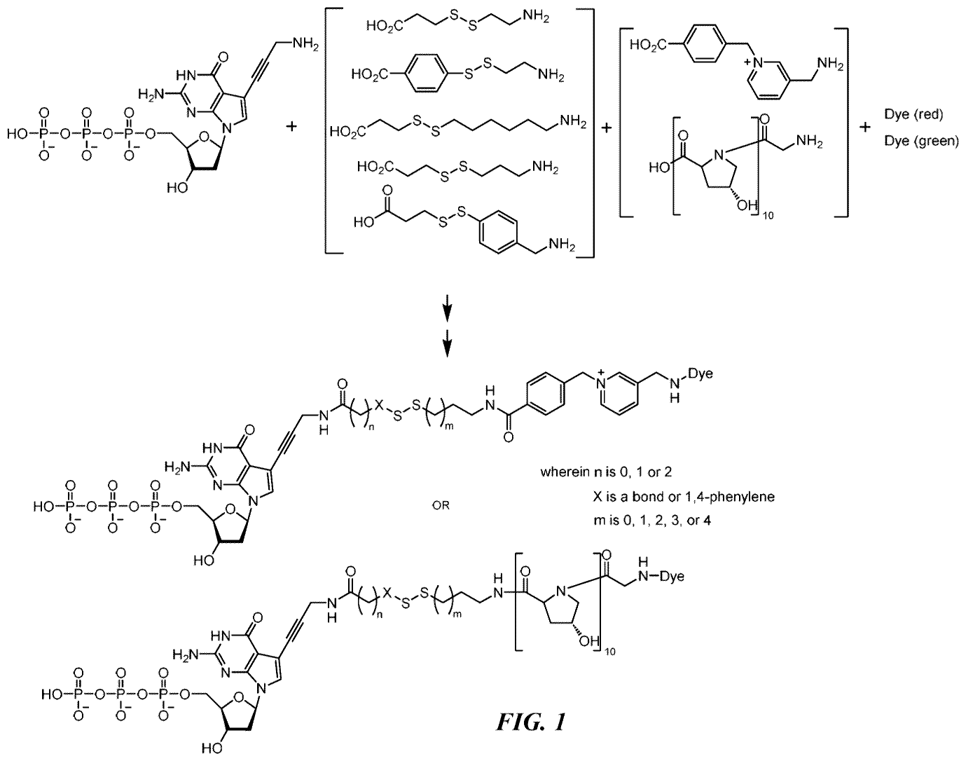

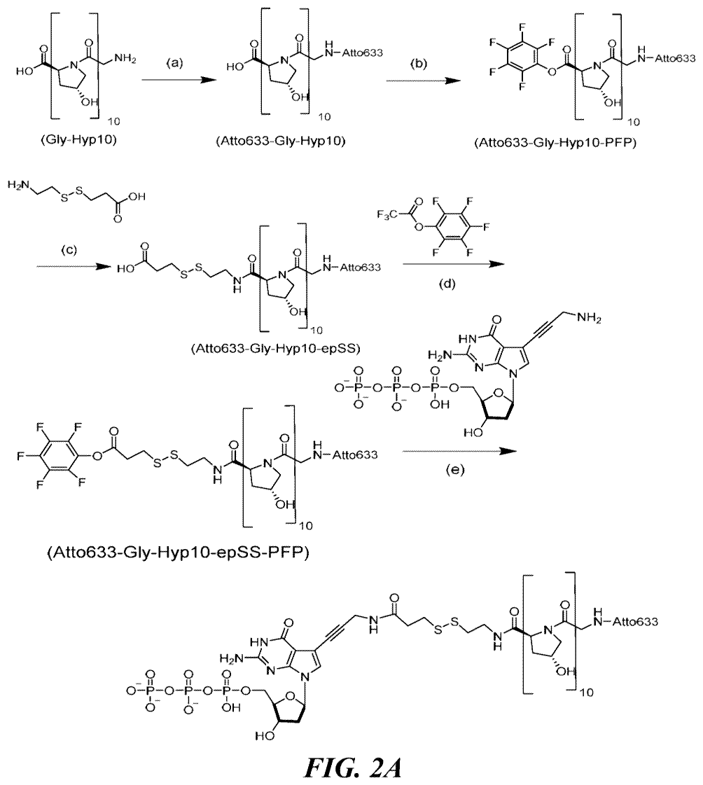

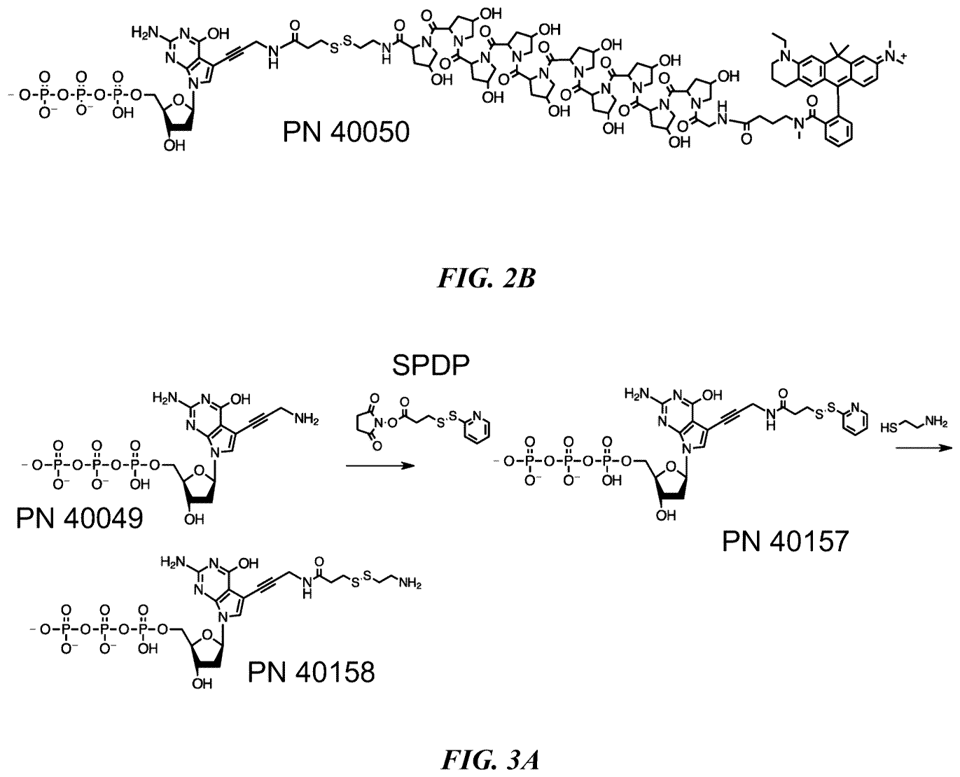

Examples of methods for constructing labeled nucleotides are shown in FIGS. 1, 2A, and 2B. As shown in FIG. 1, a labeled nucleotide may be constructed from a nucleotide, a dye, and one or more linker moieties. The one or more linker moieties together comprise a linker as described herein. A nucleotide functionalized with a propargylamino moiety can be attached to a first linker moiety via a peptide bond. This first linker moiety may comprise a cleavable moiety, such as a disulfide moiety. The first linker moiety can also be attached to one or more additional linker moieties in linear or branching fashions. For example, a second linker moiety may include two or more ring systems, wherein at least two of the two or more ring systems are separated by no more than two sp3 carbon atoms, such as by no more than two atoms. For example, at least two of the two or more ring systems may be connected to each other by a sp² carbon atom. The linker may comprise a non-proteinogenic amino acid comprising a ring system of the two or more ring systems. For example, the second linker moiety may comprise a two or more hydroxyproline moieties. An amine handle on a linker moiety may be used to attach the linker and a dye, such as a dye that fluoresces in the red or green portions of the visible electromagnetic spectrum. The labeled nucleotide generated in FIG. 1 comprises a modified deoxyadenosine triphosphate moiety, a linker comprising a first linker moiety including a disulfide moiety and a second linker moiety including at least two ring systems, and a dye.

Construction of a labeled nucleotide can begin from either the nucleotide terminus or the dye terminus. Construction from the dye terminus permits the use of unlabeled, not activated amino acid moieties, while construction from the nucleotide terminus may require amine-protected, carboxy-activated amino acid moieties.

FIGS. 2A and 2B show an example synthesis of a labeled nucleotide including a propargylamino functionalized dGTP moiety, a first linker moiety including a disulfide group, a second linker moiety that is hyp10, and the dye moiety ATTO 633. Details of this synthesis are provided in Example 2 below.

A nucleotide or nucleotide analog of a labeled nucleotide may include one or more modifications, such as one or more modifications on the nucleobase. Alternatively, a nucleotide or nucleotide analog of a labeled nucleotide may include one or more modifications not on the nucleobase. Modifications can include, but are not limited to, covalent attachment of one or more linker or label moieties, alkylation, amination, amidation, esterification, hydroxylation, halogenation, sulfurylation, and/or phosphorylation.

A nucleotide or nucleotide analog of a labeled nucleotide may include one or more modifications that are configured prevent subsequent nucleotide additions to a position adjacent to the labeled nucleotide upon its incorporation into a growing nucleic acid strand. For example, the labeled nucleotide may include a terminating or blocking group (e.g., dimethoxytrityl, phosphoramidite, or nitrobenzyl molecules). In some instances, the terminating or blocking group may be cleavable.

### Tandem Labeling

The present disclosure provides reagents and methods for tandem labeling. Tandem labeling may comprise an additional fluorescent labeling agent to a fluorescent labeling agent. Fluorescent labeling agents involved in tandem labeling or otherwise an energy transfer may be referred to herein as “tandem labeling agents.” In some cases, tandem labeling may comprise two or more tandem labeling agents. Tandem labeling may comprise an energy transfer between two tandem labeling agents. In some cases, an energy transfer between two tandem labeling agents may comprise Forster resonance energy transfer or fluorescence resonance energy transfer (FRET), resonance energy transfer (RET), or electronic energy transfer (EET). In some cases, an energy transfer between two tandem labeling agents may comprise radiationless or non-radiative energy transfer between two labeling agents. In other cases, an energy transfer between two tandem labeling agents may also comprise radiative energy transfer between two labeling agents. Any of the labeling reagents and/or labeled substrates of the present disclosure may comprise a fluorescent labeling agent and an additional fluorescent labelling agent, for example a first and second fluorescent dye. The two labeling agents or dyes may be to any of the dye attachment points of the substrates and/or linkers (e.g., hyp20, etc.) described herein. The two labeling agents or dyes may be a donor-acceptor fluorophore pair labeling agents or dyes. In some cases, the two labeling agents may be conjugated to one molecule.

### EXAMPLES

### Example 1: General Synthetic Principles

Certain examples of the following examples illustrate various methods of making linkers and labeled substrates described herein. It is understood that one skilled in the art may be able to make these compounds by similar methods or by combining other methods known to one skilled in the art. It is also understood that one skilled in the art would be able to make other compounds in a similar manner as described below by using the appropriate starting materials and modifying synthetic routes as needed. In general, starting materials and reagents can be obtained from commercial vendors or synthesized according to sources known to those skilled in the art or prepared as described herein.

Unless otherwise noted, reagents and solvents used in synthetic methods described herein are obtained from commercial suppliers. Anhydrous solvents and oven-dried glassware may be used for synthetic transformations sensitive to moisture and/or oxygen. Yields may not be optimized. Reaction times may be approximate and may not be optimized. Materials and instrumentation used in synthetic procedures may be substituted with appropriate alternatives. Column chromatography and thin layer chromatography (TLC) may be performed on reverse-phase silica gel unless otherwise noted. Nuclear magnetic resonance (NMR) and mass spectra may be obtained to characterize reaction products and/or monitor reaction progress.

### Example 2: Synthesis of dGTP-AP-SS-hyp10-Atto633

Described herein is a method for constructing the labeled nucleotide dGTP-AP-SS-hyp10-Atto633. FIG. 2A illustrates an example method for the synthesis of a fluorescently labeled dGTP reagent. FIG. 2B illustrates the full structures of the dye and linker of the resulting fluorescently labeled dGTP. The method involves formation of a covalent linkage between Gly-Hyp10 and the fluorophore Atto633 (process (a)), esterification to couple Atto633-Gly-Hyp10 with pentafluorophenol (process (b)), substitution with the linker molecule epSS (process(c)), esterification to form Atto633-Gly-Hyp10-epSS-PFP (process (d)), and substitution with dGTP to provide the fluorescently labeled nucleotide (process e). Details of the synthesis are provided below.

Preparation of Atto633-Gly-Hyp10. (FIG. 2A process (a)) A stock solution of Gly-Hyp10 (also referred to herein as “hyp10” and “Hyp10”) in bicarbonate is prepared by dissolving 25 milligrams (mg) of the 11 amino acid peptide in 500 microliters (μL) of 0.2 molar (M) sodium bicarbonate in a 1.5 milliliter (mL) Eppendorf tube. 7 mg of Atto633-NHS is weighed into another Eppendorf tube and dissolved in 200 μL of dimethylformamide (DMF). A volume of 300 μL of the peptide solution is added to the solution containing Atto633-NHS. The resulting solution is mixed and heated to 50° C. for 20 minutes (min). The extent of the reaction is followed with reverse-phase thin layer chromatography (TLC). A 1 μL aliquot of the reaction solution is removed and dissolved in 40 μL water and spotted on reverse phase TLC. A co-spot with Atto633 acid is included, and Atto633 is also run alone. The plate is eluted with a 2:1 solution of acetonitrile 0.1 M triethylammonium bicarbonate (TEAB). Atto633 acid and Atto633-NHS both have an R_{f} of zero, while Gly-Hyp10 has an R_{f} of 0.4. The product is purified by injecting the solution onto a C18 reverse phase column using the gradient 20%→50% acetonitrile vs. 0.1M TEAB over 16 minutes at 2.5 mL/min. The desired product is the major product, Atto633-Gly-Hyp10, eluting at 15.2 minutes. The fractions containing the desired material are collected in Eppendorf tubes and dried, yielding a blue solid. A major peak was observed on ESI mass spec: m/z calculated for C₈₇H₁₁₅N₁₄O₂₄⁺, [M]⁺=1739.8; found: 1740.6.

Preparation of Atto633-Gly-Hyp10-PFP. (FIG. 2A process (b)) Atto633-Gly-Hyp10 is suspended in 100 μL DMF in a 1.5 mL Eppendorf tube. Pyridine (20 μL) and pentafluorophenyl trifluoroacetate (PFP-TFA, 20 μL) are added to the tube. The reaction mixture is warmed to 50° C. in a heat block for 20 min. The reaction is monitored by removing 1 μL aliquots and adding to 1 mL of dilute HCl (0.4%). When the reaction is complete the aqueous solution is colorless. After 10 min the dilute HCl solution is light blue. Additional PFP-TFA (30 μL) is added. After another 100 min at 50° C. a retest of precipitation gives a colorless solution. The remaining reaction mixture is precipitated into 1 mL dilute HCl in 20 μL portions. 20 μL is added to 1 mL dilute HCl, the tube spun down, and aqueous solution discarded. The process is repeated until all of the product is precipitated. The residue is thoroughly dried. After drying, the solid is washed twice with 1 mL methyl tert-butyl ether (MTBE). The product is a dark blue powder. The product gives a major peak on electrospray ionization (ESI)-mass spectrometry (MS): m/z calculated for C₉₃H₁₁₅F₅N₁₄O₂₄²⁺, [M+H]²⁺=1906.8/2=953.4; found: 953.4.

Preparation of Atto633-Gly-Hyp10-epSS. (FIG. 2A process (c)) Atto633-Gly-Hyp10-PFP (1.6 micromoles (μmol)) is dissolved in 100 μL DMF in an Eppendorf tube. A solution of aminoethyl-SS-propionic acid (Broadpharm; 6 mg in 200 μL 0.1 M bicarbonate) is mixed with the Atto633-gly-hyp10-PFP and heated to 50° C. in a heat block for 20 min. Atto633-Gly-Hyp10-epSS is purified from the resulting reaction mixture by reverse phase HPLC using a gradient of 20%→50% acetonitrile over 16 min. Atto633-Gly-Hyp10 elutes at 15 min and Atto633-Gly-Hyp10-epSS elutes at 15.6 min. The fractions containing the product, Atto633-Gly-Hyp10-epSS, are combined and dried. The product has a major peak on ESI-MS: m/z calculated for C₉₂H₁₂₄N₁₅O₂₅S₂⁺, [M]⁺=1902.8; Found: 1902.6.

Preparation of Atto633-Gly-Hyp10-epSS-PFP. (FIG. 2A process (d)) Atto633-Gly-Hyp10-epSS is dissolved in 100 μL DMF in an Eppendorf tube. Pyridine (20 μL) and PFP-TFA (20 μL) are added and the mixture is heated to 50° C. in a heat block for 20 min. A test aliquot (1 μL) in dilute HCl gives a colorless solution and a blue precipitate. The reaction is precipitated in 20 μL aliquots in 1 mL dilute HCl, the tube spun down, and the aqueous solution discarded. The process is repeated until all the PFP ester is precipitated. The residue is thoroughly dried under vacuum and washed with MTBE.

Preparation of dGTP-AP—SS-Atto633. (FIG. 2A process (e)) A solution of aminopropargyl dGTP (Trilink; 1 μmol in 100 μL of 0.2 M bicarbonate) is added to 50 μL of a DMF solution comprising Atto633-gly-hyp10-epSS-PFP. The mixture is heated to 50° C. for 10 min. The product, dGTP-AP-epSS-Atto633, is purified by reverse-phase HPLC using a gradient of 20%→50% acetonitrile 16 min. The product elutes at 15.3 min. Preparative HPLC provides 0.65 μmol. The product gives a major peak on ESI-MS: m/z calculated for C₁₀₆H₁₃₉N₂₀O₃₇P₃S₂²⁻, [M−H]²⁻, 1220.4; found: 1220.6.

While synthesis of dGTP-Atto633-Gly-Hyp10-epSS-PFP is described, a skilled practitioner will recognize that other fluorescently labeled nucleotides can be produced in a similar manner using appropriate starting materials.

### Example 3: Preparation of Dye-Labeled Nucleotides

A set of dye-labeled nucleotides designed for excitation at about 530 nm is prepared. Excitation at 530 nm may be achieved using a green laser, which may be readily available, high-powered, and stable. There are many commercially available fluorescent dyes with excitation at or near 530 nm that are inexpensive and have a variety of properties (hydrophobic, hydrophilic, positively charged, negatively charged). Synthetic routes to such dyes may be shorter and cheaper than those for longer wavelength dyes. Moreover, certain green dyes may have significantly less self-quenching than red dyes, potentially allowing for the use of higher labeling fractions (e.g., as described herein).

A viable reagent set for use in, e.g., a sequencing application consists of each of four canonical nucleotides or analogs thereof with cleavable green dyes that perform well in sequencing. An optimal set may be prepared by varying each component of a labeled nucleotide structure to obtain an array of candidate labeled nucleotides with varying properties. The resultant nucleotides are evaluated (e.g., as described below), and certain labeled nucleotides are optimized for concentration and labeling fraction (the ratio of labeled to unlabeled nucleotide in a flow).

FIG. 4 shows a variety of components that may be used in the construction of detectably labeled nucleotides. A nucleotide can be modified with a cleavable linker moiety, a semi-rigid linker moiety such a linker moiety comprising one or more amino acids, and a fluorescent dye moiety. The nucleotides shown in FIG. 4 are propargylamino functionalized nucleotides (A, C, G, T, and U), but any other useful nucleotide or nucleotide analog with any other useful chemical handle can be used. Cleavable linker moieties include, for example, the structures shown as “Q,” “E,” “B,” “Y,” and “P”. Each cleavable linker moiety includes a cleavable group (e.g., as described herein). For example, cleavable linker moieties Q, E, B, Y, and P include disulfide bonds. A linker moiety (e.g., a semi-rigid linker moiety) may comprise one or more amino acid moieties, including, for example, one or more hydroxyproline moieties (e.g., as described herein). For example, a linker moiety may comprise a hydroxyproline linker (Hypₙ). The “H” linker moiety illustrated in FIG. 4 is hyp10 moiety. In some cases, a fluorescently labeled nucleotide may comprise multiple hyp10 moieties in the same or different regions of the chemical structure. For example, a linker moiety may comprise 2 or more hyp10 moieties (e.g., a hyp20 (e.g., a “HH” moiety in FIG. 4) or hyp30 moiety, each of which may include 10 hydroxyproline moieties and, in some cases, another moiety such as a glycine moiety, as described herein) in sequence, which moieties may be separated by one or more other moieties or features. In some cases, a linker moiety may comprise cysteic acid (e.g., the “Cy” moiety in FIG. 4) or two cysteic acids (e.g., the “CyCy” moiety in FIG. 4), 5-amino-5-carboxy-N,N,N-trimethylpentan-1-aminium or a salt thereof (e.g., the “L” moiety in FIG. 4) or two 5-amino-5-carboxy-N,N,N-trimethylpentan-1-aminium or a salt thereof (e.g., the “LL” moiety in FIG. 4). In some cases, a linker moiety may comprise 6-aminohexanoic acid (e.g., the “Am” moiety in FIG. 4). In some cases, a linker moiety may comprise the “C” moiety shown in FIG. 4. In some cases, a linker moiety may comprise the “V” moiety or “W” moiety (comprising quaternary amines) shown in FIG. 4. A linker may include multiple different portions including multiple different amino acid sequences including 2 or more amino acids (e.g., as described herein). In some cases, a nucleotide may also not comprise a linker described herein. In some cases, a fluorescently labeled nucleotide may comprise a branched or dendritic structure (e.g., as described herein) comprising multiple linker moieties (e.g., multiple sets of hydroxyproline moieties connected at different branch points to a central structure), which linker moieties may be the same or different. In some cases, a fluorescently labeled nucleotide may comprise multiple dyes attached to different locations of a hydroxyproline moiety. A fluorescently labeled nucleotide may also include one or more fluorescent dye moieties. A fluorescent dye moiety may be a structure shown in FIG. 4 as “Kam”, “*,” “#,” “$,” “\hat{ }\hat{ },” or any other useful structure, such as any of the dyes or labels described elsewhere herein. Throughout the application, these labels are used to refer to specific dye structures. However, wherever such labels are used, any other dye moiety may be substituted, including any other fluorescent dye moiety described herein. In some cases, a dye may be represented as “‡,” which symbol is intended to represent any useful dye moiety or combination of dye moieties (e.g., dye pairs). Such dyes may fluoresce at or near 530 nm, or in any other useful range of the electromagnetic spectrum (e.g., as described herein). For example, red-fluorescing dyes may also be utilized. In another example, green-fluorescing dyes may also be utilized. Additional examples of dye moieties are included throughout the application. There are numerous possible variations of fluorescently labeled nucleotides. Some example combinations are included in FIG. 4. For example, a fluorescently labeled nucleotide may be U*-YH (e.g., a fluorescently labeled uracil-containing nucleotide comprising a Y cleavable linker and a hyp10 moiety and a * fluorescent dye moiety), U*-YHH (e.g., a fluorescently labeled uracil-containing nucleotide comprising a Y cleavable linker and two hyp10 moieties and a * fluorescent dye moiety), U #-E (e.g., a fluorescently labeled uracil-containing nucleotide comprising an E cleavable linker and a #fluorescent dye moiety and lacking a hyp10 or similar moiety), a G*-B (e.g., a fluorescently labeled guanine-containing nucleotide comprising a B cleavable linker and a * fluorescent dye moiety and lacking a hyp10 or similar moiety), etc. Labeled nucleotides may be prepared according to synthetic routes and principles described herein. In some cases, a nucleotide may not comprise a detectable label or fluorescent dye moiety (e.g., an unlabeled nucleotide).

### Example 4: Dye-Labeled Nucleotides Including Guanine or Analogs Thereof

Nucleotides including guanine or analogs thereof may perform more poorly in sequencing applications (e.g., as described herein) in base-calling accuracy. This may be related to photoinduced electron transfer from the nucleobase to a dye linked to the nucleobase, which may quench signal emitted by the dye and thus less dynamic range of signal. Accordingly, various dye-labeled nucleotides including guanine or analogs thereof are prepared and evaluated as provided herein. Examples of such dye-labeled nucleotides include:

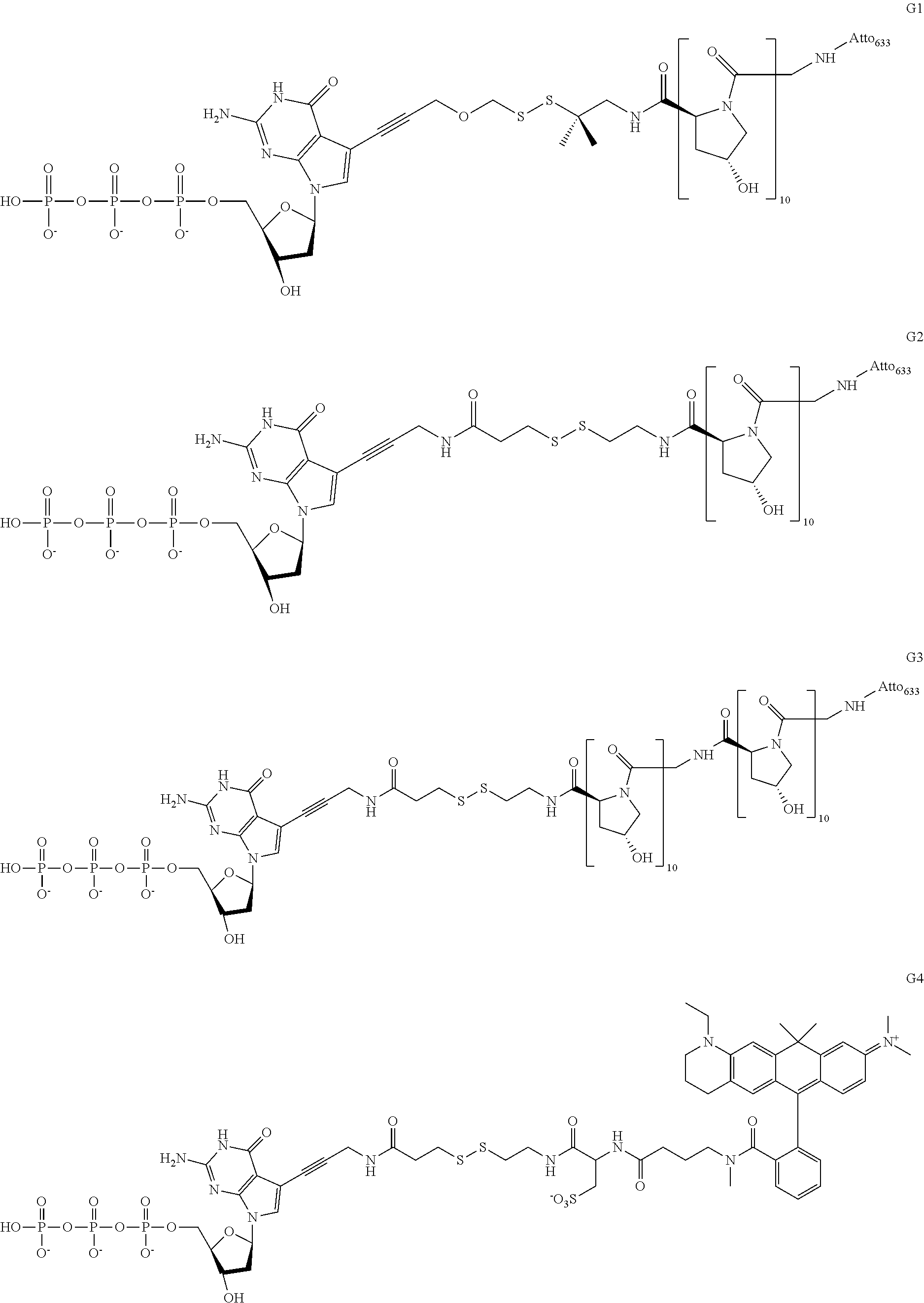

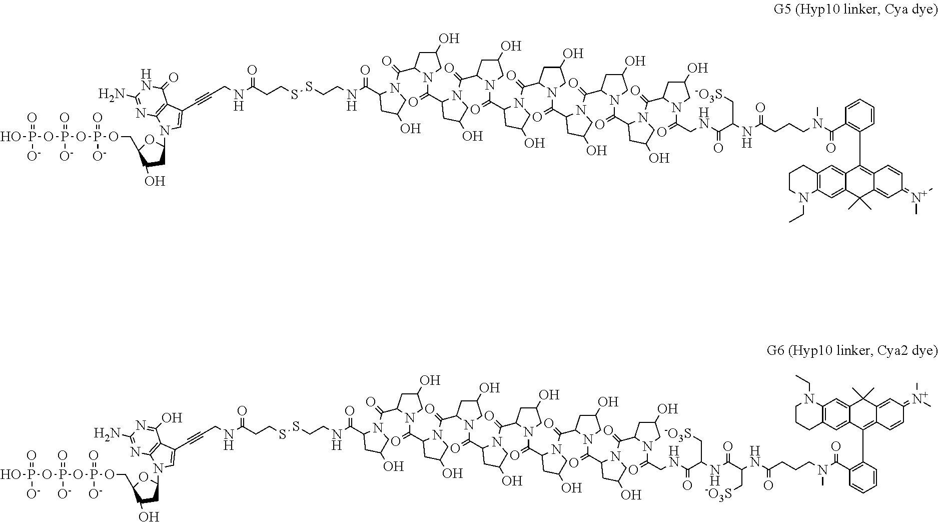

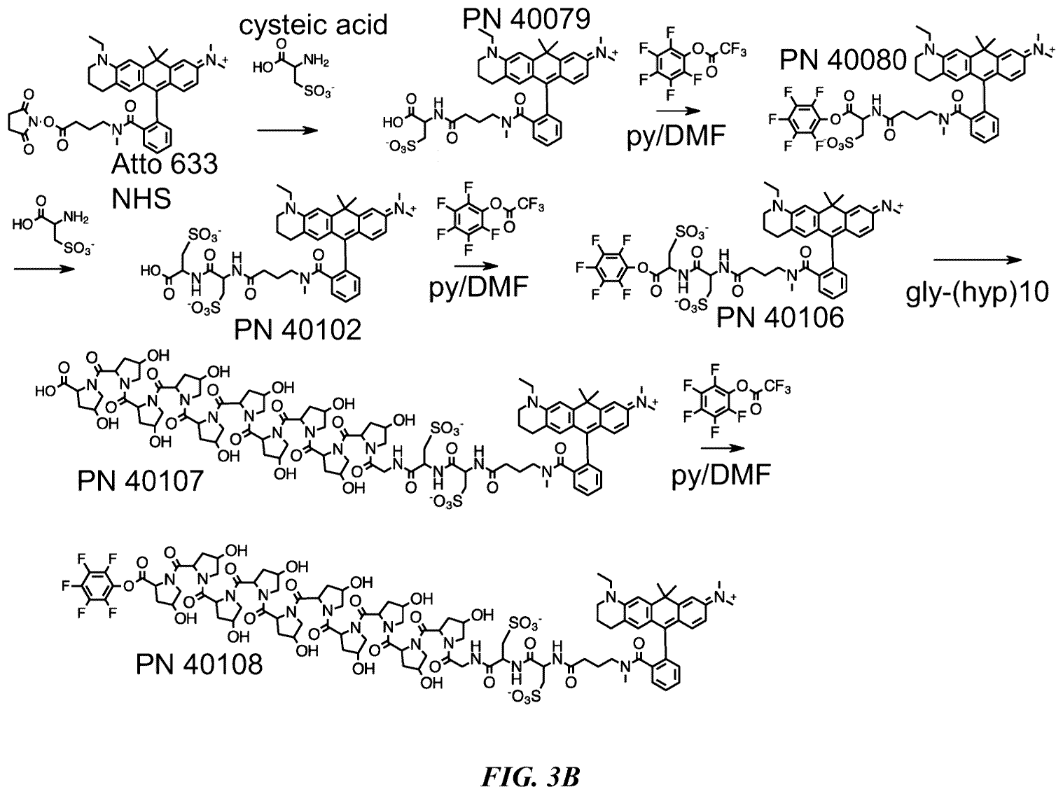

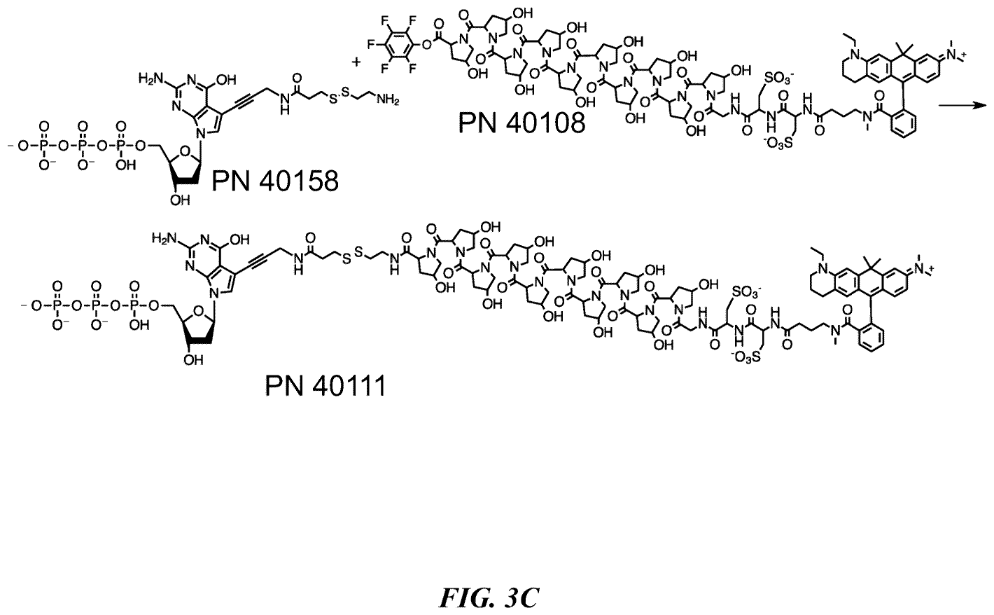

Several of the structures shown above include the hyp10 linker which includes the sequence Gly-Hyp-Hyp-Hyp-Hyp-Hyp-Hyp-Hyp-Hyp-Hyp-Hyp from the N-terminal end. G4, which lacked the hyp10 linker, is highly quenched. The remaining dye-labeled nucleotides are evaluated in a sequencing assay, as described herein. Of the structures shown, G6 provides the highest accuracy. A synthetic route for preparation of G6 is shown in FIGS. 3A-3C. Additional structures including different numbers of hydroxyprolines, including hyp20 and hyp30 moieties, may also be incorporated into fluorescent labeling reagents.

### Example 5: Evaluation of Dye-Labeled Nucleotides

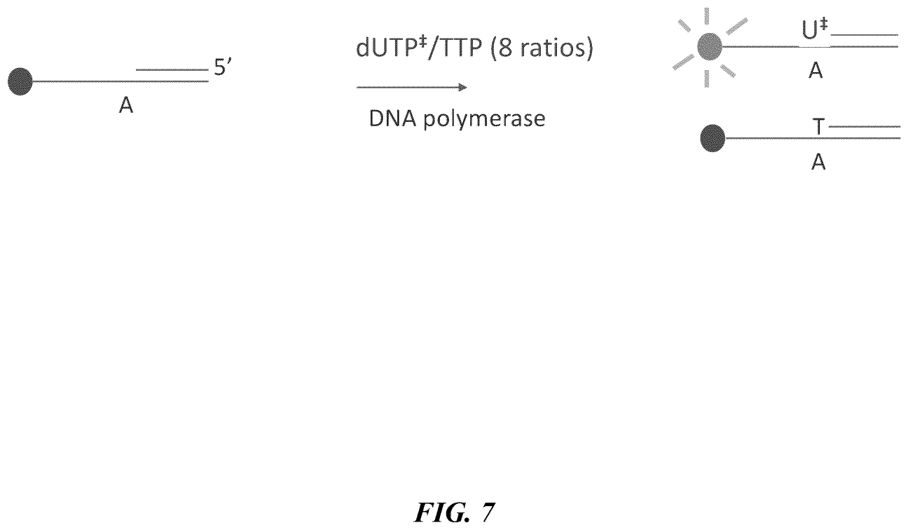

A bead-based assay is used to evaluate dye-labeled nucleotides of Example 4. A streptavidin bead is prepared with a 5′-biotinylated template strand annealed to a primer strand. The primer strand is designed so that the next cognate base incorporated by a DNA polymerase is a thymidine. A DNA polymerase is bound to the bead complex. Various mixtures containing different ratios of the dye-labeled nucleotide (dUTP*) and the natural base (TTP) is then presented to the beads. After washing away excess reagent, the fluorescence of the beads is read on a flow cytometer using the PE channel (excitation=488 nm, emission=580 nm). A schematic of this assay is shown in FIG. 7.

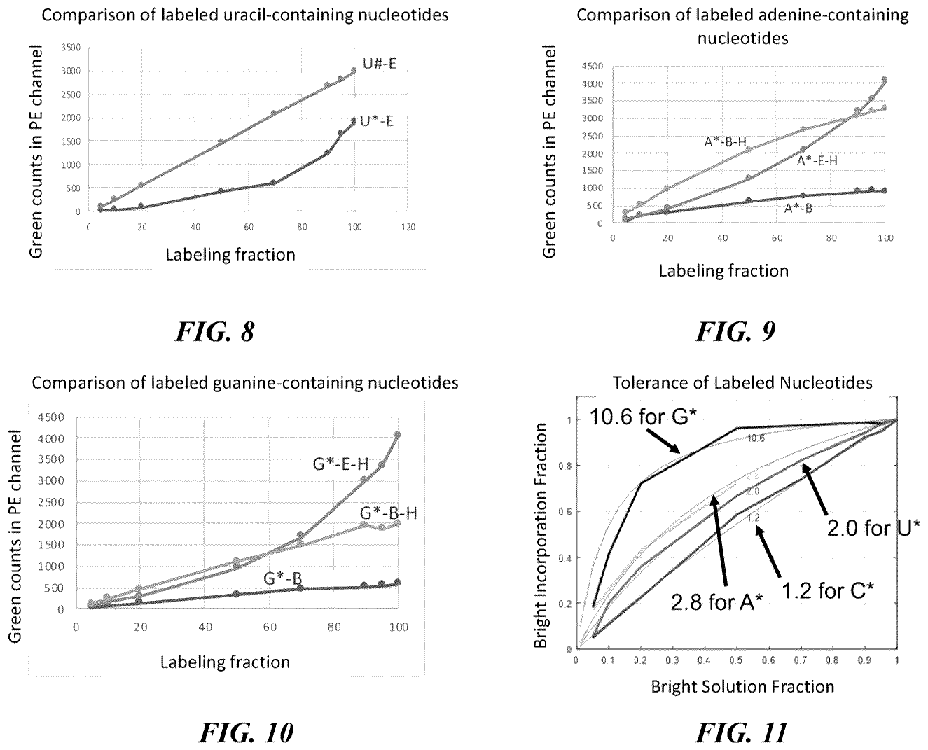

The results of the bead assay for different labeled dUTPs are shown in FIG. 8. The total concentration of the sum of the nucleotides is maintained at 2 μM; a labeling fraction of 10% means 0.2 μM of dUTP* and 1.8 μM of TTP. The behavior for the two nucleotides is noticeably different: U #-E has a “tolerance” of about one, meaning that there is no difference in incorporation of the dye-labeled vs the natural nucleotide over all the ratios tested; i.e., a 50% labeling fraction results in 50% of the beads getting labeled. U*-E, on the other hand, has a negative tolerance, meaning that at every ratio it falls below the line drawn between zero and the signal at 100% labeled. A negative tolerance suggests that the dye-label makes the nucleotide a worse substrate than the natural substrate. This result is consistent with the observation that negatively charged dyes such as ATTO 532 (the dye denoted by U*-E) inhibit incorporation by many polymerases while dyes such as 5-carboxyrhodamine-6G (the dye denoted by U #-E) are zwitterionic and are known to be good substrates.

Additional labeled nucleotides were evaluated using a similar assay. FIG. 9 shows the result of the bead assay for labeled dATPs. FIG. 10 shows the result of the bead assay for labeled dGTPs. For labeled dATPs, very low fluorescence is observed at 100% labeling for A*-B compared to A*-B-H and A*-E-H. This indicates that the hydroxyproline linker (H) relieves quenching of the dye by the nucleotide. A similar result is observed for labeled dGTPs. This result is expected for labeled dGTP, as G quenching via photoinduced electron transfer is well known. A quenching effect from the disulfide linker, B, may also contribute to the lower fluorescence observed for labeled dATPs and dGTPs.

### Example 6: Sequencing Using Dye-Labeled Nucleotides

A nucleic acid sequencing assay may be used to evaluate dye-labeled nucleotides (e.g., as described herein). An example procedure is shown in FIG. 6.

Sequencing may be performed using an instrument outfitted with a light emitting device (LED) and/or a laser. Each nucleotide evaluated may include a dye that is configured for excitement and emission over similar wavelengths (e.g., all red or all green emission). One or more different nucleotide types may be coupled to different dyes. Sequencing performance may be evaluated based on base calling quality, phase lag, phase lead, and homopolymer completion.

Beads with amplified templates are primed, immobilized on a support, and incubated with a tight-binding DNA polymerase. Beads are then subjected to multiple cycles of sequencing. Each sequencing cycle may comprise incubation with U*/T (a fixed ratio of dye-labeled and natural TTP), a “chase” process (TTP alone), imaging, and a cleavage process (10 mM tris(hydroxypropyl)phosphine (THP)) to release the dye. Each process may have a wash process in between. This process may be repeated for A, C, and G-including nucleotides or nucleotide analogs. This sequencing procedure may effectively identify homopolymeric regions of at least 2, 3, 4, 5, 6, 7, 8, or more nucleotides.

Sequencing is also evaluated for an all hyp-linker set in which dye-labeled nucleotides including each canonical nucleotide include the hyp10 or hyp20 linker. This evaluation is performed to identify a set where higher fractions may be used with minimal quenching. Higher quenching may lead to higher scarring (e.g., as described herein), which may reduce incorporation efficiency by a polymerase enzyme. However, family B enzymes such as PolD may perform well with scars. Sequencing may be evaluated with 2.5% and 20% labeling fractions with a dye such as ATTO 633.

Sequencing may be used to evaluate the tolerance for various labeled nucleotides. FIG. 11 shows normalized bead data for nucleotides labeled with a red-emitting dye. Bright solution fraction (b_{f}) is plotted against bright incorporation fraction (bᵢ). The curves are fitted to the following equation:

$${b}_{i} = \frac{tol ( {b}_{f} / {d}_{f} )}{1 + tol ( {b}_{f} / {d}_{f} )}$$

in which d_{f} is the dark solution fraction. In FIG. 11, the calculated tolerances are 10.6 for G*, 2.8 for A*, 2.0 for U*, and 1.2 for C*. The positive tolerance numbers indicate that at 50% labeling fraction, more than 50% is labeled. Reagents with a tolerance of 1 may have the least “context” in sequencing. Reagents with a very negative tolerance (e.g., tolerance<<1) may have issues with uniform incorporation across a plurality of templates coupled to a support because they must be used at such low concentrations that they may fall below saturation and be consumed at an uneven rate.

### Example 7: Evaluation of Quenching

The dye-labeled nucleotides provided herein may improve quenching between nucleobases and the dyes to which they are attached and/or between dyes in a nucleic acid molecule (e.g., a growing nucleic acid strand), such as in a homopolymeric region of a nucleic acid molecule. Quenching may be evaluated in an enzyme-independent manner.

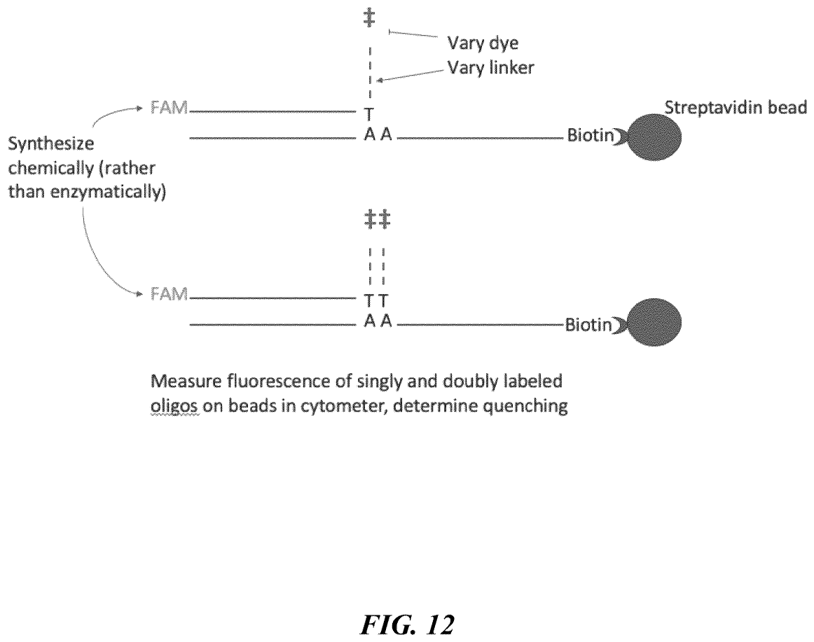

FIG. 12 shows a schematic for evaluating quenching. Synthetic oligos are constructed with one or two “linker arm nucleotides”. Linker arm nucleotides are thymidine analogs with a linker arm containing a primary amine. The oligonucleotide containing the linker arm nucleotide can be labeled with linkers and dyes and HPLC purified. The advantage of using the bead-labeled assay is that exact quantitation of the reagents is not necessary; a large excess can be used in each step and the beads washed, ensuring that only stoichiometric amounts of oligonucleotides are bound to the template. Each dye-linker is put on both oligonucleotides. The beads are measured on the flow cytometer in the APC (red) channel. The percent quenching is determined by the formula: % quenching=100×(1−Fl_{bis}/(2*Flₘₒₙₒ)).

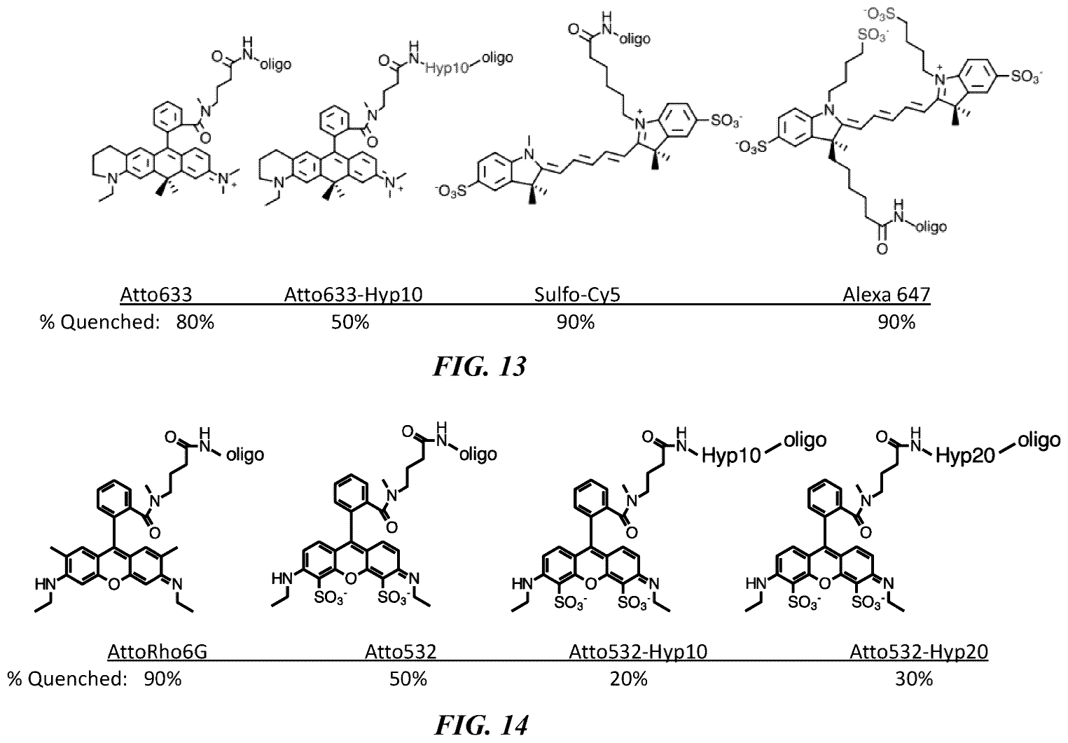

FIGS. 13 and 14 show quenching results for red dye linkers (FIG. 13) and green dye linkers (FIG. 14). The results show that the nature of the dye affects quenching. Negative charge (see Atto532 vs AttoRho6G) can improve quenching but if the dye is extremely large and flat (see Cy5, Alexa 647) quenching may not be improved. The hyp10 or hyp20 linkers improve quenching. As shown in FIG. 13, hyp10 improves quenching with Atto633, and cyanine dyes quench even with four sulfonic acid groups. As shown in FIG. 14, sulfonic acid groups on Atto532 improve quenching, and the combination of Atto532 and hyp10 also improves quenching.

### Example 8: Sequencing by Synthesis Using 100% Labeled Nucleotides

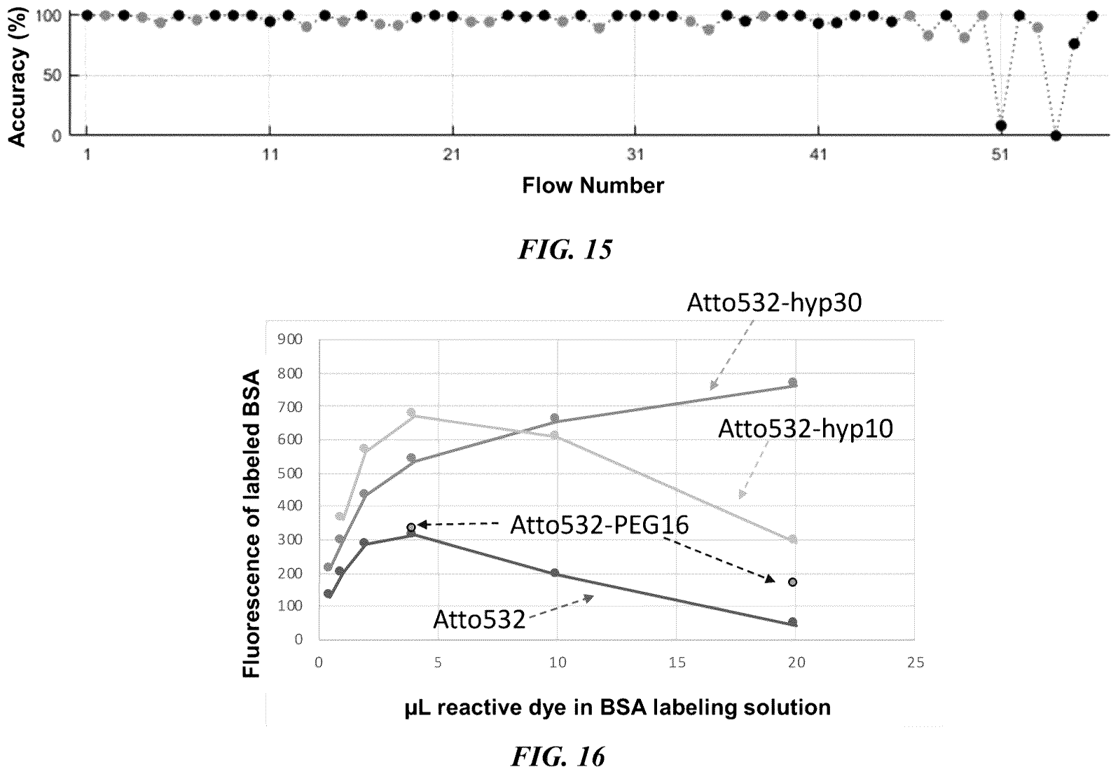

A template nucleic acid having a length of at least 30 nucleotides is sequenced using a plurality of nucleotide flow cycles (see e.g., the schematic in FIG. 6), with solutions in which 100% of the nucleotides are labeled in each flow. In FIG. 15, black circles indicate that a nucleotide base was incorporated in a given flow cycle, while gray circles indicate that a base was not incorporated in a given flow cycle. As can be seen from FIG. 15, the sequencing method can be used to detect base incorporation through at least 50 flow cycles.

### Example 9: Protein Labeling

A protein is labeled with a plurality of optical (e.g., fluorescent) labeling reagents (e.g., as described herein). For example, the protein may be labeled with three or more optical labeling reagents. The optical labeling reagents associated with the protein may all comprise a fluorescent dye of the same type. The optical labeling reagents associated with the protein may all comprise a linker of the same type. In another example, the protein may be labeled with a multi-labeled optical labeling reagent, as described elsewhere herein. The protein may be labeled with any one or more, or combination of the optical labeling reagents described herein. The protein may be an antibody, such as a monoclonal antibody.

The protein is used to label a cell. The cell may be a component of sample, which sample may comprise a plurality of cells. The cells of the sample may be analyzed and sorted using flow cytometry. Flow cytometric analysis may identify the cell as being labeled with the protein associated with the plurality of optical labeling reagents. In some cases, a plurality of cells of a sample may be labeled with optical labeling reagents (e.g., as described herein). For example, cells comprising a particular cell surface feature (e.g., an antigen) configured to associate with a protein (e.g., a protein labeled with a plurality of optical labeling reagents, such as an antibody labeled with a plurality of optical labeling reagents) may be labeled with labeled proteins and analyzed and/or sorted using flow cytometry. Analyzed and/or sorted cells may be subjected to further downstream analysis and processing, including, for example, nucleic acid sequencing, staining, imaging, function assays, immunoassays, isolation/expansion, additional labeling, immunoprecipitation, etc.

### Example 10: Effect of Separation of Dye and Substrate

The effect of functional separation between an optically detectable moiety (e.g., fluorescent dye) and a substrate was investigated using bovine serum albumin (BSA). BSA was fluorescently labeled with ATTO 532 according to the following schemes: in the absence of a linker providing separation between the BSA and ATTO 532 moieties (“Atto532”), using PEG16 as a linker to provide separation between the BSA and ATTO 532 moieties (“Atto532-PEG16”), using a hyp10 moiety to provide separation between the BSA and ATTO 532 moieties (“Atto532-hyp10”), and using a hyp30 moiety to provide separation between the BSA and ATTO 532 moieties (“Atto532-hyp30”). Labeled BSA was purified from free dye using Millipore centrifugal filters. As shown in FIG. 16, the Atto532-hyp30 labeling scheme does not demonstrate self-quenching on the BSA protein. Atto532-hyp30 performed better than Atto532-hyp10, demonstrating that the added physical separation between the BSA and the dye moiety may be useful in reducing quenching. Atto532-PEG16 did not improve quenching over ATTO 532 alone, demonstrating that rigid linker moieties may be preferred for reducing quenching.

### Example 11: Cleavable Linker Moieties

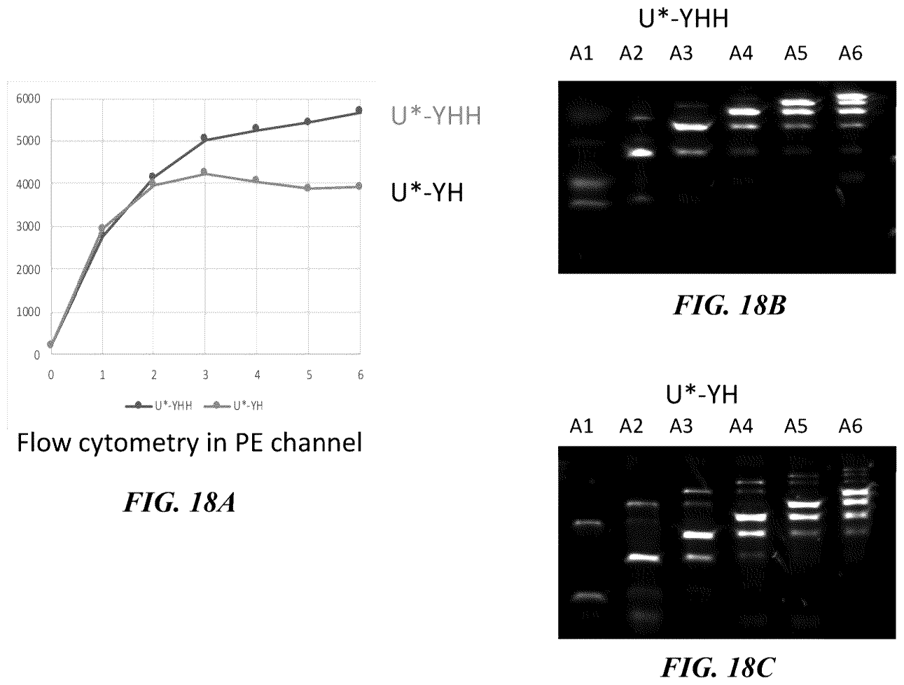

As described herein, a labeling reagent may include a cleavable moiety comprising a cleavable group. The inclusion of a cleavable moiety in a labeling reagent may facilitate separation of the labeling reagent or a portion thereof from a substrate to which it is coupled. The performance of two labeled uracil-containing nucleotides including the same cleavable linker moieties and different semi-rigid portions was also compared; see FIGS. 18A-18C. Sequencing assays were performed using U*-YH and U*-YHH (e.g., a uracil-containing nucleotide labeled with a labeling agent comprising a * dye, a Y cleavable linker, and two hyp10 moieties). Flow cytometry and gel-based analyses were used to evaluate the brightness of signal corresponding to each assay. As shown in FIG. 18A, U*-YHH provided a brighter signal than U*-YH (left panel). As shown in FIGS. 18B and 18C, for a template including six consecutive A's (e.g., a homopolymeric region into which 6 uracils should incorporate), a range of products were measured using each labeled nucleotide. However, U*-YHH was less quenched than U*YH.

### Example 12: Varying Labeling Fractions

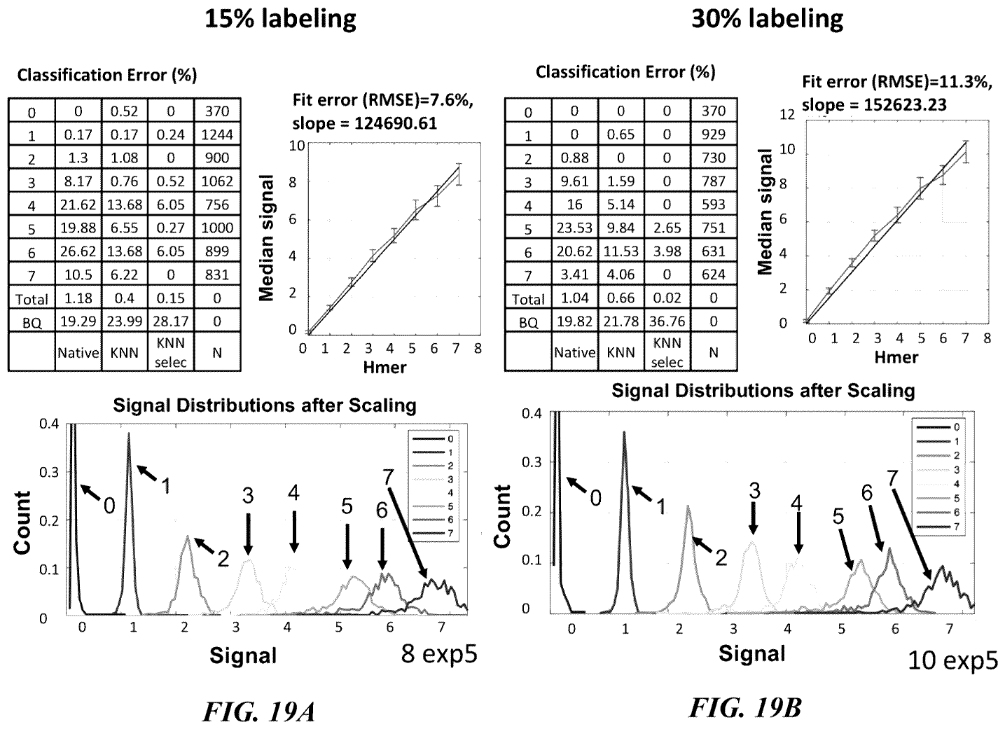

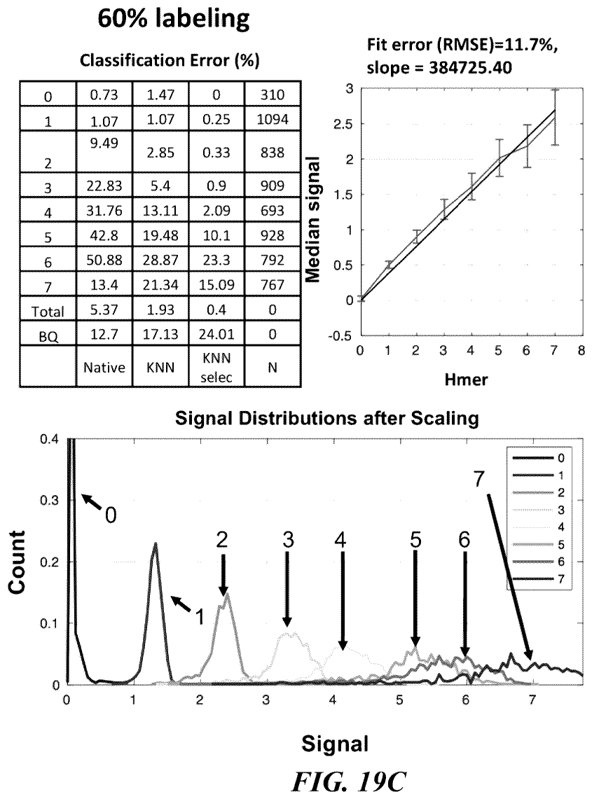

Labeled nucleotides were evaluated at different labeling fractions. The labeled nucleotide U*-EPH was used in a sequencing assay at 15%, 30%, and 60% labeling fractions. As shown in FIGS. 19A, 19B, and 19C, labeling remained approximately linear for homopolymers through eight bases at 60% labeling fraction.

### Example 13: Sequencing by Synthesis with Labeled Nucleotides Comprising Non-Proteinogenic Amino Acids

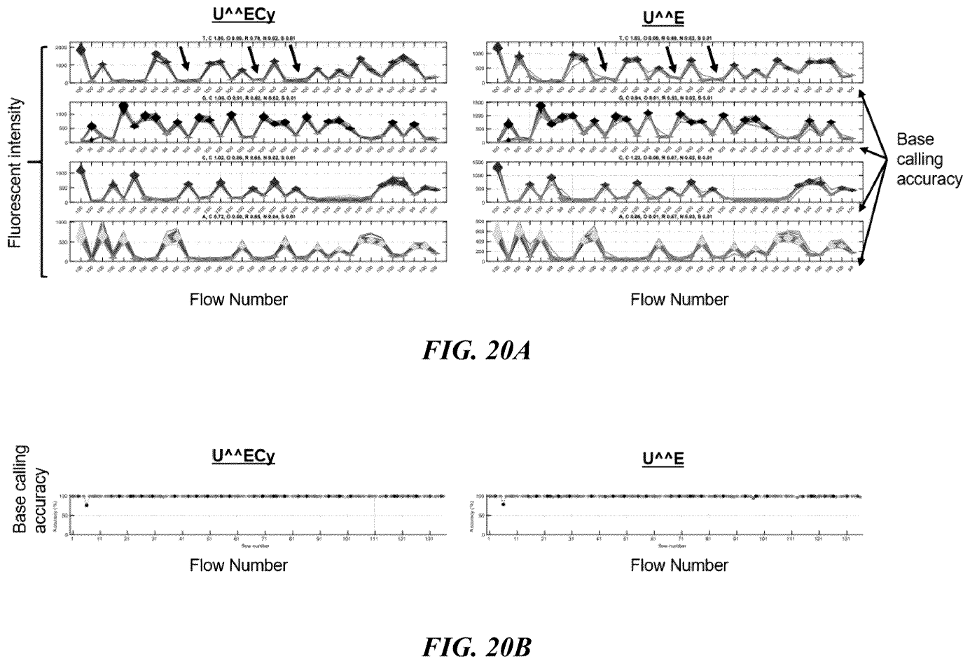

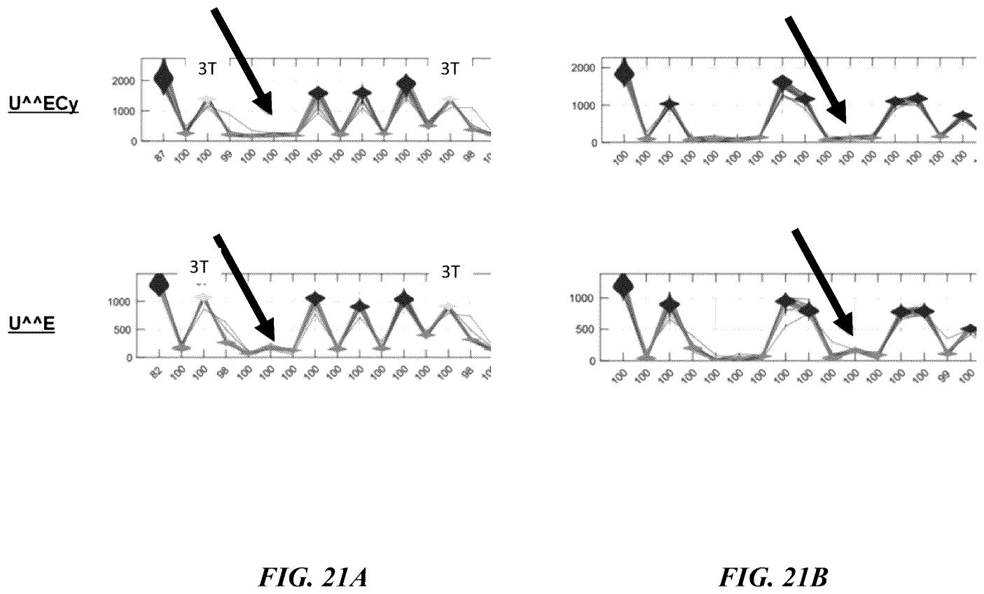

A template nucleic acid having a length of at least about 100 nucleotides was sequenced using the procedures and detectably labeled nucleotides described herein. FIGS. 20A-20B summarize sequencing experiments using a labeled nucleotide comprising cysteic acid. In FIG. 20A, the fluorescent intensity (Y-axis) detected for each of the four labeled nucleotides (from top: uracil (U; corresponding to T), guanine (G), cytosine (C), and adenosine (A)) in each flow cycle is plotted against the flow cycles (X-axis). The accuracies of the base-calling for each flow cycle are listed. U was fluorescently labeled as U\hat{ }\hat{ }ECy (the left panel). In the control experiment (the right panel), U was fluorescently labeled as U\hat{ }\hat{ }EAm. A was fluorescently labeled as A\hat{ }\hat{ }ECy; C was fluorescently labeled as C\hat{ }\hat{ }E, and G was fluorescently labeled as G\hat{ }\hat{ }EHCyCy or G\hat{ }\hat{ }EHCy. In flow cycles without incorporation of a labeled nucleotide, the expected signal is about 0. In some cases, a background signal (e.g., “floor signal”) can be detected even if a complementary labeled nucleotide is not being incorporated (or if a non-complementary nucleotide is incorporated; see arrows in FIG. 20A). As shown in FIG. 20A, when using U\hat{ }\hat{ }ECy, the floor signal is closer to the expected signal (i.e., the fluorescent intensity detected is 0) relative to that of the control. Similar results were obtained using U\hat{ }\hat{ }EAm in sequencing reactions of two other template nucleic acids, as shown in FIGS. 21A-21B in which the arrows indicate the floor signals.

As shown in FIG. 201B, the use of a labeled nucleotide comprising cysteic acid (e.g., U\hat{ }\hat{ }ECy) does not affect the base-calling accuracy of the sequencing reaction. The base-calling of other nucleotides (G, C, and A) not comprising the cysteic acid linker are not affected (not shown).

The detectably labeled nucleotides or labeling reagent described herein can also be used to accurately determine homopolymer sequences. When analyzing homopolymer sequences, the accuracy of base-callings using U\hat{ }\hat{ }ECy is comparable to that of the control, as shown in FIGS. 21A-21B (see the label “3T”). Tables 1 and 2 summarize base-calling error rates and other parameters when analyzing various lengths of homopolymer sequences using U\hat{ }\hat{ }ECy.

TABLE 1
Base-calling error rates (%) for different lengths of homopolymer
sequences performed with U\hat{ }\hat{ }ECy

| Total length of | Total base- | Base-calling | Total base- | Base-calling |
|---|---|---|---|---|
| homopolymer | calling error | error for U | calling error | error with |
| sequences | with U\hat{ }\hat{ }ECy | with U\hat{ }\hat{ }ECy | with U\hat{ }\hat{ }EAm | U\hat{ }\hat{ }EAm |
| Total (length of | 0.73 | 0.83 | 0.72 | 0.79 |
| homopolymer | | | | |
| sequence: 0-12) | | | | |
| 0 | 0.29 | 0.31 | 0.29 | 0.34 |
| 1 | 0.6 | 0.43 | 0.59 | 0.48 |
| 2 | 1.6 | 1.3 | 1.71 | 1.2 |
| 3 | 2.42 | 2.88 | 2.36 | 2.5 |
| 4 | 4.36 | 5.41 | 4.14 | 5.06 |
| 5 | 6.92 | 8.42 | 5.89 | 6.76 |
| 6 | 13.3 | 19.0 | 8.87 | 9.6 |
| 7 | 15.2 | 21.4 | 10.2 | 11.6 |
| 8 | 30.4 | 39.8 | 20.2 | 21.7 |

TABLE 2
A summary of parameters of the sequencing reactions using U\hat{ }\hat{ }ECy

| | U\hat{ }\hat{ }ECy | U\hat{ }\hat{ }EAm |
|---|---|---|
| Lag phasing | 0.34 | 0.33 |
| Lead phasing | 0.24 | 0.25 |
| Fluorescent intensity of | 1682.1 | 1049.4 |
| homopolymer length = 1 | | |
| (Arbitrary unit) | | |
| T-context | 0.978 | 0.926 |

These data show that the base-calling error rates and various sequencing reaction parameters using U\hat{ }\hat{ }ECy are comparable to those of the control, suggesting that the homopolymer length can be accurately identified using the fluorescently labeled nucleotides described herein. Additionally, Table 2 shows that U\hat{ }\hat{ }ECy can produce higher detectable fluorescent intensity in sequencing reactions when compared to that of the control (see Fluorescent intensity of homopolymer length=1 of Table 2).

### Example 14: Example Synthesis of Kam Fluorophore

FIG. 22 illustrates an example process for synthesizing a Kam fluorophore (PN 40289). The Kam fluorophore may be used in conjunction with any nucleotide and/or linker disclosed herein, for example, with any nucleotide, cleavable group, amino acid linker, and/or combination thereof depicted in FIG. 4.

Aminophenol hemisulfate (17.04 gram, 45.8 mmol, TCI E0534), phthalic anhydride (6.74 gram, 45.4 mmol), and sulfolane (34 mL) is combined in a flask equipped with a Teflon stir-bar and placed under an inert nitrogen atmosphere. The temperature of the flask is raised to 170° C. After 8 hours at 170° C. under nitrogen, the dark liquid is transferred in equal amounts to nine 50 mL centrifuge tubes, with the aid of 12 mL of methanol. To each tube is added 1 mL of triethylamine, followed by 1 mL MeOH. The tubes are then diluted to 45 mL with water, and the precipitated product is collected by centrifugation. The precipitates in each tube are washed by suspending in solutions of 1 mL of triethylamine in 30 mL water, collecting the precipitates by centrifugation, and drying the combined precipitates to constant weight at high vacuum. The consequent dark red solid is transferred to three 50 mL centrifuge tubes with 15 mL of methanol. The suspensions are each diluted to 45 mL with 2% (vol/vol) of triethylamine in water, and the resulting precipitates are collected by centrifugation and dried at high vacuum for 2 hr.

The wet solid from above is diluted with 50 mL of acetic acid; this suspension is brought completely into solution by heating, and the acetic acid is distilled off, and the residue is dried under vacuum. The residue is resuspended and dissolved in 50 mL of acetic acid, followed by removal of the acetic acid as above. The dried material is transferred in equal amounts to four 50 mL centrifuge tubes, using 10 mL of methanol to aid the transfer. The suspensions are each diluted to 45 mL with water, sonicated for 20 minutes, and centrifuged; after drying to constant weight, 7.74 gram (35% yield) of dark powder is recovered.

The total product obtained above is placed in a flask equipped with a Teflon stir bar and a Teflon stopper, then diluted with 60 gram of 20% fuming sulfuric acid. After sealing the flask, the suspension is slowly dissolved over 30 minutes with vigorous stirring yielding a dark solution. After 5 hours at room temperature, the homogeneous solution is diluted with 180 mL of dioxane and allowed to stand for 2 hours. The crude product is precipitated from the dioxane supernatant with the addition of ethyl ether (5 mL of dioxane diluted to 45 mL ethyl ether) in centrifuge tubes. An initial purification is performed by suspending the ether precipitate with 40 mL of ethanol and collecting ethanol insoluble material by centrifuge. Purified product is obtained by column chromatography on reverse phase C18 silica gel (40 gram of silica gel/gram of crude product) eluting with 4:1 of 0.1M triethylammonium carbonate:acetonitrile. The purified product is obtained as a fast-running red band. Evaporation and drying of the fractions containing the red band yielded 4.5 grams of red glassy solid PN 40289.

Examples 15-26 below describe example synthesis procedures of various detectably labeled nucleotides and their intermediate compounds, for example, whose components are illustrated in FIG. 4.

### Example 15: Example Synthesis of Compound 40517

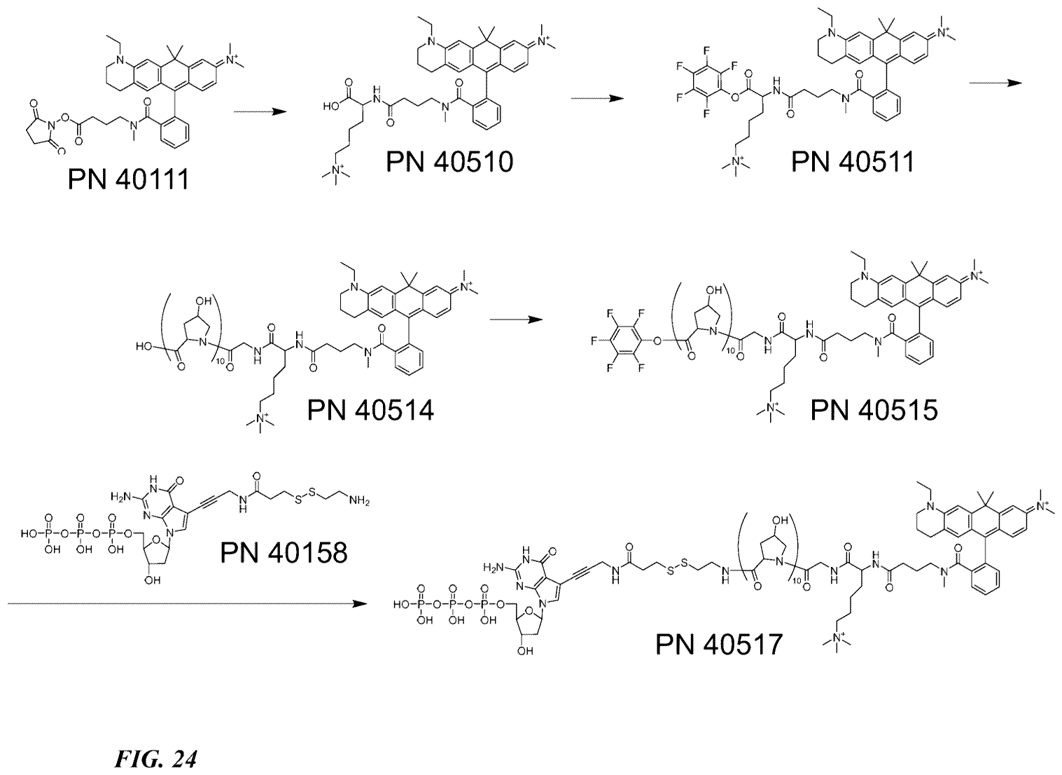

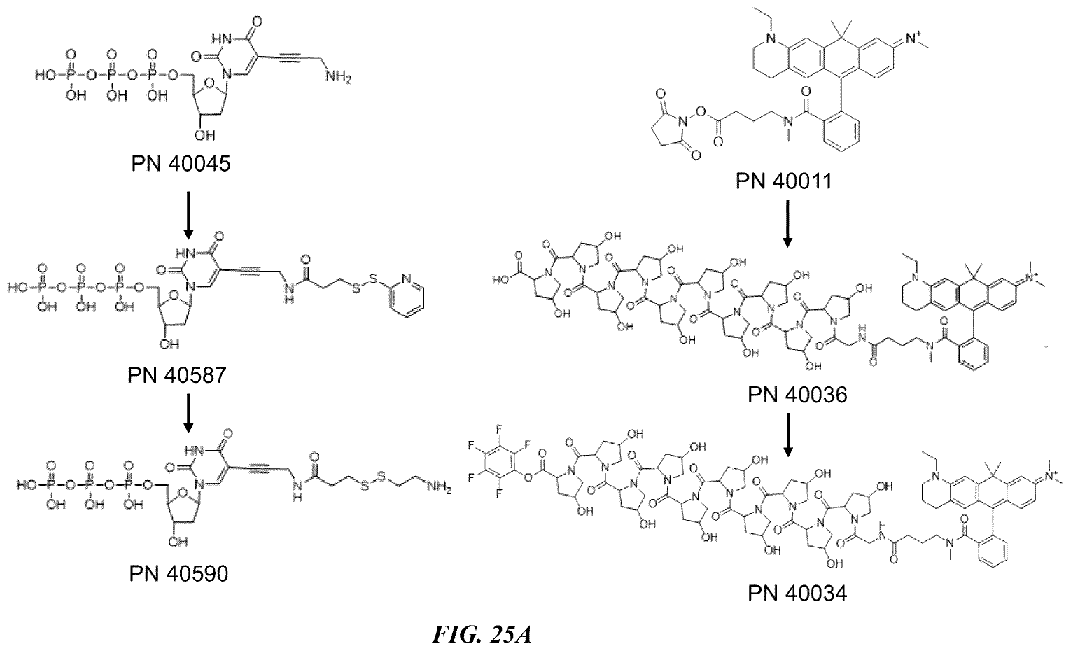

Compound PN 40517 shown in FIG. 24 is an ATTO 633-labeled dGTP, or the following combination compound in FIG. 4: G-EGlyHyp10L\hat{ }\hat{ }.

Preparation of PN 40510. As illustrated in FIG. 24, a solution of ATTO 633-NHS (150 mg, 231 μmol, PN 40011, Atto-Tec GmbH) in DMF (4 mL) is added to a solution of H-Lys(Me)₃-OH chloride (150 mg, 667 μmol, Bachem Americas), saturated aqueous sodium bicarbonate (3 mL) and water (6 mL). The combined mixture is stirred for 2 hours at room temperature. The reaction mixture is purified by PREP-LC (250×100 mm 5A Gemini column, gradient of 20% to 60% acetonitrile/0.1 M TEAB buffer, 34 mL/min, 80 minutes). This gives product PN 40510 (192 μmol) with a m/z (LCMS positive mode)=722.

Preparation of PN 40511. PN 40510 (192 μmol) is combined with dry acetonitrile (20 mL), dry pyridine (0.5 mL, 6.2 mmol) and pentafluorophenyl trifluoroacetate (1 mL, 5.8 mmol, Sigma-Aldrich). The solution is stirred at room temperature for 1 hour. The reaction mixture is concentrated on a rotary evaporator, reconstituted with dry acetonitrile, and concentrated again to give PN 40511 (192 μmol) with a m/z (LCMS positive mode)=889.

Preparation of PN 40514. PN 40511 (192 μmol) is dissolved in DMF (6 mL). Gly-Hyp10 (300 mg, 249 μmol, Genscript) is dissolved in saturated aqueous sodium bicarbonate (3 mL) and water (6 mL). The two solutions are combined and stirred at room temperature for 2 hours. The reaction mixture is purified by PREP-LC (250×50 mm 5A Gemini column, gradient of 20% to 60% acetonitrile/0.1 M TEAB buffer, 7 mL/min, 80 minutes). This gives product PN 40514 (100 μmol) with a m/z (LCMS negative mode)=1908.

Preparation of PN 40515. PN 40514 (100 μmol) is combined with dry DMF (20 mL), dry pyridine (0.5 mL, 6.2 mmol) and pentafluorophenyl trifluoroacetate (1 mL, 5.8 mmol, Sigma-Aldrich). The solution is stirred at room temperature for 1 hour. The reaction mixture is concentrated on a rotary evaporator, reconstituted with dry acetonitrile and concentrated again to give PN 40515 (100 μmol).

Preparation of PN 40157. As illustrated in FIG. 3A, 7-Deaza-7-Propargylamino-2′-deoxyguanosine-5′-Triphosphate (33 μmol, PN 40049, MyChem LLC), saturated aqueous sodium bicarbonate (0.4 mL) and water (0.8 mL) are combined. Succinimidyl 3-(2-pyridyldithio)propionate (30 mg, 96 μmol, Thermo Scientific) is dissolved in DMF (0.4 mL). The two solutions are combined and mixed for 1 hour at room temperature. The reaction mixture is purified by PREP-LC (250×100 mm 5A Gemini column, gradient of 20% to 60% acetonitrile/0.1 M TEAB buffer, 34 mL/min, 80 minutes). This gives product PN 40157 (30 μmol) with a m/z (LCMS negative mode)=755.

Preparation of PN 40158. PN 40157 (30 μmol), cysteamine hydrochloride (20 mg, 176 μmol), saturated aqueous sodium bicarbonate (0.4 mL) and water (0.8 mL) are combined and stirred for 30 minutes. The reaction mixture is purified by PREP-LC (250×50 mm 5A Gemini column, gradient of 2% to 60% acetonitrile/0.1 M TEAB buffer, 7 mL/min, 80 minutes). This gives PN 40158 (13 μmol) with a m/z (LCMS negative mode)=721.

Preparation of PN 40517. As illustrated in FIG. 24, a first solution is prepared by dissolving PN 40515 (35 μmol) in DMF (5 mL). A second solution is prepared by dissolving PN 40158 (46 μmol) in saturated aqueous sodium bicarbonate (2 mL) and water (5 mL). The first and second solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives product PN 40517 (4.9 μmol) with a m/z (LCMS negative mode, double charge)=1306.

### Example 16: Example Synthesis of Compound 40111

Preparation of PN 40079. As illustrated in FIG. 3B, a solution of ATTO 633-NHS (280 mg, 431 μmol, PN 40011, Atto-Tec GmbH) in DMF (5.2 mL) is added to a solution of L-cysteic acid monohydrate (280 mg, 1497 μmol, Sigma Aldrich), saturated aqueous sodium bicarbonate (5.2 mL) and water (8 mL). The combined mixture is stirred for 2 hours at room temperature. The reaction mixture is purified by PREP-LC (250×100 mm 5A Gemini column, gradient of 20% to 60% acetonitrile/0.1 M TEAB buffer, 34 mL/min, 80 minutes). This gives PN 40079 (204 μmol) with a m/z (LCMS positive mode)=702.

Preparation of PN 40080. PN 40079 (204 μmol) is combined with dry acetonitrile (8.5 mL), dry pyridine (0.2 mL, 6. mmol) and pentafluorophenyl trifluoroacetate (0.8 mL, 2.5 mmol, Sigma-Aldrich). The solution is stirred at room temperature for 1 hour. The reaction mixture is concentrated on a rotary evaporator, reconstituted with dry acetonitrile, and concentrated again to give PN 40080 (204 μmol) with a m/z (LCMS positive mode)=869.

Preparation of PN 40102. A solution of PN 40080 (225 μmol) and acetonitrile (10 mL) is added to a solution of L-cysteic acid monohydrate (280 mg, 1497 μmol, Sigma Aldrich), DMF (5 mL) and N,N-diisopropylethylamine (750 uL). The combined mixture is stirred for 2 hours at 50 C. The reaction mixture is purified by PREP-LC (250×100 mm 5A Gemini column, gradient of 20% to 60% acetonitrile/0.1 M TEAB buffer, 34 mL/min, 80 minutes). This gives PN 40102 (69 μmol) with a m/z (LCMS negative mode)=852.

Preparation of PN 40106 PN 400102 (69 μmol) is combined with dry acetonitrile (7 mL), dry pyridine (0.1 mL, 1.2 mmol) and pentafluorophenyl trifluoroacetate (0.4 mL, 2.3 mmol, Sigma-Aldrich). The solution is stirred at room temperature for 1 hour. The reaction mixture is concentrated on a rotary evaporator, reconstituted with dry acetonitrile, and concentrated again to give PN 40106 (69 μmol) with a m/z (LCMS positive mode)=869.

Preparation of PN 40107. PN 40106 (69 μmol) is dissolved in DMF (6 mL). Gly-Hyp10 (140 mg, 116 μmol, Genscript) is dissolved in saturated aqueous sodium bicarbonate (1 mL) and water (2 mL). The two solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives product PN 40107 (54 μmol) with a m/z (LCMS negative mode, double charge)=1020.

Preparation of PN 40108. PN 40107 (54 μmol) is combined with dry DMF (6.1 mL), dry pyridine (0.1 mL, 1.2 mmol) and pentafluorophenyl trifluoroacetate (0.4 mL, 2.3 mmol, Sigma-Aldrich). The solution is stirred at room temperature for 1 hour. The reaction mixture is concentrated on a rotary evaporator, reconstituted with dry acetonitrile, and concentrated again to give PN 40108 (54 μmol) with a m/z (LCMS positive mode, double charge)=1104.

Preparation of PN 40111. As illustrated in FIG. 3C, PN 40515 (15 μmol) is dissolved in DMF (1.6 mL). PN 40158 (15 μmol) is dissolved in saturated aqueous sodium bicarbonate (1 mL), and water (1.6 mL). The two solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives product PN 40517 (6.5 μmol) with a m/z (LCMS negative mode, double charge)=1372.

### Example 17: Example Synthesis of Compound 40589

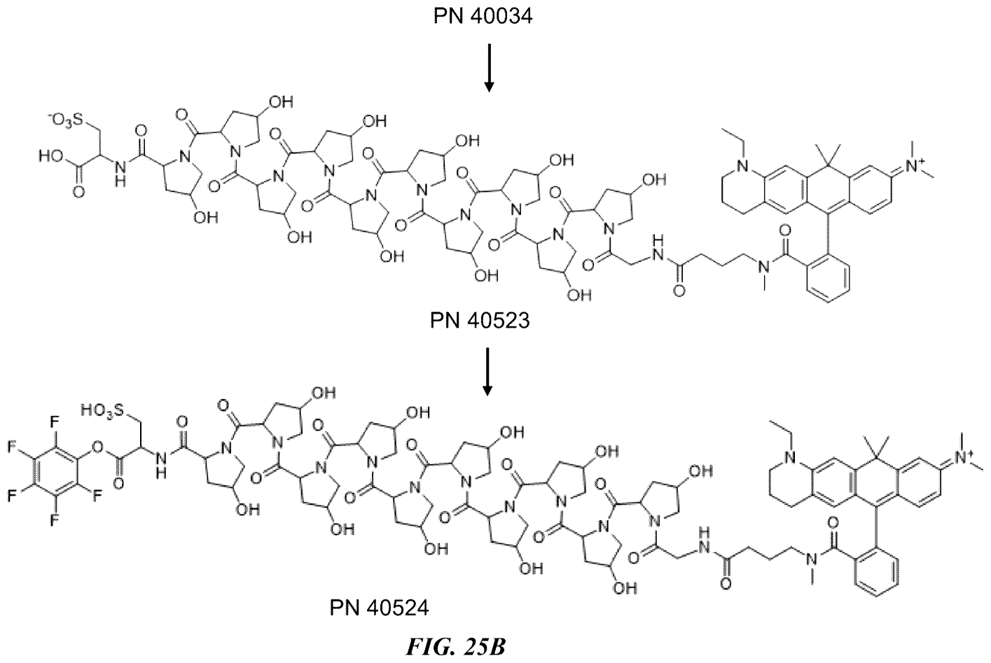

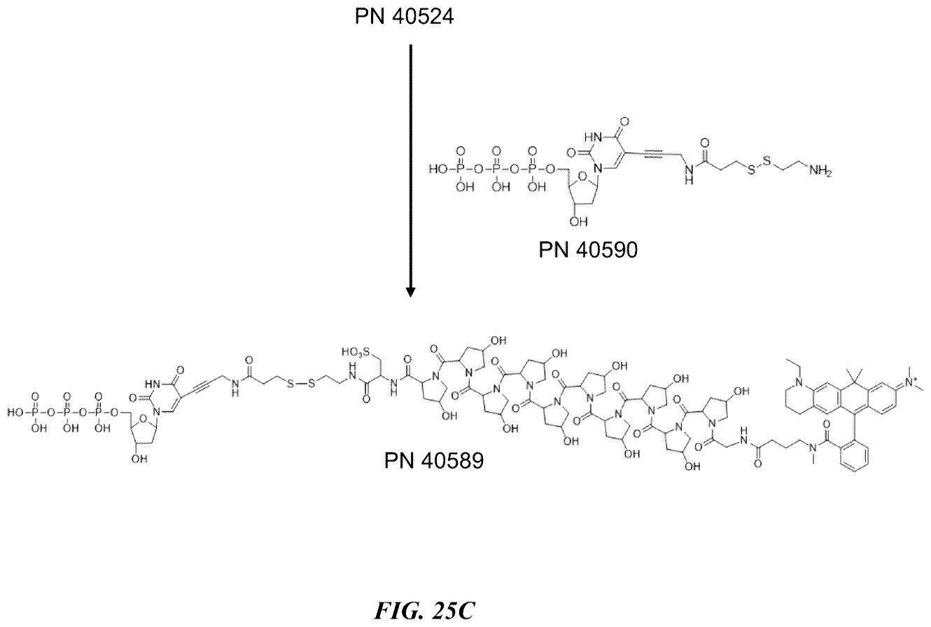

FIGS. 25A-25C show an example process for synthesizing a fluorescently labeled dUTP nucleotide with a cysteic acid at the C-terminus end of GlyHyp10. Compound PN 40589 shown in FIG. 25C is an ATTO 633-labeled dUTP, or the following combination compound in FIG. 4: U-ECyGlyHyp10\hat{ }\hat{ }.

Preparation of PN 40036 As illustrated in FIGS. 25A-25C, a solution of ATTO 633-NHS (100.0 mg, 154.1 μmol, PN 40011, Atto-Tec GmbH) in DMF (3.0 mL) is added to a solution of Gly-Hyp10 (400.0 mg, 331.6 μmol, GenScript), saturated aqueous sodium bicarbonate (2.0 mL) and water (6.0 mL). The combined mixture is stirred for 2 hours at room temperature. The reaction mixture is purified by PREP-LC (250×100 mm 5A Gemini column, gradient of 20% to 60% acetonitrile/0.1 M TEAB buffer, 34 mL/min, 80 minutes). This gives PN 40036 with a m/z (LCMS positive mode)=1739.8.

Preparation of PN 40034. PN 40036 (150.0 μmol) is combined with dry DMF (8.0 mL), dry pyridine (0.5 mL, 6.1 mmol) and pentafluorophenyl trifluoroacetate (1.0 mL, 11.6 mmol, Sigma-Aldrich). The solution is stirred at room temperature for 1 hour. The reaction mixture is concentrated on a rotary evaporator, reconstituted with dry acetonitrile, and concentrated again to give PN 40034 (150.0 μmol).

Preparation of PN 40523. PN 40034 (91.0 μmol) is dissolved in the mixture of DMF (3.0 mL) and acetonitrile (5.0 mL). L-Cysteic acid (150 mg, 802 μmol, Sigma Aldrich) is dissolved in DMF (3.0 mL) and N,N-Diisopropylethylamine (0.4 mL) with sonication and vortexing. The two solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives PN 40523 (45.0 μmol) with a m/z (LCMS negative mode)=1890.

Preparation of PN 40524. PN 40523 (45.0 μmol) is combined with dry DMF (6.0 mL), dry pyridine (0.2 mL, 2.4 mmol) and pentafluorophenyl trifluoroacetate (0.3 mL, 1.7 mmol, Sigma-Aldrich). The solution is stirred at room temperature for 1 hour. The reaction mixture is concentrated on a rotary evaporator, reconstituted with dry acetonitrile and concentrated again to give PN 40524 (45 μmol).

Preparation of PN 40587. A first solution is prepared by combining 5-Propargylamino-2′-deoxyuridine-5′-Triphosphate (215.0 μmol, PN 40045, MyChem LLC), saturated aqueous sodium bicarbonate (3.0 mL) and water (3.0 mL). A second solution is prepared by dissolving succinimidyl 3-(2-pyridyldithio)propionate (300.0 mg, 960.0 μmol, Thermo Scientific) in DMF (5.0 mL). The first and second solutions are combined and mixed for 1 hour at room temperature. The reaction mixture is purified by PREP-LC (250×100 mm 5A Gemini column, gradient of 2% to 60% acetonitrile/0.1 M TEAB buffer, 34 mL/min, 80 minutes). This gives PN 40587 (132.0 μmol) with a m/z (LCMS negative mode)=716.9.

Preparation of PN 40590. PN 40587 (132 μmol), cysteamine hydrochloride (91.0 mg, 800.0 μmol), saturated aqueous sodium bicarbonate (2.0 mL) and water (10.0 mL) are combined and stirred for 30 minutes. The reaction mixture is purified by PREP-LC (250×100 mm 5 A Gemini column, gradient of 2% to 60% acetonitrile/0.1 M TEAB buffer, 34 mL/min, 80 minutes). This gives PN 40590 (13 μmol) with a m/z (LCMS negative mode)=683.

Preparation of PN 40589. PN 40524 (43.0 μmol) is dissolved in DMF (4.0 mL). PN 40590 (55.9 μmol) is dissolved in saturated aqueous sodium bicarbonate (1.2 mL), and water (7.5 mL). The two solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives PN 40589 (5.2 μmol) with a m/z (LCMS negative mode, double charge)=1277.

### Example 18: Example Synthesis of Compound 40096

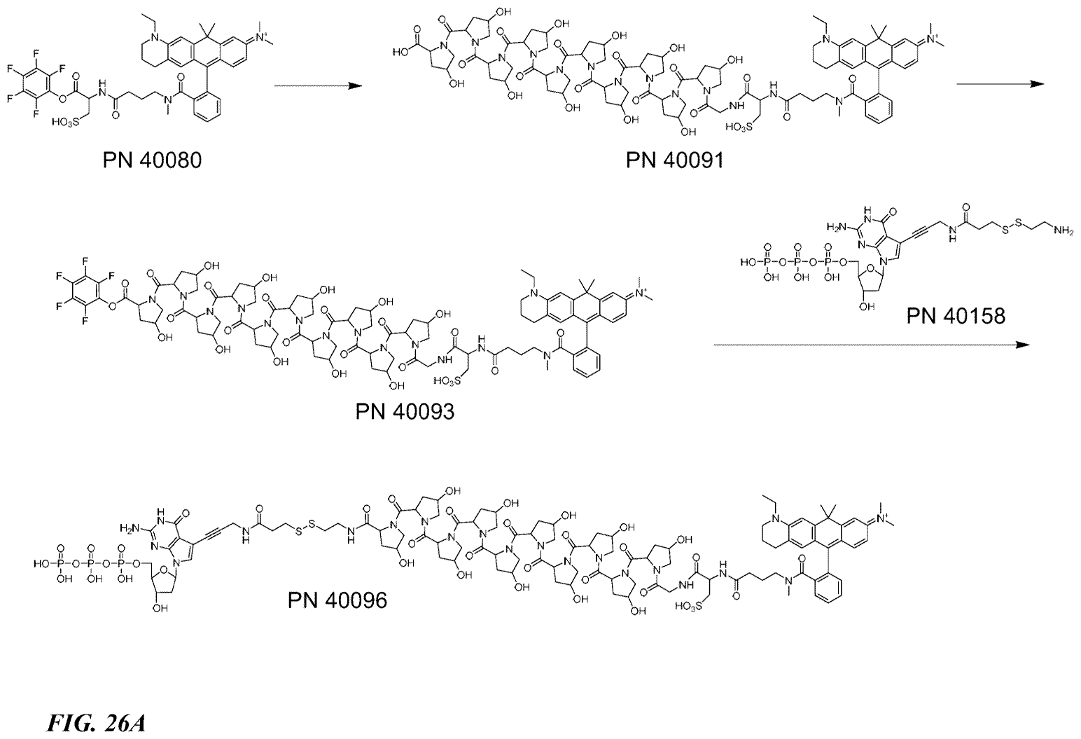

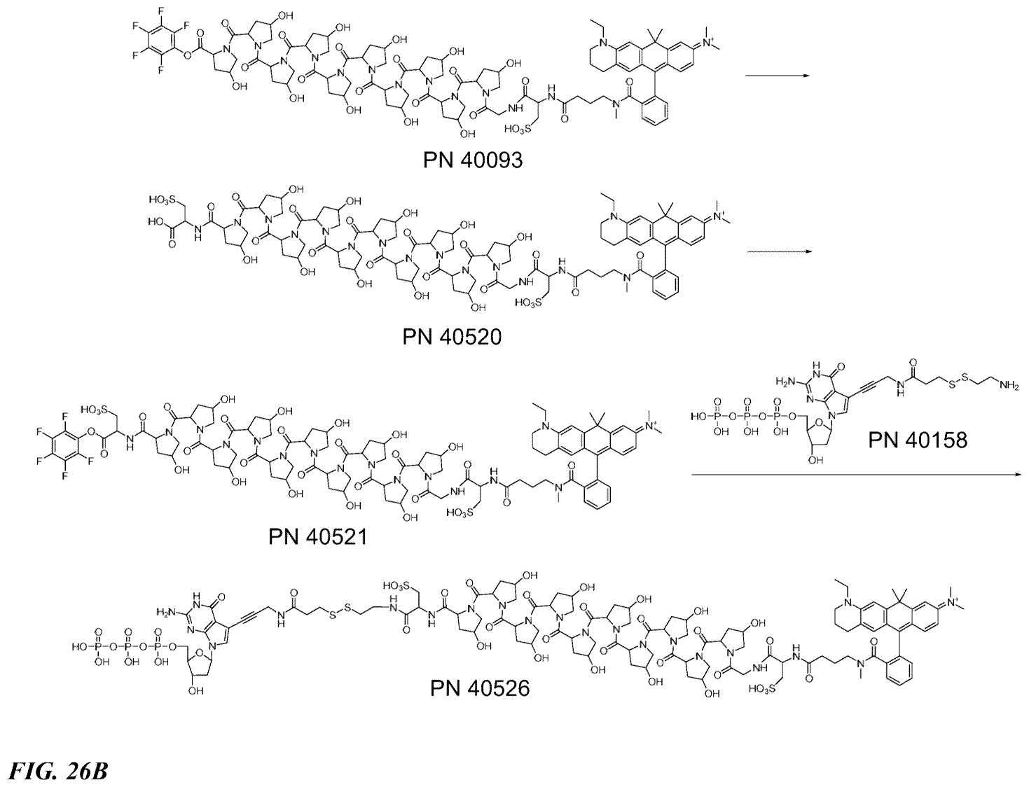

FIG. 26A shows an example process for synthesizing a fluorescently labeled dGTP nucleotide with a cysteic acid at the N-terminus end of GlyHyp10. Compound PN 40096 shown in FIG. 26A is an ATTO 633-labeled dGTP, or the following combination compound in FIG. 4: G-EGlyHyp10Cy\hat{ }\hat{ }.

Preparation of PN 40091. As illustrated in FIG. 26A, PN 40080 (306.5 μmol) is dissolved in the DMF (17.0 mL). Gly-Hyp10 (800.0 mg, 663.2 μmol, GenScript) saturated aqueous sodium bicarbonate (5.2 mL) and water (8.0 mL). The two solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives PN 40091 (145.0 μmol) with a m/z (LCMS positive mode)=1891.7.

Preparation of PN 40093. PN 40091 (113.2 μmol) is combined with dry DMF (18.0 mL), dry pyridine (0.3 mL, 3.6 mmol) and pentafluorophenyl trifluoroacetate (1.1 mL, 6.4 mmol, Sigma-Aldrich). The solution is stirred at room temperature for 1 hour. The reaction mixture is concentrated on a rotary evaporator, reconstituted with dry acetonitrile and concentrated again to give PN 40093 (113 μmol).

Preparation of PN 40096 PN 40093 (170.0 μmol) is dissolved in DMF (13.0 mL). PN 40158 (215.0 μmol) is dissolved in saturated aqueous sodium bicarbonate (7.2 mL), and water (10.0 mL). The two solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives PN 40096 (78.3 μmol) with a m/z (LCMS negative mode, double charge)=1296.6.

### Example 19: Example Synthesis of Compound 40526

FIG. 26B shows an example process for synthesizing a fluorescently labeled dGTP nucleotide with a cysteic acid at both the C- and N-termini ends of GlyHyp10. Compound PN 40526 shown in FIG. 26B is an ATTO 633-labeled dGTP, or the following combination compound in FIG. 4: G-ECyGlyHyp10Cy\hat{ }\hat{ }.

Preparation of PN 40520. As illustrated in FIG. 26B, a solution of PN 40093 (70.0 μmol) in DMF (3.0 mL) is added to a solution of L-cysteic acid monohydrate (100.0 mg, 0.53 mmol, Sigma Aldrich), saturated aqueous sodium bicarbonate (3.0 mL) and water (6.0 mL). The combined mixture is stirred for 2 hours at room temperature. The reaction mixture is purified by PREP-LC (250×100 mm 5A Gemini column, gradient of 20% to 60% acetonitrile/0.1 M TEAB buffer, 34 mL/min, 80 minutes). This gives PN 40520 (9.0 μmol) with a m/z (LCMS negative mode, double charge)=1020.4.

Preparation of PN 40521. PN 40520 (9.0 μmol) is combined with dry acetonitrile (5.0 mL), dry pyridine (4.9 mmol) and pentafluorophenyl trifluoroacetate (9.3 mmol, Sigma-Aldrich). The solution is stirred at room temperature for 1 hour. The reaction mixture is concentrated on a rotary evaporator, reconstituted with dry acetonitrile, and concentrated again to give PN 40521 (9.0 μmol).

Preparation of PN 40526 PN 40521 (9.0 μmol) is dissolved in DMF (3.0 mL). PN 40158 (31.0 μmol) is dissolved in saturated aqueous sodium bicarbonate (2.0 mL), and water (3.0 mL). The two solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives PN 40526 (0.36 μmol) with a m/z (LCMS negative mode, double charge)=1372.3

### Example 20: Example Synthesis of Compound 40559

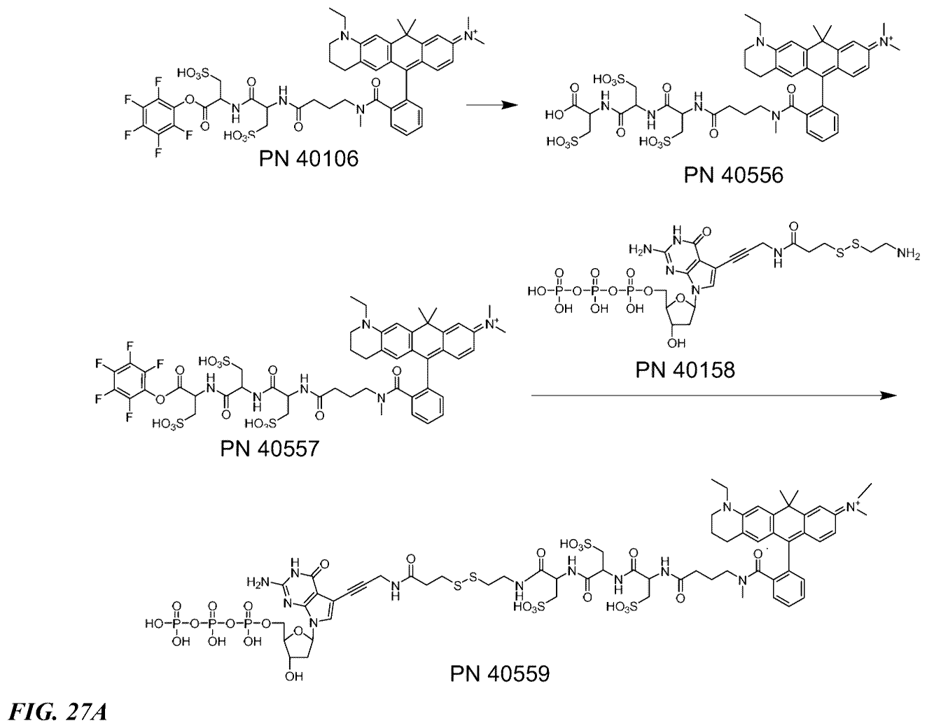

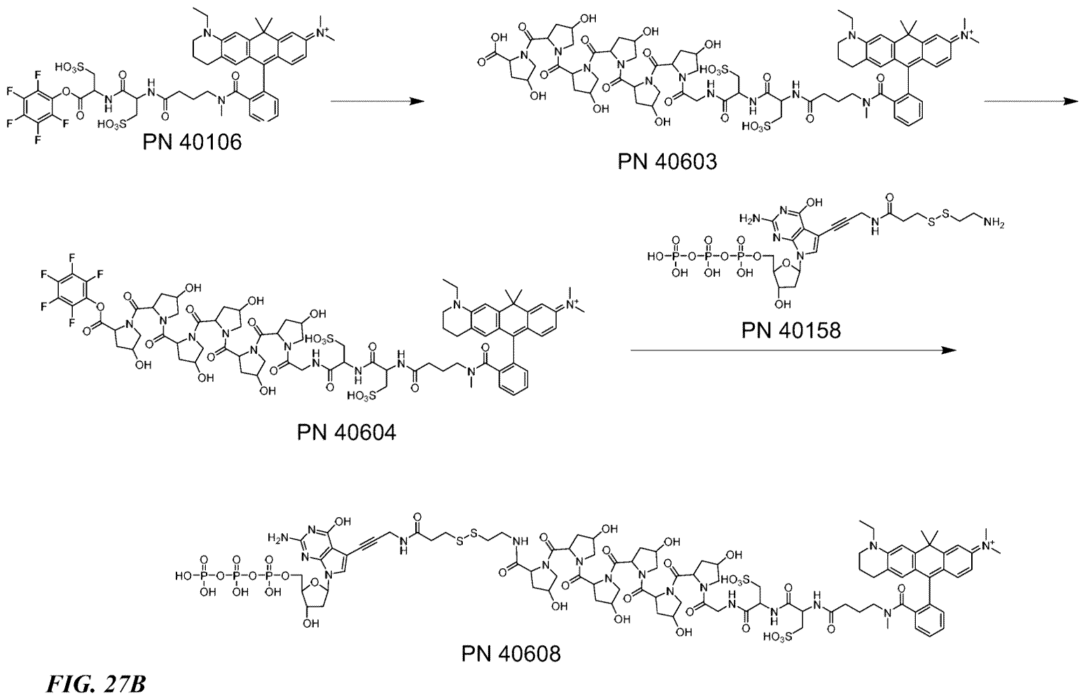

FIG. 27A shows an example process for synthesizing a fluorescently labeled dGTP nucleotide with 3 cysteic acids. Compound PN 40559 shown in FIG. 27A is an ATTO 633-labeled dGTP, or the following combination compound in FIG. 4: G-ECyCyCy\hat{ }\hat{ }.

Preparation of PN 40556 As illustrated in FIG. 27A, PN 40106 (87.3 μmol) is dissolved in dry acetonitrile (10.0 mL) is added to a solution of L-cysteic acid monohydrate (98.0 mg, 0.5 mmol, Sigma Aldrich), in dry DMF (5.0 mL) and DIEA (0.3 mL). The combined mixture is stirred for 2 hours at room temperature. The reaction mixture is purified by PREP-LC (250×100 mm 5A Gemini column, gradient of 20% to 60% acetonitrile/0.1 M TEAB buffer, 34 mL/min, 80 minutes). This gives PN 40556 (43.9 μmol) with a m/z (LCMS positive mode)=704.4.

Preparation of PN 40557. PN 40556 (43.9 μmol) is combined with dry acetonitrile (8.0 mL), dry pyridine (65.0 uL, 0.8 mmol) and pentafluorophenyl trifluoroacetate (0.26 mL, 1.5 mmol, Sigma-Aldrich). The solution is stirred at room temperature for 1 hour. The reaction mixture is concentrated on a rotary evaporator, reconstituted with dry acetonitrile, and concentrated again to give PN 40557 (43.0 μmol) with a m/z (LCMS negative mode)=1169.2.

Preparation of PN 40559. PN 40556 (15.0 μmol) is dissolved in DMF (1.0 mL). PN 40158 (19.5 μmol) is dissolved in saturated aqueous sodium bicarbonate (0.3 mL), and water (1.0 mL). The two solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives PN 40559 (3.8 μmol) with a m/z (LCMS negative mode)=1708.4.

### Example 21: Example Synthesis of Compound 40608

FIG. 27B shows an example process for synthesizing a fluorescently labeled dGTP nucleotide with 2 cysteic acids at N-termini ends of Gly-Hyp6. Compound PN 40608 shown in FIG. 27B is an ATTO 633-labeled dGTP, or the following combination compound in FIG. 4: G-EGlyHyp6CyCy\hat{ }\hat{ }.

Preparation of PN 40603. As illustrated in FIG. 27B, PN 40106 (57.0 μmol) is dissolved in the DMF (2.5 mL). Gly-Hyp6 (55.9 mg, 74.1 μmol, GenScript), saturated aqueous sodium bicarbonate (1.5 mL) and water (2.5 mL). The two solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives PN 40603 (35.0 μmol) with a m/z (LCMS negative mode)=1588.6.

Preparation of PN 40604. PN 40603 (35.0 μmol) is combined with dry DMF (7.2 mL), dry pyridine (0.1 mL, 1.2 mmol) and pentafluorophenyl trifluoroacetate (0.2 mL, 1.1 mmol, Sigma-Aldrich). The solution is stirred at room temperature for 1 hour. The reaction mixture is concentrated on a rotary evaporator, reconstituted with dry acetonitrile and concentrated again to give PN 40604 (33 μmol).

Preparation of PN 40608. PN 40604 (33.0 μmol) is dissolved in DMF (4.7 mL). PN 40158 (48.0.0 μmol) is dissolved in saturated aqueous sodium bicarbonate (1.0 mL), and water (4.7 mL). The two solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives PN 40608 (4.8 μmol) with a m/z (LCMS negative mode, double charge)=1145.9.

### Example 22: Example Synthesis of Compound 40679

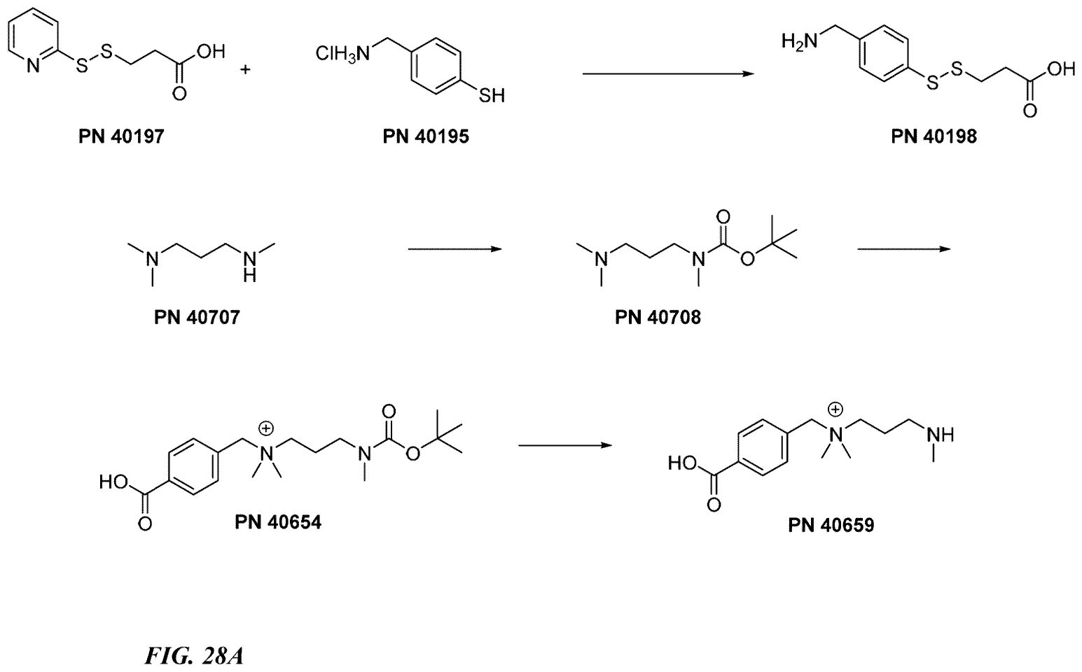

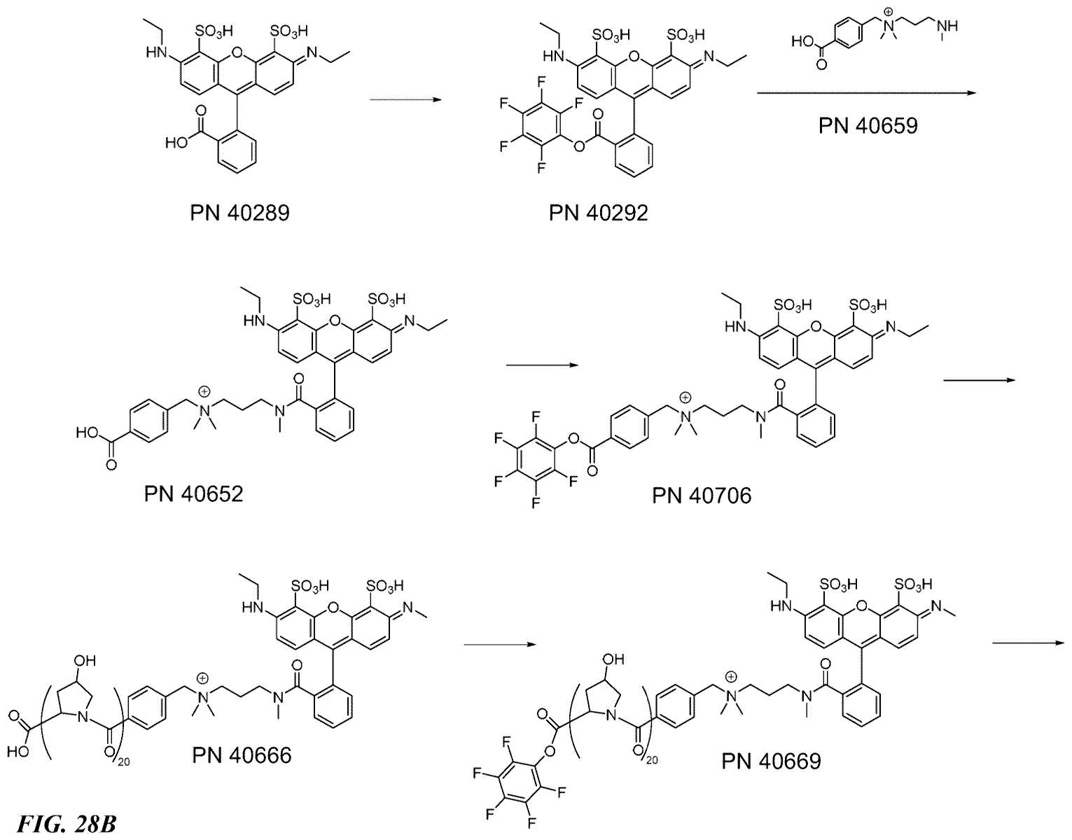

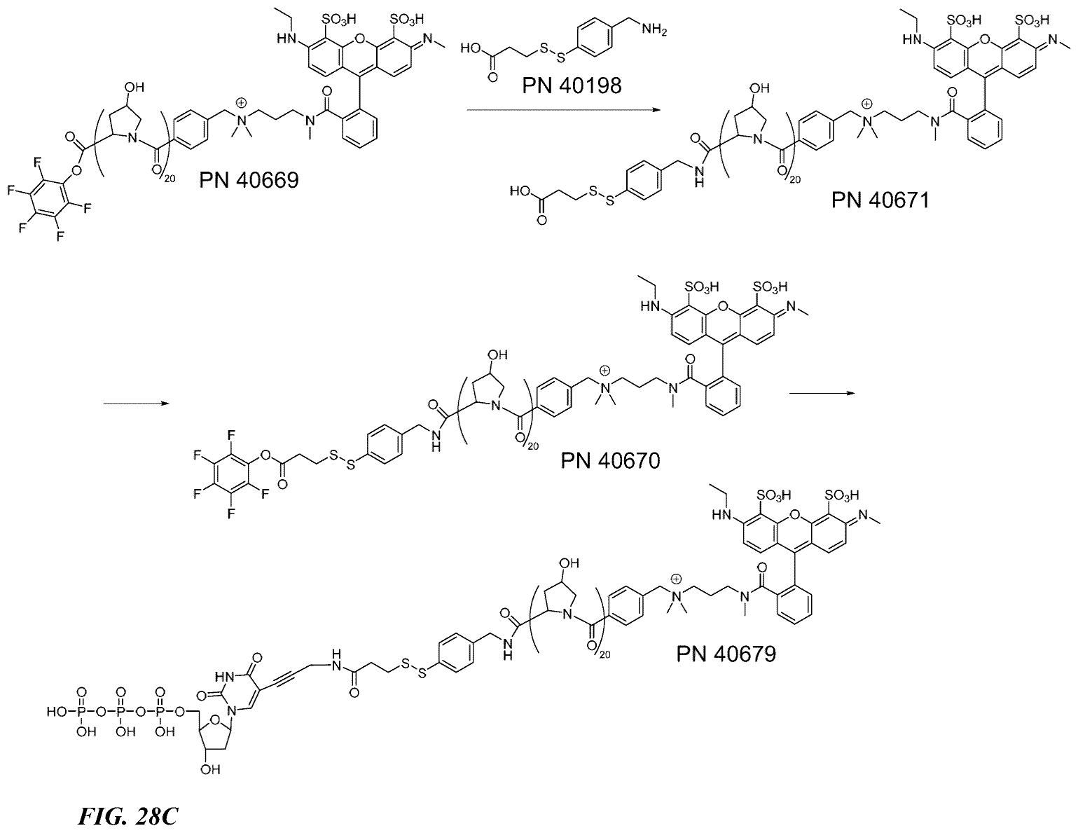

FIG. 28A-28C show an example process for synthesizing a fluorescently labeled dUTP nucleotide with a dimethyl ammonium. Compound PN 40679 shown in FIG. 28C is a Kam-labeled dUTP, or the following combination compound in FIG. 4: U-YHyp20VKam.

Preparation of PN 40198. As illustrated in FIG. 28A, a first solution is prepared by dissolving 3-(2-Pyridinyldithio)propanoic acid (1.0 g, 4.6 mmol, Combi-blocks, PN 40197) in methanol (10 mL) and acetic acid (1.0 mL). A second solution is prepared by dissolving 4-(Aminomethyl)benzenethiol hydrochloride (1.0 g, 4.6 mmol, Enamine, PN 40195) in methanol with sonication. The two solutions are combined and stirred at room temperature for 12 hours. The mixed is concentrated under reduced pressure until a yellow solid formed. The crude material is treated with MTBE (45 mL), vortexed, centrifuged, and the supernatant was discarded. Repeating the washing with MTBE 4 more times, and purification by PREP-LC (250×100 mm 5A Gemini column, gradient of 20% to 60% acetonitrile/0.1 M TEAB buffer, 34 mL/min, 80 minutes), gave 500 mg of PN 40198 with a m/z (LCMS negative mode)=242.

Preparation of PN 40708. To a solution of dichloromethane (15 mL) and N,N,N′-trimethyl-1,3-propanediamine (PN 40707, Sigma-Aldrich) (2.00 g, 19.6 mmol) is added to a dichloromethane solution of di-tertbutyl dicarbonate (Sigma-Aldrich) (4.97 g 22.8 mmol) in 15 mL dichloromethane over 3 minutes. After the evolution of gas stopped, the solution is allowed to stand overnight at room temperature. The resulting clear solution is evaporated under reduced pressure and evacuated for 4 hr. at high vacuum (<0.1 mm), yielding ˜4 gram of t-Boc protected diamine PN 40708 as a mobile oil. This material is carried onto the next step.

Preparation of PN 40654 The t-Boc protected diamine PN 40708 (2.1 g, 9.72 mmol) and methyl 4-(chloromethyl) benzoate (Sigma-Aldrich) (2.03 g, 11.0 mmol) are combined, diluted with 10 mL of anhydrous DMSO, and heated overnight at 70 C. One mL portions of the resulting solution are precipitated with 40 mL methyl-t-butyl ether (MTBE), the insoluble material being collected and combined by centrifuge. After drying over night at high vacuum, the gluey mass is precipitated by the addition of 20 mL of ethyl acetate; the initial precipitate is collected by centrifuge and washed with an additional 20 mL of ethyl acetate. After drying for 12 hr. at high vacuum, 3.81 g of PN 40654 is obtained as a tan powder with a m/z (LCMS positive mode)=351.2.

Preparation of PN 40659. PN 40654 (3.0 gram) is dissolved in 4M HCl (25 mL), with constant sonication until evolution of gas subsided (˜20 minutes). After 5 hr. at room temperature, the solvent is removed by evaporation at reduced pressure followed by evacuation at high vacuum, yielding ˜2.5 gram of crude PN 40659 as a white solid. Purification of is accomplished by dissolving 2 g of crude PN 40659 in 30 mL of refluxing ethanol, followed by precipitating by cooling to room temperature. The white solid is collected by centrifuge and dried at high vacuum yielding 0.6 from of white powder. The ethanol supernatant from the initial precipitation is diluted to 200 mL with isopropyl alcohol and allowed to stand at room temperature overnight. The resulting white solid from isopropyl alcohol is collected by centrifuge and dried at high vacuum, yielding 0.9 gram of PN 40659 as a white powder with a m/z (LCMS positive mode)=251.

Preparation of PN 40652. As illustrated in FIG. 28B, to a suspension of PN 40289 (100 mg, 0.183 mmol) in anhydrous DMF (0.8 mL) are added 90 uL of anhydrous pyridine followed by 120 uL of pentafluorophenyl trifluoroacetate (PFP-TFA). After homogenization, the clear mixture stored in the dark for 1.5 hr. Isolation of PN 40292 is accomplished by initial precipitation of the reaction mixture with 40 mL of dibutyl ether, serial washing of the insoluble product with 40 mL of dibutyl ether and 3 times with 40 mL portions of MTBE, and drying for 1 hr. at high vacuum. To the dried active ester PN 40292 is added PN 40659 (60 mg, 0.239 mmol) followed by anhydrous DMSO (1.5 mL). After homogenization, anhydrous triethylamine (150 uL) is added to the reaction mixture, and the clear solution is placed in the dark for 5 hr. Isolation of PN 40652 is accomplished by initial precipitation of the reaction mixture with 40 mL of ethyl acetate, followed by serial washing of the insoluble product with 2 times with 40 mL portions of ethyl acetate, and drying for 1 hr at high vacuum. The product is purified by preparative HPLC on reverse phase support using acetonitrile-0.1M triethylammonium carbonate gradient to give PN 40652 with a m/z (LCMS positive mode)=779.

Preparation of PN 40666 To a suspension of PN 40652 (100 mg, 0.128 mmol) in anhydrous DMF (1.0 mL) is added 90 uL of anhydrous pyridine followed by 120 uL of PFP-TFA. After homogenization, the clear mixture is stored in the dark for 3.5 hr. Isolation of the active ester PN 40706 is accomplished by initial precipitation of the reaction mixture with 40 mL of dibutyl ether, followed by serial washing of the insoluble product with 40 mL of dibutyl ether, washing 3 times with 40 mL portions of MTBE, and drying for 1 hr at high vacuum. To the dried active ester PN 40706 is added Hyp20 (319 mg, 0.140 mmol) followed by anhydrous DMSO (3.0 mL). After mixing, anhydrous triethylamine (150 uL) is added to the reaction mixture; the turbid solution clarified after 10 minutes of additional agitation and is placed in the dark for 3.5 hr. Isolation of PN 40666 is accomplished by initial precipitation with 40 mL of ethyl acetate, followed by serial washing of the insoluble product 2 times with 40 mL portions of ethyl acetate, and drying for 1 hr at high vacuum (<0.1 mm). The product was purified by preparative HPLC on reverse phase support using acetonitrile-0.1M triethylammonium carbonate gradient, to give PN 40666 with a m/z (LCMS positive mode, double charge)=1513

Preparation of PN 40671. As illustrated in FIG. 28B and FIG. 28C, to a suspension of PN 40666 (55 mg, 0.0.018 mmol) in anhydrous DMF (0.70 mL) is added 80 uL of anhydrous pyridine followed by 100 uL of PFP-TFA. Extensive vortexing and sonication yielded a turbid solution, which are placed on a rotator in the dark for 1.5 hr. Isolation of the active ester PN 40669 is accomplished by initial precipitation of the reaction mixture with 5 mL of dibutyl ether, followed by serial washing of the insoluble product with 5 mL of dibutyl ether and 3 times, washing with 5 mL portions of MTBE, and drying for 1 hr. at high vacuum. To the dried active ester is added PN 40198 (12 mg, 0.049 mmol) followed by anhydrous DMSO (0.8 mL); mixing yielded a turbid solution. Anhydrous triethylamine (20 uL) is added to the reaction mixture and the vessel is placed on a rotator; the turbid solution clarified after 10 minutes and is placed in the dark for 2.5 hr. Isolation of PN 40671 is accomplished by initial precipitation with 4 mL of ethyl acetate, followed by serial washing of the insoluble product with 2 times with 4 mL portions of ethyl acetate, and drying for 1 hr. at high vacuum. The product is purified by preparative HPLC on reverse phase support using acetonitrile-0.1M triethylammonium carbonate gradient to give PN 40671 with a m/z (LCMS positive mode, double charge)=3265.

Preparation of PN 40679. To a suspension of PN 40671 (20 mg, 0.0061 mmol) in anhydrous DMF (0.50 mL) is added 30 uL of anhydrous pyridine followed by 50 uL of PFP-TFA. The clear solution is placed in the dark for 1.5 hr. Isolation of the active ester PN 40670 was accomplished by initial precipitation of the reaction mixture with 5 mL of dibutyl ether, followed by serial washing of the insoluble product with 5 mL of dibutyl ether, washing 3 times with 5 mL portions of MTBE, and drying for 1 hr. at high vacuum. The PN 40670 is dissolved in 1 mL of anhydrous DMSO. To this solution is added a solution comprising 0.5 mL of 30 mM 5′-propargylamino-dUTP (CAS 179101-49-6) and 2 mL of saturated sodium bicarbonate. After standing in the dark for 10 hr., the clear reaction mixture purified by preparative HPLC on reverse phase support using acetonitrile-0.1M triethylammonium carbonate gradient, to give PN 40679 with a m/z (LCMS positive mode, double charge)=3766.

### Example 23: Example Synthesis of Compound 40673

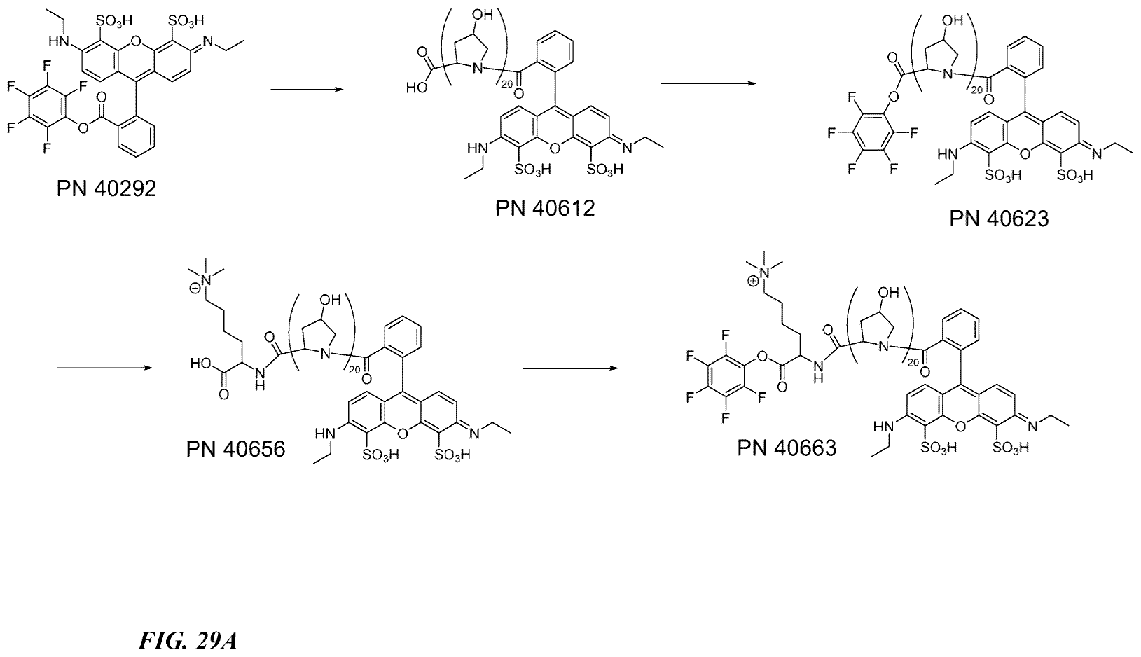

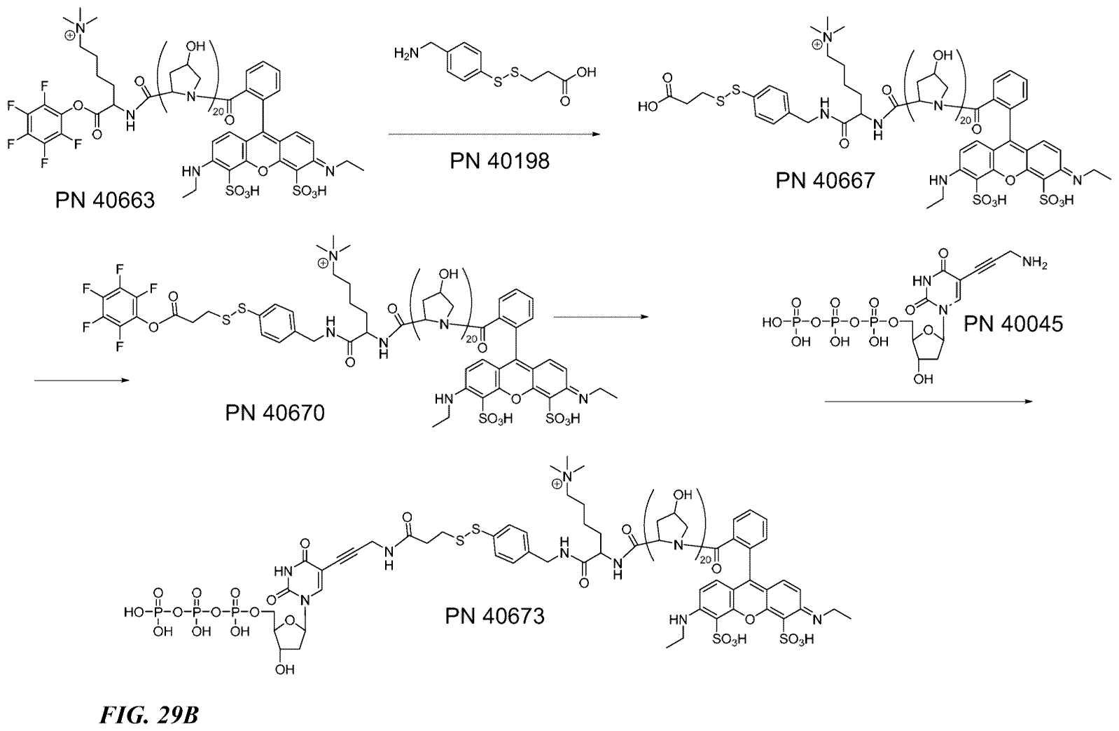

FIGS. 29A and 29B show an example process for synthesizing a fluorescently labeled dUTP nucleotide with a trimethyl ammonium lysine. Compound PN 40673 shown in FIG. 29B is a Kam-labeled dGTP, or the following combination compound in FIG. 4: U-YLHyp20Kam.

Preparation of PN 40612. As illustrated in FIG. 29A, PN 40292 (250 mg, 351 μmol) is dissolved in DMSO (10 mL). Hyp20 (900 mg, 395 μmol, GenScript) and triethylamine (1.25 mL, 9.0 mmol) are added. The mixture is stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives PN 40612 (164 μmol) with a m/z (LCMS negative mode, double charge)=1403.

Preparation of PN 40623. PN 40612 (164 μmol) is combined with dry DMF (15 mL), dry pyridine (1.0 mL, 12 mmol) and pentafluorophenyl trifluoroacetate (0.6 mL, 3.5 mmol, Sigma-Aldrich). The solution is stirred at room temperature for 2 hours. The reaction mixture is concentrated on a rotary evaporator, triturated with dibutyl ether (2 times), and triturated with MTBE (2 times) to give PN 40623 (150 μmol).

Preparation of PN 40656. A first solution is prepared by mixing PN 40623 (70 mg, 23.5 μmol) and DMF (1.5 mL). A second solution is prepared by mixing 1-Pentanaminium, 5-amino-5-carboxy-N,N,N-trimethyl-, chloride (15 mg, 67 μmol, Combi-blocks), saturated aqueous sodium bicarbonate (1.5 mL) and water (1.5 mL). The first and second solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives PN 40656 (17 μmol) with a m/z (LCMS negative mode, double charge)=1488.

Preparation of PN 40663. PN 40656 (50 μmol) is combined with dry DMF (10 mL), dry pyridine (0.4 mL, 4.9 mmol) and pentafluorophenyl trifluoroacetate (0.5 mL, 2.9 mmol, Sigma-Aldrich). The solution is stirred at room temperature for 2 hours. The reaction mixture is concentrated on a rotary evaporator, triturated with dibutyl ether (2 times), and triturated with MTBE (2 times) to give PN 40663 (50 μmol).

Preparation of PN 40667. As illustrated in FIG. 29A-29B, PN 40663 (50 mg, 15.9 μmol) and PN 40198 (43 mg, 177 μmole) is dissolved in anhydrous DMSO (4 mL) and triethylamine (500 uL, 3.6 mmol). The reaction mixed was allowed to stir for 2 hours at room temperature. The reaction mixture is purified by PREP-LC (250×100 mm 5A Gemini column, gradient of 20% to 60% acetonitrile/0.1 M TEAB buffer, 34 mL/min, 80 minutes). This gives PN 40667 (9 mg, 2.8 μmol) with a m/z (LCMS negative mode, double charge)=1600.

Preparation of PN 40670. PN 40667 (9 mg, 2.8 μmol) is combined with dry DMF (2 mL), dry pyridine (0.1 mL, 1.2 mmol) and pentafluorophenyl trifluoroacetate (0.2 mL, 1.2 mmol, Sigma-Aldrich). The solution is stirred at room temperature for 2 hours. The reaction mixture is concentrated on a rotary evaporator, triturated with dibutyl ether (2 times), and triturated with MTBE (2 times) to give PN 40670 (2.8 μmol).

Preparation of PN 40673. PN 40670 (2.8 μmol) is dissolved in DMSO (1 mL). 5-Propargylamino-2′-deoxyuridine-5′-Triphosphate (8.3 μmol, PN 40045, MyChem LLC) is dissolved in saturated aqueous sodium bicarbonate (0.5 mL). The two solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives PN 40673 (0.81 μmol) with a m/z (LCMS negative mode, double charge)=1852.

### Example 24: Synthesizing Hypn

A large order hydroxyproline moiety, Hypn (e.g., n>=20, 30, 40, 50, etc.), as used and described herein, may be synthesized by adding two or more smaller order hydroxyproline moieties, Hypn (e.g., n>=20, 15, 10, 9, 8, 7, 6, 5, 4, 3, etc.). For example, a Hyp30 is created by adding a Hyp10 and Hyp20. In another example, a Hyp40 is created by adding two Hyp20's. In another example, a Hyp12 is created by adding two Hyp6's. As seen from these examples, the two or more smaller order Hypn moieties may or may not be the same lengths.

### Example 25: Example Synthesis of Multi-Dye Labeled Compounds and Compound 40737

A multi-dye labeled substrate may be synthesized by assembling one or more dye segments with a substrate (e.g., nucleotide base, protein, etc.). The one or more dye segments may be assembled or linked together to form a final linker, with dyes attached at one or more locations on the final linker. As used herein, the term “dye segment” generally refers to a dye-attached linker segment that can be assembled with other linker segments, including dye-attached linker segments and non-dye-attached linker segments. The one or more dye segments may comprise a terminal dye segment and/or a non-terminal dye segment. As used herein, the term “terminal” dye segment generally refers to a dye-attached linker segment that can be assembled with other linker segments such that the dye of the terminal dye segment is attached to a distal end of a final linker relative to the substrate (e.g., nucleotide base). Where the final linker comprises a plurality of repeating units (e.g., polyproline or poly-hydroxyproline), the dye of a terminal dye segment may be attached to the last repeating unit of the linker. As used herein, the term “non-terminal” dye segment generally refers to a dye-attached linker segment that can be assembled with other linker segments such that the dye in the non-terminal dye segment is not attached to a distal end of a final linker relative to the substrate (e.g., nucleotide base). Where the final linker comprises a plurality of repeating units (e.g., polyproline or poly-hydroxyproline), the dye of the non-terminal dye segment may be attached between repeating units of the final linker. A final linker may comprise any number of, one of, and/or combination of terminal dye segments and non-terminal dye segments. A terminal dye segment and a non-terminal dye segment of the same length, or of the same number of repeating units, may have different structures.

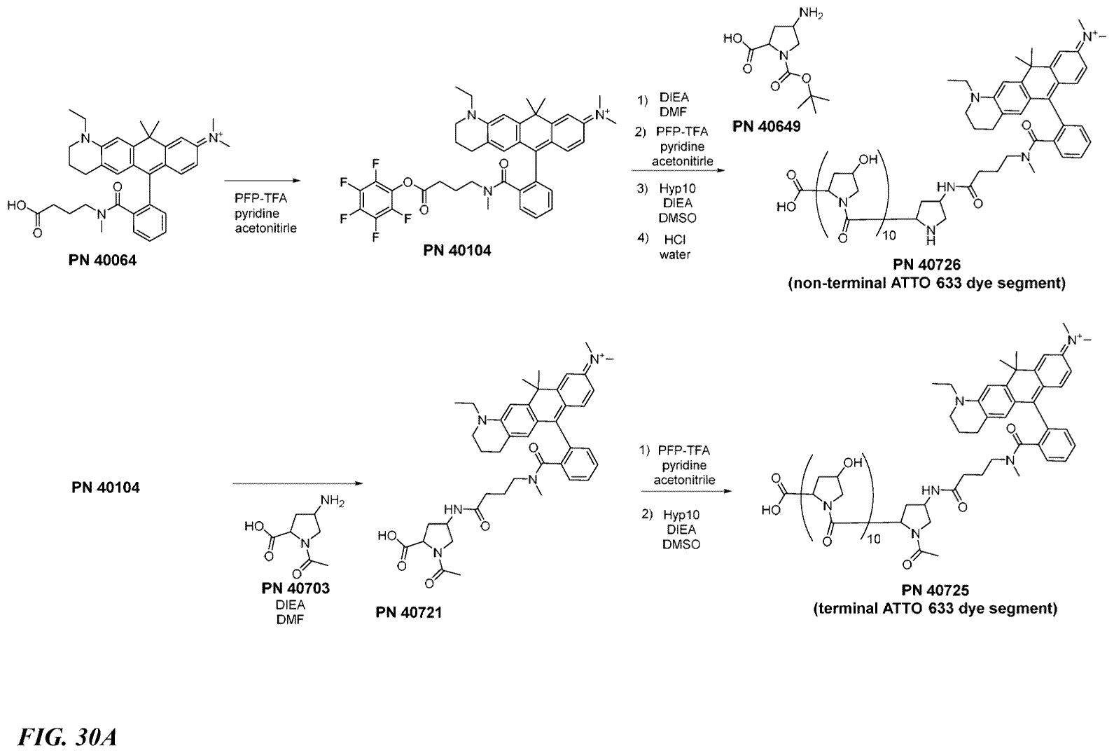

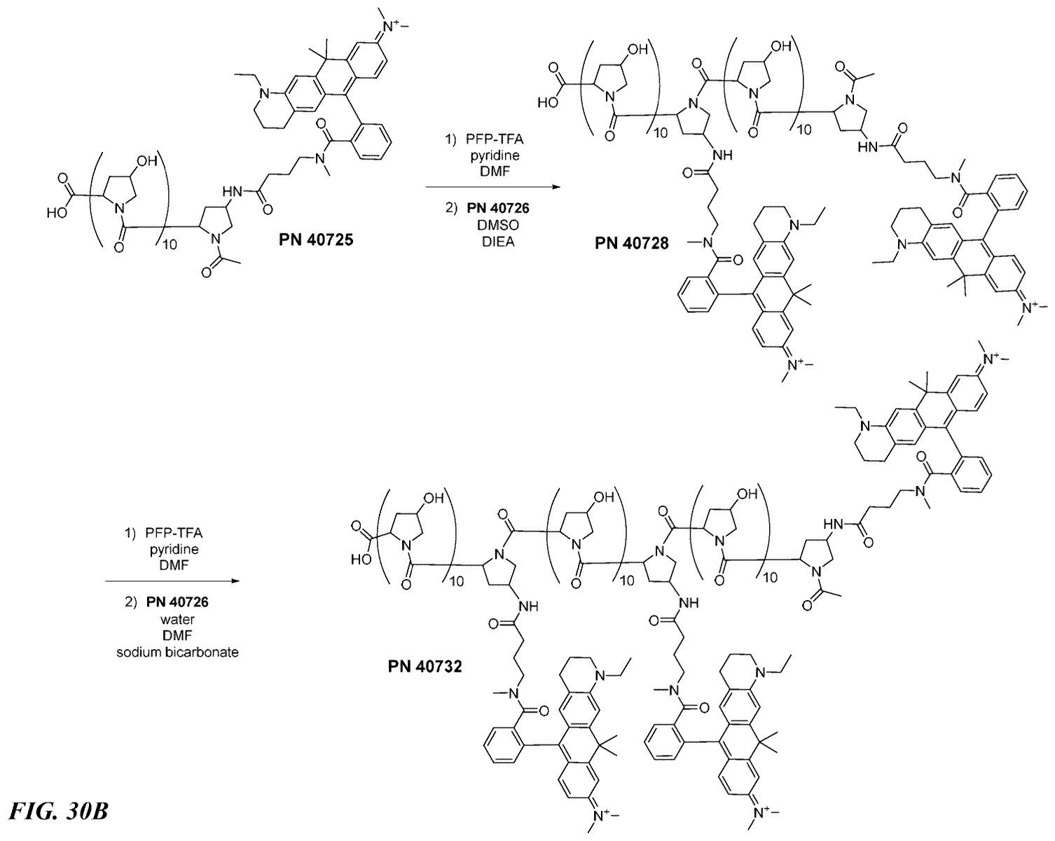

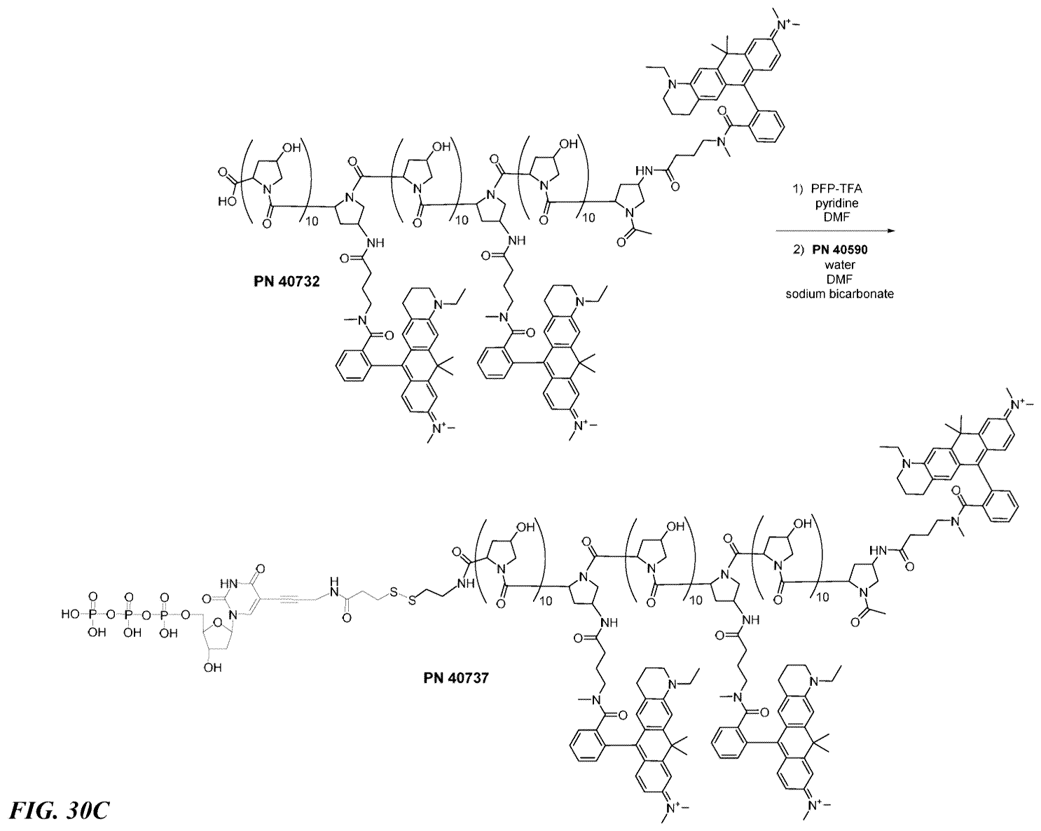

FIGS. 30A-C show an example process for synthesizing a multi-fluorescently labeled dUTP nucleotide with an E-cleavable linker (see FIG. 4), 2 non-terminal ATTO 633 dye segments (PN 40726) and a terminal ATTO 633 dye segment (PN 40725), each separated by a Hyp10.

Preparation of PN 40104. As illustrated in FIGS. 30A-C, Atto633-CO₂H (PN 40064, 1.2 g, 2.1 mmol, Atto-Tec GmbH) is combined with dry acetonitrile (20.0 mL), dry pyridine (2.6 mL, 32.0 mmol) and pentafluorophenyl trifluoroacetate (3.6 mL, 21.2 mmol, Sigma-Aldrich). The solution is stirred at room temperature for 1 hour. The reaction mixture is concentrated on a rotary evaporator, reconstituted with dry acetonitrile, and concentrated again to give PN 40104 (2.1 mmol) with a m/z (LCMS positive mode)=718. 3.

Preparation of PN 40726. PN 40104 (1.6 mmol) is then dissolved in dry DMF (24.0 mL). (2S, 4R)-1-Boc-4-amino-pyrrolidine-2-carboxylic acid (790.0 mg, 3.4 mmol, AchemBlock) is dissolved in mixture of dry DMF (16.0 mL) and N, N-diisopropylethylamine (10.0 mL) with sonication and vortexing. The two solutions are combined and stirred at room temperature for 2 hours. The reaction mixture is purified by PREP-LC (250×100 mm 5A Gemini column, gradient of 5% to 60% acetonitrile/0.1 M TEAB buffer, 34 mL/min, 80 minutes). This gives the aminopyridine Atto633 compound (1.0 mmol) with a m/z (LCMS positive mode)=765.4. The aminopyridine Atto633 compound (1.0 mmol) is then combined with dry acetonitrile (20.0 mL), dry pyridine (1.2 mL, 14.8 mmol) and pentafluorophenyl trifluoroacetate (1.8 mL, 10.4 mmol, Sigma-Aldrich). The solution is stirred at room temperature for 1 hour. The reaction mixture is concentrated on a rotary evaporator, reconstituted with dry acetonitrile, and concentrated again to give aminopyridine Atto633-PFP compound (1.0 mmol). The aminopyridine Atto633-PFP compound (440.0 umol) is dissolved in the DMSO (6.0 mL). Hyp10 (910.1 mg, 792.0 umol, GenScript) is dissolved in DMSO (5.0 mL) and DIEA (3.0 mL). The two solutions are combined and stirred at room temperature for 2 hours. The reaction mixture is purified by PREP-LC (250×100 mm 5A Gemini column, gradient of 20% to 60% acetonitrile/0.1 M TEAA buffer, 34 mL/min, 80 minutes). This gives aminopyridine Atto633-PFP-Hyp10 (325.0 umol) with a m/z (LCMS positive mode)=1895.8. The aminopyridine Atto633-PFP-Hyp10 (200.0 umol) is dissolved in water (10.0 mL) and 3 M HCl (18.0 mL) is added. The reaction mixture is stirred for 3 hours at room temperature and then quenched with 1M TEAB. The reaction mixture is concentrated under reduced pressure. The reaction mixture is purified by PREP-LC (250×100 mm 5A Gemini column, gradient of 2% to 60% acetonitrile/0.1 M TEAB buffer, 34 mL/min, 80 minutes). This gives PN 40726 (150.0 umol) with a m/z (LCMS positive mode)=1795.6, a non-terminal dye segment.

Preparation of PN 40721. PN 40104 (565.0 mmol) is then dissolved in dry DMF (10.0 mL). (2S, 4R)-1-acetyl-4-amino-pyrrolidine-2-carboxylic acid (790.0 mg, 3.4 mmol, Enamine) is dissolved in mixture of dry DMF (5.0 mL) and N, N-diisopropylethylamine (3.6 mL) with sonication and vortexing. The two solutions are combined and stirred at room temperature for 2 hours. The reaction mixture is purified by PREP-LC (250×100 mm 5A Gemini column, gradient of 5% to 60% acetonitrile/0.1 M TEAB buffer, 34 mL/min, 80 minutes). This gives PN 40721 (273.0 umol) with a m/z (LCMS positive mode)=707.4.

Preparation of PN 40725. PN 40721 (273.0 umol) is then combined with dry acetonitrile (9.0 mL), dry pyridine (0.6 mL, 7.4 mmol) and pentafluorophenyl trifluoroacetate (0.8 mL, 4.6 mmol, Sigma-Aldrich). The solution is stirred at room temperature for 1 hour. The reaction mixture is concentrated on a rotary evaporator, reconstituted with dry acetonitrile, and concentrated again to give PFP ester of PN 40721 (273.0 umol). The PFP ester of PN 40721 (273.0 umol) is dissolved in the DMSO (8.0 mL). Hyp10 (471.2 mg, 410.0 umol, GenScript) in DMSO (4.0 mL) and DIEA (2.0 mL). The two solutions are combined and stirred at room temperature for 2 hours. The reaction mixture is purified by PREP-LC (250×100 mm 5A Gemini column, gradient of 20% to 60% acetonitrile/0.1 M TEAA buffer, 34 mL/min, 80 minutes). This gives PN 40725 (97.4 umol) with a m/z (LCMS positive mode)=1837.7, a terminal dye segment.

Preparation of PN 40728. PN 40725 (97.4 mmol) is then combined with dry DMF (4.0 mL), dry pyridine (0.1 mL, 1.2 mmol) and pentafluorophenyl trifluoroacetate (0.3 mL, 1.7 mmol, Sigma-Aldrich). The solution is stirred at room temperature for 1 hour. The reaction mixture is concentrated on a rotary evaporator, reconstituted with dry acetonitrile, and concentrated again to give PFP ester of PN 40725 (97.0 umol). PFP ester of PN 40725 (97.0 umol) is then dissolved in dry DMSO (2.0 mL). The PN 40726 (152.0 umol) is dissolved in mixture of dry DMSO (4.0 mL) and N, N-diisopropylethylamine (1.0 mL) with sonication and vortexing. The two solutions are combined and stirred at room temperature for 2 hours. The reaction mixture is purified by PREP-LC (250×100 mm 5A Gemini column, gradient of 5% to 60% acetonitrile/0.1 M TEAB buffer, 34 mL/min, 80 minutes). This gives PN 40728 (48.0 umol) with a m/z (LCMS positive mode, double charge)=1808.0.

Preparation of PN 40732. PN 40728 (48.0 mmol) is then combined with dry DMF (3.0 mL), dry pyridine (0.2 mL, 2.4 mmol) and pentafluorophenyl trifluoroacetate (0.35 mL, 2.0 mmol, Sigma-Aldrich). The solution is stirred at room temperature for 1 hour. The reaction mixture is concentrated on a rotary evaporator, reconstituted with dry acetonitrile, and concentrated again to give PFP ester of PN 40728 (48.0 umol). PFP ester of PN 40728 (48.0 umol) is then dissolved in dry DMSO (1.0 mL). The PN 40726 (152.0 umol) is dissolved in mixture of dry DMSO (1.5 mL) and N, N-diisopropylethylamine (1.0 mL) with sonication and vortexing. The two solutions are combined and stirred at room temperature for 2 hours. The reaction mixture is purified by PREP-LC (250×100 mm 5A Gemini column, gradient of 5% to 60% acetonitrile/0.1 M TEAB buffer, 34 mL/min, 80 minutes). This gives PN 40732 (33.0 umol) with a m/z (LCMS positive mode, triple charge)=1797.8.

Preparation of PN 40737. PN 40732 (33.0 mmol) is then combined with dry DMF (3.5 mL), dry pyridine (0.3 mL, 3.7 mmol) and pentafluorophenyl trifluoroacetate (0.5 mL, 2.9 mmol, Sigma-Aldrich). The solution is stirred at room temperature for 1 hour. The reaction mixture is concentrated on a rotary evaporator, reconstituted with dry acetonitrile, and concentrated again to give PFP ester of PN 4032 (22.0 umol). PFP ester of PN 40732 (33.0 umol) is dissolved in dry DMF (6.0 mL). PN 40590 (104.0 umol) is dissolved in saturated aqueous sodium bicarbonate (4.0 mL), and water (4.0 mL). The two solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives PN 40737 (9.0 umol) with a m/z (LCMS positive mode, four charge)=1515.2

### Example 26: Example Synthesis of Compound 40736

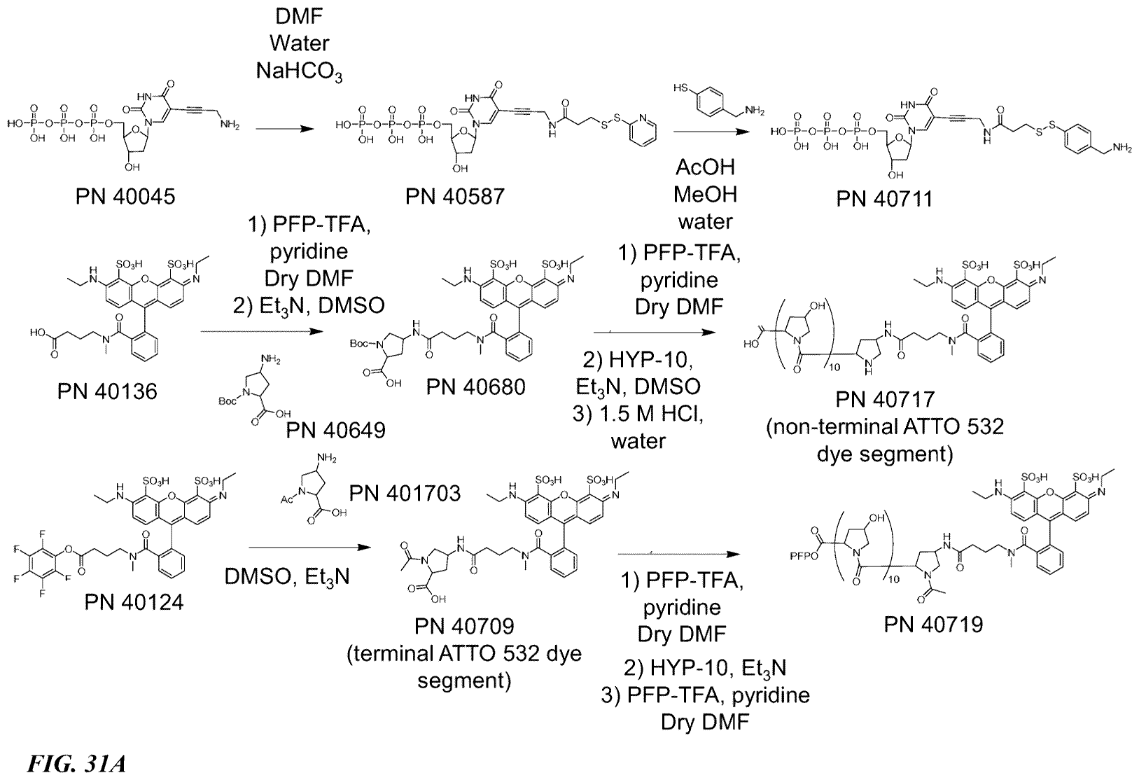

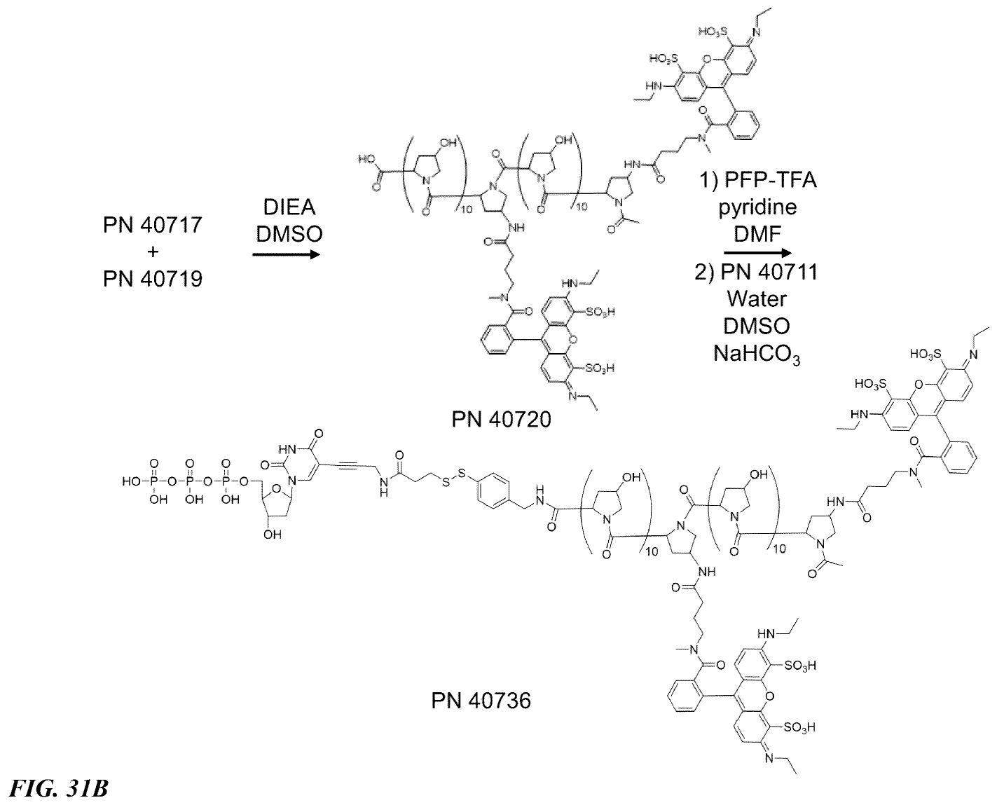

FIG. 31A-B show an example process for synthesizing a multi-fluorescently labeled dUTP nucleotide with a Y-cleavable linker (see FIG. 4), a non-terminal ATTO 532 dye segment (PN 40717) and a terminal ATTO 532 dye segment (PN 40709), each separated by a Hyp10.

Preparation of PN 40680. As illustrated in FIG. 31A-B, a solution of ATTO 532-CO₂H (850.0 mg, 1.3 mmol, PN 40136) is combined with dry DMF (15.0 mL), dry pyridine (2.0 mL, 24.7 mmol) and pentafluorophenyl trifluoroacetate (2.0 mL, 11.6 mmol, Sigma-Aldrich). The solution is stirred at room temperature for 2 hours. The reaction mixture is concentrated on a rotary evaporator until the volume is reduced to 3.0 mL. The concentrated reaction mixture is then added to dibutylether (50.0 mL) followed by vortexing, sonicating, centrifuging to give dark colored pellet, supernatant is discarded. The insoluble pellet is then suspended in MTBE, followed by vortexing, sonicating and centrifuging. The supernatant is discarded, and this process is repeated thrice to give ATTO 532-PFP (PN 40124) as a red solid. ATTO 532-PFP (250.0 mg, 351.0 umol) is then dissolved in dry DMSO (5.0 mL). (2S, 4R)-1-Boc-4-amino-pyrrolidine-2-carboxylic acid (170.0 mg, 738.7 umol, AchemBlock) is dissolved in mixture of DCM (3.0 mL) and triethylamine (2.0 mL) with sonication and vortexing. The two solutions are combined and stirred at room temperature for 2 hours. The reaction mixture is purified by PREP-LC (250×100 mm 5A Gemini column, gradient of 5% to 60% acetonitrile/0.1 M TEAB buffer, 34 mL/min, 80 minutes). This gives PN 40680 (119.0 umol) with a m/z (LCMS negative mode)=856.3.

Preparation of PN 40717. A solution of PN 40680 (119.0 umol) is combined with dry DMF (15.0 mL), dry pyridine (1.0 mL, 12.3 mmol) and pentafluorophenyl trifluoroacetate (0.2 mL, 1.2 mmol, Sigma-Aldrich). The solution is stirred at room temperature for 2 hours. The reaction mixture is concentrated on a rotary evaporator until the volume is reduced to 3.0 mL. The concentrated reaction mixture is then added to dibutylether (50.0 mL) followed by vortexing, sonicating, centrifuging to give dark colored pellet, supernatant is discarded. The insoluble pellet is then suspended in MTBE, followed by vortexing, sonicating and centrifuging. The supernatant is discarded, and this process is repeated thrice to give PFP ester of PN 40680 as a red solid. PFP ester of PN 40680 (57.6 umol) is dissolved in the DMSO (5.0 mL). Hyp10 (130.0 mg, 113.1 umol, GenScript) is dissolved in mixture of DMSO (1.0 mL) and triethylamine (0.5 mL). The two solutions are combined and stirred at room temperature for 2 hours. The reaction mixture is purified by PREP-LC (250×100 mm 5A Gemini column, gradient of 5% to 60% acetonitrile/0.1 M TEAB buffer, 34 mL/min, 80 minutes). This gives PN 40699 (30.0 umol) with a m/z (LCMS negative mode)=1987.6. PN 40699 (30.0 umol) is dissolved in water (2.0 mL) and 1.5 M HCl (6.0 mL) is added. The reaction mixture is stirred for 3 hours at room temperature and then quenched with 1M TEAB. The reaction mixture is concentrated under reduced pressure. The reaction mixture is purified by PREP-LC (250×100 mm 5A Gemini column, gradient of 2% to 60% acetonitrile/0.1 M TEAB buffer, 34 mL/min, 80 minutes). This gives PN 40717 (20.0 umol) with a m/z (LCMS negative mode)=1887.6.

Preparation of PN 40709. PN 40124 (70.0 mg, 86.3 umol) is then dissolved in dry DMSO (5.0 mL). (2S, 4R)-1-acetyl-4-amino-pyrrolidine-2-carboxylic acid (40.0 mg, 232.3 umol, Enamine) is dissolved in mixture of DMSO (2.0 mL) and triethylamine (0.5 mL) with sonication and vortexing. The two solutions are combined and stirred at room temperature for 2 hours. The reaction mixture is purified by PREP-LC (250×100 mm 5A Gemini column, gradient of 5% to 60% acetonitrile/0.1 M TEAB buffer, 34 mL/min, 80 minutes). This gives PN 40709 (56.0 umol) with a m/z (LCMS negative mode)=798.2.

Preparation of PN 40719. A solution of PN 40709 (87.6 mmol) is combined with dry DMF (5.0 mL), dry pyridine (0.15 mL, 1.8 mmol) and pentafluorophenyl trifluoroacetate (0.15 mL, 0.9 mmol, Sigma-Aldrich). The solution is stirred at room temperature for 2 hours. The reaction mixture is concentrated on a rotary evaporator until the volume is reduced to 3.0 mL. The concentrated reaction mixture is then added to dibutylether (30.0 mL) followed by vortexing, sonicating, centrifuging to give dark colored pellet, supernatant is discarded. The insoluble pellet is then suspended in MTBE, followed by vortexing, sonicating and centrifuging. The supernatant is discarded, and this process is repeated thrice to give PFP ester of PN 40709 (87.0 umol) with a m/z (LCMS positive mode)=966.1 as a red solid. PFP ester of PN 40709 (72.5 umol) is then dissolved in the DMSO (5.0 mL). Hyp10 (130.0 mg, 113.1 umol, GenScript) is dissolved in mixture of DMSO (1.0 mL) and triethylamine (0.2 mL). The two solutions are combined and stirred at room temperature for 2 hours. The reaction mixture is purified by PREP-LC (250×100 mm 5A Gemini column, gradient of 5% to 60% acetonitrile/0.1 M TEAB buffer, 34 mL/min, 80 minutes). This gives PN 40715 (41.5 umol) with a m/z (LCMS negative)=1929.7. PN 40715 (41.5 umol) is then combined with dry DMF (3.0 mL), dry pyridine (50.0 uL, 618.2 umol) and pentafluorophenyl trifluoroacetate (0.1 mL, 582.0 umol, Sigma-Aldrich). The solution is stirred at room temperature for 2 hours. The reaction mixture is concentrated on a rotary evaporator until the volume is reduced to 0.5 mL. The concentrated reaction mixture is then added to dibutylether (15.0 mL) followed by vortexing, sonicating, centrifuging to give dark colored pellet, supernatant is discarded. The insoluble pellet is then suspended in MTBE, followed by vortexing, sonicating and centrifuging. The supernatant is discarded, and this process is repeated thrice to give PN 40719 (41.0 umol) as a red solid.

Preparation of PN 40720. As illustrated in FIG. 31A-B, a solution of PN 40719 (26.0 mg, 12.4 umol) and PN 40717 (25.0 mg, 13.2 umol) are dissolved in dry DMSO (3.0 mL). N, N-diisopropylethylamine (0.3 mL) is added to the reaction. The mixture is stirred at room temperature for 2 hours. The reaction mixture is purified by PREP-LC (250×100 mm 5A Gemini column, gradient of 5% to 60% acetonitrile/0.1 M TEAB buffer, 34 mL/min, 80 minutes). This gives PN 40720 (2.0 umol) with a m/z (LCMS negative mode, double charge)=1899.3.

Preparation of PN 40587. 5-Propargylamino-2′-deoxyuridine-5′-Triphosphate (215.0 umol, PN 40045, MyChem LLC), saturated aqueous sodium bicarbonate (3.0 mL) and water (3.0 mL) are combined. Succinimidyl 3-(2-pyridyldithio)-propionate (300.0 mg, 960.0 umol, Thermo Scientific) is dissolved in DMF (5.0 mL). The two solutions are combined and mixed for 1 hour at room temperature. The reaction mixture is purified by PREP-LC (250×100 mm 5A Gemini column, gradient of 2% to 60% acetonitrile/0.1 M TEAB buffer, 34 mL/min, 80 minutes). This gives PN 40587 (132.0 umol) with a m/z (LCMS negative mode)=716.9.

Preparation of PN 40711. PN 40587 (80 umol) is dissolved in water (6.0 mL). 4-(aminomethyl) benzene-1-thiol (40.0 mg, 227.7 umol, Enamine) is dissolved in MeOH (4.0 mL). A few drops of acetic acid are added to the reaction mixture and stirred for 1 hour at room temperature. The reaction mixture is purified by PREP-LC (250×100 mm 5A Gemini column, gradient of 2% to 60% acetonitrile/0.1 M TEAB buffer, 34 mL/min, 80 minutes). This gives PN 40711 (41.0 umol) with a m/z (LCMS negative mode)=745.0.

Preparation of PN 40736. A solution of PN 40720 (2.0 mmol) is combined with dry DMF (4.0 mL), dry pyridine (0.13 mL, 1.6 mmol) and pentafluorophenyl trifluoroacetate (0.15 mL, 0.9 mmol, Sigma-Aldrich). The solution is stirred at room temperature for 2 hours. The reaction mixture is concentrated on a rotary evaporator until the volume is reduced to 0.5 mL. The concentrated reaction mixture is then added to dibutylether (15.0 mL) followed by vortexing, sonicating, centrifuging to give dark colored pellet, supernatant is discarded. The insoluble pellet is then suspended in MTBE, followed by vortexing, sonicating and centrifuging. The supernatant is discarded, and this process is repeated thrice to give PFP ester of PN 40720 (2.0 umol) as a red solid. PFP ester of PN 40720 (2.0 umol) is dissolved in dry DMF (3.0 mL). PN 40711 (8.0 umol) is dissolved in saturated aqueous sodium bicarbonate (0.3 mL), and water (2.0 mL). The two solutions are combined and stirred at room temperature for 2 hours. The reaction mixture is purified by PREP-LC (250×100 mm 5A Gemini column, gradient of 20% to 60% acetonitrile/0.1 M TEAB buffer, 34 mL/min, 80 minutes). This gives PN 40736 (1.2 umol) with a m/z (LCMS negative mode, triple charge)=1509.1.

### Example 26: Kinetics of Quaternary Amine Linkers

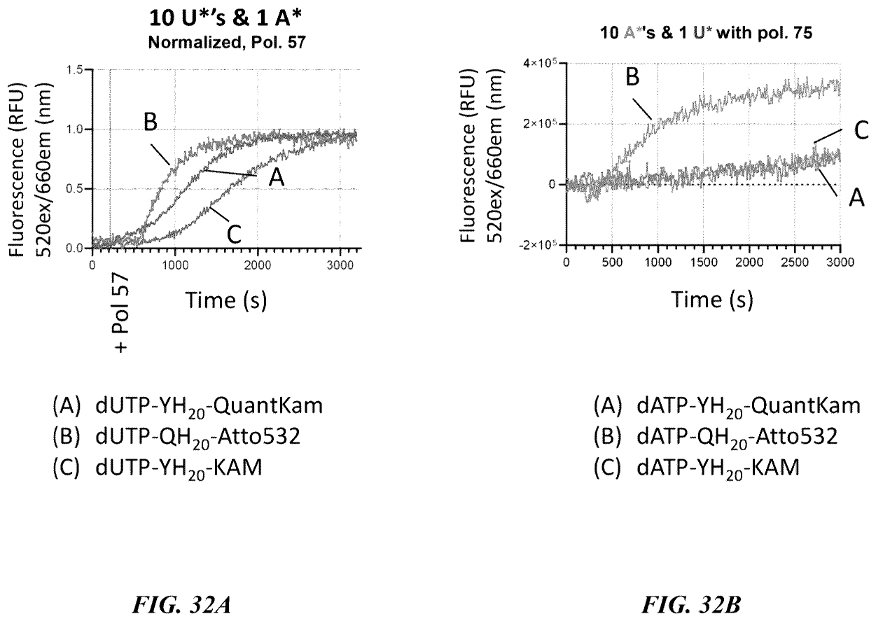

FIG. 32 shows plate-based kinetics assay data (a) in the top panel for dUTP-YH₂₀-QuatKam (A), dUTP-QH₂₀-Atto532 (B), and dUTP-YH₂₀-Kam (C) and (b) in the bottom panel for dATP-YH₂₀-QuatKam (A), dATP-QH₂₀-Atto532 (B), and dATP-YH₂₀-Kam (C). The assays demonstrate that the two different nucleotides dATPs and dUTPs behave differently with the assayed quaternary amine linker structures, with the dUTP-YH₂₀-QuatKam yielding improved lag phase incorporation rates than dUTPs labeled with non-quaternary amine linker structures.

In the first assay, corresponding to FIG. 32A, 3 types of labeled dUTPs (dUTP-YH₂₀-QuatKam (A), dUTP-QH₂₀-Atto532 (B), and dUTP-YH₂₀-Kam (C)) and 1 type of red-labeled dATP were provided to a primer-hybridized template, to extend through a A₁₀TC sequence in the template. Each of the graphs in the top panel and bottom panel plots Fluorescence (RFU) vs. Time (s). As seen in the top panel, all 3 labeled nucleotide types show biphasic behavior, which indicate lag phase associated with the incorporation of 10 U's. Based on lag phase rate of incorporation of 10 U's, the performance of the 3 types of labeled dUTPs are ranked: dUTP-YH₂₀-QuatKam>dUTP-QH₂₀-Atto532>dUTP-YH₂₀-Kam. Based on time to completion (including incorporation of the red dATP after the homopolymer stretch), the performance of the 3 types of labeled dUTPs are ranked: dUTP-QH₂₀-Atto532>dUTP-YH₂₀-QuatKam>dUTP-YH₂₀-Kam.

In the second assay, corresponding to FIG. 32B, 3 types of labeled dATPs (dATP-YH₂₀-QuatKam (A), dATP-QH₂₀-Atto532 (B), and dATP-YH₂₀-Kam (C)) and 1 type of dUTP were provided to a primer-hybridized template, to extend through a T₁₀AG sequence in the template. As seen in the bottom panel, in the given time frame, the dATP-YH₂₀-QuatKam and dATP-YH₂₀-Kam were able to fully extend a fraction of the templates, while the dATP-QH₂₀-Atto532 was able to fully extend all of the templates. The performance of the 3 types of labeled dATPs are thus ranked dATP-QH₂₀-Atto532>dATP-YH₂₀-QuatKam≈dATP-YH₂₀-Kam.

### Example 27: Fluorescence Comparison of Multi-Labeled Optical Labeling Agents

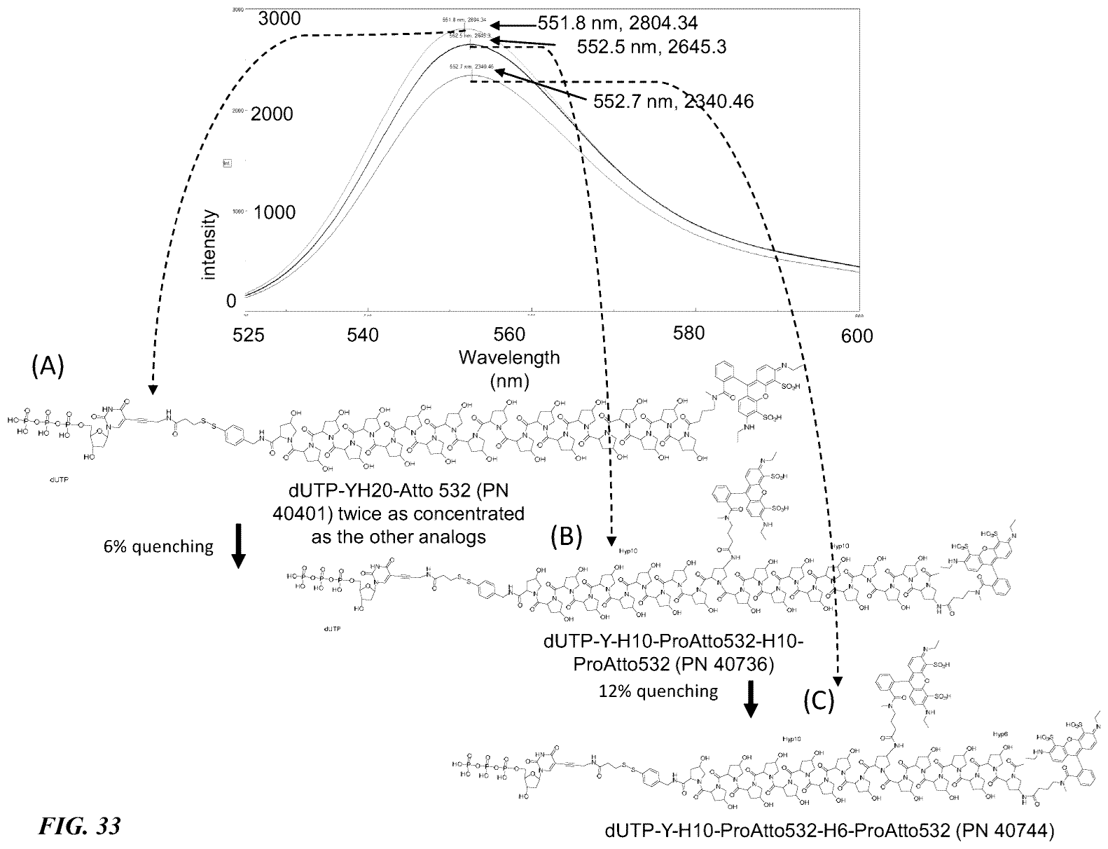

FIG. 33 illustrates a fluorescence assay for three different labeled dUTP compounds, with the graph plotting intensity vs. wavelength (nm). The three compounds, whose structures are illustrated in FIG. 33, are (A) dUTP-YH20-Atto532 (PN 40401), where a single dye is attached with a H20 linker between the substrate and the dye, (B) dUTP-Y-H10-ProAtto532-H10-ProAtto532 (PN 40736), where two dyes are attached, a first dye attached with a H10 linker between the substrate and the first dye, and a second dye attached with a H10 linker between the first dye and the second dye, and (C) dUTP-Y-H10-ProAtto532-H6-ProAtto532 (PN 40744), where two dyes are attached, a first dye attached with a H10 linker between the substrate and the first dye, and a second dye attached with a H6 linker between the first dye and the second dye.

Each sample has the same absorbance measurement (0.278 Au at 432 nm), and compound A (with the single dye) is twice as concentrated. Thus, if there were no quenching between multiple dyes in the same compound, all 3 samples theoretically would have the same fluorescence measurements in addition to the same absorbance measurements. However, as seen in the plot in FIG. 33, quenching is observed. The peaks are read at: Compound (A): 2804.34 units at 551.8 nm; Compound (B): 2645.3 units at 552.5 nm; Compound (C): 2340.46 units at 552.7 nm. Thus ˜6% quenching ((2804.34-2645.3)/2804.34) was observed in Compound (B) compared to Compound (A), and ˜12% quenching ((2645.3-2340.46)/2645.3) was observed in Compound (C) compared to Compound (B). The data confirms that multi-dye linkers are plausible without significant quenching issues. Further, the data demonstrates that dyes separated by the longer Hyp10 linker (˜33 angstrom spacing) in Compound (B) has significantly reduced quenching (by ˜12%) than dyes separated by the shorter Hyp6 linker (˜21 angstrom spacing) in Compound (C). The observed reduced quenching effect may be due to the difference in spacing between the two dyes, and/or due to angular separation between the two dyes.

### Example 28: Evaluating Quenching Levels Between Different Length Linkers

This example illustrates the effect of the length of the hydroxyproline linker on the intensity of a Atto532 dye attached to one end of a molecule, where a Atto633 dye is attached to the other end. The first molecule has a length of 10 hydroxyproline residues separating the two dyes. The second molecule has a length of 20 hydroxyproline residues separating the two dyes (e.g., see FIG. 34B). The two derivatives were dissolved to equal concentrations and their fluorescence intensities were measured at 560 nm (Atto532 emission) and 660 nm (Atto633 emission) using 520 nm as the excitation wavelength. The data is provided below.

TABLE 3
Effect of length of hydroxyproline
linkers on fluorescence intensities

| Length of | 560 nm | 660 nm | |
|---|---|---|---|
| hydroxyproline linker | intensity | intensity | Ratio |
| 10 | 135000 | 447000 | 0.30 |
| 20 | 700000 | 352000 | 1.99 |

As shown in Table 3, for the first molecule with the shorter distance between the two dyes (10 hydroxyproline residues), the ratio of signal intensities between the 560 nm (Atto532 emission) and 660 nm (Atto633 emission) measurement was 135,000:447,000, at approximately 0.30. For the second molecule with the longer distance between the two dyes (20 hydroxyproline residues), the ratio of signal intensities between the 560 nm and 660 nm measurement was 700,000:352,000, at approximately 1.99. The data demonstrates that the Atto532 dye was significantly quenched more in the first molecule with the shorter distance between the two dyes than in the second molecule with the longer distance between the two dyes.

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

1. A labeling reagent comprising: (a) a detectable moiety; and (b) a linker that is coupled to said detectable moiety, wherein said linker comprises at least one cleavable group and at least one non-proteinogenic amino acid, wherein said at least one non-proteinogenic amino acid does not comprise hydroxyproline, and wherein when said at least one non-proteinogenic amino acid comprises 6-aminohexanoic acid, said detectable moiety is not a Cyanide 5 (Cy5) or an ATTO 647 NHS Ester (ATTO 647N).

2. The labeling reagent of embodiment 1, said detectable moiety does not comprise said Cy5 or said ATTO 647N. 3. The labeling reagent of embodiment 1 or 2, wherein said at least one non-proteinogenic amino acid comprises at most about 50 atoms. 4. The labeling reagent of any one of embodiments 1-3, wherein said at least one non-proteinogenic amino acid comprises at most about 20 atoms. 5. The labeling reagent of any one of embodiments 1-4, wherein said at least one non-proteinogenic amino acid comprises about 10-20 atoms. 6. The labeling reagent of any one of embodiments 1-5, wherein said at least one non-proteinogenic amino acid comprises cysteic acid. 7. The labeling reagent of any one of embodiments 1-5, wherein said at least one non-proteinogenic amino acid comprises 5-amino-5-carboxy-N,N,N-trimethylpentan-1-aminium or a salt thereof. 8. The labeling reagent of any one of embodiments 1-7, wherein said at least one non-proteinogenic amino acid comprises 6-aminohexanoic acid. 9. The labeling reagent of any one of embodiments 1-8, wherein said detectable moiety comprises a fluorescent dye. 10. The labeling reagent of embodiment 9, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, or Kam. 11. The labeling reagent of embodiment 10, wherein said fluorescent dye comprises ATTO 633. 12. The labeling reagent of any one of embodiments 1-11, wherein said at least one cleavable group is configured to be cleaved to separate a portion of said detectable moiety from said labeling reagent. 13. The labeling reagent of any one of embodiments 1-12, wherein said at least one cleavable group is selected from the group consisting of an azidomethyl group, a disulfide bond, a hydrocarbyldithiomethyl group, and a 2-nitrobenzyloxy group. 14. The labeling reagent of embodiment 13, wherein said at least one cleavable group is said disulfide bond. 15. The labeling reagent of any one of embodiments 1-14, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof. 16. The labeling reagent of any one of embodiments 1-15, wherein said labeling reagent comprises a moiety selected from the group consisting of

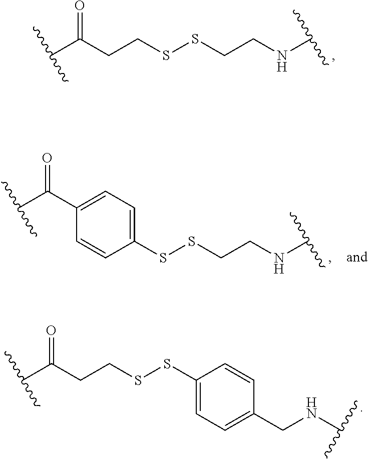 [SMILES: *NCCSSCCC(*)=O ; *NCCSSc1ccc(C(*)=O)cc1 ; *NCc1ccc(SSCCC(*)=O)cc1]

17. A labeling reagent comprising a compound of Formula I:

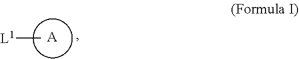

wherein: A is a detectable moiety; and L1 is a linker comprising at least one cleavable group and at least one non-proteinogenic amino acid, wherein said non-proteinogenic amino acid does not comprise hydroxyproline, and wherein when said at least one non-proteinogenic amino acid comprises 6-aminohexanoic acid, said detectable moiety is not a Cyanide 5 (Cy5) or an ATTO 647 NHS Ester (ATTO 647N).

18. The labeling reagent of embodiment 17, wherein said detectable moiety does not comprise said Cy5 or said ATTO 647N. 19. The labeling reagent of embodiment 17 or 18, wherein said at least one non-proteinogenic amino acid comprises at most about 50 atoms. 20. The labeling reagent of any one of embodiments 17-19, wherein said at least one non-proteinogenic amino acid comprises at most about 20 atoms. 21. The labeling reagent of any one of embodiments 17-20, wherein said at least one non-proteinogenic amino acid comprises about 10-20 atoms. 22. The labeling reagent of any one of embodiments 17-21, wherein said at least one non-proteinogenic amino acid comprises cysteic acid. 23. The labeling reagent of any one of embodiments 17-21, wherein said at least one non-proteinogenic amino acid comprises 5-amino-5-carboxy-N,N,N-trimethylpentan-1-aminium or a salt thereof. 24. The labeling reagent of any one of embodiments 17-23, wherein said at least one non-proteinogenic amino acid comprises 6-aminohexanoic acid. 25. The labeling reagent of any one of embodiments 17-24, wherein said detectable moiety comprises a fluorescent dye. 26. The labeling reagent of embodiment 25, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, or Kam. 27. The labeling reagent of embodiment 26, wherein said fluorescent dye comprises ATTO 633. 28. The labeling reagent of any one of embodiments 17-27, wherein said at least one cleavable group is configured to be cleaved to separate a portion of said detectable moiety from said labeling reagent. 29. The labeling reagent of any one of embodiments 17-28, wherein said at least one cleavable group is selected from the group consisting of an azidomethyl group, a disulfide bond, a hydrocarbyldithiomethyl group, and a 2-nitrobenzyloxy group. 30. The labeling reagent of embodiment 29, wherein said at least one cleavable group is said disulfide bond. 31. The labeling reagent of any one of embodiments 17-30, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof 32. The labeling reagent of any one of embodiments 17-31, wherein said labeling reagent comprises a moiety selected from the group consisting of

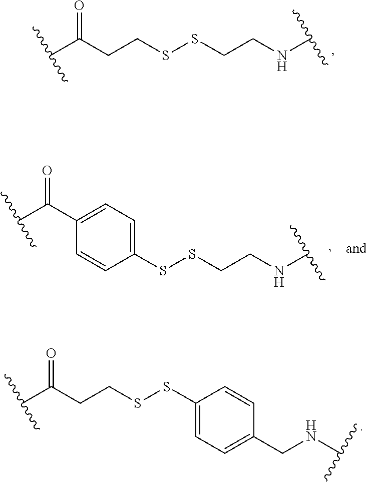 [SMILES: *NCCSSCCC(*)=O ; *NCCSSc1ccc(C(*)=O)cc1 ; *NCc1ccc(SSCCC(*)=O)cc1]

33. A labeling reagent comprising: (a) a detectable moiety; and (b) a linker that is coupled to said detectable moiety, wherein said linker comprises at least one cleavable group and at least one non-proteinogenic amino acid, wherein said at least one non-proteinogenic amino acid does not comprise hydroxyproline, and wherein when said at least one non-proteinogenic amino acid comprises 6-aminohexanoic acid, said linker is not coupled to

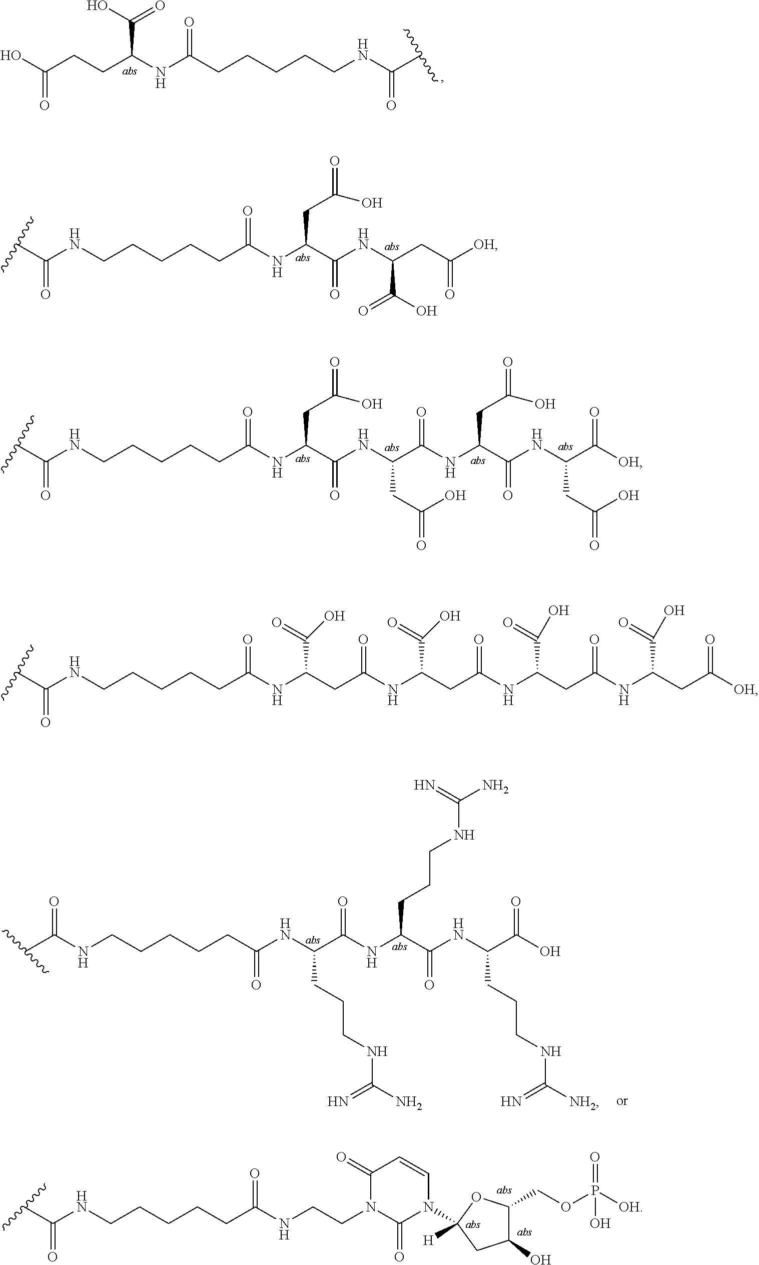 [SMILES: *C(=O)NCCCCCC(=O)N[C@@H](CCC(=O)O)C(=O)O ; *C(=O)NCCCCCC(=O)N[C@@H](CC(=O)O)C(=O)N[C@@H](CC(=O)O)C(=O)O ; *C(=O)NCCCCCC(=O)N[C@@H](CC(=O)O)C(=O)N[C@@H](CC(=O)O)C(=O)N[C@@H](CC(=O)O)C(=O)N[C@@H](CC(=O)O)C(=O)O ; *C(=O)NCCCCCC(=O)N[C@@H](CC(=O)N[C@@H](CC(=O)N[C@@H](CC(=O)N[C@@H](CC(=O)O)C(=O)O)C(=O)O)C(=O)O)C(=O)O ; *C(=O)NCCCCCC(=O)N[C@@H](CCCNC(=N)N)C(=O)N[C@@H](CCCNC(=N)N)C(=O)N[C@@H](CCCNC(=N)N)C(=O)O ; *C(=O)NCCCCCC(=O)NCCn1c(=O)ccn([C@H]2C[C@H](O)[C@@H](COP(=O)(O)O)O2)c1=O]

34. The labeling reagent of embodiment 33, wherein said linker is not coupled to a terminator group. 35. The labeling reagent of embodiment 33 or 34, wherein said at least one non-proteinogenic amino acid comprises at most about 50 atoms. 36. The labeling reagent of any one of embodiments 33-35, wherein said at least one non-proteinogenic amino acid comprises at most about 20 atoms. 37. The labeling reagent of any one of embodiments 33-35, wherein said at least one non-proteinogenic amino acid comprises about 10-20 atoms. 38. The labeling reagent of any one of embodiments 33-37, wherein said at least one non-proteinogenic amino acid comprises cysteic acid. 39. The labeling reagent of any one of embodiments 33-37, wherein said at least one non-proteinogenic amino acid comprises 5-amino-5-carboxy-N,N,N-trimethylpentan-1-aminium or a salt thereof 40. The labeling reagent of any one of embodiments 33-39, wherein said at least one non-proteinogenic amino acid comprises 6-aminohexanoic acid. 41. The labeling reagent of any one of embodiments 33-40, wherein said detectable moiety comprises a fluorescent dye. 42. The labeling reagent of embodiment 41, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, Cy5, or Kam. 43. The labeling reagent of embodiment 42, wherein said fluorescent dye comprises ATTO 633. 44. The labeling reagent of any one of embodiments 33-43, wherein said at least one cleavable group is configured to be cleaved to separate a portion of said detectable moiety from said labeling reagent. 45. The labeling reagent of any one of embodiments 33-44, wherein said at least one cleavable group is selected from the group consisting of an azidomethyl group, a disulfide bond, a hydrocarbyldithiomethyl group, and a 2-nitrobenzyloxy group. 46. The labeling reagent of embodiment 45, wherein said at least one cleavable group is said disulfide bond. 47. The labeling reagent of any one of embodiments 33-46, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof. 48. The labeling reagent of any one of embodiments 33-47, wherein said labeling reagent comprises a moiety selected from the group consisting of

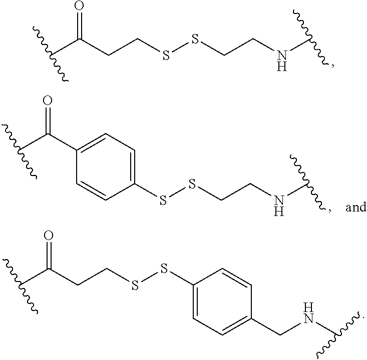

49. A labeling reagent comprising a compound of Formula I:

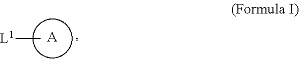

wherein: A is a detectable moiety; and L1 is a linker comprising at least one cleavable group and at least one non-proteinogenic amino acid, wherein said at least one non-proteinogenic amino acid does not comprise hydroxyproline, and wherein when said at least one non-proteinogenic amino acid comprises 6-aminohexanoic acid, said linker is not coupled to

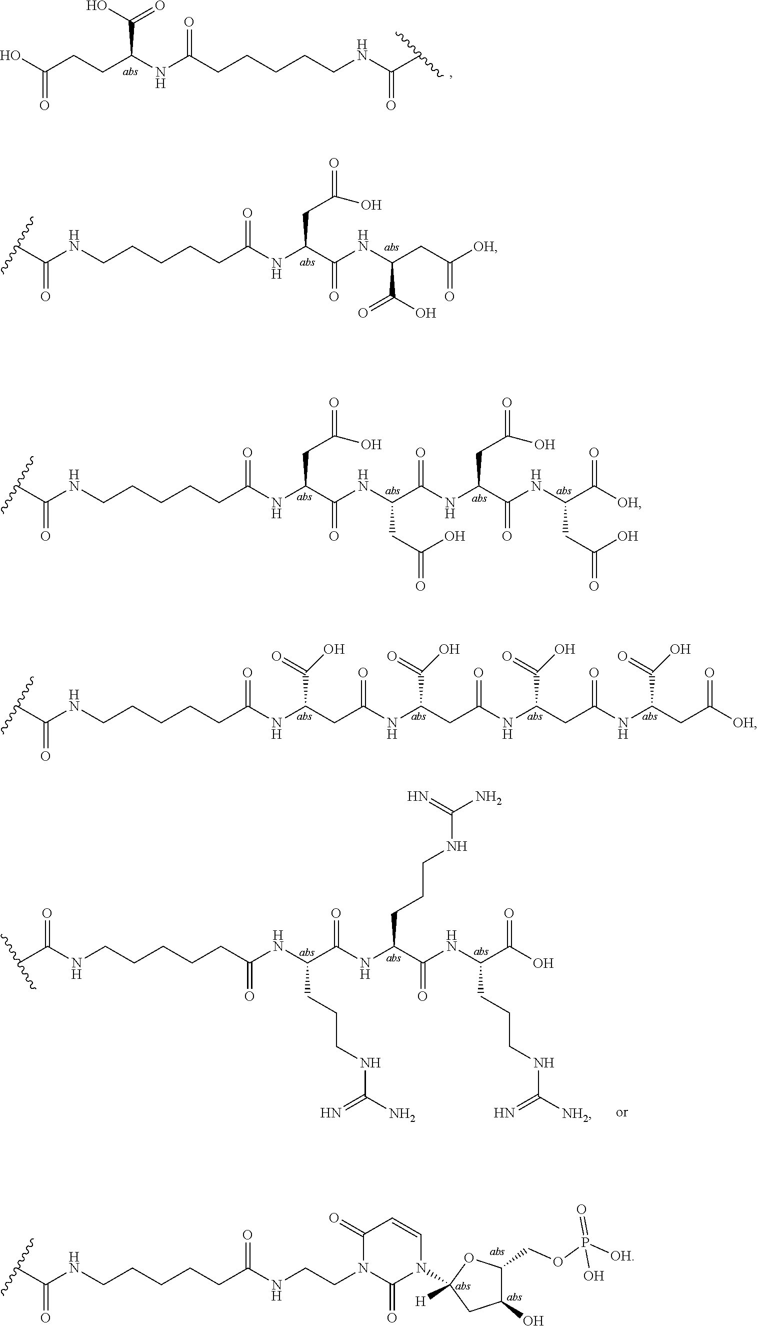 [SMILES: *C(=O)NCCCCCC(=O)N[C@@H](CCC(=O)O)C(=O)O ; *C(=O)NCCCCCC(=O)N[C@@H](CC(=O)O)C(=O)N[C@@H](CC(=O)O)C(=O)O ; *C(=O)NCCCCCC(=O)N[C@@H](CC(=O)O)C(=O)N[C@@H](CC(=O)O)C(=O)N[C@@H](CC(=O)O)C(=O)N[C@@H](CC(=O)O)C(=O)O ; *C(=O)NCCCCCC(=O)N[C@@H](CC(=O)N[C@@H](CC(=O)N[C@@H](CC(=O)N[C@@H](CC(=O)O)C(=O)O)C(=O)O)C(=O)O)C(=O)O ; *C(=O)NCCCCCC(=O)N[C@@H](CCCNC(=N)N)C(=O)N[C@@H](CCCNC(=N)N)C(=O)N[C@@H](CCCNC(=N)N)C(=O)O ; *C(=O)NCCCCCC(=O)NCCn1c(=O)ccn([C@H]2C[C@H](O)[C@@H](COP(=O)(O)O)O2)c1=O]

50. The labeling reagent of embodiment 49, wherein said linker is not coupled to a terminator group. 51. The labeling reagent of embodiment 49 or 50, wherein said at least one non-proteinogenic amino acid comprises at most about 50 atoms. 52. The labeling reagent of any one of embodiments 49-51, wherein said at least one non-proteinogenic amino acid comprises at most about 20 atoms. 53. The labeling reagent of any one of embodiments 49-52, wherein said at least one non-proteinogenic amino acid comprises about 10-20 atoms. 54. The labeling reagent of any one of embodiments 49-53, wherein said at least one non-proteinogenic amino acid comprises cysteic acid. 55. The labeling reagent of any one of embodiments 49-53, wherein said at least one non-proteinogenic amino acid comprises 5-amino-5-carboxy-N,N,N-trimethylpentan-1-aminium or a salt thereof 56. The labeling reagent of any one of embodiments 49-55, wherein said at least one non-proteinogenic amino acid comprises 6-aminohexanoic acid. 57. The labeling reagent of any one of embodiments 49-56, wherein said detectable moiety comprises a fluorescent dye. 58. The labeling reagent of embodiment 57, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, Cy5, or Kam. 59. The labeling reagent of embodiment 58, wherein said fluorescent dye comprises ATTO 633. 60. The labeling reagent of any one of embodiments 49-59, wherein said at least one cleavable group is configured to be cleaved to separate a portion of said detectable moiety from said labeling reagent. 61. The labeling reagent of any one of embodiments 49-60, wherein said at least one cleavable group is selected from the group consisting of an azidomethyl group, a disulfide bond, a hydrocarbyldithiomethyl group, and a 2-nitrobenzyloxy group. 62. The labeling reagent of embodiment 61, wherein said at least one cleavable group is said disulfide bond. 63. The labeling reagent of any one of embodiments 49-62, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof 64. The labeling reagent of any one of embodiments 49-63, wherein said labeling reagent comprises a moiety selected from the group consisting of

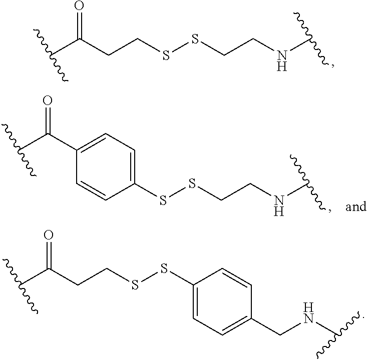

65. A labeling reagent comprising: a) a detectable moiety; and b) a linker that is coupled to said detectable moiety, wherein said linker comprises at least one cleavable group and at least one non-proteinogenic amino acid, wherein said at least one non-proteinogenic amino acid comprises cysteic acid, 5-amino-5-carboxy-N,N,N-trimethylpentan-1-aminium or a salt thereof, or 6-aminohexanoic acid, or a combination thereof, wherein when said at least one non-proteinogenic amino acid comprises said cysteic acid, said linker does not comprise hydroxyproline, and wherein when said at least one non-proteinogenic amino acid comprises said 6-aminohexanoic acid, said detectable moiety is not a Cyanide 5 (Cy5) or an ATTO 647 NHS Ester (ATTO 647N).

66. The labeling reagent of embodiment 65, said detectable moiety does not comprise said Cy5 or said ATTO 647N. 67. The labeling reagent of embodiment 65 or 66, wherein said at least one non-proteinogenic amino acid comprises said cysteic acid. 68. The labeling reagent of any one of embodiments 65-66, wherein said at least one non-proteinogenic amino acid comprises said 5-amino-5-carboxy-N,N,N-trimethylpentan-1-aminium. 69. The labeling reagent of any one of embodiments 65-68, wherein said at least one non-proteinogenic amino acid comprises said 6-aminohexanoic acid. 70. The labeling reagent of any one of embodiments 65-69, wherein said detectable moiety comprises a fluorescent dye. 71. The labeling reagent of embodiment 70, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, or Kam. 72. The labeling reagent of embodiment 71, wherein said fluorescent dye comprises ATTO 633. 73. The labeling reagent of any one of embodiments 65-72, wherein said at least one cleavable group is configured to be cleaved to separate a portion of said detectable moiety from said labeling reagent. 74. The labeling reagent of any one of embodiments 65-73, wherein said at least one cleavable group is selected from the group consisting of an azidomethyl group, a disulfide bond, a hydrocarbyldithiomethyl group, and a 2-nitrobenzyloxy group. 75. The labeling reagent of embodiment 74, wherein said at least one cleavable group is said disulfide bond. 76. The labeling reagent of any one of embodiments 65-75, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof 77. The labeling reagent of any one of embodiments 65-76, wherein said labeling reagent comprises a moiety selected from the group consisting of

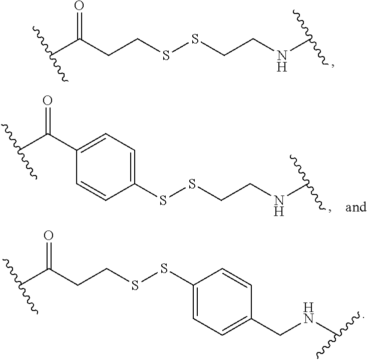

78. A labeling reagent comprising a compound of Formula I:

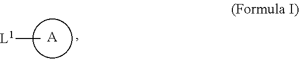

wherein: A is a detectable moiety; and L1 is a linker comprising at least one cleavable group and at least one non-proteinogenic amino acid, wherein said at least one non-proteinogenic amino acid non-proteinogenic amino acid comprises cysteic acid, 5-amino-5-carboxy-N,N,N-trimethylpentan-1-aminium or a salt thereof, or 6-aminohexanoic acid, or a combination thereof, wherein when said at least one non-proteinogenic amino acid comprises said cysteic acid, L1 does not comprise hydroxyproline, and wherein when said at least one non-proteinogenic amino acid comprises said 6-aminohexanoic acid, said detectable moiety is not a Cyanide 5 (Cy5) or an ATTO 647 NHS Ester (ATTO 647N).

79. The labeling reagent of embodiment 78, wherein said detectable moiety does not comprise said Cy5 or said ATTO 647N. 80. The labeling reagent of embodiment 78 or 79, wherein said at least one non-proteinogenic amino acid comprises said cysteic acid. 81. The labeling reagent of embodiment 78 or 79, wherein said at least one non-proteinogenic amino acid comprises said 5-amino-5-carboxy-N,N,N-trimethylpentan-1-aminium. 82. The labeling reagent of any one of embodiments 78-81, wherein said at least one non-proteinogenic amino acid comprises said 6-aminohexanoic acid. 83. The labeling reagent of any one of embodiments 78-82, wherein said detectable moiety comprises a fluorescent dye. 84. The labeling reagent of embodiment 83, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, or Kam. 85. The labeling reagent of embodiment 84, wherein said fluorescent dye comprises ATTO 633. 86. The labeling reagent of any one of embodiments 78-85, wherein said at least one cleavable group is configured to be cleaved to separate a portion of said detectable moiety from said labeling reagent. 87. The labeling reagent of any one of embodiments 78-86, wherein said at least one cleavable group is selected from the group consisting of an azidomethyl group, a disulfide bond, a hydrocarbyldithiomethyl group, and a 2-nitrobenzyloxy group. 88. The labeling reagent of embodiment 87, wherein said at least one cleavable group is said disulfide bond. 89. The labeling reagent of any one of embodiments 78-88, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof 90. The labeling reagent of any one of embodiments 78-89, wherein said labeling reagent comprises a moiety selected from the group consisting of

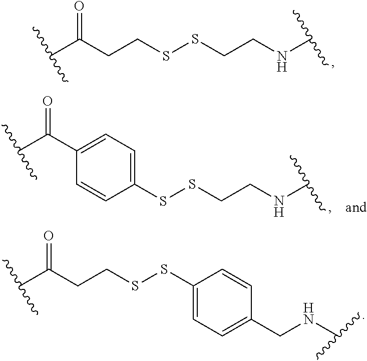

91. A labeling reagent comprising: a) a detectable moiety; and b) a linker that is coupled to said detectable moiety, wherein said linker comprises at least one cleavable group and at least one non-proteinogenic amino acid, wherein said at least one non-proteinogenic amino acid comprises cysteic acid, 5-amino-5-carboxy-N,N,N-trimethylpentan-1-aminium or a salt thereof, or 6-aminohexanoic acid, or a combination thereof, wherein when said at least one non-proteinogenic amino acid comprises said cysteic acid, said linker does not comprise hydroxyproline, and wherein when said at least one non-proteinogenic amino acid comprises said 6-aminohexanoic acid, said linker is not coupled to

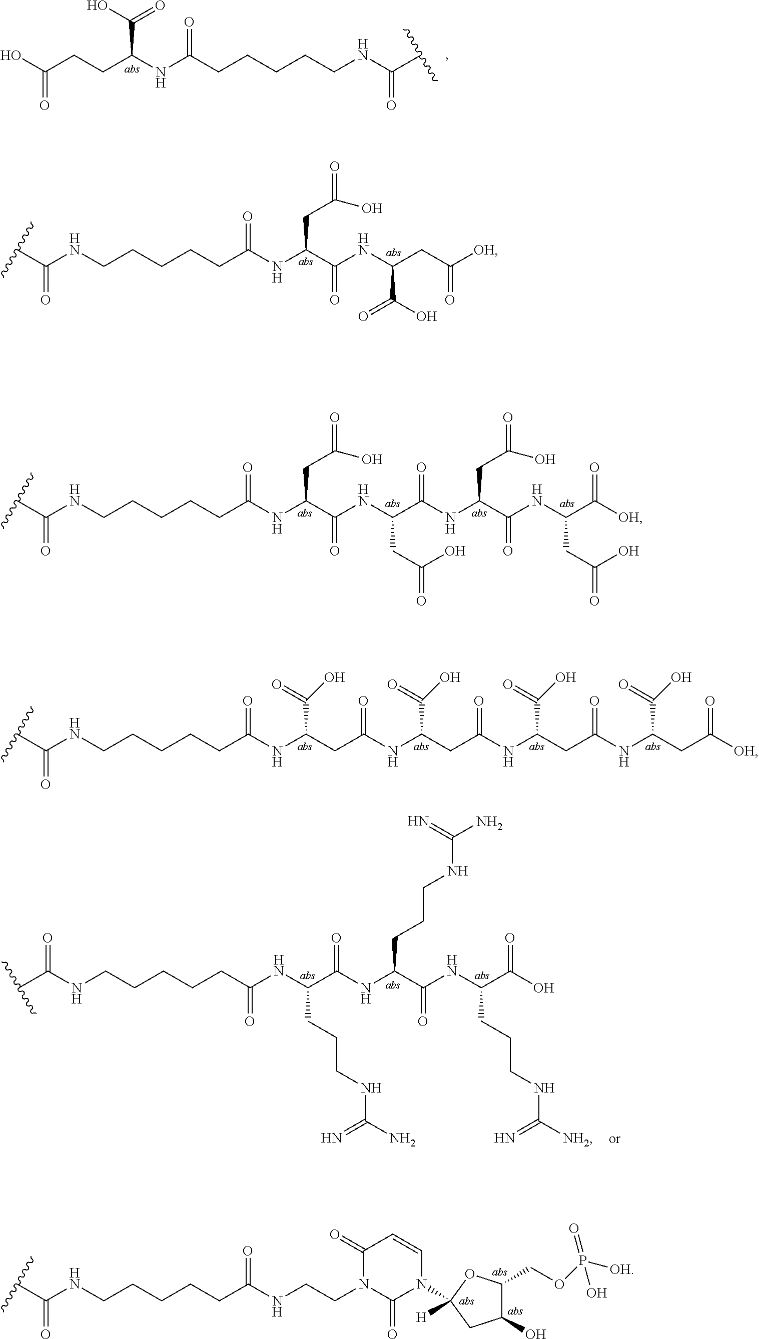 [SMILES: *C(=O)NCCCCCC(=O)N[C@@H](CCC(=O)O)C(=O)O ; *C(=O)NCCCCCC(=O)N[C@@H](CC(=O)O)C(=O)N[C@@H](CC(=O)O)C(=O)O ; *C(=O)NCCCCCC(=O)N[C@@H](CC(=O)O)C(=O)N[C@@H](CC(=O)O)C(=O)N[C@@H](CC(=O)O)C(=O)N[C@@H](CC(=O)O)C(=O)O ; *C(=O)NCCCCCC(=O)N[C@@H](CC(=O)N[C@@H](CC(=O)N[C@@H](CC(=O)N[C@@H](CC(=O)O)C(=O)O)C(=O)O)C(=O)O)C(=O)O ; *C(=O)NCCCCCC(=O)N[C@@H](CCCNC(=N)N)C(=O)N[C@@H](CCCNC(=N)N)C(=O)N[C@@H](CCCNC(=N)N)C(=O)O ; *C(=O)NCCCCCC(=O)NCCn1c(=O)ccn([C@H]2C[C@H](O)[C@@H](COP(=O)(O)O)O2)c1=O]

92. The labeling reagent of embodiment 91, wherein said linker is not coupled to a terminator group. 93. The labeling reagent of embodiment 91 or 92, wherein said at least one non-proteinogenic amino acid comprises said cysteic acid. 94. The labeling reagent of embodiment 91 or 92, wherein said at least one non-proteinogenic amino acid comprises said 5-amino-5-carboxy-N,N,N-trimethylpentan-1-aminium. 95. The labeling reagent of any one of embodiments 91-94, wherein said at least one non-proteinogenic amino acid comprises said 6-aminohexanoic acid. 96. The labeling reagent of any one of embodiments 91-95, wherein said detectable moiety comprises a fluorescent dye. 97. The labeling reagent of embodiment 96, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, Cy5, or Kam. 98. The labeling reagent of embodiment 97, wherein said fluorescent dye comprises ATTO 633. 99. The labeling reagent of any one of embodiments 91-98, wherein said at least one cleavable group is configured to be cleaved to separate a portion of said detectable moiety from said labeling reagent. 100. The labeling reagent of any one of embodiments 91-99, wherein said at least one cleavable group is selected from the group consisting of an azidomethyl group, a disulfide bond, a hydrocarbyldithiomethyl group, and a 2-nitrobenzyloxy group. 101.

The labeling reagent of embodiment 100, wherein said at least one cleavable group is said disulfide bond. 102. The labeling reagent of any one of embodiments 91-101, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof. 103. The labeling reagent of any one of embodiments 91-102, wherein said labeling reagent comprises a moiety selected from the group consisting of

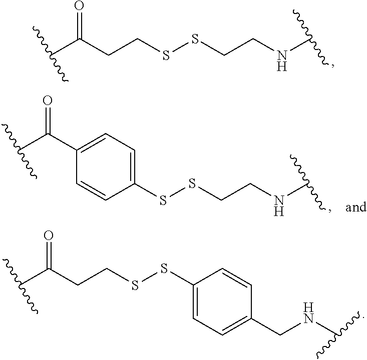

104. A labeling reagent comprising a compound of Formula I:

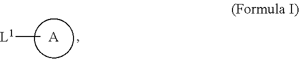

wherein: A is a detectable moiety; and L1 is a linker comprising at least one cleavable group and at least one non-proteinogenic amino acid, wherein said at least one non-proteinogenic amino acid comprises cysteic acid, 5-amino-5-carboxy-N,N,N-trimethylpentan-1-aminium, or 6-aminohexanoic acid, or a combination thereof, wherein when said at least one non-proteinogenic amino acid comprises said cysteic acid, L1 does not comprise hydroxyproline, and wherein when said at least one non-proteinogenic acid comprises said 6-aminohexanoic acid, L1 is not coupled to

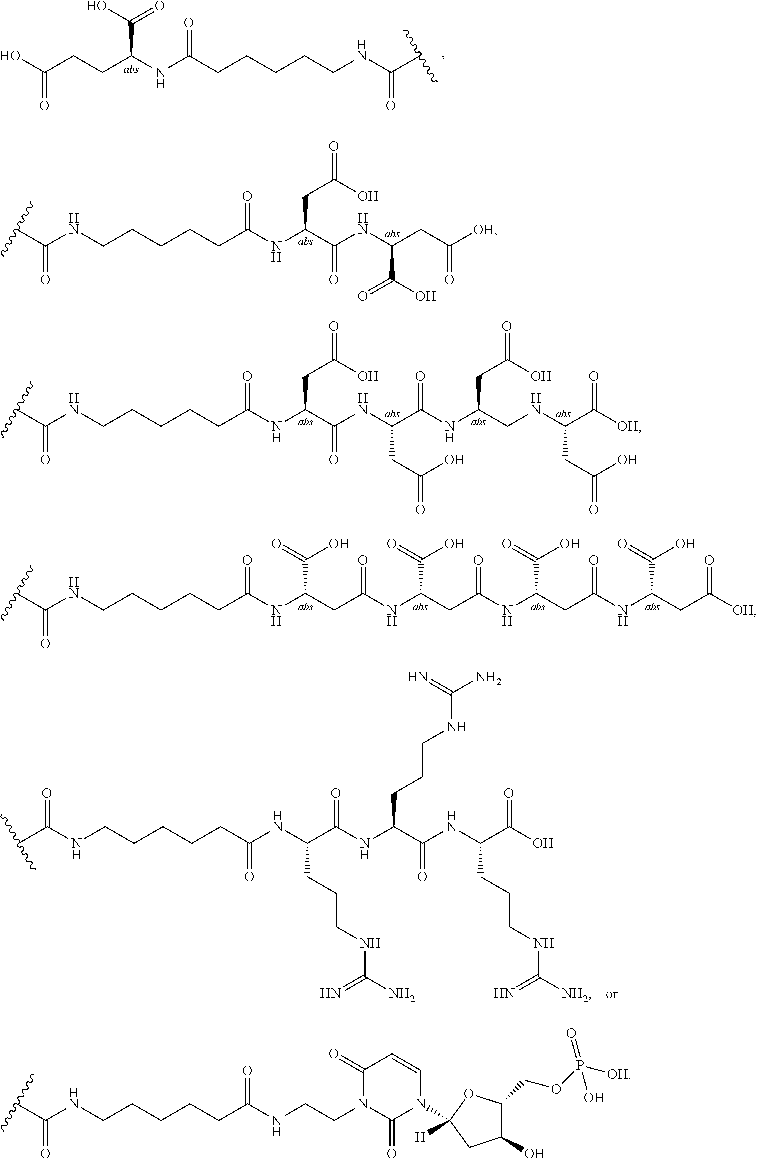

105. The labeling reagent of embodiment 104, wherein L1 is not coupled to a terminator group. 106. The labeling reagent of embodiment 104 or 105, wherein said at least one non-proteinogenic amino acid comprises said cysteic acid. 107. The labeling reagent of embodiment 104 or 105, wherein said at least one non-proteinogenic amino acid comprises said 5-amino-5-carboxy-N,N,N-trimethylpentan-1-aminium. 108. The labeling reagent of any one of embodiments 104-107, wherein said at least one non-proteinogenic amino acid comprises said 6-aminohexanoic acid. 109. The labeling reagent of any one of embodiments 104-108, wherein said detectable moiety comprises a fluorescent dye. 110. The labeling reagent of embodiment 109, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, Cy5, or Kam. 111. The labeling reagent of embodiment 110, wherein said fluorescent dye comprises ATTO 633. 112. The labeling reagent of any one of embodiments 104-111, wherein said at least one cleavable group is configured to be cleaved to separate a portion of said detectable moiety from said labeling reagent. 113. The labeling reagent of any one of embodiments 104-112, wherein said at least one cleavable group is selected from the group consisting of an azidomethyl group, a disulfide bond, a hydrocarbyldithiomethyl group, and a 2-nitrobenzyloxy group. 114. The labeling reagent of embodiment 113, wherein said at least one cleavable group is said disulfide bond. 115. The labeling reagent of any one of embodiments 104-114, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof. 116. The labeling reagent of any one of embodiments 104-115, wherein said labeling reagent comprises a moiety selected from the group consisting of

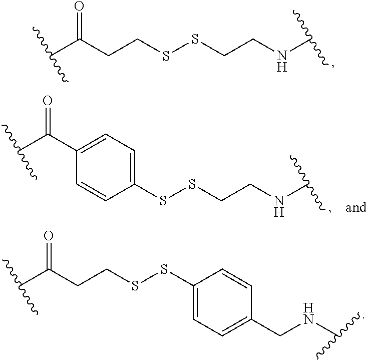

117. A detectably labeled substrate comprising a compound of any one of embodiments 17, 49, 78, and 104, wherein the compound is a compound of Formula Ia:

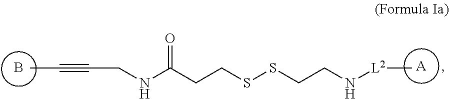

wherein: B is a substrate, A is the detectable moiety, and L2 comprises said at least one non-proteinogenic amino acid.

118. A detectably labeled substrate comprising: a) a detectable moiety; b) a linker that is coupled to said detectable moiety, wherein said linker comprises at least one non-proteinogenic amino acid, wherein said at least one non-proteinogenic amino acid comprises cysteic acid, 5-amino-5-carboxy-N,N,N-trimethylpentan-1-aminium or a salt thereof, or 6-aminohexanoic acid, or a combination thereof, and wherein when said at least one non-proteinogenic amino acid comprises said 6-aminohexanoic acid, said detectable moiety is not a Cyanide 5 (Cy5) or an ATTO 647 NHS Ester (ATTO 647N); and c) a substrate comprising a nucleobase, wherein said substrate is coupled to said linker, and wherein said nucleobase does not comprise guanine.

119. The detectably labeled substrate of embodiment 118, wherein said nucleobase is adenine. 120. The detectably labeled substrate of embodiment 118, wherein said nucleobase is cytosine. 121. The detectably labeled substrate of embodiment 118, wherein said nucleobase is thymine. 122. The detectably labeled substrate of embodiment 118, wherein said nucleobase is uracil. 123. The detectably labeled substrate of any one of embodiments 118-122, wherein said detectable moiety does not comprise said Cy5 or said ATTO 647N. 124. The detectably labeled substrate of any one of embodiments 118-123, wherein said linker comprises at least one cleavable group. 125. The detectably labeled substrate of any one of embodiments 118-124, wherein said at least one non-proteinogenic amino acid comprises said cysteic acid. 126. The detectably labeled substrate of any one of embodiments 118-124, wherein said at least one non-proteinogenic amino acid comprises said 5-amino-5-carboxy-N,N,N-trimethylpentan-1-aminium. 127. The detectably labeled substrate of any one of embodiments 118-126, wherein said at least one non-proteinogenic amino acid comprises said 6-aminohexanoic acid. 128. The detectably labeled substrate of any one of embodiments 118-127, wherein said detectable moiety comprises a fluorescent dye. 129. The detectably labeled substrate of embodiment 128, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, or Kam. 130. The detectably labeled substrate of embodiment 129, wherein said fluorescent dye comprises ATTO 633. 131. The detectably labeled substrate of any one of embodiments 124-130, wherein said at least one cleavable group is configured to be cleaved to separate a portion of said detectable moiety from said detectably labeled substrate. 132. The detectably labeled substrate of any one of embodiments 124-131, wherein said at least one cleavable group is selected from the group consisting of an azidomethyl group, a disulfide bond, a hydrocarbyldithiomethyl group, and a 2-nitrobenzyloxy group. 133. The detectably labeled substrate of embodiment 132, wherein said at least one cleavable group is said disulfide bond. 134. The detectably labeled substrate of any one of embodiments 124-133, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof. 135. The detectably labeled substrate of any one of embodiments 118-134, wherein said detectably labeled substrate comprises a moiety selected from the group consisting of

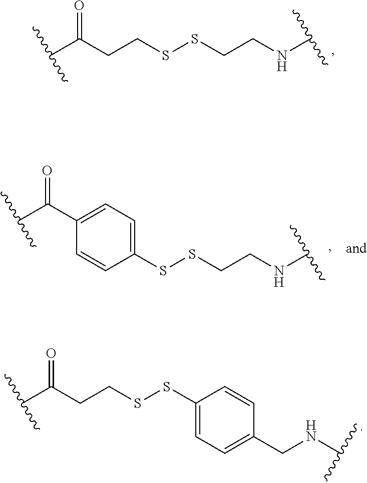 [SMILES: *NCCSSCCC(*)=O ; *NCCSSc1ccc(C(*)=O)cc1 ; *NCc1ccc(SSCCC(*)=O)cc1]

136. A detectably labeled substrate comprising a compound of Formula II:

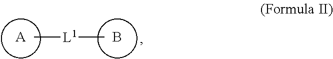

wherein: A comprises a nucleobase, wherein said nucleobase is not guanine; B is a detectable moiety; and L1 is a linker comprising at least one non-proteinogenic amino acid, wherein said at least one non-proteinogenic amino acid comprises cysteic acid, 5-amino-5-carboxy-N,N,N-trimethylpentan-1-aminium or a salt thereof, or 6-aminohexanoic acid, or a combination thereof, and wherein when said at least one non-proteinogenic amino acid comprises said 6-aminohexanoic acid, said detectable moiety is not a Cyanide 5 (Cy5) or an ATTO 647 NHS Ester (ATTO 647N).

137. The detectably labeled substrate of embodiment 136, wherein said nucleobase is adenine. 138. The detectably labeled substrate of embodiment 136, wherein said nucleobase is cytosine. 139. The detectably labeled substrate of embodiment 136, wherein said nucleobase is thymine. 140. The detectably labeled substrate of embodiment 136, wherein said nucleobase is uracil. 141. The detectably labeled substrate of any one of embodiments 136-140, wherein said detectable moiety does not comprise said Cy5 or said ATTO 647N. 142. The detectably labeled substrate of any one of embodiments 136-141, wherein said linker comprises at least one cleavable group. 143. The detectably labeled substrate of any one of embodiments 136-142, wherein said at least one non-proteinogenic amino acid comprises said cysteic acid. 144. The detectably labeled substrate of any one of embodiments 136-142, wherein said at least one non-proteinogenic amino acid comprises said 5-amino-5-carboxy-N,N,N-trimethylpentan-1-aminium. 145. The detectably labeled substrate of any one of embodiments 136-144, wherein said at least one non-proteinogenic amino acid comprises said 6-aminohexanoic acid. 146. The detectably labeled substrate of any one of embodiments 136-145, wherein said detectable moiety comprises a fluorescent dye. 147. The detectably labeled substrate of embodiment 146, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, or Kam. 148. The detectably labeled substrate of embodiment 147, wherein said fluorescent dye comprises ATTO 633. 149. The detectably labeled substrate of any one of embodiments 136-148, wherein said at least one cleavable group is configured to be cleaved to separate a portion of said detectable moiety from said detectably labeled substrate. 150. The detectably labeled substrate of any one of embodiments 136-149, wherein said at least one cleavable group is selected from the group consisting of an azidomethyl group, a disulfide bond, a hydrocarbyldithiomethyl group, and a 2-nitrobenzyloxy group. 151. The detectably labeled substrate of embodiment 150, wherein said at least one cleavable group is said disulfide bond. 152. The detectably labeled substrate of any one of embodiments 136-151, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof. 153. The detectably labeled substrate of any one of embodiments 135-152, wherein said detectably labeled substrate comprises a moiety selected from the group consisting of

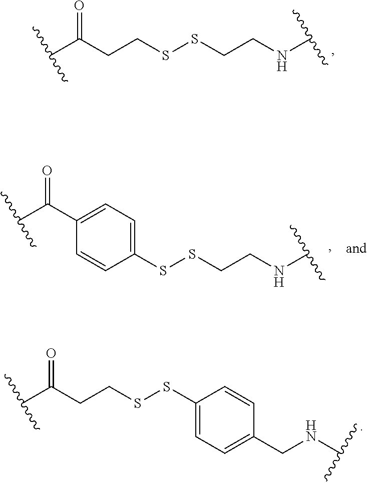 [SMILES: *NCCSSCCC(*)=O ; *NCCSSc1ccc(C(*)=O)cc1 ; *NCc1ccc(SSCCC(*)=O)cc1]

154. The detectably labeled substrate of any one of embodiments 136-153, wherein said detectably labeled substrate comprises a compound of Formula IIa:

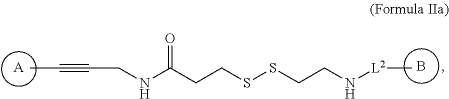

wherein: A is a deoxyribose nucleotide triphosphate; B is a detectable moiety; and L2 comprises said at least one non-proteinogenic amino acid. 155. The detectably labeled substrate of embodiment 154, wherein said detectably labeled substrate is a compound of Formula IIb, Formula IIc, Formula IId, Formula IIe, Formula IIf, or Formula IIg:

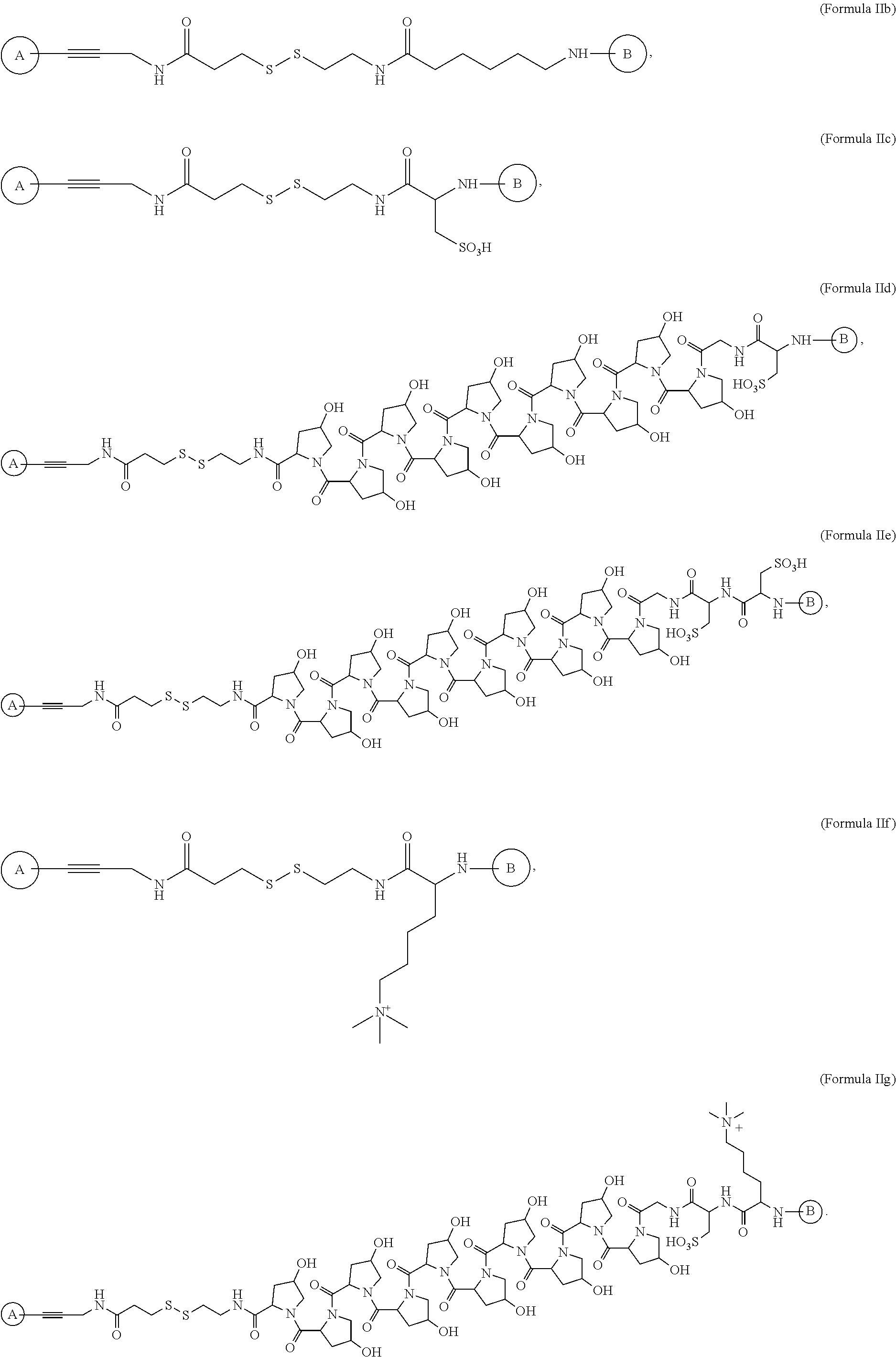

156. A substrate comprising: a) a nucleobase, wherein said nucleobase is not a guanine; and b) a linker coupled to said nucleobase, wherein said linker comprises at least a first non-proteinogenic amino acid and at least a second non-proteinogenic amino acid, wherein said first non-proteinogenic amino acid and said second non-proteinogenic amino acid are different. 157. The substrate of embodiment 156, wherein said first non-proteinogenic amino acid comprises hydroxyproline. 158. The substrate of embodiment 157, wherein said first non-proteinogenic amino acid comprises at least about 5 hydroxyprolines. 159. The substrate of embodiment 158, wherein said first non-proteinogenic amino acid comprises at least about 10 hydroxyprolines. 160. The substrate of embodiment 159, wherein said first non-proteinogenic amino acid comprises about 10 to about 20 hydroxyprolines. 161. The substrate of embodiment 156, wherein said second non-proteinogenic amino acid comprises cysteic acid, 5-amino-5-carboxy-N,N,N-trimethylpentan-1-aminium or a salt thereof, or 6-aminohexanoic acid, or a combination thereof. 162. The substrate of embodiment 161, wherein said second non-proteinogenic amino acid comprises said cysteic acid. 163. The substrate of embodiment 161, wherein said second non-proteinogenic amino acid comprises said 5-amino-5-carboxy-N,N,N-trimethylpentan-1-aminium. 164. The substrate of embodiment 162 or embodiment 163, wherein said second non-proteinogenic amino acid comprises said 6-aminohexanoic acid. 165. The substrate of embodiment 156, wherein said first non-proteinogenic amino acid comprises hydroxyproline and said second non-proteinogenic amino acid comprises cysteic acid. 166. The substrate of embodiment 156, wherein said first non-proteinogenic amino acid comprises hydroxyproline and said second non-proteinogenic amino acid comprises 5-amino-5-carboxy-N,N,N-trimethylpentan-1-aminium. 167. The substrate of embodiment 156, wherein said first non-proteinogenic amino acid comprises hydroxyproline and said second non-proteinogenic amino acid comprises 6-aminohexanoic acid. 168. The substrate of any one of embodiments 165-167, wherein said first non-proteinogenic amino acid comprises at least about 10 hydroxyprolines. 169. The substrate of embodiment 168, wherein said first non-proteinogenic amino acid comprises about 10 to about 20 hydroxyprolines. 170. The substrate of any one of embodiments 156-169, wherein said nucleobase comprises adenine, cytosine, thymine, or uracil. 171. The substrate of embodiment 170, wherein said nucleobase is adenine. 172. The substrate of embodiment 170, wherein said nucleobase is cytosine. 173. The substrate of embodiment 170, wherein said nucleobase is thymine. 174. The substrate of embodiment 170, wherein said nucleobase is uracil. 175. The substrate of any one of embodiments 156-174, wherein said linker comprises at least one cleavable group. 176. The substrate of embodiment 175, wherein said linker comprises one said cleavable group. 177. The substrate of any one of embodiments 156-176, wherein said detectable moiety comprises at least one fluorescent dye. 178. The substrate of embodiment 177, wherein said detectable moiety comprises one said fluorescent dye. 179. The substrate of embodiment 178, wherein said fluorescent dye comprises ATTO 633, ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, Cy5, or Kam. 180. The substrate of embodiment 179, wherein said fluorescent dye comprises ATTO 633. 181. The substrate of any one of embodiments 156-180, wherein said at least one cleavable group is configured to be cleaved to separate a portion of said detectable moiety from said substrate. 182. The substrate of any one of embodiments 156-181, wherein said at least one cleavable group is selected from the group consisting of an azidomethyl group, a disulfide bond, a hydrocarbyldithiomethyl group, and a 2-nitrobenzyloxy group. 183. The substrate of embodiment 182, wherein said at least one cleavable group is said disulfide bond. 184. The substrate of any one of embodiments 156-183, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof 185. The substrate of any one of embodiments 156-184, wherein said substrate comprises a moiety selected from the group consisting of

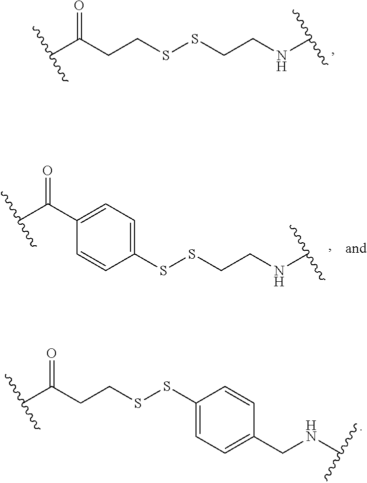 [SMILES: *NCCSSCCC(*)=O ; *NCCSSc1ccc(C(*)=O)cc1 ; *NCc1ccc(SSCCC(*)=O)cc1]

186. A substrate comprising a compound of Formula III:

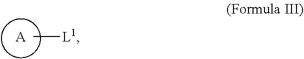

wherein: A comprises a nucleobase; and L1 is a linker comprising at least a first non-proteinogenic amino acid and at least a second non-proteinogenic amino acid, wherein said nucleobase is not a guanine, and wherein said first non-proteinogenic amino acid and said second non-proteinogenic amino acid are different.

187. The substrate of embodiment 186, wherein said first non-proteinogenic amino acid comprises hydroxyproline. 188. The substrate of embodiment 187, wherein said first non-proteinogenic amino acid comprises at least about 5 hydroxyprolines. 189. The substrate of embodiment 188, wherein said first non-proteinogenic amino acid comprises at least about 10 hydroxyprolines. 190. The substrate of embodiment 189, wherein said first non-proteinogenic amino acid comprises about 10 to about 20 hydroxyprolines. 191. The substrate of embodiment 186, wherein said second non-proteinogenic amino acid comprises cysteic acid, 5-amino-5-carboxy-N,N,N-trimethylpentan-1-aminium or a salt thereof, or 6-aminohexanoic acid, or a combination thereof. 192. The substrate of embodiment 191, wherein said second non-proteinogenic amino acid comprises said cysteic acid. 193. The substrate of embodiment 191, wherein said second non-proteinogenic amino acid comprises said 5-amino-5-carboxy-N,N,N-trimethylpentan-1-aminium. 194. The substrate of embodiment 191, wherein said second non-proteinogenic amino acid comprises said 6-aminohexanoic acid. 195. The substrate of embodiment 186, wherein said first non-proteinogenic amino acid comprises hydroxyproline and said second non-proteinogenic amino acid comprises cysteic acid. 196. The substrate of embodiment 186, wherein said first non-proteinogenic amino acid comprises hydroxyproline and said second non-proteinogenic amino acid comprises 5-amino-5-carboxy-N,N,N-trimethylpentan-1-aminium. 197. The substrate of embodiment 186, wherein said first non-proteinogenic amino acid comprises hydroxyproline and said second non-proteinogenic amino acid comprises 6-aminohexanoic acid. 198. The substrate of any one of embodiments 195-197, wherein said first non-proteinogenic amino acid comprises at least about 10 hydroxyprolines. 199. The substrate of embodiment 198, wherein said first non-proteinogenic amino acid comprises about 10 to about 20 hydroxyprolines. 200. The substrate of any one of embodiments 186-199, wherein said nucleobase comprises adenine, cytosine, thymine, or uracil. 201. The substrate of embodiment 200, wherein said nucleobase is adenine. 202. The substrate of embodiment 200, wherein said nucleobase is cytosine. 203. The substrate of embodiment 200, wherein said nucleobase is thymine. 204. The substrate of embodiment 200, wherein said nucleobase is uracil. 205. The substrate of any one of embodiments 186-204, wherein said linker comprises at least one cleavable group. 206. The substrate of embodiment 205, wherein said linker comprises one said cleavable group. 207. The substrate of any one of embodiments 186-206, detectable moiety comprises at least one fluorescent dye. 208. The substrate of embodiment 207, wherein said detectable moiety comprises one said fluorescent dye. 209. The substrate of embodiment 208, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, UG 524.2, UG 524. 1, UG_dye105, UG_dye106, KK1119, KK9046, Abberior STAR635, Abberior STAR635P, Cy5, or Kam. 210. The substrate of embodiment 209, wherein said fluorescent dye comprises ATTO 633. 211. The substrate of any one of embodiments 205-210, wherein said at least one cleavable group is configured to be cleaved to separate a portion of said detectable moiety from said substrate. 212. The substrate of any one of embodiments 205-211, wherein said at least one cleavable group is selected from the group consisting of an azidomethyl group, a disulfide bond, a hydrocarbyldithiomethyl group, and a 2-nitrobenzyloxy group. 213. The substrate of embodiment 212, wherein said at least one cleavable group is said disulfide bond. 214. The substrate of any one of embodiments 205-213, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof 215. The substrate of any one of embodiments 186-214, wherein said substrate comprises a moiety selected from the group consisting of

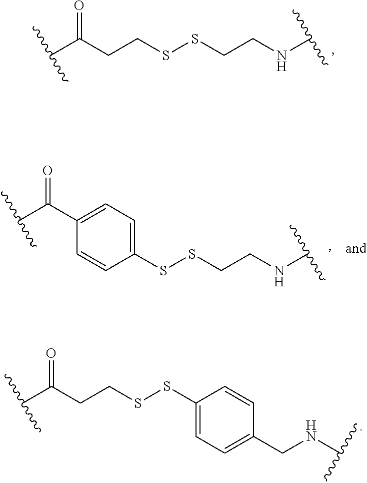 [SMILES: *NCCSSCCC(*)=O ; *NCCSSc1ccc(C(*)=O)cc1 ; *NCc1ccc(SSCCC(*)=O)cc1]

216. A detectably labeled substrate comprising the substrate of any one of embodiments 186-215, wherein said detectably labeled substrate comprises a compound of Formula IIIa:

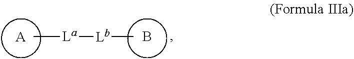

wherein: A comprises said nucleobase; B comprises a detectable moiety; La is a first linker; and Lb is a second linker.

217. The detectably labeled substrate of embodiment 216, wherein Lb comprises said first non-proteinogenic amino acid or said second non-proteinogenic amino acid. 218. The detectably labeled substrate of embodiment 216, wherein Lb comprises said first non-proteinogenic amino acid and said second non-proteinogenic amino acid. 219. The detectably labeled substrate of any one of embodiments 216-218, wherein La comprises at least one cleavable group. 220. The detectably labeled substrate of any one of embodiments 216-219, wherein said detectably labeled substrate comprises a compound of Formula IIIb or a compound of Formula IIIc:

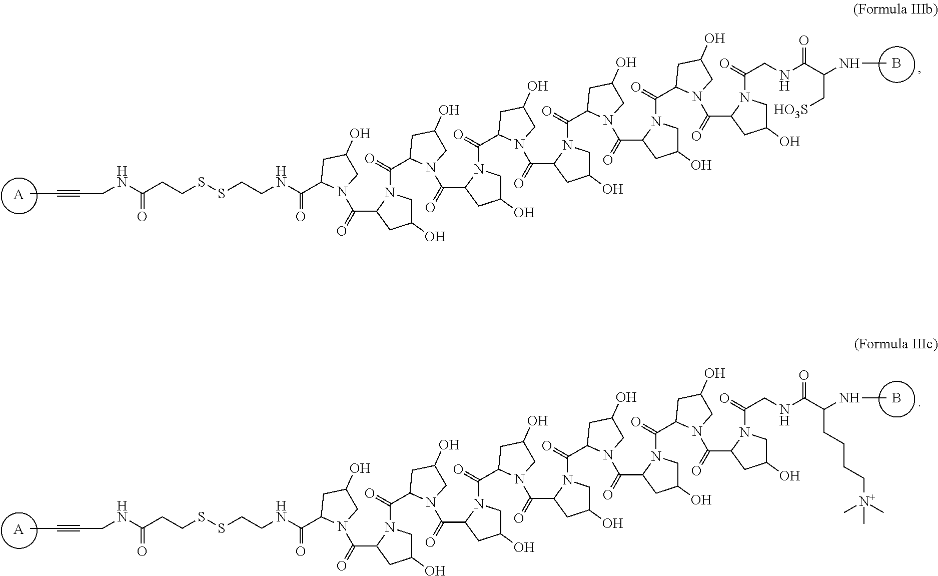

221. A substrate comprising: a) a nucleobase wherein said nucleobase is not a guanine; and b) a linker coupled to said nucleobase, wherein said linker comprises at least two non-proteinogenic amino acids, wherein said at least two non-proteinogenic amino acids are a same type.

222. The substrate of embodiment 221, wherein said at least two non-proteinogenic amino acids are cysteic acids. 223. The substrate of embodiment 221, wherein said at least two non-proteinogenic amino acids are 5-amino-5-carboxy-N,N,N-trimethylpentan-1-aminiums. 224. The substrate of any one of embodiments 221-223, further comprising a third non-proteinogenic amino acid different from said at least two non-proteinogenic amino acids. 225. The substrate of embodiment 224, wherein said third non-proteinogenic amino acid comprises hydroxyproline. 226. The substrate of embodiment 225, wherein said third non-proteinogenic amino acid comprises at least about 5 hydroxyprolines. 227. The substrate of embodiment 226, wherein said third non-proteinogenic amino acid comprises at least about 10 hydroxyprolines. 228. The substrate of embodiment 227, wherein said third non-proteinogenic amino acid comprises about 10 to about 20 hydroxyprolines. 229. The substrate of any one of embodiments 221-228, wherein said nucleobase comprises adenine, cytosine, thymine, or uracil. 230. The substrate of embodiment 229, wherein said nucleobase is said adenine. 231. The substrate of embodiment 229, wherein said nucleobase is said cytosine. 232. The substrate of embodiment 229, wherein said nucleobase is said thymine. 233. The substrate of embodiment 229, wherein said nucleobase is said uracil. 234. The substrate of any one of embodiments 221-233, wherein said linker comprises at least one cleavable group. 235. The substrate of embodiment 234, wherein said linker comprises one of said cleavable group. 236. The substrate of embodiment 221, wherein said at least two non-proteinogenic amino acids comprise 6-aminohexanoic acid. 237. The substrate of any one of embodiments 221-236, wherein said detectable moiety comprises a fluorescent dye. 238. The substrate of embodiment 237, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, Cy5, or Kam. 239. The substrate of embodiment 238, wherein said fluorescent dye comprises ATTO 633. 240. The substrate of any one of embodiments 221-239, wherein said at least one cleavable group is configured to be cleaved to separate a portion of said detectable moiety from said substrate. 241. The substrate of any one of embodiments 221-240, wherein said at least one cleavable group is selected from the group consisting of an azidomethyl group, a disulfide bond, a hydrocarbyldithiomethyl group, and a 2-nitrobenzyloxy group. 242. The substrate of embodiment 241, wherein said at least one cleavable group is said disulfide bond. 243. The substrate of any one of embodiments 221-242, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof 244. The substrate of any one of embodiments 221-243, wherein said substrate comprises a moiety selected from the group consisting of

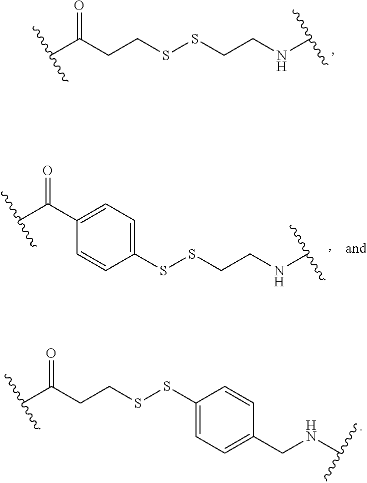 [SMILES: *NCCSSCCC(*)=O ; *NCCSSc1ccc(C(*)=O)cc1 ; *NCc1ccc(SSCCC(*)=O)cc1]

245. A substrate comprising a compound of Formula IV:

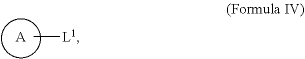

wherein: A comprises a nucleobase, wherein said nucleobase is not a guanine; and L1 is a linker comprising at least two non-proteinogenic amino acids, wherein said at least two non-proteinogenic amino acids are a same type.

246. The substrate of embodiment 245, wherein said at least two non-proteinogenic amino acids are cysteic acids. 247. The substrate of embodiment 245, wherein said at least two non-proteinogenic amino acids are 5-amino-5-carboxy-N,N,N-trimethylpentan-1-aminiums. 248. The substrate of any one of embodiments 245-247, further comprising a third non-proteinogenic amino acid different from said at least two non-proteinogenic amino acids. 249. The substrate of embodiment 248, wherein said third non-proteinogenic amino acid comprises hydroxyproline. 250. The substrate of embodiment 249, wherein said third non-proteinogenic amino acid comprises at least about 5 hydroxyprolines. 251. The substrate of embodiment 250, wherein said third non-proteinogenic amino acid comprises at least about 10 hydroxyprolines. 252. The substrate of embodiment 251, wherein said third non-proteinogenic amino acid comprises about 10 to about 20 hydroxyprolines. 253. The substrate of any one of embodiments 245-252, wherein said nucleobase comprises adenine, cytosine, thymine, or uracil. 254. The substrate of embodiment 253, wherein said nucleobase is said adenine. 255. The substrate of embodiment 253, wherein said nucleobase is said cytosine. 256. The substrate of embodiment 253, wherein said nucleobase is said thymine. 257. The substrate of embodiment 253, wherein said nucleobase is said uracil. 258. The substrate of any one of embodiments 245-257, wherein said linker comprises at least one cleavable group. 259. The substrate of embodiment 258, wherein said linker comprises one said cleavable group. 260. The substrate of any one of embodiments 245-259, wherein said at least two non-proteinogenic amino acids comprise 6-aminohexanoic acid. 261. The substrate of any one of embodiments 245-260, wherein said detectable moiety comprises a fluorescent dye. 262. The substrate of embodiment 261, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, Cy5, or Kam. 263.

The substrate of embodiment 262, wherein said fluorescent dye comprises ATTO 633. 264. The substrate of any one of embodiments 245-263, wherein said at least one cleavable group is configured to be cleaved to separate a portion of said detectable moiety from said substrate. 265. The substrate of any one of embodiments 245-264, wherein said at least one cleavable group is selected from the group consisting of an azidomethyl group, a disulfide bond, a hydrocarbyldithiomethyl group, and a 2-nitrobenzyloxy group. 266. The substrate of embodiment 265, wherein said at least one cleavable group is said disulfide bond. 267. The substrate of any one of embodiments 245-266, wherein said at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof 268. The substrate of any one of embodiments 245-267, wherein said substrate comprises a moiety selected from the group consisting of

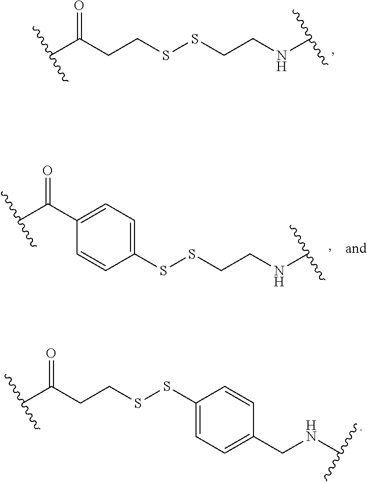 [SMILES: *NCCSSCCC(*)=O ; *NCCSSc1ccc(C(*)=O)cc1 ; *NCc1ccc(SSCCC(*)=O)cc1]

269. A detectably labeled substrate comprising the substrate of any one of embodiments 245-268, wherein said detectably labeled substrate is a compound of Formula IVa:

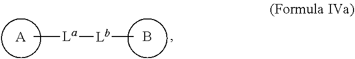

wherein: A comprises said nucleobase, wherein said nucleobase is not a guanine; B comprises a detectable moiety; La is a first linker; and Lb is a second linker.

270. The detectably labeled substrate of embodiment 269, wherein Lb comprises said at least two non-proteinogenic amino acids. 271. The detectably labeled substrate of embodiment 269 or embodiment 270, wherein Lb comprises said cysteic acid. 272. The detectably labeled substrate of embodiment 269 or embodiment 270, wherein Lb comprises said 5-amino-5-carboxy-N,N,N-trimethylpentan-1-aminium. 273. The detectably labeled substrate of any one of embodiments 269-272, wherein Lb comprises a third non-proteinogenic amino acid. 274. The detectably labeled substrate of any one of embodiments 269-273, wherein La comprises at least one cleavable group. 275. The detectably labeled substrate of any one of embodiments 269-274, wherein said detectably labeled substrate is a compound of Formula IVc or a compound of Formula IVd:

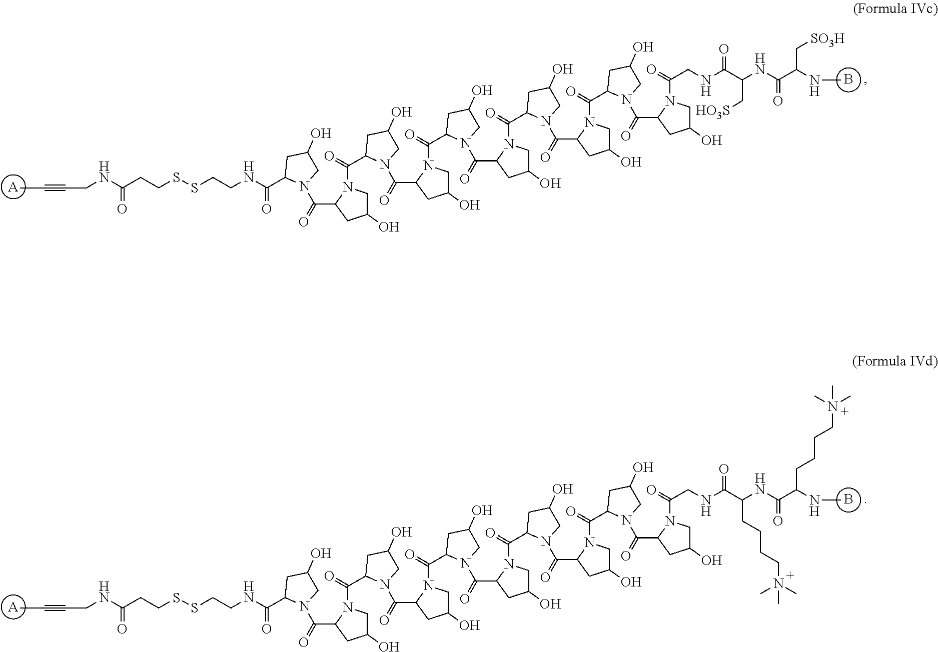

276. A detectably labeled substrate, comprising the labeling reagent of any one of embodiments 1-116 coupled to a substrate. 277. A detectably labeled substrate, comprising the substrate of any one of embodiments 156-215 and 216-261 coupled to a labeling reagent. 278. The detectably labeled substrate of any one of embodiments 117-155, 216-220, and 269-277, wherein said detectably labeled substrate comprises a nucleotide. 279. The detectably labeled substrate of embodiment 278, wherein said labeling reagent is coupled to said detectably labeled substrate via a nucleobase of said nucleotide. 280. A composition comprising a solution comprising a plurality of said detectably labeled substrates of embodiment 278 or embodiment 279. 281. The composition of embodiment 280, wherein said solution further comprises a plurality of unlabeled substrates, wherein each substrate of said plurality of unlabeled substrates is of a same type as each said substrate of said plurality of said detectably labeled substrates. 282. The composition of embodiment 281, wherein a ratio of said plurality of said detectably labeled substrates to said plurality of unlabeled substrates in said solution is at least about 10:1. 283. The composition of embodiment 282, wherein said ratio is at least about 5:1. 284. The composition of embodiment 283, wherein said ratio is at least about 3:1.

285. A labeled substrate, comprising: a substrate; a linker; and a plurality of dye moieties attached to the substrate via the linker, wherein the linker comprises a cleavable portion and a poly-hydroxyproline portion, wherein the poly-hydroxyproline portion comprises a first hydroxyproline portion comprising a first one or more hydroxyproline residues, a set of hydroxyproline or amino-proline residues that attach to the plurality of dye moieties, and one or more second hydroxyproline portions each comprising a second one or more hydroxyproline residues, each of the one or more second hydroxyproline portions disposed between different hydroxyproline or amino-proline residues of the set of hydroxyproline or amino-proline residues.

286. A labeling reagent, comprising: a linker; and a plurality of dye moieties attached to linker, wherein the linker comprises a cleavable portion and a poly-hydroxyproline portion, wherein the poly-hydroxyproline portion comprises a first hydroxyproline portion comprising a first one or more hydroxyproline residues, a set of hydroxyproline or amino-proline residues that attach to the plurality of dye moieties, and one or more second hydroxyproline portions each comprising a second one or more hydroxyproline residues, each of the one or more second hydroxyproline portions disposed between different hydroxyproline or amino-proline residues of the set of hydroxyproline or amino-proline residues.

287. A method, comprising: a) providing a primer-hybridized template nucleic acid molecule; and b) contacting the primer-hybridized template nucleic acid molecule with a mixture of nucleotides, wherein the mixture of nucleotides comprises a first type of labeled nucleotide and a second type of labeled nucleotide, wherein the first type of labeled nucleotide comprises a first linker of a first length or which provides a first distance between a substrate and a label of the first type of labeled nucleotide and the second type of labeled nucleotide comprises a second linker of a second length or which provides a second distance between a substrate and a label of the second type of labeled nucleotide, the first length different from the second length and the first distance different from the second distance.

288. The method of embodiment 287, further comprising (c) detecting one or more signals from the primer-hybridized template nucleic acid molecule.

289. A method, comprising: a) providing a primer-hybridized template nucleic acid molecule; and b) contacting the primer-hybridized template nucleic acid molecule with a mixture of terminated nucleotides, wherein the mixture of terminated nucleotides comprises a first type of labeled nucleotide comprising a first number of dyes and a second type of labeled nucleotide comprising a second number of dyes, wherein the first number is different than the second number, wherein the first type of labeled nucleotide is a first canonical base and the second type of labeled nucleotide is a second canonical base different from the first canonical base, and wherein the first type of labeled nucleotide and the second type of labeled nucleotide are detectable at a same or substantially same frequency.

290. The method of embodiment 289, further comprising (c) detecting one or more signals from the primer-hybridized template nucleic acid molecule. 291. The method of any one of embodiments 289-290, wherein the mixture of terminated nucleotides further comprises a third type of labeled nucleotide of a third canonical base comprising a third number of dyes, wherein the third number is different from the first number and the second number, and wherein the third canonical base is different from the first canonical base and the second canonical base. 292. The method of embodiment 291, wherein the mixture of terminated nucleotides further comprises a fourth type of labeled nucleotide of a fourth canonical base comprising a fourth number of dyes, wherein the fourth number is different from the first number, the second number, and the third number, and wherein the fourth canonical base is different from the first canonical base, the second canonical base, and the third canonical base. 293. The method of embodiment 291, wherein the mixture of terminated nucleotides further comprises a fourth type of unlabeled nucleotide of a fourth canonical base type, wherein the fourth canonical base is different from the first canonical base, the second canonical base, and the third canonical base.

294. A method, comprising: a) providing a primer-hybridized template nucleic acid molecule; and b) contacting the primer-hybridized template nucleic acid molecule with a mixture of terminated nucleotides, wherein the mixture of terminated nucleotides comprises a first type of labeled nucleotide and a second type of labeled nucleotide, wherein the first type of labeled nucleotide is a first canonical base and the second type of labeled nucleotide is a second canonical base different from the first canonical base, wherein the first type of labeled nucleotide and the second type of labeled nucleotide are detectable at different signal intensities, and wherein the first type of labeled nucleotide and the second type of labeled nucleotide are detectable at a same or substantially same frequency. 295. The method of embodiment 294, further comprising (c) detecting one or more signals form the primer-hybridized template nucleic acid molecule.

296. A labeled substrate, comprising: a substrate; a linker; and a plurality of dye moieties attached to said substrate via said linker, wherein said linker comprises a cleavable portion and a poly-hydroxyproline portion, wherein said poly-hydroxyproline portion comprises a first hydroxyproline portion comprising a first one or more hydroxyproline residues, a set of hydroxyproline or amino-proline residues that attach to said plurality of dye moieties, and one or more second hydroxyproline portions each comprising a second one or more hydroxyproline residues, each of said one or more second hydroxyproline portions disposed between different hydroxyproline or amino-proline residues of said set of hydroxyproline or amino-proline residues.

297. The labeled substrate of embodiment 296, wherein said substrate comprises a nucleotide base. 298. The labeled substrate of embodiment 296, wherein said substrate comprises a protein. 299. The labeled substrate of any one of embodiments 296-298, wherein a second hydroxyproline portion of said one or more second hydroxyproline portions comprises at least two hydroxyproline residues. 300. The labeled substrate of any one of embodiments 296-299, wherein a second hydroxyproline portion of said one or more second hydroxyproline portions comprises at least ten hydroxyproline residues. 301. The labeled substrate of any one of embodiments 296-300, wherein each second hydroxyproline portion of said one or more second hydroxyproline portions comprises a number of hydroxyproline residue that is not 3 or an integer multiple of 3.

302. A labeling reagent, comprising: a linker; and a plurality of dye moieties attached to linker, wherein said linker comprises a cleavable portion and a poly-hydroxyproline portion, wherein said poly-hydroxyproline portion comprises a first hydroxyproline portion comprising a first one or more hydroxyproline residues, a set of hydroxyproline or amino-proline residues that attach to said plurality of dye moieties, and one or more second hydroxyproline portions each comprising a second one or more hydroxyproline residues, each of said one or more second hydroxyproline portions disposed between different hydroxyproline or amino-proline residues of said set of hydroxyproline or amino-proline residues.

303. The labeling reagent of embodiment 302, wherein a second hydroxyproline portion of said one or more second hydroxyproline portions comprises at least two hydroxyproline residues. 304. The labeling reagent of any one of embodiments 302-303, wherein a second hydroxyproline portion of said one or more second hydroxyproline portions comprises at least ten hydroxyproline residues. 305. The labeling reagent of any one of embodiments 302-304, wherein each second hydroxyproline portion of said one or more second hydroxyproline portions comprises a number of hydroxyproline residue that is not 3 or an integer multiple of 3.

306. A method, comprising: a) providing a primer-hybridized template nucleic acid molecule; and b) contacting said primer-hybridized template nucleic acid molecule with a mixture of nucleotides, wherein said mixture of nucleotides comprises a first type of labeled nucleotide and a second type of labeled nucleotide, wherein said first type of labeled nucleotide comprises a first linker of a first length or which provides a first distance between a substrate and a label of said first type of labeled nucleotide and said second type of labeled nucleotide comprises a second linker of a second length or which provides a second distance between a substrate and a label of said second type of labeled nucleotide, and wherein said first length is different from said second length and said first distance is different from said second distance.

307. The method of embodiment 306, further comprising (c) detecting one or more signals from said primer-hybridized template nucleic acid molecule. 308. The method of any one of embodiments 306-307, wherein said first type of labeled nucleotide and said second type of labeled nucleotide are of a same canonical base type.

309. A method, comprising: a) providing a primer-hybridized template nucleic acid molecule; and b) contacting said primer-hybridized template nucleic acid molecule with a mixture of terminated nucleotides, wherein said mixture of terminated nucleotides comprises a first type of labeled nucleotide comprising a first number of dyes and a second type of labeled nucleotide comprising a second number of dyes, wherein said first number is different than said second number, wherein said first type of labeled nucleotide is a first canonical base and said second type of labeled nucleotide is a second canonical base different from said first canonical base, and wherein said first type of labeled nucleotide and said second type of labeled nucleotide are detectable at a same or substantially same frequency.

310. The method of embodiment 309, wherein said first type of labeled nucleotide comprises said labeled substrate of embodiment 296, wherein said substrate is a terminated nucleotide. 311. The method of any one of embodiments 309-310, further comprising (c) detecting one or more signals from said primer-hybridized template nucleic acid molecule. 312. The method of any one of embodiments 309-311, wherein said mixture of terminated nucleotides further comprises a third type of labeled nucleotide of a third canonical base comprising a third number of dyes, wherein said third number is different from said first number and said second number, and wherein said third canonical base is different from said first canonical base and said second canonical base. 313. The method of embodiment 312, wherein said mixture of terminated nucleotides further comprises a fourth type of labeled nucleotide of a fourth canonical base comprising a fourth number of dyes, wherein said fourth number is different from said first number, said second number, and said third number, and wherein said fourth canonical base is different from said first canonical base, said second canonical base, and said third canonical base. 314. The method of embodiment 312, wherein said mixture of terminated nucleotides further comprises a fourth type of unlabeled nucleotide of a fourth canonical base type, wherein said fourth canonical base is different from said first canonical base, said second canonical base, and said third canonical base.

315. A method, comprising: a) providing a primer-hybridized template nucleic acid molecule; and b) contacting said primer-hybridized template nucleic acid molecule with a mixture of terminated nucleotides, wherein said mixture of terminated nucleotides comprises a first type of labeled nucleotide and a second type of labeled nucleotide, wherein said first type of labeled nucleotide is a first canonical base and said second type of labeled nucleotide is a second canonical base different from said first canonical base, wherein said first type of labeled nucleotide and said second type of labeled nucleotide are detectable at different signal intensities, and wherein said first type of labeled nucleotide and said second type of labeled nucleotide are detectable at a same or substantially same frequency.

316. The method of embodiment 315, wherein said first type of labeled nucleotide comprises said labeled substrate of embodiment 296, wherein said substrate is a terminated nucleotide. 317. The method of any one of embodiments 315-316, further comprising (c) detecting one or more signals form said primer-hybridized template nucleic acid molecule.

318. A labeling reagent comprising: (a) a detectable moiety; and (b) a linker that is coupled to said detectable moiety, wherein said linker comprises at least one cleavable group and at least one non-proteinogenic amino acid, wherein said at least one non-proteinogenic amino acid does not comprise hydroxyproline, and wherein when said at least one non-proteinogenic amino acid comprises 6-aminohexanoic acid, said detectable moiety is not a Cyanide 5 (Cy5) or an ATTO 647 NHS Ester (ATTO 647N).

319. A labeling reagent comprising a compound of Formula I:

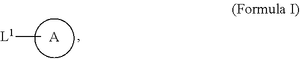

wherein: A is a detectable moiety; and L¹ is a linker comprising at least one cleavable group and at least one non-proteinogenic amino acid, wherein said non-proteinogenic amino acid does not comprise hydroxyproline, and wherein when said at least one non-proteinogenic amino acid comprises 6-aminohexanoic acid, said detectable moiety is not a Cyanide 5 (Cy5) or an ATTO 647 NHS Ester (ATTO 647N).

320. A labeling reagent comprising: (a) a detectable moiety; and (b) a linker that is coupled to said detectable moiety, wherein said linker comprises at least one cleavable group and at least one non-proteinogenic amino acid, wherein said at least one non-proteinogenic amino acid does not comprise hydroxyproline, and wherein when said at least one non-proteinogenic amino acid comprises 6-aminohexanoic acid, said linker is not coupled to

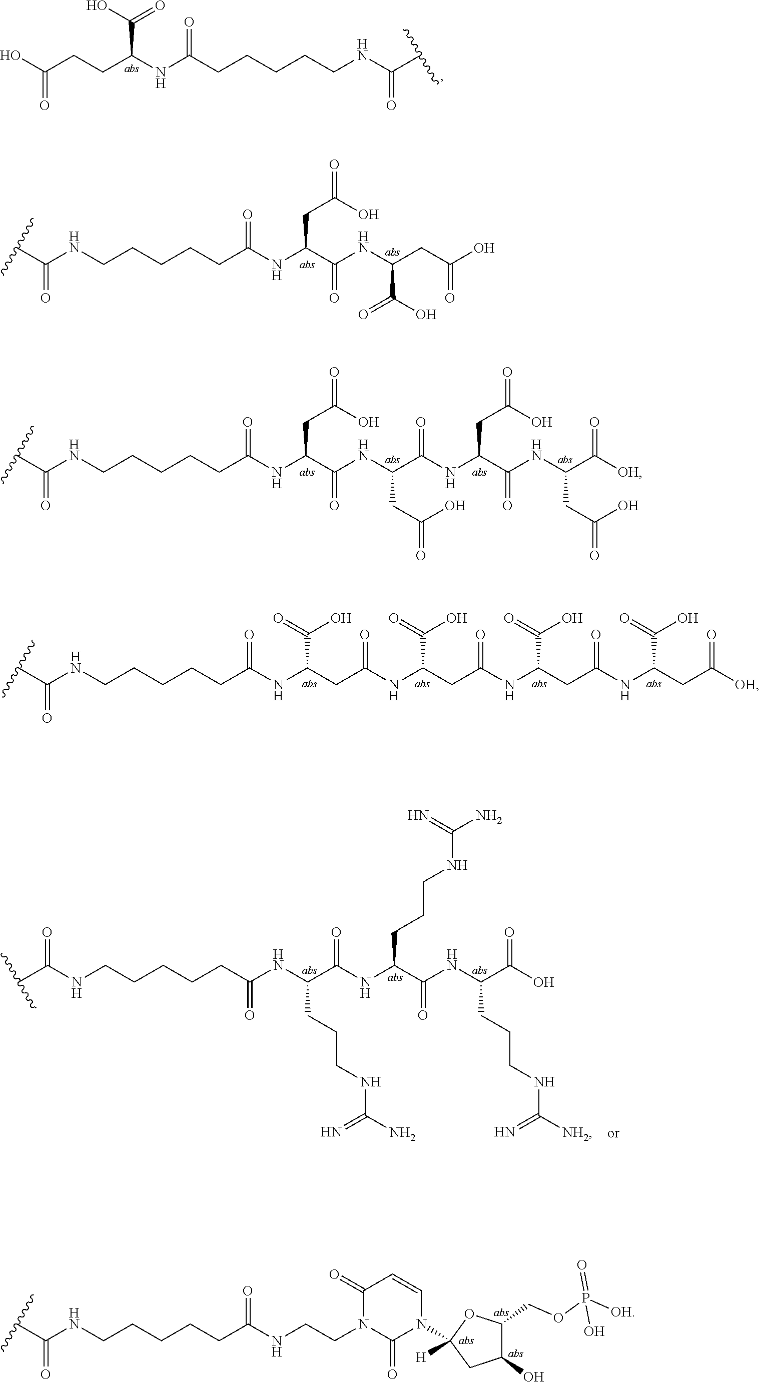 [SMILES: *C(=O)NCCCCCC(=O)N[C@@H](CCC(=O)O)C(=O)O ; *C(=O)NCCCCCC(=O)N[C@@H](CC(=O)O)C(=O)N[C@@H](CC(=O)O)C(=O)O ; *C(=O)NCCCCCC(=O)N[C@@H](CC(=O)O)C(=O)N[C@@H](CC(=O)O)C(=O)N[C@@H](CC(=O)O)C(=O)N[C@@H](CC(=O)O)C(=O)O ; *C(=O)NCCCCCC(=O)N[C@@H](CC(=O)N[C@@H](CC(=O)N[C@@H](CC(=O)N[C@@H](CC(=O)O)C(=O)O)C(=O)O)C(=O)O)C(=O)O ; *C(=O)NCCCCCC(=O)N[C@@H](CCCNC(=N)N)C(=O)N[C@@H](CCCNC(=N)N)C(=O)N[C@@H](CCCNC(=N)N)C(=O)O ; *C(=O)NCCCCCC(=O)NCCn1c(=O)ccn([C@H]2C[C@H](O)[C@@H](COP(=O)(O)O)O2)c1=O]

321. A labeling reagent comprising a compound of Formula I:

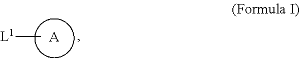

wherein: A is a detectable moiety; and L¹ is a linker comprising at least one cleavable group and at least one non-proteinogenic amino acid, wherein said at least one non-proteinogenic amino acid does not comprise hydroxyproline, and wherein when said at least one non-proteinogenic amino acid comprises 6-aminohexanoic acid, said linker is not coupled to

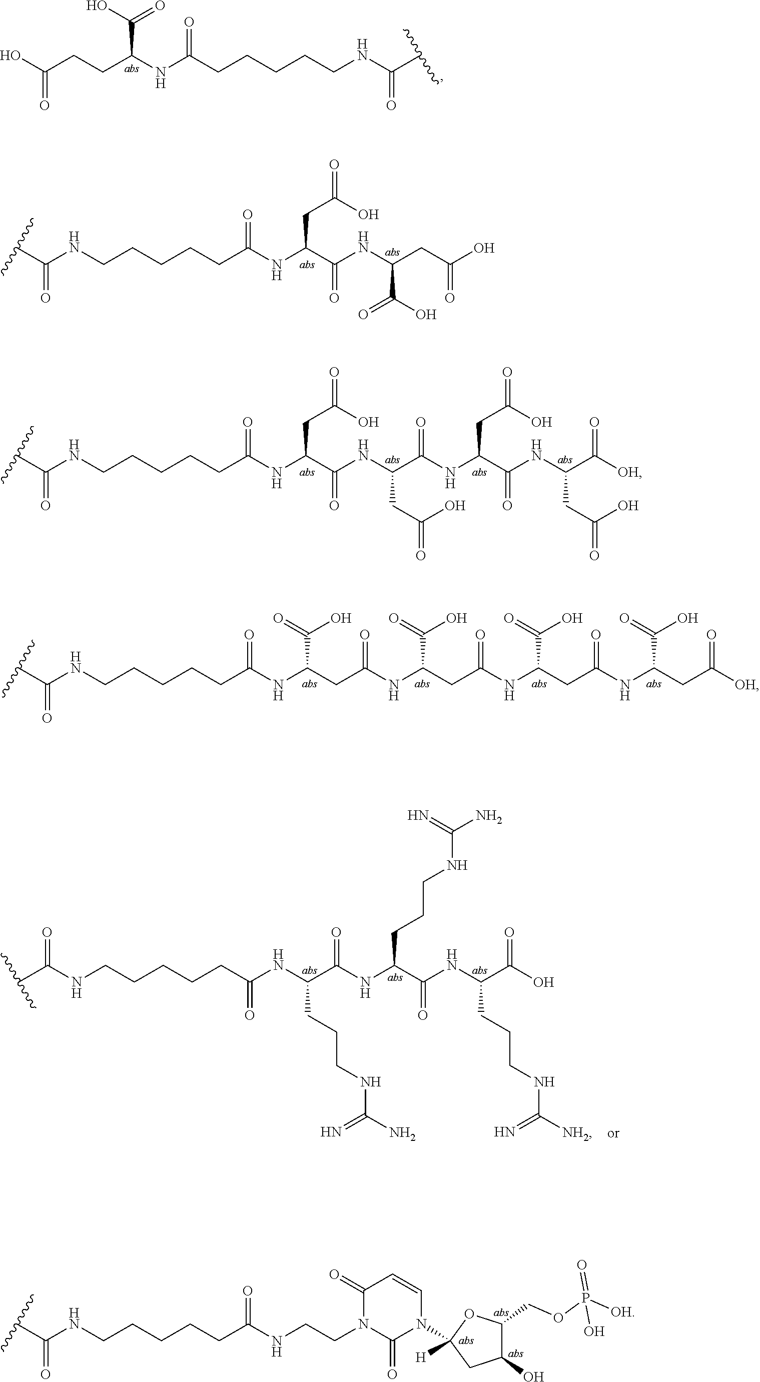 [SMILES: *C(=O)NCCCCCC(=O)N[C@@H](CCC(=O)O)C(=O)O ; *C(=O)NCCCCCC(=O)N[C@@H](CC(=O)O)C(=O)N[C@@H](CC(=O)O)C(=O)O ; *C(=O)NCCCCCC(=O)N[C@@H](CC(=O)O)C(=O)N[C@@H](CC(=O)O)C(=O)N[C@@H](CC(=O)O)C(=O)N[C@@H](CC(=O)O)C(=O)O ; *C(=O)NCCCCCC(=O)N[C@@H](CC(=O)N[C@@H](CC(=O)N[C@@H](CC(=O)N[C@@H](CC(=O)O)C(=O)O)C(=O)O)C(=O)O)C(=O)O ; *C(=O)NCCCCCC(=O)N[C@@H](CCCNC(=N)N)C(=O)N[C@@H](CCCNC(=N)N)C(=O)N[C@@H](CCCNC(=N)N)C(=O)O ; *C(=O)NCCCCCC(=O)NCCn1c(=O)ccn([C@H]2C[C@H](O)[C@@H](COP(=O)(O)O)O2)c1=O]

322. A labeling reagent comprising: (a) a detectable moiety; and (b) a linker that is coupled to said detectable moiety, wherein said linker comprises at least one cleavable group and at least one non-proteinogenic amino acid, wherein said at least one non-proteinogenic amino acid comprises cysteic acid, 5-amino-5-carboxy-N,N,N-trimethylpentan-1-aminium or a salt thereof, or 6-aminohexanoic acid, or a combination thereof, wherein when said at least one non-proteinogenic amino acid comprises said cysteic acid, said linker does not comprise hydroxyproline, and wherein when said at least one non-proteinogenic amino acid comprises said 6-aminohexanoic acid, said detectable moiety is not a Cyanide 5 (Cy5) or an ATTO 647 NHS Ester (ATTO 647N).

323. A labeling reagent comprising a compound of Formula I:

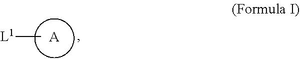

wherein: A is a detectable moiety; and L¹ is a linker comprising at least one cleavable group and at least one non-proteinogenic amino acid, wherein said at least one non-proteinogenic amino acid non-proteinogenic amino acid comprises cysteic acid, 5-amino-5-carboxy-N,N,N-trimethylpentan-1-aminium or a salt thereof, or 6-aminohexanoic acid, or a combination thereof, wherein when said at least one non-proteinogenic amino acid comprises said cysteic acid, L¹ does not comprise hydroxyproline, and wherein when said at least one non-proteinogenic amino acid comprises said 6-aminohexanoic acid, said detectable moiety is not a Cyanide 5 (Cy5) or an ATTO 647 NHS Ester (ATTO 647N).

324. A labeling reagent comprising: (a) a detectable moiety; and (b) a linker that is coupled to said detectable moiety, wherein said linker comprises at least one cleavable group and at least one non-proteinogenic amino acid, wherein said at least one non-proteinogenic amino acid comprises cysteic acid, 5-amino-5-carboxy-N,N,N-trimethylpentan-1-aminium or a salt thereof, or 6-aminohexanoic acid, or a combination thereof, wherein when said at least one non-proteinogenic amino acid comprises said cysteic acid, said linker does not comprise hydroxyproline, and wherein when said at least one non-proteinogenic amino acid comprises said 6-aminohexanoic acid, said linker is not coupled to

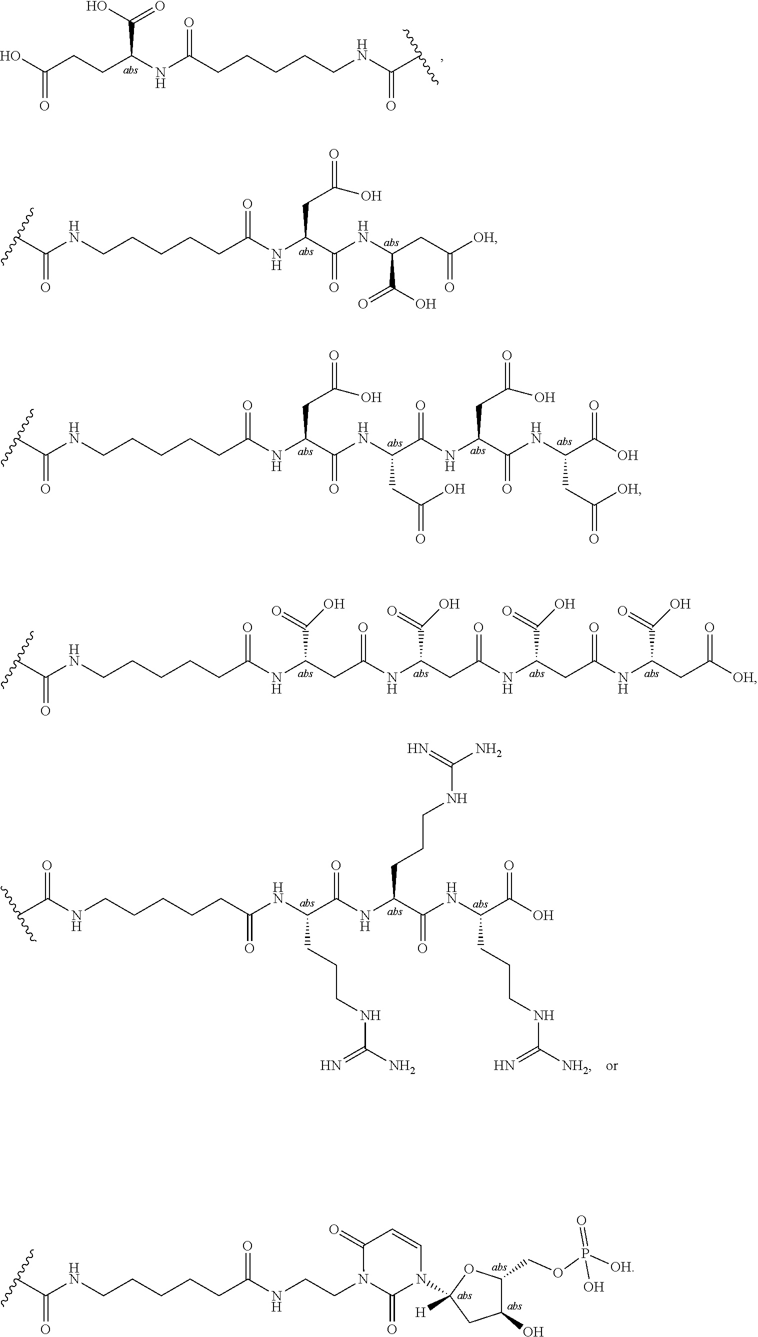 [SMILES: *C(=O)NCCCCCC(=O)N[C@@H](CCC(=O)O)C(=O)O ; *C(=O)NCCCCCC(=O)N[C@@H](CC(=O)O)C(=O)N[C@@H](CC(=O)O)C(=O)O ; *C(=O)NCCCCCC(=O)N[C@@H](CC(=O)O)C(=O)N[C@@H](CC(=O)O)C(=O)N[C@@H](CC(=O)O)C(=O)N[C@@H](CC(=O)O)C(=O)O ; *C(=O)NCCCCCC(=O)N[C@@H](CC(=O)N[C@@H](CC(=O)N[C@@H](CC(=O)N[C@@H](CC(=O)O)C(=O)O)C(=O)O)C(=O)O)C(=O)O ; *C(=O)NCCCCCC(=O)N[C@@H](CCCNC(=N)N)C(=O)N[C@@H](CCCNC(=N)N)C(=O)N[C@@H](CCCNC(=N)N)C(=O)O ; *C(=O)NCCCCCC(=O)NCCn1c(=O)ccn([C@H]2C[C@H](O)[C@@H](COP(=O)(O)O)O2)c1=O]

325. A labeling reagent comprising a compound of Formula I:

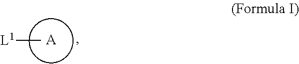

wherein: A is a detectable moiety; and L¹ is a linker comprising at least one cleavable group and at least one non-proteinogenic amino acid, wherein said at least one non-proteinogenic amino acid comprises cysteic acid, 5-amino-5-carboxy-N,N,N-trimethylpentan-1-aminium, or 6-aminohexanoic acid, or a combination thereof, wherein when said at least one non-proteinogenic amino acid comprises said cysteic acid, L¹ does not comprise hydroxyproline, and wherein when said at least one non-proteinogenic acid comprises said 6-aminohexanoic acid, L¹ is not coupled to

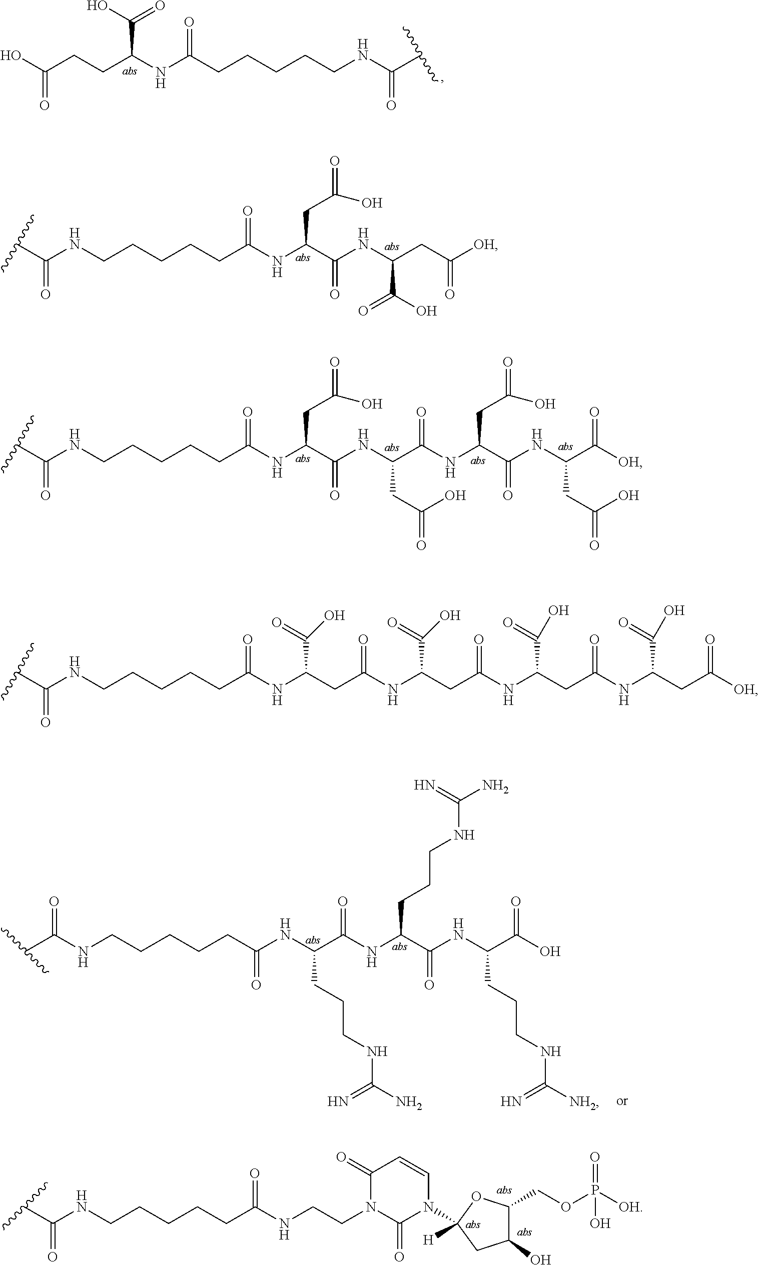 [SMILES: *C(=O)NCCCCCC(=O)N[C@@H](CCC(=O)O)C(=O)O ; *C(=O)NCCCCCC(=O)N[C@@H](CC(=O)O)C(=O)N[C@@H](CC(=O)O)C(=O)O ; *C(=O)NCCCCCC(=O)N[C@@H](CC(=O)O)C(=O)N[C@@H](CC(=O)O)C(=O)N[C@@H](CC(=O)O)C(=O)N[C@@H](CC(=O)O)C(=O)O ; *C(=O)NCCCCCC(=O)N[C@@H](CC(=O)N[C@@H](CC(=O)N[C@@H](CC(=O)N[C@@H](CC(=O)O)C(=O)O)C(=O)O)C(=O)O)C(=O)O ; *C(=O)NCCCCCC(=O)N[C@@H](CCCNC(=N)N)C(=O)N[C@@H](CCCNC(=N)N)C(=O)N[C@@H](CCCNC(=N)N)C(=O)O ; *C(=O)NCCCCCC(=O)NCCn1c(=O)ccn([C@H]2C[C@H](O)[C@@H](COP(=O)(O)O)O2)c1=O]

326. The labeling reagent of any one of embodiments 318-325, wherein said linker is not coupled to a terminator group. 327. The labeling reagent of any one of embodiments 318-326, wherein said detectable moiety does not comprise said Cy5 or said ATTO 647N. 328. The labeling reagent of any one of embodiments 318-327, wherein said at least one non-proteinogenic amino acid comprises at most about 50 atoms. 329. The labeling reagent of any one of embodiments 318-328, wherein said at least one non-proteinogenic amino acid comprises at most about 20 atoms. 330. The labeling reagent of any one of embodiments 318-329, wherein said at least one non-proteinogenic amino acid comprises about 10-20 atoms. 331. The labeling reagent of any one of embodiments 318-330, wherein said at least one non-proteinogenic amino acid comprises cysteic acid. 332. The labeling reagent of any one of embodiments 318-331, wherein said at least one non-proteinogenic amino acid comprises 5-amino-5-carboxy-N,N,N-trimethylpentan-1-aminium or a salt thereof 333. The labeling reagent of any one of embodiments 318-332, wherein said at least one non-proteinogenic amino acid comprises 6-aminohexanoic acid. 334. The labeling reagent of any one of embodiments 318-333, wherein said at least one non-proteinogenic amino acid comprises a quaternary amine. 335. The labeling reagent of any one of embodiments 318-334, wherein said detectable moiety comprises a fluorescent dye. 336. The labeling reagent of embodiment 335, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, or Kam. 337. The labeling reagent of embodiment 336, wherein said fluorescent dye comprises ATTO 633. 338. The labeling reagent of any one of embodiments 318-337, wherein said at least one cleavable group is configured to be cleaved to separate a portion of said detectable moiety from said labeling reagent. 339. The labeling reagent of any one of embodiments 318-338, wherein said at least one cleavable group is selected from said group consisting of an azidomethyl group, a disulfide bond, a hydrocarbyldithiomethyl group, and a 2-nitrobenzyloxy group. 340. The labeling reagent of embodiment 339, wherein said at least one cleavable group is said disulfide bond. 341. The labeling reagent of any one of embodiments 318-340, wherein said at least one cleavable group is cleavable by application of one or more members of said group consisting of tris(2-carboxyethyl)phosphine (TCEP), dithiothreitol (DTT), tetrahydropyranyl (THP), ultraviolet (UV) light, and a combination thereof 342. The labeling reagent of any one of embodiments 318-341, wherein said labeling reagent comprises a moiety selected from said group consisting of

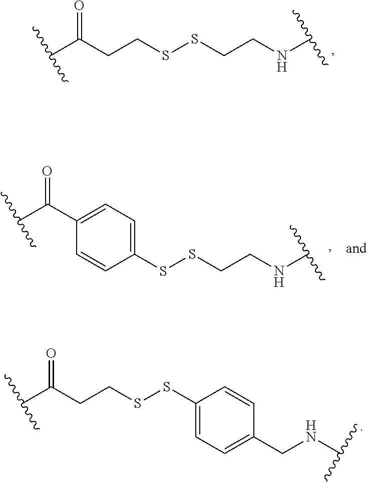 [SMILES: *NCCSSCCC(*)=O ; *NCCSSc1ccc(C(*)=O)cc1 ; *NCc1ccc(SSCCC(*)=O)cc1]

343. A detectably labeled substrate comprising a compound of any one of embodiments 318-325, wherein said compound is a compound of Formula Ia:

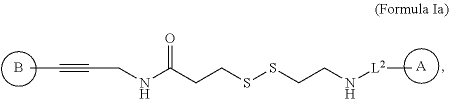

wherein: B is a substrate, A is said detectable moiety, and L² comprises said at least one non-proteinogenic amino acid.

344. A detectably labeled substrate comprising: (a) a detectable moiety; (b) a linker that is coupled to said detectable moiety, wherein said linker comprises at least one non-proteinogenic amino acid, wherein said at least one non-proteinogenic amino acid comprises cysteic acid, 5-amino-5-carboxy-N,N,N-trimethylpentan-1-aminium or a salt thereof, or 6-aminohexanoic acid, or a combination thereof, and wherein when said at least one non-proteinogenic amino acid comprises said 6-aminohexanoic acid, said detectable moiety is not a Cyanide 5 (Cy5) or an ATTO 647 NHS Ester (ATTO 647N); and (c) a substrate comprising a nucleobase, wherein said substrate is coupled to said linker, and wherein said nucleobase does not comprise guanine.

345. A detectably labeled substrate comprising a compound of Formula II:

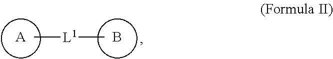

wherein: A comprises a nucleobase, wherein said nucleobase is not guanine; B is a detectable moiety; and L¹ is a linker comprising at least one non-proteinogenic amino acid, wherein said at least one non-proteinogenic amino acid comprises cysteic acid, 5-amino-5-carboxy-N,N,N-trimethylpentan-1-aminium or a salt thereof, or 6-aminohexanoic acid, or a combination thereof, and wherein when said at least one non-proteinogenic amino acid comprises said 6-aminohexanoic acid, said detectable moiety is not a Cyanide 5 (Cy5) or an ATTO 647 NHS Ester (ATTO 647N).

346. The detectably labeled substrate of any one of embodiments 344-345, wherein said nucleobase is adenine, cytosine, thymine, or uracil. 347. The detectably labeled substrate of any one of embodiments 344-346, wherein said detectable moiety does not comprise said Cy5 or said ATTO 647N. 348. The detectably labeled substrate of any one of embodiments 344-347, wherein said linker comprises at least one cleavable group. 349. The detectably labeled substrate of any one of embodiments 344-348, wherein said at least one non-proteinogenic amino acid comprises said cysteic acid. 350. The detectably labeled substrate of any one of embodiments 344-349, wherein said at least one non-proteinogenic amino acid comprises said 5-amino-5-carboxy-N,N,N-trimethylpentan-1-aminium. 351. The detectably labeled substrate of any one of embodiments 344-350, wherein said at least one non-proteinogenic amino acid comprises said 6-aminohexanoic acid. 352. The detectably labeled substrate of any one of embodiments 344-351, wherein said at least one non-proteinogenic amino acid comprises a quaternary amine. 353. The detectably labeled substrate of any one of embodiments 344-352, wherein said detectable moiety comprises a fluorescent dye. 354. The detectably labeled substrate of embodiment 353, wherein said fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, or Kam. 355. The detectably labeled substrate of embodiment 354, wherein said fluorescent dye comprises ATTO 633. 356. The detectably labeled substrate of any one of embodiments 344-355, wherein said at least one cleavable group is configured to be cleaved to separate a portion of said detectable moiety from said detectably labeled substrate. 357. The detectably labeled substrate of any one of embodiments 344-356, wherein said at least one cleavable group is selected from said group consisting of an azidomethyl group, a disulfide bond, a hydrocarbyldithiomethyl group, and a 2-nitrobenzyloxy group. 358. The detectably labeled substrate of embodiment 357, wherein said at least one cleavable group is said disulfide bond. 359. The detectably labeled substrate of any one of embodiments 344-358, wherein said at least one cleavable group is cleavable by application of one or more members of said group consisting of tris(2-carboxyethyl)phosphine (TCEP), dithiothreitol (DTT), tetrahydropyranyl (THP), ultraviolet (UV) light, and a combination thereof. 360. The detectably labeled substrate of any one of embodiments 344-359, wherein said detectably labeled substrate comprises a moiety selected from said group consisting of

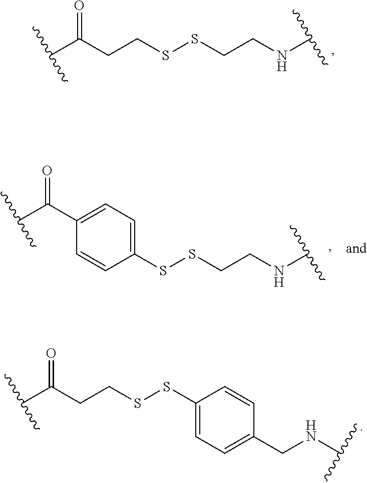 [SMILES: *NCCSSCCC(*)=O ; *NCCSSc1ccc(C(*)=O)cc1 ; *NCc1ccc(SSCCC(*)=O)cc1]

361. The detectably labeled substrate of any one of embodiments 344-360, wherein said detectably labeled substrate comprises a compound of Formula IIa:

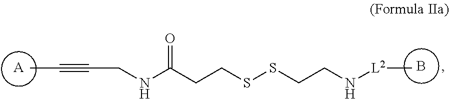

wherein: A is a deoxyribose nucleotide triphosphate; B is a detectable moiety; and L² comprises said at least one non-proteinogenic amino acid. 362. The detectably labeled substrate of embodiment 361, wherein said detectably labeled substrate is a compound of Formula IIb, Formula IIc, Formula IId, Formula IIe, Formula IIf, or Formula IIg:

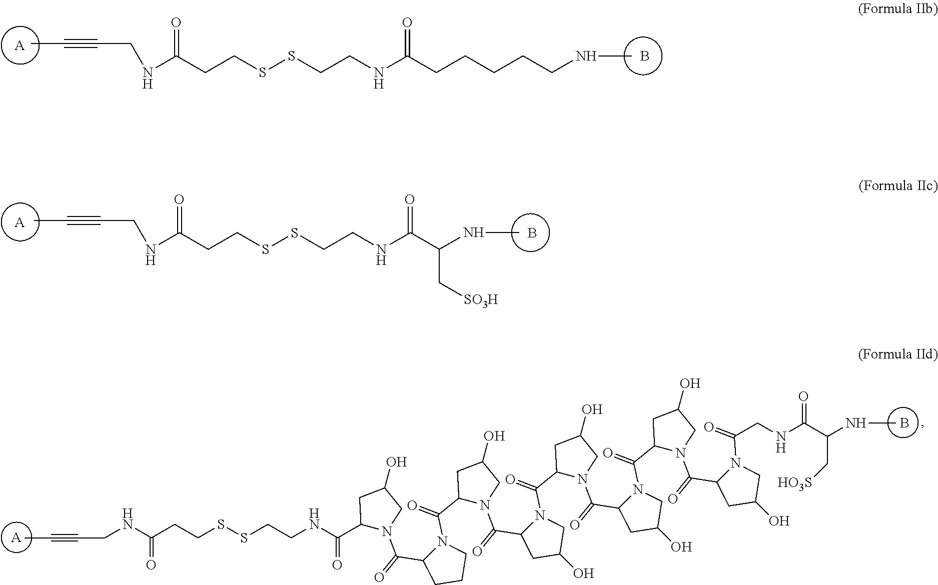

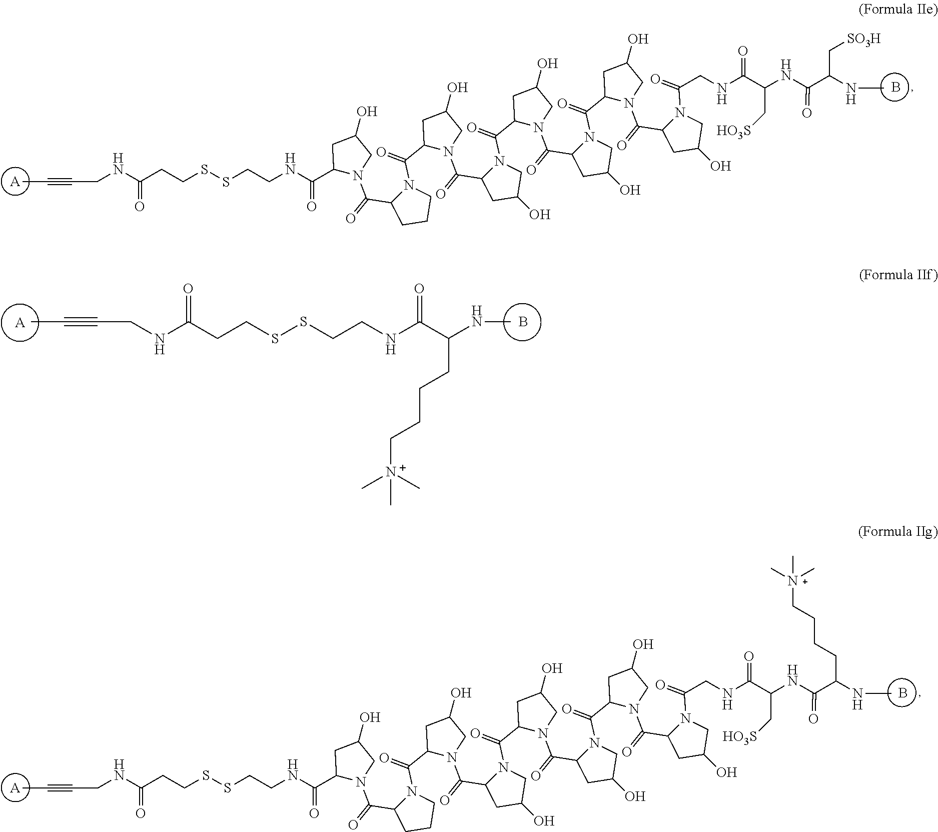

363. A substrate comprising: (a) a nucleobase, wherein said nucleobase is not a guanine; and (b) a linker coupled to said nucleobase, wherein said linker comprises at least a first non-proteinogenic amino acid and at least a second non-proteinogenic amino acid, wherein said first non-proteinogenic amino acid and said second non-proteinogenic amino acid are different.

364. A substrate comprising a compound of Formula III:

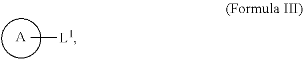

wherein: A comprises a nucleobase; and L¹ is a linker comprising at least a first non-proteinogenic amino acid and at least a second non-proteinogenic amino acid, wherein said nucleobase is not a guanine, and wherein said first non-proteinogenic amino acid and said second non-proteinogenic amino acid are different.

365. The substrate of any one of embodiments 363-364, wherein said first non-proteinogenic amino acid comprises hydroxyproline. 366. The substrate of embodiment 365, wherein said first non-proteinogenic amino acid comprises at least about 10 hydroxyprolines. 367. The substrate of any one of embodiments 363-366, wherein said second non-proteinogenic amino acid comprises cysteic acid, 5-amino-5-carboxy-N,N,N-trimethylpentan-1-aminium or a salt thereof, a quaternary amine, or 6-aminohexanoic acid, or a combination thereof 368. The substrate of any one of embodiments 363-367, wherein said nucleobase comprises adenine, cytosine, thymine, or uracil. 369. The substrate of any one of embodiments 363-368, wherein said linker comprises at least one cleavable group. 370. The substrate of any one of embodiments 363-369, wherein said detectable moiety comprises at least one fluorescent dye. 371. The substrate of any one of embodiments 363-370, wherein said substrate comprises a moiety selected from said group consisting of

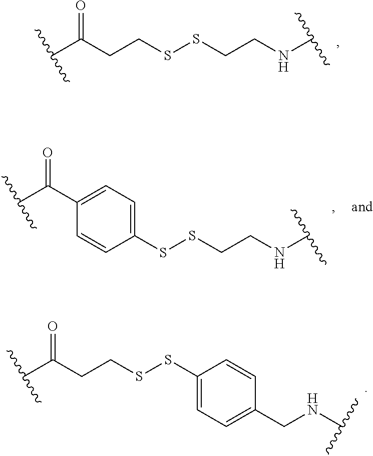 [SMILES: *NCCSSCCC(*)=O ; *NCCSSc1ccc(C(*)=O)cc1 ; *NCc1ccc(SSCCC(*)=O)cc1]

372. A detectably labeled substrate comprising said substrate of any one of embodiments 363-371, wherein said detectably labeled substrate comprises a compound of Formula IIIa:

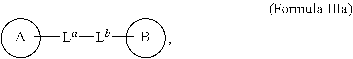

wherein: A comprises said nucleobase; B comprises a detectable moiety; L^{a} is a first linker; and L^{b} is a second linker. 373. The detectably labeled substrate of embodiment 372, wherein L^{b} comprises said first non-proteinogenic amino acid or said second non-proteinogenic amino acid. 374. The detectably labeled substrate of embodiment 373, wherein L^{b} comprises said first non-proteinogenic amino acid and said second non-proteinogenic amino acid. 375. The detectably labeled substrate of any one of claims 372-374, wherein L^{a} comprises at least one cleavable group. 376. The detectably labeled substrate of any one of claims 372-375, wherein said detectably labeled substrate comprises a compound of Formula IIIb or a compound of Formula IIIc:

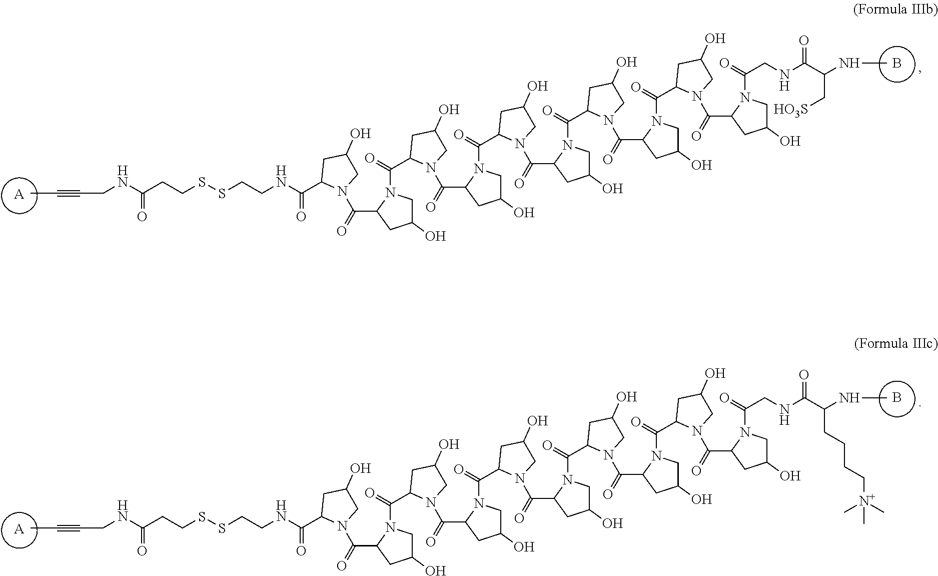

377. A substrate comprising: (a) a nucleobase wherein said nucleobase is not a guanine; and (b) a linker coupled to said nucleobase, wherein said linker comprises at least two non-proteinogenic amino acids, wherein said at least two non-proteinogenic amino acids are a same type.

378. A substrate comprising a compound of Formula IV:

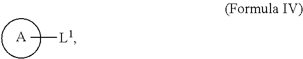

wherein: A comprises a nucleobase, wherein said nucleobase is not a guanine; and L¹ is a linker comprising at least two non-proteinogenic amino acids, wherein said at least two non-proteinogenic amino acids are a same type.

379. The substrate of any one of embodiments 377-378, wherein said at least two non-proteinogenic amino acids are cysteic acids. 380. The substrate of any one of embodiments 377-378, wherein said at least two non-proteinogenic amino acids are 5-amino-5-carboxy-N,N,N-trimethylpentan-1-aminiums. 381. The substrate of any one of embodiments 377-380, further comprising a third non-proteinogenic amino acid different from said at least two non-proteinogenic amino acids. 382. The substrate of embodiment 381, wherein said third non-proteinogenic amino acid comprises hydroxyproline. 383. The substrate of any one of embodiments 377-382, wherein said nucleobase comprises adenine, cytosine, thymine, or uracil. 384. The substrate of any one of embodiments 377-383, wherein said linker comprises at least one cleavable group. 385. The substrate of any one of embodiments 377-384, wherein said detectable moiety comprises a fluorescent dye. 386. The substrate of any one of embodiments 377-385, wherein said substrate comprises a moiety selected from said group consisting of

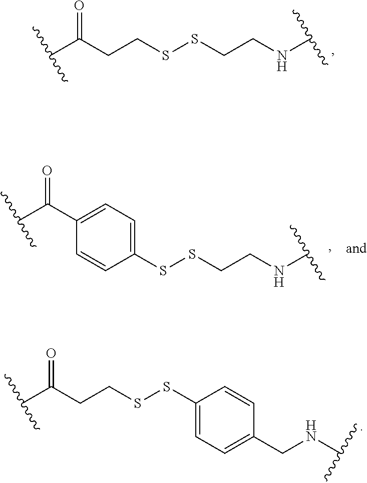 [SMILES: *NCCSSCCC(*)=O ; *NCCSSc1ccc(C(*)=O)cc1 ; *NCc1ccc(SSCCC(*)=O)cc1]

387. A detectably labeled substrate comprising said substrate of any one of embodiments 377-386, wherein said detectably labeled substrate is a compound of Formula IVa:

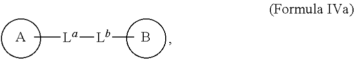

wherein: A comprises said nucleobase, wherein said nucleobase is not a guanine; B comprises a detectable moiety; L^{a} is a first linker; and L^{b} is a second linker. 388. The detectably labeled substrate of embodiment 387, wherein L^{b} comprises said at least two non-proteinogenic amino acids. 399. The detectably labeled substrate of any one of embodiments 387-388, wherein L^{b} comprises a third non-proteinogenic amino acid. 400. The detectably labeled substrate of any one of embodiments 387-389, wherein said detectably labeled substrate is a compound of Formula IVc or a compound of Formula IVd

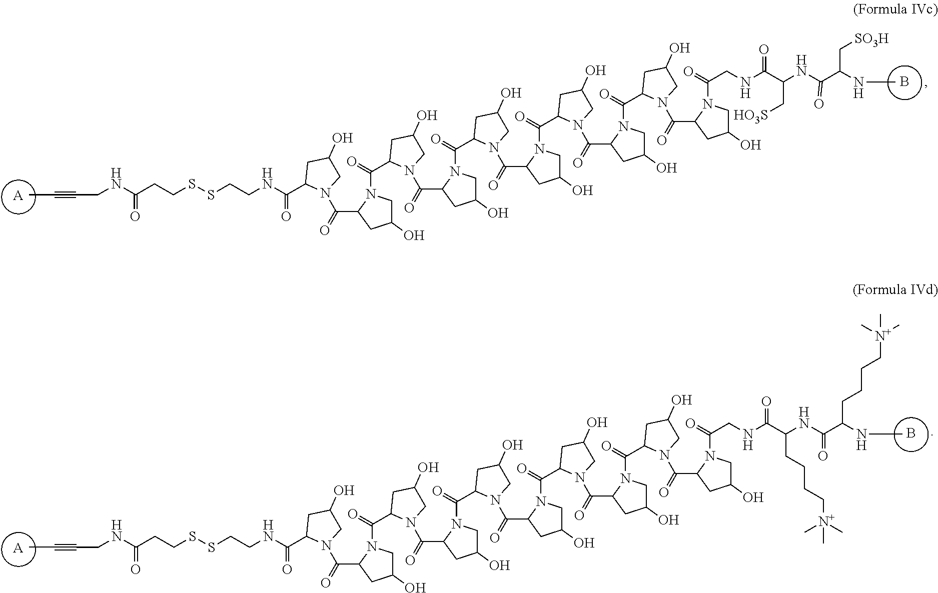

401. A composition comprising a solution comprising a plurality of said labeled substrate, labeling reagent, and/or detectably labeled substrate of any one of claims 296-390. 402. The composition of embodiment 391, wherein said solution further comprises a plurality of unlabeled substrates, wherein each substrate of said plurality of unlabeled substrates is of a same type as each said labeled substrate, labeling reagent, and/or detectably labeled substrate. 403. The composition of embodiment 392, wherein a ratio of said plurality of said labeled substrate, labeling reagent, and/or detectably labeled substrate to said plurality of unlabeled substrates in said solution is at least about 10:1. 404. The composition of embodiment 393, wherein said ratio is at least about 5:1. 405 The composition of embodiment 394, wherein said ratio is at least about 3:1.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-100. (canceled)

101. A labeled substrate, comprising:
a substrate;
a linker; and
a plurality of dye moieties attached to the substrate via the linker,
wherein the linker comprises a cleavable portion and a poly-hydroxyproline portion,
wherein the poly-hydroxyproline portion comprises a first hydroxyproline portion comprising a first one or more hydroxyproline residues, a set of hydroxyproline or amino-proline residues that attach to the plurality of dye moieties, and one or more second hydroxyproline portions each comprising a second one or more hydroxyproline residues, each of the one or more second hydroxyproline portions disposed between different hydroxyproline or amino-proline residues of the set of hydroxyproline or amino-proline residues.

102. The labeled substrate of claim 101, wherein the substrate comprises a nucleotide base.

103. The labeled substrate of claim 101, wherein the substrate comprises a protein.

104. The labeled substrate of claim 101, wherein a second hydroxyproline portion of the one or more second hydroxyproline portions comprises at least two hydroxyproline residues.

105. The labeled substrate of claim 101, wherein a second hydroxyproline portion of the one or more second hydroxyproline portions comprises at least ten hydroxyproline residues.

106. The labeled substrate of claim 101, wherein each of the one or more second hydroxyproline portions comprises a number of hydroxyproline residue that is not 3 or an integer multiple of 3.

107. The labeled substrate of claim 101, wherein the plurality of dye moieties comprises a fluorescent dye.

108. The labeled substrate of claim 107, wherein the fluorescent dye comprises ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, UG 524.2, UG 524.1, UG_dye105, UG_dye106, KK1119, KK9046, Abberior STAR635, Abberior STAR635P, or Kam.

109. The labeled substrate of claim 101, wherein the cleavable portion comprises at least one cleavable group that is configured to be cleaved to separate the plurality of dye moieties from the substrate.

110. The labeled substrate of claim 109, wherein the at least one cleavable group is selected from the group consisting of an azidomethyl group, a disulfide bond, a hydrocarbyldithiomethyl group, and a 2-nitrobenzyloxy group.

111. The labeled substrate of claim 110, wherein the at least one cleavable group is the disulfide bond.

112. The labeled substrate of claim 110, wherein the at least one cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof.

113. The labeled substrate of claim 101, wherein the linker comprises a moiety selected from the group consisting of

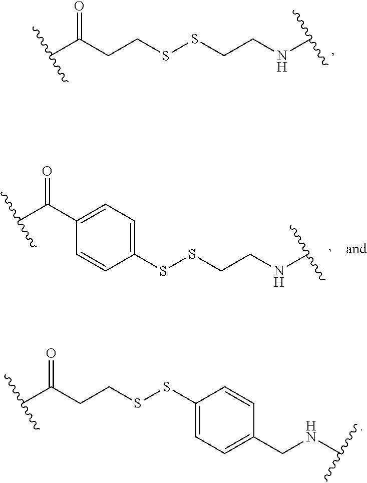 [SMILES: *NCCSSCCC(*)=O ; *NCCSSc1ccc(C(*)=O)cc1 ; *NCc1ccc(SSCCC(*)=O)cc1]

114. The labeled substrate of claim 101, wherein the set of hydroxyproline or amino-proline residues comprises an aminoproline residue that is attached to a dye moiety of the plurality of dye moieties.

115. The labeled substrate of claim 101, wherein the set of hydroxyproline or amino-proline residues comprises a hydroxyproline residue that is attached to a dye moiety of the plurality of dye moieties.

116. The labeled substrate of claim 101, wherein the plurality of dye moieties comprises at least 2 dye moieties.

117. The labeled substrate of claim 101, wherein the plurality of dye moieties comprises at least 3 dye moieties.

118. A labeling reagent, comprising:
a linker; and
a plurality of dye moieties attached to linker,
wherein the linker comprises a cleavable portion and a poly-hydroxyproline portion,
wherein the poly-hydroxyproline portion comprises a first hydroxyproline portion comprising a first one or more hydroxyproline residues, a set of hydroxyproline or amino-proline residues that attach to the plurality of dye moieties, and one or more second hydroxyproline portions each comprising a second one or more hydroxyproline residues, each of the one or more second hydroxyproline portions disposed between different hydroxyproline or amino-proline residues of the set of hydroxyproline or amino-proline residues.

119. The labeling reagent of claim 118, wherein a second hydroxyproline portion of the one or more second hydroxyproline portions comprises at least two hydroxyproline residues.

120. The labeling reagent of claim 118, wherein each of the one or more second hydroxyproline portions comprises a number of hydroxyproline residue that is not 3 or an integer multiple of 3.
